(19) United States
(12) Patent Application Publication
(10) Pub. No.: US 2023/0049887 A1
(43) Pub. Date: Feb. 16, 2023

(54) COMPOSITIONS AND METHODS FOR PRODUCING FOOD PRODUCTS WITH RECOMBINANT ANIMAL PROTEIN

(71) Applicant: Bond Pet Foods, Inc., Boulder, CO (US)

(72) Inventors: Karin Pernilla Turner Audibert, Golden, CO (US); Richard W. Kelleman, II, Denver, CO (US); Anthony George Day, San Francisco, CA (US); Julie Marie Struble, Bothell, WA (US); Ryan Michael Yamka, Trumbull, CT (US); Catherine Asleson Dundon, Englewood, CO (US); Luis N. Brandao, Aurora, CO (US)

(21) Appl. No.: 17/427,046

(22) PCT Filed: Jan. 29, 2020
(86) PCT No.: PCT/US20/15734

§ 371 Date: Jul. 29, 2021

Related U.S. Application Data
(60) Provisional application No. 62/798,447, filed on Jan. 29, 2019.

(51) Int. Cl. A23J 3/04; C07K 14/47; A23L 13/00; C12N 15/85; A23K 10/20

(52) U.S. Cl. CPC A23J 3/04; C07K 14/47; A23L 13/00; C12N 15/85; A23K 10/20

(57) ABSTRACT

The invention relates to methods and compositions for the recombinant production of animal protein for use in animal food, particularly pet food.

### 1. CROSS REFERENCE TO RELATED APPLICATIONS

This application claims priority under 35 USC § 119(e) to U.S. Provisional Patent Application No. 62/798,447 filed on Jan. 29, 2019, which is incorporated by reference in its entirety.

### 2. SEQUENCE LISTING

Incorporated by reference in its entirety herein is a computer-readable nucleotide/amino acid sequence listing submitted herewith and identified as follows: 1,777,447 Byte ASCII (Text) file name “513482_ST25” created on Mar. 18, 2022.

### 3. BACKGROUND

Currently, the production of high-quality protein foods and feed includes animal meat. As the global human and companion animal populations increase, the demand for high-quality protein food is expected to increase. However, obtaining proteins from animal meat for food production is an environmentally demanding, and potentially destructive, process.

While plant sources, e.g. legumes, contain a significant amount of protein, they often lack one or more essential amino acids for many mammalian diets [4] or they not as bioavailable as animal protein, making plant protein insufficient or sub-optimal alternative for many food applications. In fact, tryptophan and lysine are scarce in corn, lysine in wheat and other cereals, and methionine in soybeans and other legumes [6]. In addition, plant sources also contain anti-nutritional factors like fiber, phytate, and protease inhibitors, that limit digestion and absorption [1],[2]. Soybean, a commonly used protein source, decreases the digestibility in canine foods when present in concentrations over 15% [3]. Moreover, humans and companion animals have different amino acid requirements.

Hence, there remains a need for a source of proteins that do not come from animal meat yet satisfy the growing demand for high-quality protein food products and deliver on a multitude of nutritional needs.

### 4. SUMMARY

Disclosed herein are food compositions made with recombinantly produced animal proteins and methods for producing the recombinant animal proteins. Nucleic acid molecules encoding the animal proteins, expression vectors, recombinant host cells, and methods for making the animal proteins are also provided.

The recombinantly-produced animal proteins of the disclosure can be incorporated into food or feed product as whole cells, protein concentrates from cell lysates and/or cell supernatants, or as protein isolates to make various food products (e.g., primary diet foods, secondary diet foods), intermediate food products, supplements, and pharmaceutical compositions. The recombinant animal protein compositions may be mixed with other ingredients, shaped into a suitable form factor, to generate food products with a taste and mouthfeel suitable for humans or companion animals (e.g., dogs, cats, ferrets and the like).

Disclosed herein are improved methods and compositions for manufacturing food for animals, particularly companion animals. In certain embodiments, the methods entail producing animal proteins recombinantly in a microbial host, as described herein. The recombinant proteins produced by the method can provide equivalent or better nutrition than conventionally harvested animal proteins or plant-derived proteins, without the associated deficiencies described above. In certain embodiments, the recombinant animal proteins described herein can also be incorporated into or serve as food for humans, wild animals, livestock, domestic pets, companion animals, and/or zoo animals. In preferred embodiments, the food composition is substantially free of antibiotics, animal growth hormones, and/or meat from farmed, caught or slaughtered animals.

One or a plurality of recombinant proteins can be produced in one organism, or one strain, thereby allowing the amino acid profile to be tailored to the particular nutritional needs of targeted companion and other animals, including humans. Alternatively, a single recombinant animal protein can be produced in one strain (or organism) and mixed with a protein or proteins produced in a different strain (or organism) to yield a final product with the desired proportions of amino acids and other nutrients. Thus, the amino acid profile (and/or the profile of other nutrients) can be customized for the targeted animal, including pets and humans.

### 5. BRIEF DESCRIPTION OF THE DRAWINGS

FIG. 1 is a photograph of an SDS-PAGE gel of proteins extracted from an S. cerevisiae host cell strain that expresses a chicken cofilin-2 protein, with the chicken cofilin-2 band identified.

FIG. 2 shows the growth curves of an S. cerevisiae host cell strain that expresses chicken cofilin-2, and a control S. cerevisiae strain that does not express chicken cofilin-2, each grown under two different media conditions, (i) raffinose alone or (ii) raffinose with galactose to induce protein expression.

FIG. 3 shows maximum specific growth rates (μmax [h⁻¹]) from the experiments shown in FIG. 2. The error bars shown are standard deviation.

FIG. 4 is a picture of an SDS-PAGE gel of proteins extracted from an S. cerevisiae host cell strain that expresses chicken profilin protein, with the profilin band identified.

FIG. 5 is a picture of an SDS-PAGE gel of proteins extracted from an S. cerevisiae host cell strain that expresses chicken profilin protein that was used to make theoretical calculation shown in Table 4.

FIG. 6 shows the growth curve of an S. cerevisiae host cell strain expressing chicken profilin, and a control S. cerevisiae strain that does not express chicken profilin, each grown under two different media conditions, (i) raffinose alone or (ii) raffinose with galactose to induce protein expression.

FIG. 7 shows maximum specific growth rates (μmax [h⁻¹]) from the experiments shown in FIG. 6. The error bars are standard deviation. The asterisk denotes P value <0.05

FIG. 8 shows a picture of dried pellet from whole-cells expressing a recombinant profilin protein from chicken.

FIG. 9 shows a picture of the mixed, dry ingredients with a recombinant chicken profilin protein, processed into a powder.

FIGS. 10A-10B show pictures of processing a dough containing a recombinant animal protein into a food product. 10A shows a picture of processing of the wet and dry ingredients into a dough. 10B shows a picture of the molding the dough into a form factor of a treat.

FIGS. 11A-C show a picture of the dried and packaged treat with different protein content.

FIG. 12 shows a picture of an SDS-PAGE gel of a chicken coronin protein expressed in an S. cerevisiae host cell strain.

FIG. 13 shows a picture of an SDS-PAGE gel of a turkey myozenin-1 protein expressed in an S. cerevisiae host cell strain.

FIG. 14 shows a picture of an SDS-PAGE gel of a pig troponin C protein expressed in an S. cerevisiae host cell strain.

FIG. 15 shows a picture of an SDS-PAGE gel of a chicken cofilin-2 protein expressed in a K. phaffii host cell strain.

FIG. 16 shows a picture of an SDS-PAGE gel of a chicken profilin protein expressed in a K. phaffi host cell strain.

FIG. 17 shows a picture of an SDS-PAGE gel of chicken profilin protein expressed in a K. lactis host cell strain.

FIG. 18 shows a picture of an SDS-PAGE gel of a chicken profilin protein expressed in an S. pombe host cell strain.

### 6. DETAILED DESCRIPTION OF THE INVENTION

### 6.1. Definitions

Unless otherwise defined herein, all technical and scientific terms used herein have the meaning commonly understood by a person skilled in the art to which this invention pertains.

The term “ameliorating” refers to any therapeutically beneficial result in the treatment of a disease state, e.g., a nutritional deficiency disease state, including prophylaxis, lessening in the severity or progression, remission, or cure thereof.

The term “mammal” as used herein includes both humans and non-humans and includes but is not limited to humans, non-human primates, canines, felines, murines, bovines, equines, birds, and porcines.

The term “percent identity”, in the context of two or more nucleic acid or polypeptide sequences, refers to a specified percentage of nucleotides or amino acid residues that are identical as between or as among the sequences when aligned for maximum correspondence. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra) Unless otherwise specified, “percent identity” is assessed herein using the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/), and unless otherwise specified, “percent identity” is measured using BLASTP or BLASTN with default parameters at (www.ncbi.nlm.nih.gov). Depending on the application, the percent “identity” can exist over a region (e.g. a fragment) of the sequence being compared, e.g., over a functional domain, or, alternatively, exist over the full length of the two sequences to be compared.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

The term “nutritional supplement,” as used herein, generally refers to a substance capable of supplementing a diet of a human, dog, cat, or other animal. A nutritional supplement may provide essential nutrients (e.g., vitamins, minerals, macronutrients, trace nutrients, and/or cofactors). A nutritional supplement may be a dietary supplement.

The term “flavoring agent,” as used herein, generally refers to a substance capable of altering a flavor of a food product. A flavoring agent may include a flavoring molecule(s) or precursor(s), such as, for example, carbohydrates (e.g., sugar), sweeteners, or salts.

The term “recombinant host cell” as used herein refers to a host cell(s) that have been genetically modified to express or overexpress endogenous polynucleotides, to express heterologous polynucleotides or polypeptides, such as those included in an expression vector, in an integration construct, or which have an alteration in expression of an endogenous gene. By “alteration” it is meant that the expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration. For example, the term “alter” can mean “inhibit,” but the use of the word “alter” is not limited to this definition.

The term “heterologous” as used herein indicates molecules that are expressed in an organism other than the organism from which they originated or are found in nature. The molecule can have a coding region that is different from the host cell or a promoter region that is different from the host cell, or both.

On the other hand, the term “native” or “endogenous” as used herein indicates molecules that are expressed in the organism in which they originated or are found in nature, independently of the level of expression that can be lower, equal or higher than the level of expression of the molecule in the native host cell. It is understood that expression of wild-type enzymes or polynucleotides may be modified in recombinant host cells.

The term “transformation” refers to the transfer of a nucleic acid fragment into a host organism, resulting in genetic inheritance. Genetic inheritance can be stable or unstable. Host cells (e.g., eukaryotic cells) containing the transformed nucleic acid fragments are referred to as “transgenic” or “recombinant” or “transformed”.

The term “primary food product” or “primary diet food product” as used herein indicates a food product that is the core source of daily nutrition such as a complete meal or feed.

The term “secondary food product” or “secondary diet food product” as used herein indicates a food product that is generally not the core source of daily nutrition. By way of example, a secondary food product can be a snack, a treat, or an edible toy.

The term “intermediate food product” as used herein indicates a food product that is added to make the ultimate ingestible food composition. The intermediate food product is typically in a format that allows it to be mixed, coated, soaked, or injected to make the ultimate ingestible food composition.

The term “supplement” as used herein indicates a nutritional product that is intended to add or enhance the nutrient intake. The supplement can typically be in the form of a pill, a capsule, a tablet, a liquid, a soup, broth, or a dissolvable powder.

The term “substantially free” refers to a composition that comprises a desired compound, desired compounds, and inert compounds and is free of significant quantities of an undesired compound or undesired compounds. A typical substantially free composition comprises greater than about 80% by weight of the desired compound, desired compounds, and inert compounds and less than about 20% by weight of one or more other undesired compounds, more preferably greater than about 90% by weight of the desired compound, desired compounds, and inert compounds and less than about 10% by weight of one or more other undesired compounds, even more preferably greater than about 95% by weight of the desired compound, desired compounds, and inert compounds and less than about 5% by weight of one or more other undesired compounds, and most preferably greater than about 97% by weight of the desired compound, desired compounds, and inert compounds and less than about 3% by weight of one or more other undesired compounds.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “robust protein expression” is used herein to mean an increase in protein yield. Robust protein expression can arise from modifications in the protein itself or the host cell it is expressed by (also called biological or genetic robustness), or a combination of both.

The term “non-recombinant protein” or “supplementary protein” is used herein to mean a protein that is not produced by a recombinant technology such as for example, inserting a heterologous gene in a host cell to have the host cell produce the heterologous amino acid sequence, peptide, protein or fragment thereof.

It must be noted that, as used in the specification and the appended claims, the singular forms “a,” “an” and “the” include plural referents unless the context clearly dictates otherwise.

### 6.2. Recombinant Protein Compositions

6.2.1. Animal Proteins

The disclosure provides various recombinant animal proteins for the inclusion into food for primarily humans and pets. It is contemplated that any recombinant animal protein can be used with the methods and compositions of the disclosure. Often the recombinant animal protein is a heterologous protein.

The recombinant animal protein used with the methods and compositions of the disclosure may be a full-length protein, a truncated protein, or a fragment of a protein. A fragment (or portion of a protein) is an amino acid sequence that has at least three amino acids of the full-length protein. In some embodiments, the full-length protein is produced by expressing fragments that cover the full-length protein.

In some embodiments, the amino acid sequence of the animal proteins may be modified by replacing one or more amino acids with a different amino acid (e.g., by changing the nucleotide sequence of the recombinant gene encoding the protein).

Such amino acid modifications may improve the yield of the animal protein (e.g., by more robust protein expression) produced by the host cell that has been engineered to express the protein. Any amino acid modification can be made that improves or enhances the production of the animal proteins. In some embodiments the modification is made in the protein encoding region of the animal protein. In other embodiments, the modification is made in a regulatory element that controls or modifies the expression of the animal protein. Non-limiting examples of such amino acid modifications are: improving the efficiency of transcription and/or translation of the animal protein, improving the stability of the animal protein, altering the rate at which the protein is secreted by the host cell or by changing the activity of the animal protein so any deleterious effects on the expression of the animal protein are minimized.

In some embodiments, the animal protein has a higher percentage of essential amino acids compared to other animal tissue proteins. In some embodiments, the animal protein comprises more than 1%, 2%, 3%, 4%, 5%, 6%, 7%, 8%, 9%, 10%, 11%. 12%, 13%, 14%, 15%, 20%, 25%, 30%, 31%, 32%, 33%, 34%, 35%, 36%, 37%, 38%, 39% or 40% essential amino acids compared to other animal tissue proteins.

Depending on the host cell used, it may be helpful to select an animal protein that has a certain percentage of sequence identity to the proteins derived from the host cell. In some embodiments, the animal protein has 0%, 1%, 2%, 3%, 4%, 5%, 6%. 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% sequence identity to a gene or a region of a gene derived from a host cell. In some embodiments the animal protein has 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% sequence identity to a protein or a fragment of a protein derived from a host cell.

Non-limiting examples of animal proteins that can be used with the disclosure are: troponin I, actin, myosin, alpha-actinin-2, alpha-actinin-3, titin, receptor tyrosine protein kinase skeletal muscle, myosin binding protein C, F-actin-capping protein, Myosin-binding protein H, troponin T, myotubularin 1, myozenin-1, beta-enolase, cofilin-2, PDZ and LIM domain protein 7, twinfilin-2, telethonin, M-protein striated muscle, coronin, nebulin-related-anchoring protein, myopalladin, tensin, gelsolin, dystroglycan, profilin, myozenin-2, calsarcin 1, myotilin, paxillin, integrin alpha-7, integrin beta-1, dystrophin, ankyrin, paranemin, myomesin (skelemin), alpha sarcoglycan, gamma sarcoglycan, or calponin.

Examples of animal muscle proteins (or relatives of those proteins) that can be used with the disclosure include but are not limited to: thymosin beta 4, metavinculin, parvalbumin beta, tripartite motif-containing protein 54, obscurin, muscle M-line assembly protein unc-89, muscle-type aldolase, SERCA1, calponin homology-associated smooth muscle protein, skeletal muscle ankyrin repeat protein, calpain-3, atrogin-1, striated muscle-specific serine/threonine-protein kinase, skeletal muscle LIM-protein 2, glycogen phosphorylase, serpin A3-1, cadherin, beta-taxilin, density-regulated protein, synaptopodin, ARP2/3, WASP, SCAR/WAVE, IQGAP, AbpI, cortactin, drebrin, ENA/VASP, annexin II, BPAG, ERM protein, Sla2, utrophin, Srv2/CAP, verprolin, formins, capZ, fragmin, villin, AIP1, adducin, MACF, MAP2, tau, fimbrin, scruin, espin, fascin, actinfilin, actinogelin, Arklp, Prklp, actobindin, actolinkin, alpha-parvin, actophorin, acumentin, scinderin, afadin, AFAP-110, affixin, aginactin, angiogenin, dystonin, anilin, archvillin, cortactin, caltropin, CARMIL, caerin-1.16, dematin, diaphanous, EF-1a, EF-1b, LIM domain and actin-binding protein, elongation factor 2, epsin, proheparin-binding EGF-like growth factor, Mitogen-activated protein kinase, frabin, four and a half LIM domains protein 3, FH1/FH2 domain-containing protein 3, GAS2-like protein 2, kettin, Kelch protein, limatin, PDZ and LIM domain protein 1, synaptopodin-2, prefoldin, presenilin I, receptor tyrosine-protein kinase erbB-2, protein kinase C, striated muscle-specific serine/threonine-protein kinase, rapsyn, shroom, smitin, smoothelin, or serine/threonine-protein phosphatase, laminin, sarcospan, dystrobrevin, syntrophin, dysbindin, dysferlin, or fukutin.

Preferred animal protein sequences are listed in Table 1. They are grouped according to the tissue in which they are highly expressed (known). If it is not known in what tissue a protein is expressed, the protein is grouped according to the tissue for which its expression is required (e.g., for normal development of the tissue). For example, it is known that myotubularin is required for normal skeletal muscle growth. Thus, it is grouped with the skeletal muscle proteins. Persons skilled in the art will appreciate that in some cases a protein can be expressed in one or more tissue types.

In certain embodiments, the food compositions described herein comprise one or more of the animal proteins set forth in Table 1. In related embodiments, the food compositions described herein additionally, or alternatively, comprise one more recombinantly expressed homologs of the animal proteins set forth in Table 1.

In other related embodiments, the food compositions described herein comprise one or more animal proteins that are at least 50%, 60%, 70%, 80%, 85%, 90%, or 95% identical, but less than 100% identical, to the proteins set forth in Table 1 (i.e., the protein sequences are modified to alter their amino acid content, e.g., to improve nutrition, to improve digestibility, to optimize expression or to optimize secretion).

In other related embodiments, the food compositions described herein comprise one or more animal skeletal muscle tissue proteins of Table 1, or one or more cardiac muscle tissue proteins of Table 1, or one or more smooth muscle tissue proteins of Table 1, or one or more of the skeletal/cardiac muscle tissue proteins of Table 1, or one or more of the skeletal/smooth muscle tissue proteins of Table 1, or one or more of the cardiac/smooth muscle tissue proteins of Table 1, or one or more of the skeletal/cardiac/smooth muscle tissue proteins of Table 1. In yet other related embodiments, the food compositions described herein comprise proteins from two or more of the above-mentioned categories of proteins described in Table 1.

In some embodiments, the animal protein is an actin cytoskeleton protein. In some embodiments, the actin cytoskeleton protein is a filament protein, a capping protein, an actin-binding protein, an actin-bundling protein, a monomer binding protein, a cytoskeletal linker protein, a membrane anchor protein, a stabilizing protein, a sidebinder protein, a signaling protein, a capping protein, a severing protein, or a myosin.

6.2.2. Nucleic Acids Encoding the Animal Proteins

Production of a recombinant animal protein of the disclosure can be achieved by the manipulation of a gene that encodes an animal protein, which is then inserted in a host cell expression system such that it expresses large amounts of a recombinant gene that is converted into an animal protein using the host cell expression system. This process can include the transcription of the recombinant DNA to messenger RNA (mRNA), the translation of mRNA into polypeptide chains, which are ultimately folded into functional proteins and may be targeted to specific subcellular or extracellular locations depending on the sequence. However, an animal protein need not be folded or targeted to add to the nutritional value of a food product. Where the animal protein is a fragment or portion of an animal protein it may not be folded.

Genes encoding recombinant animal proteins can be obtained by taking a sample from an animal and extracting nucleic acids, such as mRNA, from that sample and then amplifying the gene by reverse transcription followed by PCR. The sample could be a tissue sample (e.g., muscle), a blood sample, mucus, skin, saliva, or hair. Another option is to have the gene synthesized by a company that performs such work.

Alternatively, where the genome sequence of the animal has been determined, the gene sequences (DNA/nucleotide sequences) or protein sequences of an animal can be obtained by searching appropriate databases (e.g., UniProtKB and NCBI). A polynucleotide can be obtained using chemical synthesis, molecular cloning or recombinant methods, DNA or gene assembly methods, artificial gene synthesis, PCR, or any combination of those.

In the case that there are not sequences available for an animal protein of interest, conserved regions can be used to amplify segments of the genes and the flanking regions can be sequenced in order to obtain the full-length sequence. Multiple sequence alignments of a specific protein in several different organisms will show where the conserved regions lie, and which are the most suitable stretches to use for primer design. Primers with alternative nucleotides can be used when needed.

The present invention provides codon-optimized nucleic acid encoding an animal protein for expression in a host cell. Codon-optimization for expression in a particular host cell can be determined by codon usage tables or by using a program that is instructed by an algorithm that identifies a region of sequence that can be optimized for protein expression in the host cell. Any commercially available optimization algorithm or any publicly available algorithms can be used with the disclosure. Using such programs, various improvements can be achieved to enhance expression of a recombinant animal protein as discussed herein. Specific examples of codon-optimization of animal protein gene sequences for certain host cells are provided herein.

The gene sequences that can be used with the methods and compositions of the disclosure are those encoding the types of proteins described herein. In some embodiments, the gene sequence may include non-coding introns. In some embodiments, the gene sequences may not include non-coding introns.

Depending on what method is used to produce a recombinant animal protein, a gene encoding the animal protein may further comprises one or more regulatory elements. Non-limiting examples of regulatory elements include but are not limited to such as a restriction enzyme site, a promoter, an enhancer, a signal sequence, a terminator, or a combination thereof

6.2.2.1. Origin

The identification and cloning of an animal protein are discussed herein. The origin of the recombinantly expressed protein sequence (i.e., the species of animal from which the sequence to be recombinantly expressed is found in nature) can be any species within the biological kingdom of Animalia. Preferably, the origin of the recombinantly expressed protein sequence is a vertebrate animal, which can be a fish, a bird, a mammal, an amphibian, or a reptile. The origin may be a placental mammal, monotreme mammal, or marsupial mammal (metatheria). The origin may furthermore be a bird or another vertebrate from the reptile clade.

In some embodiments, the gene origin is a placental mammal, including but not limited to carnivores (including lion, bear, weasel, seal, wolf, coyote, fox), equidae (including horse and donkey), even-toed ungulates (including pig, camel, cattle, and deer), Afrotheria (including elephants, woolly mammoth, golden moles, and manatees), and Boreoeutheria (including primates, rabbits, hares, pikas, rodents, moles, whales, bats, dogs, cats, seals, and hoofed mammals).

In some embodiments, the origin is a monotreme mammal, including but not limited to platypus and echidna. In some embodiments, the origin is a marsupial mammal, including but not limited to koala, possums, tapirs, kangaroos, wallabies, and marsupial lions.

In some embodiments, the origin is a hoofed mammal, including but not limited to cattle, antelope, deer, reindeer, elk, sheep, goat, camels, carabao, yak, bison, buffalo, caribou, water buffalo, pig, horse, and donkey. In some embodiments, the origin is an endothermic vertebrate, classified as Ayes, including but not limited to chicken, turkey, duck, pigeon, penguin, ostrich, goose, pheasant, and quail.

In some embodiments, the gene origin is a reptile, including but not limited to alligators and crocodiles.

In some embodiments, the gene origin is an aquatic animal, including but not limited to shark, tuna, trout, salmon, herring, jacks, carp, catfish, cod, flounder, bass, tilapia, sturgeon, crab, lobster, shrimp, prawns, oysters, mussels, eels, shellfish, cuttlefish, starfish, crayfish, and jellyfish.

In some embodiments, the gene origin is an amphibian, including but not limited to frogs, salamanders, and toads. In some embodiments, the gene origin is an insect.

6.2.2.2. Tissue Source

The recombinant animal protein may be from any organ or tissue of an animal, including, but not limited to proteins expressed in the brain, skin, scales, feathers, eyes, shells, hair, horns, ears, liver, heart, kidney, stomach, intestines, and muscle tissue (e.g., skeletal, smooth or cardiac).

In preferred embodiments, the recombinant animal proteins are muscle proteins. In some embodiments, the recombinant animal protein is cytoskeletal. In some embodiments, the actin cytoskeleton protein is a filament protein, a capping protein, an actin-binding protein, an actin-bundling protein, a monomer binding protein, a cytoskeletal linker protein, a membrane anchor protein, a stabilizing protein, a sidebinder protein, a signaling protein, a capping protein, a severing protein, or a myosin. In some embodiments, the recombinant animal protein is a myosin. In some embodiments, the recombinant animal protein is an actin.

The animal muscle proteins include those proteins normally found in animal muscle tissue (or relatives of those proteins). In addition to myosin and actin, these proteins include but are not limited to troponin, tropomyosin, alpha-actinin, beta-actinin, titin, connectin, skeletal receptor, myosin-binding protein, desmin, leiomodin, tubulin, myotubularin, myozenin, telethonin, calsarcin, myotilin, nebulin, nebulin-related anchoring protein, myomesin, vinculin, paxillin, beta-enolase, myotubularin, calponin, caldesmon, transgelin, tropomodulin, supervillin, gelsolin, twinfilin, profilin, caveolin, catenin, cofilin, capping protein, leiomodin, tensin, M-protein, radixin, filamin, keratin, myopalladin, calsequestrin, caveolae-associated protein, nebulette, coronin, talin, dystrophin, dystroglycan, integrin, ankyrin, syncoilin, smoothelin-like-1, spectrin, synemin, paranemin, ponsin, plectin, skelemin, sarcoglycan, LIM protein, myoblast determination protein, myocyte-specific enhancer, and myocilin.

6.2.3. Expression Vectors

The disclosure also provides various expression vectors (e.g., constructs) comprising a genetic element (e.g., DNA, or cDNA) encoding for a protein derived from an animal. Depending on the host cell used for protein expression, a person skilled in the art of biotechnology will know the appropriate expression vector to use (e.g., plasmid, virus) with the regulatory elements (e.g., transcriptional start site, promoter, and the like) and genetic elements required for protein expression in a particular host cell. Specific examples of expression vectors that can be used with the disclosure are provided herein.

A genetic element is any coding or non-coding nucleic acid sequence. A genetic element can be a nucleic acid that codes for an amino acid, a peptide or a protein. Genetic elements may be operons, genes, gene fragments, promoters, exons, introns, regulatory sequences, or any combination of those. A genetic element includes an entire open reading frame of a protein, or the entire open reading frame and one or more (or all) regulatory sequences associated therewith. The genes may be codon-optimized for expression in a particular recombinant host cell (e.g., codon-optimized for yeast, insect, or mammalian host cell).

In some embodiments, an expression vector can comprise one genetic element. In some embodiments, an expression vector can comprise at least 2, 3, 4, 5, or 6 genetic elements. In some embodiments, an expression vector can comprise one regulatory element. In some embodiments, an expression vector can comprise at least 2, 3, 4, 5, or 6 regulatory elements. A person skilled in the art knows the payload limitations (e.g. kilobase pairs) for certain various expression vectors (e.g., cosmids, plasmids, etc).

The term “engineered” or “recombinant” refers to a cell into which a recombinant gene, such as, for example, a gene encoding an animal protein, or part of an animal protein, has been introduced. Therefore, engineered cells are distinguishable from naturally occurring cells that do not contain a recombinant gene that is introduced by transfection, transformation, cell fusion, mating or other techniques. Recombinantly introduced genes will either be in the form of a cDNA (i.e., they will not contain introns), a copy of a cDNA gene, genomic DNA (with or without introns; for expression in prokaryotic hosts, the DNA should be without introns), or will include DNA sequences positioned next to a promoter not naturally associated with the particularly introduced gene.

6.2.3.1. Promoters

Disclosed herein are expression vectors comprising a genetic element encoding an animal protein or part of an animal protein and the use thereof for the recombinant expression of the animal protein.

The expression vector may further comprise a promoter. The promoter may be a constitutive promoter, an inducible promoter, or a hybrid promoter. Where overexpression of a protein is toxic to a host cell (e.g., reduces growth of the cell, kills the cell, or reduces protein expression) it may be preferable to use an inducible promoter.

In the expression vector, the gene construct and the method, the promoter may be a viral promoter, a prokaryotic promoter or a eukaryotic promoter. The promoter may be a synthetic promoter from a promoter library. The promoter may be any scientifically known promoter or a novel promoter. The promoter may be an engineered form of a known promoter or a hybrid promoter.

The eukaryotic promoter may be a fungi promoter, a plant promoter, or an animal promoter. The fungi promoter may be the promoter of the genes phosphoglycerate kinase (PGK, PGK1, PGK3), enolase (ENO, ENOl), glyceraldehyde-3-phosphate dehydrogenase (gpdA, GAP, GAPDH), hexokinase, pyruvate decarboxylase, phosphofructokinase, glucose-6-phosphate isomerase, 3-phosphoglycerate mutase, pyruvate kinase, triosephosphate isomerase, phosphoglucose isomerase, glucokinase, alcohol dehydrogenase promoter (ADH1, ADH2, ADH4), isocytochrome C, acidic phosphatase, galactose metabolism enzymes, GAL (GAL1, GAL2, GAL3, GAL4, GAL5, GAL6, GAL7, GAL8, GAL9, GAL10), alternative oxidase (AOD), alcohol oxidase 1 (AOX1)), alcohol oxidase 2 (AOX2), CUP1, AHSB4m, adhl+, AINV, alcA, AXDH, cellobiohydrolase I (cbhl), ccg-1, cDNAl, cellular filament polypeptide (cfp), cpc-2, ctr4+, dihydroxyacetone synthase (DAS), FMD, formate dehydrogenase (FMDH), formaldehyde dehydrogenase (FLD1), GAA, GCW14, glucoamylase (glaA, gla-1), invl, isocitrate lyase (ICL1), glycerol kinase (GUT1), acetohydroxy acid isomeroreductase (ILV5), β-galactosidase (lac4), LEU2, melO, MET3, MET25, KAR2, KEX2, methanol oxidase (MOX), nmtl, peroxin 8 (PEX8), pcbC, PET9, PH05, PH089, PYK1, phosphatidylinositol synthase (PIS1), RPS7, TEF, translation elongation factor 1 alpha (TEF1), sorbitol dehydrogenase (SDH), SSA4, THI11, homoserine kinase, XRP2, TPI, and YPT1, PHOS, CYC1, HIS3, ADC1, TAP1, URA3, LEU2, TP1, TDH1, TDH3, FBA1, ADR1, TPI1, or any combination of those.

The plant promoter may be the promoter of the gene phol, TPI, TPS1, and any combination of these.

The animal promoter may be a heat-shock protein promoter, proactin promoter, immunoglobulin promoter, or the promoter of the gene B2, HSP82, Ser1, triose phosphate isomerase (TPI1), or any combination of those. However, any promoters can be used if they drive the expression of recombinant proteins in a particular host cell.

6.2.3.2. Selection Gene Marker

The expression vector may include a selection gene marker. For example, an expression vector may comprise an auxotrophic marker. Non-limiting examples of auxotrophic markers that can be used with the disclosure include trp1, leu2, his3, adel, arg4, his4, ura3, and/or met2. In some embodiments, more than one selection gene marker may be used.

In some embodiments, the expression vector may comprise a selectable marker, which may be an antibiotic resistance gene. The resistance gene may confer resistance to drugs including, but not limited to, zeocin, ampicillin, blasticidin, kanamycin, nurseothricin, chloroamphenicol, tetracycline, triclosan, or ganciclovir. In some embodiments, more than one resistance genes may be used. Yet, for some food compositions, it may be desirable not to use an antibiotic resistance gene for selection.

In applications when there are several animal proteins expressed, it may be useful to use one or more resistance genes in combination with one or more auxotrophic markers.

6.2.3.3. Integration and Transformation

The compositions of the invention include a recombinant host cell transformed with an expression vector to express one or more recombinant animal proteins.

One or more expression vectors with the required genetic elements (e.g., regulatory elements or protein-encoding, genetic elements) may be integrated into a genome. In some applications, it may be desirable to integrate multiple copies of the same expression vector.

Alternatively, or in addition, the host cell may comprise multiple copies of an expression vector where the expression vector is not integrated into a genome.

Any small DNA molecule within a cell that is capable of being physically separated from chromosomal DNA and can replicate can be used with the methods and compositions of the disclosure. The expression vectors that can be used with the disclosure are a plasmid, a conjugative plasmid, a non-conjugative plasmid, a cosmid, a hybrid plasmid, a virus, a phage, or the like.

Host cells may be transformed or transduced to introduce the expression vector by transfection, infection, endocytosis, F-mating, mating, PEG-mediated protoplast fusion, Agrobacterium tumefaciens-mediated transformation, chemical transformation, electroporation, heat-shock transformation, biolistic transformation or any other method known in the art.

6.2.3.4. Signal Peptide Sequence

The expression vector may further comprise a signal peptide sequence. A signal peptide, also known as a, signal sequence, targeting signal, localization signal, localization sequence, secretion signal, transit peptide, leader sequence, or leader peptide, may cause extracellular secretion of a protein.

Extracellular secretion of a recombinant animal protein from a host cell simplifies protein purification. Recovery of a recombinant animal protein from a cell culture supernatant may be preferable to lysing host cells to release a complex mixture of proteins including intracellular proteins of the host cell.

For some applications, secretion may reduce harmful effects that intracellular overexpression of a recombinant animal protein may have on a host cell such as toxicity or reduced growth rate.

Secretion may produce higher amounts of an animal protein compared to intracellular expression. Secretion of a protein may also enable post-translational modification (e.g., glycosylations) or aid in folding the protein correctly and allow for the formation of disulfide bonds.

6.2.4. Host Cells

The expression vectors provided by the disclosure are transformed into host cells. Typically, the host cell is a eukaryotic host cell.

Any eukaryotic host cell known in the art can be used with the expression vectors and animal proteins provided by the disclosure to make a recombinant host cell. Examples of a eukaryotic host cell that can be used with the disclosure are an insect cell, a fungal cell, a plant cell, and a mammalian cell.

Genetic modification of the host cell is accomplished in one or more steps via the design and construction of appropriate vectors and transformation of the host cell with those vectors. Electroporation and/or chemical (such as calcium chloride- or lithium acetate-based) transformation methods can be used. Methods for transforming yeast strains are described in WO 99/14335, WO 00/71738, WO 02/42471, WO 03/102201, WO 03/102152 and WO 03/049525; these methods are generally applicable for transforming host cells in accordance with this invention. The DNA used in the transformations can either be cut with particular restriction enzymes or used as circular DNA.

The recombinant host cells can be cultured in appropriate media to produce large quantities of the recombinant animal protein.

6.2.4.1. Yeast Host Cells

In some embodiments, the host cell used to express the protein is a yeast host cell. The yeast cell can be a budding yeast, fission yeast, or a filamentous yeast. In some applications, the yeast host cell is a wild-type yeast. However, often, the yeast host cell used with the method and compositions of the disclosure is a modified yeast host cell (e.g., through mutation, genome shuffling, protoplast fusion, cytoduction, etc.) to enhance the production or yield of protein, aid selection of, or any other modification that enhances production of the animal protein such that host cell gives more robust expression (i.e., strain robustness). The modification can result in a yeast host cell that is polyploid or aneuploid. In some applications, the host cell may be modified so that it grows faster, grows to a higher cell density, is less sensitive to environmental factors in the bioproduction process fluctuations such an unexpected change in temperature or reduced nutrients.

The yeast host cell may be obtained from a variety of sources known to people skilled in the art, including commercial sources. In some embodiments, the yeast host cell may be selected from the “Saccharomyces Yeast Clade”, as described in US Publication No. 2009/0226991.

In certain embodiments, the yeast host cell is a Saccharomyces sensu stricto yeast. The term “Saccharomyces sensu stricto” taxonomy group is a cluster of yeast species that are highly related to S. cerevisiae (Rainieri et al., 2003, J. Biosci Bioengin 96: 1-9). Saccharomyces sensu stricto yeast species include but are not limited to S. cerevisiae, S. kudriavzevii, S. mikatae, S. bayanus, S. uvarum, S. carocanis and hybrids derived from these species (Masneuf et al., 1998, Yeast 7: 61-72).

An ancient whole genome duplication (WGD) event occurred during the evolution of the hemiascomycete yeast and was discovered using comparative genomic tools (Kellis et al., 2004, Nature 428: 617-24; Dujon et al., 2004, Nature 430:35-44; Langkjaer et al., 2003, Nature 428: 848-52; Wolfe et al., 1997, Nature 387: 708-13). Using this major evolutionary event, yeast can be divided into species that diverged from a common ancestor following the WGD event (termed “post-WGD yeast” herein) and species that diverged from the yeast lineage prior to the WGD event (termed “pre-WGD yeast” herein).

In some embodiments, the yeast host cell may be selected from a post-WGD yeast genus, including but not limited to Saccharomyces and Candida. In some embodiments, post-WGD yeast species include: S. cerevisiae, S. uvarum, S. bayanus, S. paradoxus, S. castelli, and C. glabrata.

In some embodiments, the yeast host cell may be selected from a pre-whole genome duplication (pre-WGD) yeast genus including but not limited to Saccharomyces, Kluyveromyces, Candida, Pichia, Issatchenkia, Debaryomyces, Hansenula, Yarrowia and, Schizosaccharomyces. Representative pre-WGD yeast species include: S. kluyveri, K thermotolerans, K. marxianus, K. waltii, K. lactis, C. tropicalis, P. pastoris, P. anomala, P. stipitis, I. orientalis, I. occidentalis, I. scutulata, D. hansenii, H anomala, Y. lipolytica, and S. pombe.

A yeast host cell used with the disclosure may be either Crabtree-negative or Crabtree-positive, as described in US Publication No. 2009/0226991. A yeast microorganism may be either Crabtree-negative or Crabtree-positive. A yeast cell having a Crabtree-negative phenotype is any yeast cell that does not exhibit the Crabtree effect. The term “Crabtree-negative” refers to both naturally occurring and genetically modified organisms. Briefly, the Crabtree effect is defined as the inhibition of oxygen consumption by a microorganism when cultured under aerobic conditions due to the presence of a high concentration of glucose (e.g., 50 g glucose L⁻¹). In other words, a yeast cell having a Crabtree-positive phenotype continues to ferment irrespective of oxygen availability due to the presence of glucose, while a yeast cell having a Crabtree-negative phenotype does not exhibit glucose mediated inhibition of oxygen consumption.

In some embodiments, the yeast host cell may be selected from yeast with a Crabtree-negative phenotype including but not limited to the following genera: Saccharomyces, Lachancea, Kluyveromyces, Pichia, Issatchenkia, Komagataella, Yarrowia, Hansenula, Debaromyces, Ogataea, Zygosaccharomyces and Candida. Crabtree-negative species include but are not limited to: L. kluyveri (fka S. kluyveri), K. lactis, K. marxianus, P. anomala, S. stipitis (fka P. stipitis), I. orientalis, D. occidentalis, P. scutulata, P. anomala, Ogataea polymorpha, Arxula adeninivorans, Cyberlindnera jadinii, K. phaffii, Y. lipolytica, Kluyveromyces fragilis, D. hansenii, P. kudriavzevii and C. utilis.

In some other embodiments, the yeast host cell may be selected from yeast with a Crabtree-positive phenotype, including but not limited to the genera Saccharomyces, Kluyveromyces, Zygosaccharomyces, Naumovozyma, Lachancea, Dekkera, Candida, Pichia and Schizosaccharomyces. Crabtree-positive yeast species include but are not limited to: S. cerevisiae, S. uvarum, S. bayanus, S. paradoxus, N. castellii, L. thermotolerans, C. glabrata, Z. bailii, Z. rouxii, D. bruxellensis and S. pombe.

Another characteristic may include the property that the host cell is non-fermenting. In other words, it cannot metabolize a carbon source anaerobically while the yeast is able to metabolize a carbon source in the presence of oxygen. Nonfermenting yeast refers to both naturally occurring yeasts as well as genetically modified yeast.

In some embodiments, the recombinant host cells may be host cells that are non-fermenting yeast host cells, including, but not limited to those classified into a genus selected from the group consisting of Tricosporon, Rhodotorula, Myxozyma, or Candida. In a specific embodiment, the non-fermenting yeast is C. xestobii.

6.2.4.2. Mammalian Host Cells

Cultured mammalian cell lines may also be used to express the animal proteins provided by the disclosure. In some embodiments, Chinese hamster ovary (CHO) can be used. In some embodiments, human cell lines such as HEK or HeLa may be used to produce protein. In some embodiments, a commercially available mammalian expression system can be used such Expi293, ExpiCHO, ExpiCHO, T-REx Expression System, Flp-In T-REx system, GeneSwitch System from Thermofisher.

### 6.3. Methods for Bioproduction

6.3.1. Cell Culture Processes and Fermentation

The bioproduction of a recombinant animal protein may be conducted by cell culture processes or by fermentation. When fermentation is used, it may be conducted aerobically, microaerobically or anaerobically.

In some embodiments, the method for producing a recombinant animal protein for a food product consumption comprises (i) providing a reactor or flask comprising a fungal colony and (ii) a feedstock comprising a nitrogen-containing material and a carbon-containing material (e.g., sugar), and permitting the fungal colony to grow in presence of the feedstock to yield the fungus-containing product comprising a recombinant animal protein. In some embodiments, a selective media or reagent can be used to select for host cells harboring the recombinant animal gene.

In some embodiments, the method for producing a recombinant animal protein for a food product consumption comprises (i) providing a reactor comprising a fungal colony and (ii) a feedstock comprising a nitrogen-containing material and a sugar-containing material, and (iii) when the fungal colony reaches the exponential growth phase and an inducing agent is added to yield the fungus-containing product comprising a recombinant animal protein.

In some embodiments, the fungal colony comprises one or more budding fungi. Examples of preferred budding fungi are Saccharomyces cerevisiae, Schizosaccharomyces pombe, Komagataella phaffii, Kluyveromyces lactis, and a derivative thereof.

In some embodiments, the genome of a budding fungi can be genetically modified in at least one gene to yield more robust protein expression. Genetic modifications that can yield more robust protein expression are discussed herein. In some embodiments, the genome of a budding fungi can be genetically modified to be protease deficient.

In some embodiments, the fungal colony does comprise one or more filamentous fungi. Non-limiting examples of filamentous fungi that can be used are Aspergillus oryzae, Trichoderma reesei, Fusarium venenatum, Geotrichum candidum, Penicillium camemberti, Penicillium roqueforti, and a derivative thereof.

In some embodiments, the genome of a filamentous fungi can be genetically modified in at least one gene to yield more robust protein expression. Genetic modifications that can yield more robust protein expression are discussed herein. In some embodiments, the genome of a filamentous fungi can be genetically modified to be protease deficient.

In some embodiments, the recombinant animal protein is produced in a recombinant host cell and expressing the recombinant animal protein intracellularly. In some embodiments, the recombinant animal protein is produced in a recombinant host cell and expressing the recombinant animal protein such that it is secreted into the culture broth.

The recombinant animal protein may be obtained by a whole-cell preparation (i.e., host cell itself, and the recombinant protein expressed within or on its surface, can be added to the food composition), a protein concentrate preparation, or by isolating an animal protein. Depending on where the protein is expressed in the cell (e.g., extracellularly or intracellularly) protein concentrate can be from a cell lysate or a cell supernatant after centrifugation.

6.3.2. Harvesting of Intermediate Food Product

The disclosure also provides methods for making an intermediate food product.

In some embodiments, the method comprises culturing eukaryotic host cell that recombinantly expresses a heterologous animal protein and harvesting the recombinant host cell, thereby making an intermediate food product.

In some embodiments, the method comprises culturing eukaryotic host cell that recombinantly expresses an animal protein, concentrating the recombinant host cell from the culture, extracting proteins in a protein concentrate from the concentrated culture, thereby making an intermediate food product.

In some embodiments, the method comprises culturing eukaryotic host cell that recombinantly expresses an animal protein, concentrating the recombinant host cell from the culture, and isolating the animal protein, thereby making an intermediate food product.

Where the animal protein is expressed intracellularly in the host cell, a cell lysate can be obtained from the eukaryotic host cell to make the intermediate food product. Where the animal protein is expressed extracellularly, the cell supernatant can be obtained the intermediate food product.

The intermediate food product can also be made in a format such that it is used to another food product. In some embodiments, the intermediate food product is harvested and made in the format an ingredient, a coating, a palatability agent, or a flavoring agent as discussed in more detail below.

### 6.4. Intermediate Food Products

The disclosure also provides various intermediary food products comprising the recombinant animal protein. The intermediary food product can be substantially free of an antibiotic, an animal growth hormone, animal meat, or proteins derived from animal meat.

The recombinant animal protein can be harvested and provided to the intermediary food product as a whole-cell food composition, a protein concentrates food composition, or as a protein isolate food composition. An intermediate food product can be mixed, coated, soaked or injected into an ultimate ingestible food product. The ultimate ingestible food product can be a commercially available feed, food, supplement, or treat.

In some embodiments, the intermediary food is a wet or dry ingredient that is added to another food product. The intermediary food can also be a coating to be added to the exterior of a food product. The coating can be soaked, brushed, or sprayed on a food product. In some embodiment the intermediary food protein can be a palatability agent that enhances the acceptance of the food product, as a flavoring agent or agent that enhances mouth-feel (e.g., texture and the like).

In some embodiments, the harvested whole cell, protein concentrate, or protein isolate can be concentrated and dried, thereby making a dry intermediate food product. A dry intermediate food product comprising the recombinant animal protein can be in the form of a powder, a granule, a pellet, a slurry or paste, of varying moisture content.

### 6.5. Food Product Compositions

The disclosure provides various food product compositions (for humans and pets) comprising the recombinant animal protein as well as supplements. The food product can be substantially free of an antibiotic, an animal growth hormone, animal meat, or proteins derived from animal meat. In some embodiments, the food product is substantially free of any other ingredient. In other embodiments, the food product is combined with other ingredients.

The recombinant animal protein-containing food product can be formulated as a primary diet food product for an animal or individual (e.g. that is, it acts as the core source of daily nutrition). Examples of a primary food product include but are not limited to a meal, a kibble, a wet food, a dry food (e.g., freeze-dried or dehydrated).

The recombinant animal protein-containing food product can be formulated as secondary diet food product (that is, it does not provide nutrients in the amounts that are required for daily nutrition for an animal or individual). Examples of a secondary diet food products are a snack, a treat, or an edible toy.

The recombinant animal protein-containing food product can also be made from an intermediary diet food product (e.g., ingredient, a coating, a palatability agent, or a flavoring agent) that is added to make an ultimate ingestible food product.

6.5.1. Dry and Wet Food Products

The recombinant animal protein is introduced into a dry or wet food composition by addition of the intermediate food product, which can be a whole-cell food product, a protein concentrate food product, or as a protein isolate food product, thereby making a dry food product. In some embodiments, the dry food product can be further processed and shaped into a kibble, a treat, a snack, a chew, or an edible toy.

In some other embodiments, intermediate food product, which can be a whole cell, protein concentrate, or protein isolate can be concentrated, dried, and then rehydrated with one or more wet ingredients thereby making a wet food product. Wet products comprising the recombinant animal protein can be in the form of a slurry, a paste, a suspension, or a liquid. The wet food composition maybe semi-moist, intermediate moist, or moist. In some embodiments, the wet food composition can be further processed and shaped into a kibble, a treat, a snack, a chew, or a toy.

Depending on the percentage of essential amino acids desired for a food composition one can determine the amount of intermediate food product needed to achieve the desired amino acid content in the final food product (e.g. dry or wet food product). For example, the contribution of amino acids from profilin can be calculated for different expression levels (Table 4).

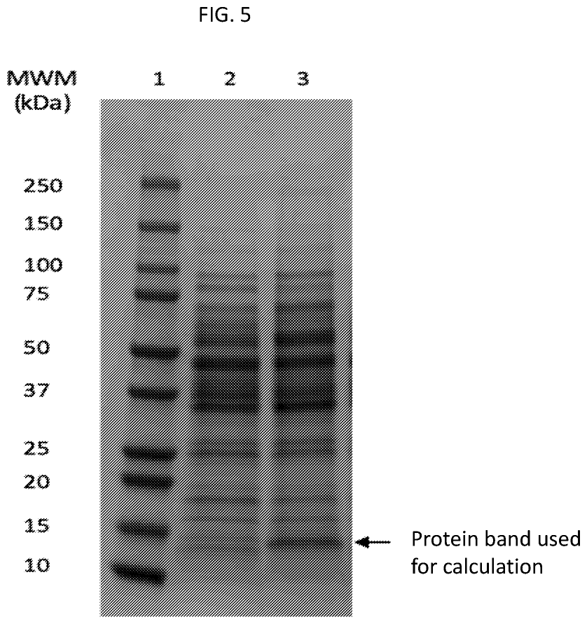

To carry out this example calculation, it was assumed that the total protein content per dry cell weight was constant. It was also assumed that profilin expression did not change the profile of endogenous cellular proteins, and that expression of endogenous proteins decreased (in percent) the same as profilin increased (on a mass basis). The current estimated expression level of profilin is 10% of the total protein. The level was calculated based on the intensity of the protein bands in FIG. 5 using the software Image J (Schneider, Rasband, & Eliceiri, 2012; NIH Image to ImageJ: 25 years of image analysis. Nature Methods, 9, 671-675).

TABLE 4
Increase in essential amino acids at different levels of profilin expression

| | Profilin expression level (wt % of total protein) | | | | | | | | | | |
|---|---|---|---|---|---|---|---|---|---|---|---|
| | 2% | 4% | 6% | 8% | 10% | 12% | 15% | 20% | 30% | 40% | 50% |
| | Increase in amino acid content per dry weight | | | | | | | | | | |
| Arginine | 0.23% | 0.47% | 0.70% | 0.93% | 1.17% | 1.40% | 1.75% | 2.34% | 3.51% | 4.67% | 5.84% |
| Histidine | −0.66% | −1.33% | −1.99% | −2.66% | −3.32% | −3.99% | −4.98% | −6.64% | −9.96% | −13.3% | −16.6% |
| Isoleucine | 0.39% | 0.79% | 1.18% | 1.57% | 1.97% | 2.36% | 2.95% | 3.93% | 5.90% | 7.86% | 9.83% |
| Leucine | −0.17% | −0.34% | −0.52% | −0.69% | −0.86% | −1.03% | −1.29% | −1.72% | −2.58% | −3.44% | −4.30% |
| Lysine | 0.15% | 0.30% | 0.44% | 0.59% | 0.74% | 0.89% | 1.11% | 1.48% | 2.22% | 2.96% | 3.70% |
| Methionine | 3.22% | 6.44% | 9.66% | 12.9% | 16.1% | 19.3% | 24.1% | 32.2% | 48.3% | 64.4% | 80.5% |
| Phenylalanine | 0.42% | 0.83% | 1.25% | 1.66% | 2.08% | 2.49% | 3.12% | 4.16% | 6.23% | 8.31% | 10.4% |
| Threonine | 0.97% | 1.94% | 2.92% | 3.89% | 4.86% | 5.83% | 7.29% | 9.72% | 14.6% | 19.4% | 24.3% |
| Tryptophan | 1.49% | 2.99% | 4.48% | 5.97% | 7.46% | 8.96% | 11.2% | 14.9% | 22.4% | 29.9% | 37.3% |
| Valine | 0.75% | 1.50% | 2.26% | 3.01% | 3.76% | 4.51% | 5.64% | 7.52% | 11.3% | 15.0% | 18.8% |

6.5.1.1. Whole-Cell Food Products

The disclosure also provides a whole-cell food product compositions. The whole-cell food product composition is made with the host cell expressing the recombinant animal protein.

Host cells expressing recombinant animal protein may be harvested by batch centrifugation, continuous flow centrifugation, filter press, flocculation, rotary drum vacuum filtration, tangential flow filtration, ultrafiltration or combination of these methods or any technique known in the art.

Cells may be lysed by raising temperature, autolysis, by high-pressure homogenization (e.g., French press), ultrasonic cavitation, bead beating, rotor-stator processors, freeze-thaw cycles, enzymatic lysis (e.g., lysozyme, lysostaphin, zymolase, cellulose, protease or glycanase), osmotic shock methods, chemical lysis (by alkaline, detergent or organic solvent) or a combination of these methods or any technique known in the art.

In some embodiments, food product comprising the recombinant animal protein is a whole-cell food product. In some embodiments, the whole-cell food composition comprises about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of recombinant animal protein by dry weight, semi-moist weight, or wet weight.

6.5.1.2. Protein Concentrate Food Products

The disclosure also provides protein concentrate food product compositions. In some embodiments, the protein concentrate food product comprising the recombinant animal protein is made from a protein concentrate from a host cell expressing the recombinant animal protein.

Depending on whether the animal protein is expressed intracellularly or extracellularly in the host cell, the animal protein can be harvested from a cell lysate or cell supernatant of the host cell, respectively.

A protein concentrate can be purified from a host cell lysate or host cell supernatant by any technique known in the art.

In some embodiments, the protein isolate food composition comprises about 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of recombinant animal protein by dry weight, semi-moist weight, or wet weight.

6.5.1.3. Protein Isolate Food Products

The disclosure also provides protein isolate food product compositions. In some embodiments, the protein isolate food product comprising the recombinant animal protein is made from a protein isolate from a host cell expressing the recombinant animal protein. Where a protein isolate is desired the gene encoding the animal protein will often further comprise a molecule tag or label that can facilitate the isolation of the animal protein. In some embodiments, one or more tags or labels can be used to isolate different animal proteins expressed in the same host cell.

Depending on if the animal protein is expressed intracellularly or extracellularly in the host cell, the animal protein can be harvested from a cell lysate or cell supernatant of the host cell, respectively. The animal proteins can be isolated using techniques known in the art.

In some embodiments, the protein isolate food composition comprises about 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of recombinant animal protein by dry weight, semi-moist weight, or wet weight.

6.5.2. Other Ingredients

A recombinant animal protein of the disclosure may be combined with other ingredients such as fats, carbohydrates, supplemental non-recombinant proteins, fiber, nutritional supplements (e.g., minerals, and vitamins) to make a food composition.

In some embodiments, the recombinant animal protein of the disclosure may be combined with other ingredients to make a food product that meets the nutritional requirements of an animal (i.e., a nutritionally balanced food product). In some embodiments, the recombinant animal protein of the disclosure may be combined with other ingredients to make a food product more palatable to an animal or an individual.

In some embodiments, the recombinant animal protein of the disclosure may be combined with other ingredients to meet the nutritional requirements of an animal and to make it more palatable to an animal or an individual.

6.5.2.1. Amino Acids

For some food compositions, such as a primary diet food, it may be desirable to combine the recombinant animal protein with additional amino acids. Any amino acid that makes a food composition nutritionally balanced for an animal can be added to a food composition of the disclosure. Examples of amino acids that can be added to a food composition of the disclosure include but are not limited to Arginine, Histidine, Isoleucine, Leucine, Lysine, Methionine, Methionine/Cystine, Phenylalanine, Phenylalanine/Tyrosine, Threonine, Tryptophan, and Valine.

6.5.2.2. Fat and Carbohydrates

For some food compositions, it may be desirable to combine the recombinant animal protein with fat and/or carbohydrates.

Fat and carbohydrates are obtained from a variety of sources including but not limited to animal fat, fish oil, vegetable oil, meat, meat by-products, grains, other animal or plant sources, or any combination thereof.

In some embodiments, the food product can comprise omega-3 polyunsaturated fatty acids such as docosahexaenoic acid (“DHA”) or eicosapentaenoic acid (“EPA”) or a mixture of DHA and EPA.

Grains include but are not limited to rice, wheat, corn, barley, buckwheat, sorghum, oats, and quinoa. Other plant sources include but are not limited to pulses (chickpeas and different beans) and edible roots (e.g., potato, sweet potato, carrot, cassava, and turnips).

6.5.2.3. Non-Recombinant Proteins

For some food compositions, it may be desirable to combine the recombinant animal protein with additional proteins (i.e., also referred to as “supplementary proteins” or “non-recombinant proteins”).

Such, supplementary proteins or non-recombinant proteins, can be obtained from a variety of sources including plants, animals, or microbes (unicellular and multicellular).

Supplemental proteins may also be obtained from an animal, which includes meat, meat by-products, dairy, and eggs. Meats include the flesh from poultry, fish, and animals such as cattle, swine, sheep, goats, deer, and the like. Meat by-products include but are not limited to kidneys, lungs, livers, stomachs, and intestines.

In some embodiments, the supplementary proteins may be free amino acids and/or peptides.

6.5.2.4. Fiber

For some food compositions, it may be desirable to combine the recombinant animal protein with fiber. Fiber can be obtained from a variety of sources such as vegetable fiber sources, including but not limited to beans, cellulose, beet pulp, parsnips, broccoli, peanut hulls, carrots, spinach, and soy fiber.

6.5.2.5. Nutritional Supplements

For some food compositions, it may be desirable to combine the recombinant animal protein with nutritional supplements. The nutritional supplement can be an antioxidant, a vitamin, a mineral, or a nutrient.

The nutritional supplements may be obtained from a variety of sources known to people skilled in the art including commercial sources. Vitamins and minerals can be added to a food product in amounts required to avoid deficiency and maintain health.

Non-limiting examples of nutrients that can be used with the disclosure include but are not limited to choline, thiamine, egg powder, manganese, methionine, cysteine, L-carnitine, lysine, and mixtures thereof.

Non-limiting examples of antioxidants include but are not limited to vitamin E, vitamin C, taurine, beta-carotene, and mixtures thereof.

Vitamins generally useful as food additives include vitamin A, vitamin B1, vitamin B2, vitamin B6, vitamin B12, vitamin D, vitamin E, biotin, vitamin K, folic acid, inositol, niacin, pantothenic acid, niacin, pyridoxine, choline, and mixtures thereof.

Minerals and trace elements useful as food additives include calcium, phosphorus, sodium, chloride, potassium, magnesium, iron, copper, zinc, selenium, iodine, and mixtures thereof. In certain embodiments, the food compositions can further comprise taurine.

6.5.2.6. Palatability Agents

The food composition of the disclosure may comprise one or more palatability agents. The palatability agents are typically added to a food composition to enhance the overall palatability of the food to overcome any negative effects to flavor or smell.

The palatability agents may be added to enhance mouth feel or attractiveness of the food product, such as dyes or any other colorant that can change the color of the food composition.

A flavoring agent may be a flavoring molecule(s) and/or flavoring precursor(s). Flavoring agents may include carbohydrates, sugars, nucleic acids (e.g., nucleotides and/or nucleosides), free fatty acids, amino acids and/or derivatives, vitamins, minerals, antioxidants, or any combination thereof.

Carbohydrates and sugars may include but are not limited to, glucose, fructose, ribose, sucrose, arabinose, inositol, maltose, molasses, maltodextrin, glycogen, glycol, galactose, lactose, sorbitol, amylose, amylopectin, xylose, or any combination thereof.

Nucleic acids may include but are not limited to, inosine, inosine monophosphate, guanosine, guanosine monophosphate, adenosine, adenosine monophosphate, or any combination thereof. Free fatty acids may include but are not limited to, arachidic acid, behenic acid, caprylic acid, capric acid, cerotic acid, erucic acid, lauric acid, linoleic acid, myristic acid, palmitic acid, palmitoleic acid, stearic acid, lignoceric acid, or any combination thereof.

Amino acids and/or amino acid derivatives may include but are not limited to, cysteine, cystine, cysteine sulfoxide, allicin, selenocystein, methionine, isoleucine, leucine, lysine, phenylalanine, threonine, tryptophan, 5-hydroxy tryptophan, valine, arginine, histidine, alanine, asparagine, aspartate, glutamate, glutamine, glycine, proline, serine, tyrosine, taurine, or any combination thereof. Amino acids may be added to the food product as free amino acids or as amino acid derivatives. For example, any amino acid may be added to the food product as a free amino acid (e.g., pre-digested amino acids without other functional groups of chemical moieties).

Flavoring agents may include, but are not limited to retinol, retinal, beta-carotene, thiamine, riboflavin, niacin, niacinamide, nicotinamide, riboside, pantothenic acid, pyridoxine, pyridoxamine, pyridoxal, biotin, folates, cyanocobalamin, hydroxocobalamin, methylcobalamin, adenosylcobalamin, ascorbic acid, cholecalciferol, ergocalciferol, tocopherols (e.g., alpha-tocopherol), tocotrienols, phylloquinone, menaquinones, potassium, chlorine, sodium, calcium, phosphorus, magnesium, iron, zinc, manganese, copper, iodine, chromium, molybdenum, selenium, cobalt, or any combination thereof.

Antioxidants may include, but are not limited to, beta-carotene, alpha-tocopherol, quercetin, caffeic acid, propyl gallate, epigallocatechin gallate, or any combination thereof.

In some embodiments, zeolite is added to animal food compositions in amounts sufficient to enhance palatability. Preferably in amounts of zeolite that can be added to a food composition range from about 0.01% to about 4% by weight of the food composition.

6.5.3. Pet Food and Feed Compositions

Various pet foods (companion animals) and animal feed (livestock, zoo animal) compositions are also provided. A pet food or animal feed composition can be made by combining a recombinant animal protein provided herein with a variety of other ingredients (as provide in Section 6.5.2) and/or additives or preservatives to generate a pet food or feed product. The one or more ingredients may be a wet ingredient, a dry ingredient, or other ingredients as provided herein, or any combination thereof. The pet food can be in various formats such as a kibble, a freeze-dried food product, a dehydrated food product, a baked food product, or raw formats.

6.5.3.1. Food or Feed Formulations

The food or feed product can be made in various formulations. The amount of the other ingredients can be mixed with the recombinant animal protein to make the food or feed formulation will depend on the dietary requirements of a companion animal, livestock, zoo animal, which can depend on the species, age, size, weight, growth stage, health condition, and/or organ function (e.g., liver, heart, join, hip, or brain) of the animal.

In some embodiments, the pet food of feed comprising a recombinant animal protein is formulated to be nutritionally balanced. As used herein, the term “nutritionally balanced,” with reference to the pet food or feed composition, means that the composition has known required nutrients based on recommendations of recognized authorities in the field of pet nutrition.

For example, the recommended nutrients and their amounts have been established for various animals. See, National Research Council (NRC) provides recommended amounts of such nutrients for farm animals; nutrient Requirements of Swine (11th Rev. Ed., National Academy Press, Wash. D.C., 2012); Nutrient Requirements of Poultry (9th Rev. Ed., National Academy Press, Wash. D.C., 1994); Nutrient Requirements of Horses (6th Rev. Ed., National Academy Press, Wash. D.C., 2007), each of which are incorporated in their entirety.

The American Feed Control Officials (AAFCO) provides recommended amounts of such nutrients for dogs and cats. See American Feed Control Officials, Inc. (Official publication, 2018). In some embodiments, the food product comprises the AAFCO nutrient profile established for a dog. In some embodiments, the food product comprises the AAFCO nutrient profile established for a cat.

In some embodiments, the feed comprises at least the minimum or the maximum nutrient concentrations as established by NRC for various farm animals, pig, sheep, chicken, horse, goat, and the like.

Preferably, the food composition will include, by mass, 5-50% protein, 0.01-1.5% sodium, 0.01-1.5% potassium, 0-50% fat, 0-75% carbohydrate, 0-40% dietary fiber, and 0-15% of other nutrients.

The food product comprising a recombinant protein composition can be formulated into a breed-specific food formulation. In some embodiments, the proteins for breed-specific food formulations can be based on growth rate. See for example U.S. Pat. No. 5,851,573, which is hereby incorporated by reference in its entirety. In some embodiments, the proteins for breed-specific food formulations can be based on phenotypic characteristics of the animal. See for example U.S. Pat. No. 6,669,975, which is hereby incorporated by reference in its entirety. In some embodiments, the proteins for breed-specific food formulations can be based on genomic methods. See for example US Publication No. 20060045909, which is hereby incorporated by reference in its entirety.

In some embodiments, the food or feed product can be formulated into a product that improves health or wellness. In some embodiments, the food or feed further comprises a compound that improves joint function, skin health, coat or hair, brain development, or improves stool quality and/or stool frequency.

6.5.3.2. Form and Shape

The pet food or feed product (dry or wet) can be in any form useful for feeding the food composition to an animal. The food product may be a shaped and/or molded or non-shaped product. For example, the food product may comprise shaped treats, kibble, edible granules, or made into a toy-shaped food product.

The pet food or feed product may be formulated for mouthfeel. Mouthfeel of the pet food product may be formulated according to its structure, dryness, density, adhesiveness, bounce, chewiness, coarseness, cohesiveness, fracturability, graininess, gumminess, hardness, heaviness, moisture adsorption, moisture release, mouthcoating, roughness, slipperiness, smoothness, springiness, uniformity, and viscosity.

The pet food or feed product may be formulated to have a porous, fibrous, or amorphous structure. In an example, the pet food product has a fibrous structure. The pet food product may be formulated for fracturability such that the product crumbles, cracks, or shatters. Fracturability may encompass crumbliness, crispness, crunchiness, and brittleness.

6.5.3.3. Dry Pet Food and Feed

In some embodiments, the food product is a dry pet food or feed product for a companion animal, or dry feed for livestock, zoo animal or a pet.

The dry pet food or feed product can be made completely of the recombinant animal protein. In some other embodiments, the dry pet food can comprise about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of the recombinant animal protein.

A dry pet food or feed product can be prepared by adding one or more dry ingredients. Other ingredients that can be added to a dry food product include but are not limited to the ingredients provided in Section 6.5.2.

The dry pet food or feed product can be freeze-dried, dehydrated, or air-dried. In some embodiments, the recombinant animal protein can be a coating on another dry food product. In some embodiments, the dry food product is a kibble.

The dry pet food or feed can have the nutrient profile required for a dog or cat as provided by the AAFCO guidelines. In some embodiments, the dry feed has the nutrient profile as established by NRC for various farm animals.

Kibbles are generally formed using an extrusion process in which the mixture of dry and wet ingredients is mechanically worked at high temperature and pressure and pushed through small openings and cut off into kibble by a rotating knife. Kibble also can be made using a baking process when the mix is placed into a mold before dry-heat treatment.

In some embodiments, the recombinant animal protein composition is coated onto the dry kibble, incorporated into the kibble, or both. Other processes such as spraying, soaking, or brushing may be used to either coat the composition on the exterior or inject the recombinant animal protein composition into an existing dry kibble.

6.5.3.4. Wet Pet Food and Feed

The disclosure also provides wet pet food products for a companion animal, or wet feed for livestock or a zoo animal. A wet pet food or feed can be prepared by adding one or more wet ingredients such as water containing host cells comprising recombinant animal protein, water, oils, fats, or vegetables or a combination thereof. Other non-limiting ingredients that can be added to a dry food product are provided in Section 6.5.2. In some embodiments, the wet food product is raw.

The wet pet food or feed can be made completely of the recombinant animal protein. In some other embodiments, the wet pet food or feed can comprise about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of the recombinant animal protein.

The wet pet food or feed can have the nutrient profile required for a dog or cat as provided by the AAFCO guidelines. In some embodiments, the wet feed has the nutrient profile as established by NRC for various farm animals.

The wet kibble can be a dried kibble that is coated with one or more wet topical coatings supplied as intermediate food product of the disclosure. In some embodiments, wet kibble can be made by mixing the kibble into a gravy-like liquid supplied as an intermediate food product of the disclosure.

6.5.3.5. Pet Treats

The disclosure also provides treats for a companion animal, livestock, or a zoo animal. The treat can be a dry treat, an edible toy, or a chewable toy. The treat can be made completely of the recombinant animal protein. In some other embodiments, the treat can comprise about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, or 90% of the recombinant animal protein.

Treats of the present invention can be prepared by an extrusion or baking process similar to those used for dry food. Treats of the disclosure can be prepared by a molding process. Treats can also be in the form of a chew toy. Chewable toys can include but are not limited to, artificial bones and food compositions shaped to look like natural foods that are appealing to the animal.

Often, a pet treat will have nutritional value. Nutritional treats may contain one or more nutrients required for a primary food product. Non-nutritional treats can have minimal nutrition of a primary food product. Treat may also be mixed with other ingredients. Other non-limiting ingredients that can be added to a pet treat include provided in Section 6.5.2.

In some embodiments, the treat further comprises a compound that improves health or wellness. In some embodiments, the treat furthers comprise a compound that improves joint function, skin health, coat or hair, brain development, or improves stool quality and/or stool frequency.

In some embodiments, the recombinant animal protein composition is coated onto the treat, incorporated into the treat, or both. Other processes such as spraying, soaking, or brushing may be used to either coat the recombinant animal protein as an intermediate food product composition on the exterior of the treat or inject it into an existing treat form.

6.5.3.6. Packaging

The food compositions can be packaged in cans, trays, tubs, pouches, bags, or any other suitable container.

### 6.6. Supplements

The disclosure provides supplements for a human or animal. A dietary supplement is a product intended to supplement the diet. The recombinant animal protein can be harvested and provided to the supplement composition as a whole-cell food composition, a protein concentrate food composition, or as a protein isolate food composition.

In some embodiment the supplement is made solely from at least one animal protein provided by the disclosure. In other embodiments, the animal protein is combined with other ingredients or nutrients. Other ingredients include but are not limited to those in Section 6.5.2.

In some embodiments, a supplement can be taken by mouth. Where a supplement is formulated to be taken by mouth, it can be in the form of a pill, a capsule, a tablet, a liquid, soup, broth, or a dissolvable powder. In some embodiment, the supplement can a dry protein mixture of one or more recombinant animal proteins.

In other embodiments, a supplement can be incorporated into a commercially available food product. In some embodiments, the recombinant animal proteins is incorporated into a commercially available food product at a percentage (based on dry mass) of 0.1-95%, typically between 10% and 90%, more typically between 5% and 50%, including ranges of 5%-10%, 10-20%, 20-30%, 30-40%, 40-50%, but also including 60-70%, 70-80% and 80%-90% and combinations of these ranges (e.g., 30%-70%).

In some embodiments, the recombinant animal protein can be incorporated into commercially available food product to increase the percentage of an essential amino acid in the product. The percentage of one or more essential amino acids can be increased in a commercially available food product by about 0.1%, 0.2%, 0.3%, 0.4%, 0.5%, 0.6%. 0.7%, 0.8%, 0.9%, 1%, 2%, 3%, 4%, 5%, 6%, 7%, 8%, 9%, 10%, 20%, 30%, 40%, 50%, 60%, 70%, 80%, or 90% (based on dry mass).

### 6.7. Pharmaceutical Compositions

The disclosure also provides various pharmaceutical compositions comprising a recombinant animal protein of the disclosure that improves the health or wellness of a human or an animal.

These compositions can comprise, in addition to the recombinant animal protein, a pharmaceutically acceptable excipient, carrier, buffer, stabilizer or other materials well known to those skilled in the art. Such materials should be non-toxic and should not interfere with the efficacy of the active ingredient.

The precise nature of the carrier or other material can depend on the route of administration, e.g., oral, intravenous, cutaneous or subcutaneous, nasal, intramuscular, or intraperitoneal routes

6.7.1. Improves Health or Wellness

A pharmaceutical composition can be made by combining a recombinant animal protein provided herein with a compound known or capable of improving the health or the wellness of an animal.

In some embodiments, the pharmaceutical composition comprises a recombinant animal protein of the disclosure and a compound that improves hip function.

In some embodiments, the pharmaceutical composition comprises a recombinant animal protein of the disclosure and a compound that improves joint function. In some embodiments, the pharmaceutical composition comprises a recombinant animal protein of the disclosure and a compound that improves skin health. In some embodiments, the pharmaceutical composition a recombinant animal protein of the disclosure and a compound that improves coat or hair. In some embodiments, the pharmaceutical composition comprises a recombinant animal protein of the disclosure and a compound that improves brain development. In some embodiments, the pharmaceutical composition comprises a recombinant animal protein of the disclosure and a compound that improves stool quality and/or stool frequency.

Wellness of an animal herein encompasses all aspects of the physical, mental, and social well-being of the animal, and is not restricted to the absence of infirmity. Wellness attributes include without limitation states of disease or physiological disorder, states of parasitic infestation, hair and skin condition, sensory acuteness, dispositional and behavioral attributes, and cognitive function. Conditions adverse to wellness encompass not only existing diseases and physiological including, mental, behavioral, and dispositional disorders, but predisposition or vulnerability to such diseases or disorders. Asymptomatic are likewise encompassed.

6.7.2. Formulations

Pharmaceutical compositions for oral administration can be in tablet, capsule, powder or liquid form. A tablet can include a solid carrier such as gelatin or an adjuvant. In some embodiments, the capsule can be made from a vegetarian material such as agar, vegetable cellulose, and the like. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In some embodiments, the pharmaceutical composition can be in the form of nutritional feed, a food product, or a treat.

6.7.3. Methods of Treating

The disclosure also provides methods of treatment for an animal diagnosed or suffering from a disease or disorder.

The method can comprise administering a therapeutic-effective amount of the pharmaceutical composition provided herein alone or in combination with another agent or treatment to promote health or wellness.

In some embodiments, the method includes administering a therapeutically effective amount of the pharmaceutical composition to an animal diagnosed or suffering from a disease or disorder. In yet some other embodiments, the method includes administering a prophylactically effective amount of the pharmaceutical composition to an animal genetically predisposed to a disease or a disorder.

A genetically predisposed animal can be based on the breed, age, size, or any other physical characteristic.

6.7.4. Administration

For treatment purposes, administration of the pharmaceutical composition is preferably administered to an animal in a “therapeutically effective amount.” In some embodiments, the pharmaceutical composition is preferably administered to an animal in a “prophylactically effective amount” to the animal or individual.

The actual amount administered, and rate and time-course of administration will depend on the nature and severity of disease or disorder being treated.

Prescription of treatment, e.g., decisions on dosage, etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed.), 1980.

6.7.5. Combination Therapy

This disclosure also provides combination therapies where the pharmaceutical composition is administered in combination with another therapeutic agent or treatment.

In some embodiments, the pharmaceutical composition can be administered either simultaneously or sequentially, dependent upon the condition to be treated.

Non-limiting examples of a therapeutic treatment include physical therapy, surgery, radiation, and dietary restrictions for diseases such as diabetes.

### 6.8. Additional Aspects & Embodiments

In a first additional aspect, the disclosure provides various food compositions comprising at least one recombinant animal protein.

Embodiment 1. A food composition, wherein said food composition is formulated for a companion animal, and wherein the food composition comprises at least one recombinant animal protein.

Embodiment 2. The food composition of embodiment 1, wherein the food composition is substantially free of antibiotics, animal growth hormones, and processed animal meat.

Embodiment 3. The food composition of any of the above embodiments, wherein the at least one recombinant animal protein is a recombinant animal muscle protein.

Embodiment 4. The food composition of embodiment 3, wherein the at least one recombinant animal muscle protein is selected from the animal muscle proteins in Table 1.

Embodiment 5. The food composition of embodiment 4, wherein the food composition comprises at least two recombinant animal muscle proteins.

Embodiment 6. The food composition of any of the above embodiments, wherein at least one recombinant animal muscle protein comprises a modified amino acid sequence, wherein said modification is relative to the naturally occurring sequence of the animal muscle protein.

Embodiment 7. The food composition of embodiment 6, wherein said modified recombinant animal muscle protein comprises an amino acid sequence at least 80% identical to a sequence in Table 1.

Embodiment 8. The food composition of embodiment 6, wherein said modified recombinant animal muscle protein is a truncated form of a sequence in Table 1.

Embodiment 9. The food composition of embodiment 6, wherein said modified recombinant animal muscle protein comprises a heterologous signal peptide.

Embodiment 10. The food composition of any of the above claims, wherein the food composition consists of 5% to 95% recombinant animal protein, on a mass percentage basis.

Embodiment 11. The food composition of embodiment 10, wherein the food composition consists of 5% to 40% recombinant animal protein, on a mass percentage basis.

Embodiment 12. The food composition of embodiment any of the above claims, wherein the food composition includes 5-50% protein, 0.01-1.5% sodium, 0.01-1.5% potassium, 0-50% fat, 0-75% carbohydrate, 0-40% dietary fiber, and 0-15% of other nutrients.

Embodiment 13. The food composition of embodiment 10, wherein the food composition consists of 40% to 95% recombinant animal protein.

Embodiment 14. The food composition of embodiment 10, wherein the food composition consists of 1% to 30% recombinant animal protein.

Embodiment 15. The food composition of any of the above embodiments, wherein the food composition is formulated for a dog or a cat.

Embodiment 16. The food composition of any of the above embodiments, wherein the food composition is customized for a particular companion animal or a selected cohort of companion animals with particular dietary needs.

In a second additional aspect, the disclosure provides methods for preparing the food compositions described herein.

Embodiment 17. A method for preparing any of the food compositions described above, wherein the method comprises recombinantly expressing at least one recombinant animal protein in a eukaryotic host organism.

Embodiment 18. The method of embodiment 17, wherein the eukaryotic host organism is a yeast cell.

Embodiment 19. The method of embodiment 17 or 18, wherein the recombinantly expressed animal protein is secreted by the eukaryotic host organism.

Embodiment 20. The method of any one of embodiments 17-19, wherein the recombinantly expressed animal protein is isolated from the host organism and the growth medium before mixing with other components in the food composition.

Embodiment 21. The method of embodiment 20, further comprising mixing the at least one recombinantly expressed animal protein with one or more food components selected from the group consisting of sodium, potassium, fat, carbohydrate, and dietary fiber, and then forming the mixture into a food composition suitable for consumption by an animal.

Embodiment 22. The method of embodiment 21, wherein at least two animal proteins are recombinantly expressed in a eukaryotic host and isolated prior to mixing with the one or more food components.

Embodiment 23. The method of embodiment 17 or 18 wherein the recombinantly expressed protein is not isolated from the host organism prior to mixing with other components in the food composition.

In a third additional aspect, the disclosure provides additional methods for preparing the food compositions described herein.

Embodiment 24. A method for preparing any of the food compositions described above, wherein the method comprises mixing at least one recombinantly expressed animal muscle protein with one or more compositions selected from the group consisting of sodium, potassium, fat, carbohydrate, and dietary fiber, and then forming the mixture into a food composition suitable for consumption by an animal.

In a fourth additional aspect, the disclosure provides additional formulations of food compositions comprising at least one recombinant animal protein.

Embodiment 25. A food composition, wherein said food composition is formulated for a human, and wherein the food composition comprises at least one recombinant animal muscle protein.

### 6.9. Examples

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology used in the art. Also referred to below are the following references: (1) M. R. Green and J. Sambrook, Molecular Cloning: A Laboratory Manual, 4th Edition, Cold Spring Harbor, N.Y.: Cold Spring Harbor Laboratory Press, 2012, pp. 1009-1011; (2) G. C. U. F. T. Tool, GenScript, [Online]. Available: https://www.genscript.com/tools/codon-frequency-table. [Accessed 18 12 2018]; (3) S. Wu and L. J. Geoffrey, “High efficiency transformation by electroporation of Pichia pastoris pretreated with lithium acetate and dithiothreitol,” Drug Discovery and genomic technologies, vol. 36, no. 1, pp. 152-154, 2004; (4) S. Kawai, W. Hashimoto and K. Murata, “Transformation of Saccharomyces cerevisiae and other fungi,” Bioengineered Bugs, vol. 1, no. 6, pp. 395-403, 2010; (5) P. Manivasakam and R. H. Schiestl, “High efficiency transformation of Saccharomyces cerevisiae by electroporation,” Nucleic Acids Research, vol. 21, no. 18, pp. 4414-4415, 1993.

### 6.9.1. Example 1: Expression of Recombinant Actin Protein in a Eukaryotic Host Cell

Actin is the major component of the cytoskeleton. It exists in two different forms, a monomeric form (G-actin) and a filamentous form (F-actin). G-actin polymerizes to form F-actin, and it is primarily these filaments that participate in processes such as cell motility, transport, and cytokinesis [20]. The actin-binding domain is highly conserved amongst species. Actin-binding proteins share a common binding area on the actin surface, consistent of the cleft between actin subdomains 1 and 3 [21]. There is also a nucleotide-binding site, which is a cleft between subdomains 2 and 4. The binding of adenosine 5′-triphosphate or ATP and subsequent hydrolysis into adenosine 5′-diphosphate or ADP is known to be a critical element in controlling the association of actin with itself and with other proteins. When ATP is bound to actin it polymerizes faster and dissociates slower than ADP-actin [22].

Single and double mutants of the ATP-binding site of actin will ablate its toxicity in eukaryotic expression hosts and thus increase expression levels. The residues targeted by mutagenesis are P-72, E-74, 1-77, and T-79 (numbering for pig (UniProtKB/Swiss-Prot: P68137), chicken (UniProtKB/Swiss-Prot:P68139), and cow (UniProtKB/Swiss-Prot:P68138)). Recombinant actin protein mutated at these sites will be over-expressed in a eukaryotic host organism, isolated, and incorporated into a companion animal food product.

In one embodiment, then, the invention provides a food composition comprising a recombinant actin protein, wherein said recombinant actin protein comprises one or more mutations from the group consisting of P-72, E-74, 1-77 and T-79. In certain related embodiments, the recombinant actin protein is a fragment of actin protein comprising the aforementioned residues.

### 6.9.2. Example 2: Identification of Recombinant Actin Sequences for Over-Expression in Eukaryotes

Actin is highly conserved between widely divergent eukaryotic species. For instance, there is 87% sequence identity (325 of 374 amino acids) between yeast and human actin. Comparing chicken, cow, pig, human, and Saccharomyces cerevisiae, there are 319 conserved residues. A library of point mutations is made at each of these conserved positions and those mutations that are permissive of high levels of expression of mutant actin are identified.

### 6.9.3. Example 3: Engineered Animal Proteins for Over-Expression in Eukaryotic Cells

Error-prone PCR with/without shuffling will be used across the DNA coding sequence (cDNA) to create mutated DNAs encoding animal protein sequences. Eukaryotic hosts recombinantly expressing the mutant sequences will be screened for high growth and high expression of the target protein.

The genes and the proteins encoded by the genes may also be truncated in order to yield a high expression and fast cell growth. Modifications of the gene sequence (e.g., the addition or removal of certain amino acids) will, in some cases, increase cell viability and increase the rate of cell division. Proteins that are too large to overexpress efficiently will be truncated in order to increase the expression level.

### 6.9.4. Example 4: Insertion of a Chicken Coronin Gene into an Saccharomyces cerevisiae Strain and Extracellular Expression of the Corresponding Protein

The expression vector pD1214-FAKS (ATUM) contains the 2-micron origin of replication, which encodes proteins that allow yeast cells to maintain 20-50 copies of recombinant plasmid per cell. Because 2-micron plasmids are maintained at such high copy numbers, they provide a convenient way to monitor the effects of overproduction of a particular gene product. The plasmid also contains a bacterial origin of replication (Ori_pUC) which allows production of greater than 500 copies of plasmid per cell in Escherichia coli. It is replicated in Escherichia coli TOP10 cells grown in Low Salt Luria-Bertani medium (5 g/L NaCl) including 100 μg/mL carbenicillin as selective pressure at 37° C. The vector also contains the alpha factor, which is a secretion signal derived from the yeast mating pheromone alpha-factor in Saccharomyces cerevisiae and facilitates secretion of heterologous proteins in yeast. The plasmid is purified from E. coli by methods well known in the art, using for instance a commercially available plasmid prep kit, such as the QIAGEN Plasmid Mini Kit. The vector is linearized using a SapI restriction enzyme followed by enzymatic dephosphorylation using established molecular cloning methods. The gene encoding the protein product of interest, chicken coronin, can also be ordered in the selected vector from contract cloning vendors such as ATUM (Newark, Calif.). This plasmid contains features such as the strong constitutive promoter TEF1, encoding translation-elongation factor 1 alpha and the gene coding for ampicillin resistance (beta lactamase). The vector also contains an auxotrophic marker URA3, which encodes orotidine-5′ phosphate decarboxylase, an enzyme that is required for the biosynthesis of uracil.

Linearized plasmid is separated using agarose gel electrophoresis. An agarose gel section containing linearized plasmid is collected and the linearized plasmid is purified from the agarose using a commercially available DNA purification kit, e.g. the QIAquick Gel Extraction Kit (Qiagen).

The gene sequence for chicken coronin can be obtained from UniProt.org under accession number F1NXA5. The double-stranded DNA is constructed through chemical gene synthesis from either ATUM (Newark, Calif.), Genscript (Piscataway, N.J.), or IDT (Coralville, Iowa). It is supplied in a vector of choice. The DNA sequence can also be obtained via amplification of cDNA generated directly from a biological sample, such as a tissue or a blood sample. The gene sequence is modified to aid in cloning, gene expression, or enhance production. It is “codon optimized”, i.e., triplet DNA sequences that are not commonly used in the expression host are changed to those that are commonly used. The specific species in this case is Saccharomyces cerevisiae and the codon usage table is obtained from GenScript.

The codon optimized coronin gene (CORO6), containing exons, but no introns, is ligated to the linearized and purified vector via enzymatic ligation to generate a vector capable of being inserted into a host organism. Electroporation and other methods of transformation are well known in the art. The vector containing the ORF is transformed into the host strain (S. cerevisiae, in this example) via electroporation using of 1.5 kV, 25 μF, and 200Ω. Chemical transformation or another method can also be used. Transformed cells are plated onto minimal media lacking uracil and incubate at 30° C. until heterotrophic colonies arise in 2-3 days. Colonies are picked and transferred into cultures of minimal media or YPD and grown for 24-90 hours at 28-30° C. The successful clone is confirmed by sequencing for insert identity and copy number using established methods such as PCR, q-PCR, or Southern Blot.

The supernatant is analyzed for secreted protein expression by SDS-PAGE. Isolated clones expressing the secreted protein will be cultured, and the recombinantly expressed protein is isolated from the engineered yeast cells or, if secreted, is isolated from the medium. The secreted, recombinantly expressed protein is then formulated into a food composition for animals, preferably companion animals. In one embodiment, then, the disclosure provides a food composition comprising a recombinantly expressed chicken coronin protein. In certain embodiments, the recombinantly expressed chicken coronin protein is harvested from yeast cultures, wherein the yeast has been engineered to express the protein.

### 6.9.5. Example 5: Insertion of a Pig Myozenin Gene into a Komagataella phaffii Strain and Intracellular Expression of the Corresponding Protein

The expression vector pD902 (ATUM, Newark, Calif.) contains a bacterial origin of replication (OripUC) which allows production of greater than 500 copies of plasmid per cell in Escherichia coli. It is replicated in Escherichia coli TOP10 cells grown in Low Salt Luria-Bertani medium (5 g/L NaCl) including 25 μg/mL zeocin as selective pressure at 37° C. The plasmid is purified by a method well known in the art, using for instance a commercially available plasmid prep kit, such as the QIAGEN Plasmid Mini Kit. The vector is linearized using a SapI restriction enzyme and performing dephosphorylation using established molecular cloning methods [1]. The gene can also be ordered in the selected vector. This plasmid contains features such as the AOX1 promoter used for recombinant gene expression and the resistance marker for zeocin. Linearized plasmid is separated using agarose gel electrophoresis. An agarose gel section containing linearized plasmid is collected and the linearized plasmid is purified from the agarose using a commercially available DNA purification kit, e.g. the QIAquick Gel Extraction Kit (Qiagen).

The gene sequence for pig myozenin can be obtained from UniProt.org under accession number Q4PS85. The double-stranded DNA is constructed through chemical gene synthesis from either ATUM (Newark, Calif.), Genscript (Piscataway, N.J.), or IDT (Coralville, Iowa). It is supplied in a vector of choice. The DNA sequence can also be obtained via amplification of cDNA generated directly from a biological sample, such as a tissue or a blood sample. The gene sequence is modified to aid in cloning, gene expression, or enhance production. It is “codon optimized”, i.e. triplet DNA sequences that are not commonly used in the expression host are changed to those that are commonly used. The specific species in this case is Komagataella phaffii (previously Pichia pastoris) and the codon usage table is obtained from GenScript [2]. The strain PPS-9016 is protease-deficient (ATUM, Newark, Calif.). Other variants of Komagataella phaffii can also be used.

The codon optimized myozenin gene (MYOZ1), containing exons, but no introns, is ligated to the linearized and purified vector via enzymatic ligation to generate a vector capable of being inserted into a host organism. The method used is known in the art and the protocol can be obtained from a molecular cloning manual [1]. The vector containing the gene, also called ORF open reading frame) is linearized using the PmeI restriction enzyme. Twenty micrograms of DNA are digested using the corresponding buffer of the restriction enzyme (from e.g. NEB) in a volume of 200 μL. Five μL of digested DNA is run on a 1% agarose gel and compared with an undigested control. The digested product is ethanol precipitated using 1/10 volume of 3M sodium acetate and 2.5 volumes of 100% ethanol. It is centrifuged to pellet the DNA and pellet is washed with 70% ethanol, air dried, and suspended in 20 μL of deionized sterile water or 10 mM Tris-Cl, pH 8.0. The linearized vector containing the ORF is transformed into the host strain.

Transformation is performed via electroporation using instrument settings of 1.5 kV, 25 μF, and 186-200Ω. Electrocompetent cells are obtained via methods known in the art [3]. Chemical transformation or another method can also be used. The vector containing the ORF is integrated into the chromosome of the host organism. The vector does not contain a yeast origin of replication and selected transformants, grown at 30° C. on YPD agar plates containing 100-1000 μg/mL zeocin and 1 M sorbitol, will contain the zeocin resistance gene integrated into the genome. Multiple insertions of the gene may be used. The successful clone is confirmed by sequencing for insert identity and copy number using established methods such as PCR, q-PCR, or Southern Blot [1].

Colonies are picked into BMGY broth with 250 μg/ml zeocin and are grown at 30° C. shaking at 250 rpm. After 2 days of incubation, 300 μL of BMMY broth is added to each well, and incubation is continued for an additional 2-4 days. The cells are pelleted by centrifugation and the cell pellets are lysed by methods known in the art, e.g. by sonication [1] and analyzed for protein expression by SDS-PAGE.

### 6.9.6. Example 6: Insertion of a Pig Myozenin Gene into a Komagataella phaffii Strain and Extracellular Expression of the Corresponding Protein

The expression vector pD912 (ATUM, Newark, Calif.) contains a bacterial origin of replication (Ori_pUC) which allows production of greater than 500 copies of plasmid per cell in Escherichia coli. It is replicated in Escherichia coli TOP10 cells grow in in Low Salt Luria-Bertani medium (5 g/L NaCl) including 25 μg/mL zeocin as selective pressure at 37° C. The vector also contains the alpha factor, which is a secretion signal derived from the yeast mating pheromone alpha-factor in Saccharomyces cerevisiae and facilitates secretion of heterologous proteins in yeast. The plasmid is purified by a well-known method, using for instance a commercially available plasmid prep kit, such as the QIAGEN Plasmid Mini Kit. The vector is linearized using a SapI restriction enzyme and performing dephosphorylation using established molecular cloning methods [1]. The gene can also be ordered in the selected vector. This plasmid contains features such as the AOX1 promoter used for recombinant gene expression and the resistance marker for zeocin. Linearized plasmid is separated using agarose gel electrophoresis. An agarose gel section containing linearized plasmid is collected and the linearized plasmid is purified from the agarose using a commercially available DNA purification kit, e.g. the QIAquick Gel Extraction Kit (Qiagen).

The gene sequence for pig myozenin can be obtained from UniProt.org under accession number Q4PS85. The double-stranded DNA is constructed through chemical gene synthesis from either ATUM (Newark, Calif.), Genscript (Piscataway, N.J.), or IDT (Coralville, Iowa). It is supplied in a vector of choice. The DNA sequence can also be obtained via amplification of cDNA generated directly from a biological sample, such as a tissue or a blood sample. The gene sequence is modified to aid in cloning, gene expression, or enhance production. It is “codon optimized”, i.e. triplet DNA sequences that are not commonly used in the expression host are changed to those that are commonly used. The specific species in this case is Komagataella phaffii (previously Pichia pastoris) and the codon usage table is obtained from GenScript [2]. The strain PPS-9016 is protease-deficient (ATUM, Newark, Calif.). Other variants of Komagataella phaffii can also be used.

The codon optimized myozenin gene (MYOZ1), containing exons, but no introns, is ligated to the linearized and purified vector via enzymatic ligation to generate a vector capable of being inserted into a host organism. The method used is known in the art and the protocol can be obtained from a molecular cloning manual [1]. The vector containing the gene, also called ORF open reading frame) is linearized using the PmeI restriction enzyme. Twenty micrograms of DNA are digested using the corresponding buffer of the restriction enzyme (from e.g. NEB) in a volume of 200 μL. Five μL of digested DNA is run on a 1% agarose gel and compared with an undigested control. The digested product is ethanol precipitated using 1/10 volume of 3M sodium acetate and 2.5 volumes of 100% ethanol. It is centrifuged to pellet the DNA and pellet is washed with 70% ethanol, air dried, and suspended in 20 μL of deionized sterile water or 10 mM Tris-Cl, pH 8.0. The linearized vector containing the ORF is transformed into the host strain via electroporation using instrument settings of 1.5 kV, 25 μF, and 186-200Ω. Electrocompetent cells are obtained via methods known in the art [3]. Chemical transformation or another method can also be used. The vector containing the ORF is integrated into the chromosome of the host organism. The vector does not contain a yeast origin of replication and selected transformants, grown at 30° C. on YPD agar plates containing 100-1000 μg/mL zeocin and 1 M sorbitol, will contain the zeocin resistance gene integrated into the genome. Multiple insertions of the gene may be used. The successful clone is confirmed by sequencing for insert identity and copy number using established methods such as PCR, q-PCR, or Southern Blot [1].

Colonies are picked into BMGY broth with 250 μg/ml zeocin and are grown at 30° C. shaking at 250 rpm. After 2 days of incubation, 300 μL of BMMY broth is added to each well, and incubation is continued for an additional 2-4 days. The supernatant is analyzed for secreted protein expression by SDS-PAGE.

### 6.9.7. Example 7: Insertion of a Chicken Coronin Gene into an Saccharomyces cerevisiae Strain and Intracellular Expression of the Corresponding Protein

The expression vector pD91248 (ATUM, Newark, Calif.) contains a bacterial origin of replication (OripUC) which allows production of greater than 500 copies of plasmid per cell in Escherichia coli. It is replicated in Escherichia coli TOP10 cells grown in Low Salt Luria-Bertani medium (5 g/L NaCl) including 100 μg/mL carbenicillin as selective pressure at 37° C. The plasmid is purified by a method well known in the art, using for instance a commercially available plasmid prep kit, such as the QIAGEN Plasmid Mini Kit. The vector is linearized using a SapI restriction enzyme and performing dephosphorylation using established molecular cloning methods [1]. The gene can also be ordered in the selected vector. This plasmid contains features such as the bidirectional galactose inducible promoter cassette pGAL1/pGAL10 and the gene coding for ampicillin resistance (beta lactamase). The vector also contains an auxotrophic marker URA3, which encodes orotidine-5′ phosphate decarboxylase, an enzyme that is required for the biosynthesis of uracil.

Linearized plasmid is separated using agarose gel electrophoresis. An agarose gel section containing linearized plasmid is collected and the linearized plasmid is purified from the agarose using a commercially available DNA purification kit, e.g. the QIAquick Gel Extraction Kit (Qiagen).

The gene sequence for chicken coronin can be obtained from UniProt.org under accession number F1NXA5. The double-stranded DNA is constructed through chemical gene synthesis from either ATUM (Newark, Calif.), Genscript (Piscataway, N.J.), or IDT (Coralville, Iowa). It is supplied in a vector of choice. The DNA sequence can also be obtained via amplification of cDNA generated directly from a biological sample, such as a tissue or a blood sample. The gene sequence is modified to aid in cloning, gene expression, or enhance production. It is “codon optimized”, i.e. triplet DNA sequences that are not commonly used in the expression host are changed to those that are commonly used. The specific species in this case is Saccharomyces cerevisiae and the codon usage table is obtained from GenScript [2].

The codon optimized coronin gene (CORO6), containing exons, but no introns, is ligated to the linearized and purified vector via enzymatic ligation to generate a vector capable of being inserted into a host organism. The method used is known in the art and the protocol can be obtained from a molecular cloning manual [1]. The vector containing the gene, also called ORF (open reading frame) is linearized using the NcoI restriction enzyme. Twenty micrograms of DNA are digested using the corresponding buffer of the restriction enzyme (from e.g. NEB) in a volume of 200 μL. Five μL of digested DNA is run on a 1% agarose gel and compared with an undigested control. The digested product is ethanol precipitated using 1/10 volume of 3M sodium acetate and 2.5 volumes of 100% ethanol. It is centrifuged to pellet the DNA and pellet is washed with 70% ethanol, air dried, and suspended in 20 μL of deionized sterile water or 10 mM Tris-Cl, pH 8.0. The linearized vector containing the ORF is transformed into the host strain.

Transformation is performed via electroporation using instrument settings of 1.5 kV, 25 μF, and 186-200Ω. Electrocompetent cells are obtained via methods known in the art [3]. Chemical transformation or another method can also be used. The vector containing the ORF is integrated into the chromosome of the host organism. The vector does not contain a yeast origin of replication and selected transformants, grown at 30° C. on CM agar minus uracil will contain the URA3 gene integrated into the genome. Incubate at 30° C. until colonies arise in 2-3 days. Multiple insertions of the gene may be used. The successful clone is confirmed by sequencing for insert identity and copy number using established methods such as PCR, q-PCR, or Southern Blot [1].

Colonies are picked into YPD broth and are grown at 28-30° C. shaking at 250 rpm for 24-90 hours. The cells are pelleted by centrifugation and the cell pellets are lysed by methods known in the art, e.g. by sonication [1] and analyzed for protein expression by SDS-PAGE.

### 6.9.8. Example 8: Production of Recombinant Cofilin-2 Protein from Chicken in an Saccharomyces cerevisiae Host Cell

This study was conducted to determine if an Saccharomyces cerevisiae host cell could produce a cofilin-2 protein from chicken.

Cofilin-2 reversibly controls actin polymerization and depolymerization in a pH-sensitive manner. The particular protein used here is muscle-specific.

### Methods

Identification of the Cofilin-2 Gene Sequences from a Chicken Genome

The sequence for the cofilin-2 gene in the chicken genome was obtained by searching https://www.ncbi.nlm.nih.gov. The NCBI reference number was NP_001004406.1. The amino acid sequence for cofilin-2 was:

[SEQ ID NO: 1]
MASGVTVNDEVIKVFNDMKVRKSSTPEEIKKRKKAVLFCLSDDKKQIIV
EEATRILVGDIGDTVEDPYTAFVKLLPLNDCRYALYDATYETKESKKED
LVFIFWAPESAPLKSKMIYASSKDAIKKKFTGIKHEWQVNGLDDIKDRS
TLGEKLGGNVVVSLEGKPL

### Codon Optimization of Cofilin-2

The amino acid sequence was codon optimized for expression in S. cerevisiae using ATUM's GeneGPS™ algorithm.

The codon optimized sequence for the chicken cofilin-2 gene was:

[SEQ ID NO: 2]
ATGGCATCAGGCGTCACAGTGAACGATGAAGTTATCAAGGTTTTCAACG
ATATGAAAGTTCGTAAGTCTAGCACCCCAGAGGAAATCAAAAAGAGAAA
AAAAGCTGTCTTGTTTTGTTTATCCGATGACAAAAAGCAGATTATTGTA
GAGGAAGCTACTAGAATCCTTGTGGGTGATATAGGTGACACTGTAGAGG
ATCCTTACACTGCCTTCGTCAAGTTGTTACCATTAAATGATTGCAGATA
TGCTCTCTACGACGCTACATACGAAACCAAGGAATCTAAAAAAGAGGAC
TTGGTTTTCATCTTTTGGGCCCCTGAATCCGCGCCACTGAAGAGTAAGA
TGATATACGCATCTTCAAAGGATGCAATTAAAAAAAAGTTCACAGGTAT
TAAGCATGAATGGCAAGTTAACGGGCTTGATGATATTAAAGATAGATCT
ACATTGGGTGAAAAGCTAGGCGGAAATGTTGTGGTTTCATTGGAAGGAA
AGCCACTATAA

### Cloning of Cofilin-2 Gene

The gene was synthesized by ATUM and cloned into the pD1248 (ATUM, Newark, Calif.) expression vector, which is a yeast integrating plasmid. The resulting plasmid was designated as (“pBOND4”). The gene was amplified using the cloning primers oBOND11 oBOND12 (see Table 11).

The resulting PCR fragment was digested with restriction enzymes XhoI and EcoRI, gel purified, and then ligated with T4 DNA ligase into the pRS424 (ATCC® 77105™) expression vector, which was linearized with the same restriction enzymes and dephosphorylated by Quick CIP (New England Biolabs). This generated the expression vector (“pBOND21”) which has a 2-micron origin of replication and can be selected by complementation of tryptophan auxotrophy.

The pBOND21 expression vector was introduced into an S. cerevisiae host cell ATCC®208288™ designated as (“sBOND1”) by transformation using Zymo Research™ Frozen-EZ Yeast Transformation II kit following the manufacturer's instructions. The empty vector (“pBOND8”) was transformed into the sBOND1 strain and ran in parallel as a control.

### Cell Culture

Cells were grown in flasks on selective media (lacking tryptophan) containing 2% (w/v) raffinose until they reached an OD600 of 1 (i.e., exponential phase). During the exponential growth phase, galactose was added to the flask at a final concentration of 2% (w/v) to induce the expression of the cofilin-2 protein. After induction, the cultures were grown for another 24 hours with vigorous shaking.

### Protein Analysis

Cells were collected by centrifugation. Cell pellets were weighed, and protein extracts were prepared using the Thermo Scientific™ YPER Yeast Protein Extraction Reagent according to manufacturer's instructions. The protein extracts were quantitated using the Pierce™ BCA Protein Assay Kit according to manufacturer's guidelines. Equal amounts of total protein were loaded for each lane and then analyzed by SDS-PAGE. Proteins were visualized by Coomassie staining.

### Results

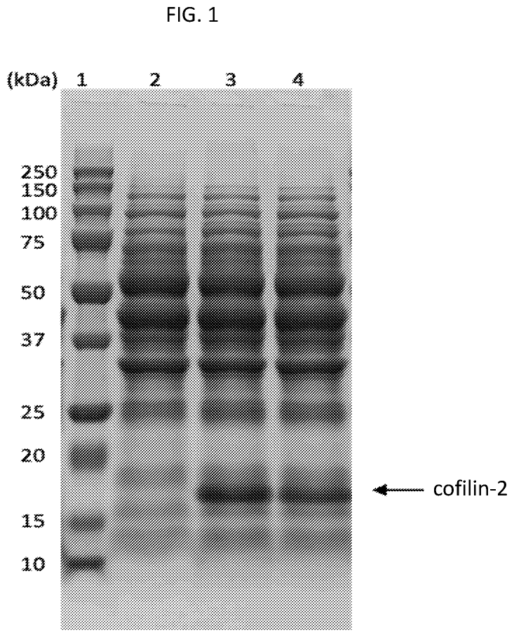

FIG. 1 is a photograph of the SDS-PAGE gel. The size of the cofilin-2 protein is 19 kDa. Lane 1 shows the molecular weight marker with the kDa sizes indicated. Lane 2 shows the host cell (sBOND1) with the empty vector (pBOND8), a control. Lane 3 shows a first clone (clone 1) of the host cell (sBOND1) with the pBOND21 vector. Lane 4 shows a second clone (clone 2) of the host cell (sBOND1) with pBOND21 vector.

We observed a 19 kDa protein strongly expressed in lane 3 and lane 4, indicating that two different clones of the S. cerevisiae host cell comprising the pBOND21 vector express the cofilin-2 protein. In contrast, no 19 kDa protein was observed in the control empty vector strain (lane 2). These results demonstrate that a chicken cofilin-2 gene can be robustly produced in an S. cerevisiae host cell.

### 6.9.9. Example 9: The Expression of a Recombinant Chicken Cofilin-2 Protein Did not Hinder the Growth of Saccharomyces cerevisiae

Overexpression of actin and actin binding-proteins (also referred to as the “actin cytoskeleton machinery”) has deleterious effects in eukaryotic cells, such as yeast. These deleterious effects can include lethality, slow growth rates (e.g., delayed progression through the cell cycle), and abnormal morphology (e.g., filamentous growth). See, Yoshikawa et al. (2011) Yeast 28: 349-361; Stevenson et al. (2001) PNAS 98(7): 3946-3951. Cofilins are actin binding proteins that drive depolymerization of actin filaments. See Winder and Ayscough, J. Cell Science (2005) 118 (4): 651-654.

This study was conducted to determine if overexpressing a chicken cofilin-2 gene in an S. cerevisiae host cell hinders the growth of the host cell.

### Methods

### Cell Culture

The strains and cell culture were as described in Example 8.

### Growth Study

The two flasks, for each strain, were grown in either raffinose only or raffinose plus galactose to induce induction of protein expression, yielding eight separate flasks (four of each strain). Samples were taken every hour for the first 10 hours and then at various subsequent time points. The OD600 was measured at each time point. The OD600 values were graphed as averaged values from two flasks.

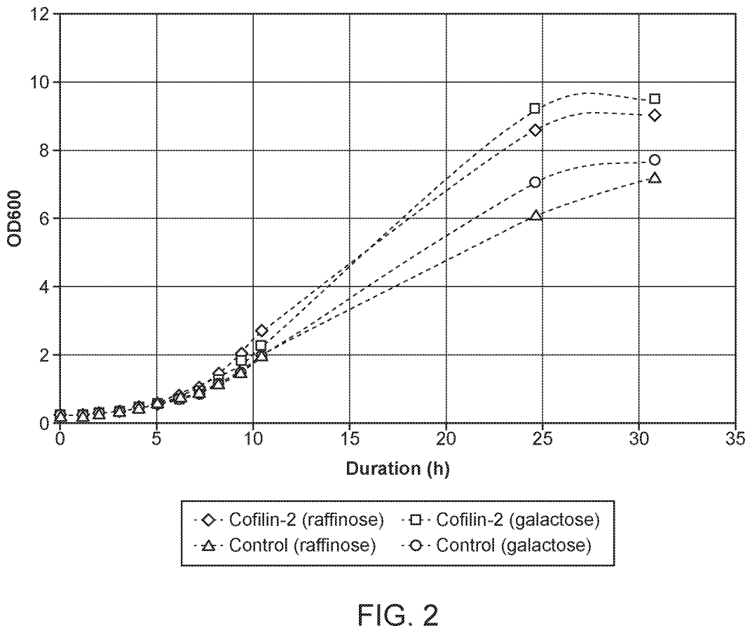

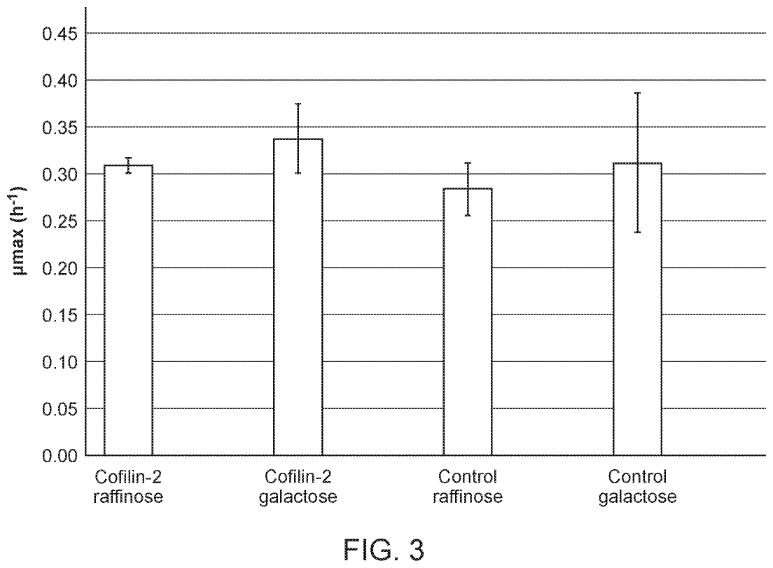

The results of the growth curves are shown in FIG. 2. The growth curves were established by plotting the average OD600 measurement over time. The maximum specific growth rate (μmax) was calculated by plotting ln(OD600) versus duration and then performing a linear regression analysis in Excel [version 16.31] from samples taken at the exponential phase. The value of μmax was determined by taking the maximum value of the slope between three time points. FIG. 3 shows the maximum specific growth rates (μmax [h-1]).

### Results

We observed no significant difference in the final cell densities (e.g., growth curve at saturation) between the recombinant yeast strain expressing cofilin-2 and the empty vector control strain, using a 95% confidence statistical threshold (one-way ANOVA). See FIG. 2. In addition, we observed no significant difference in the maximum specific growth rates between the recombinant yeast strains expressing cofilin-2 and the empty controls, using a 95% confidence statistical threshold (one-way ANOVA). See. FIG. 3. Moreover, there was no significant difference in μmax for the cofilin-2 strain with or without galactose (with/or without induction of protein expression).

These results demonstrate that an S. cerevisiae host cell expressing cofilin-2 can effectively grow and therefore efficiently produce an animal muscle protein.

### 6.9.10. Example 10: Production of Recombinant Profilin Protein from Chicken in an Saccharomyces cerevisiae Host Cell

This study was conducted to determine if a chicken profilin gene can be recombinantly produced in an S. cerevisiae host cell.

### Methods

Identification of Profilin Gene from Chicken

The sequence of chicken profilin was identified by searching Uniprot.org. The UniProt accession number was Q5ZL50. The amino acid sequence was:

[SEQ ID NO: 3]
MAGWQSYVDNLMCDGCCQEAAIVGYCDAKYVWAATAGGIFQSITPVEID
MIVGKDREGFFTNGLTLGAKKCSVIRDSLYVDGDCTMDIRTKSQGGEPT
YNVAVGRAGRVLVFVMGKEGVHGGGLNKKAYSMAKYLRDSGF

### Codon Optimization of Profilin

The amino acid sequence was codon optimized for expression in S. cerevisiae using ATUM's GeneGPS™ algorithm. The resulting gene sequence was:

[SEQ ID NO: 4]
ATGGCTGGCTGGCAATCTTATGTGGATAACTTAATGTGTGATGGATGTT
GTCAAGAGGCTGCAATCGTGGGTTACTGCGACGCAAAATACGTTTGGGC
AGCAACAGCTGGGGGCATATTCCAATCAATTACACCAGTTGAAATTGAT
ATGATCGTTGGTAAAGATAGAGAGGGATTTTTCACTAATGGTCTAACTT
TAGGTGCCAAAAAGTGCAGTGTTATCAGAGACTCACTGTACGTAGACGG
GGATTGCACCATGGATATTCGTACAAAGTCTCAGGGTGGAGAACCTACA
TACAACGTCGCGGTCGGCAGAGCCGGGAGAGTTTTGGTTTTCGTAATGG
GCAAGGAAGGTGTCCATGGTGGTGGACTTAACAAAAAGGCCTACTCTAT
GGCTAAGTACTTGAGAGATTCCGGTTTTTAA

### Cloning of Profilin Gene

The gene was synthesized by ATUM and cloned into the pD1205 (ATUM) expression vector, which is a 2-micron episomal vector that has GAL1-promoter and the TRP1 selection marker gene. The resulting expression vector was designated as (“pBOND3”).

The vector was transformed into chemically competent E. coli strain 5-alpha (New England Biolabs) by heat-shock transformation following the manufacturer's protocol and selection on Luria Bertani (LB) agar plates containing 25 μg/mL chloramphenicol.

Colonies were patched onto fresh LB plates with chloramphenicol and incubated at 37° C. Liquid cultures were inoculated and grown with shaking until saturation. The expression vector was purified using Zyppy plasmid miniprep kit (Zymo Research) by following the manufacturer's instructions.

The gene insert was verified by PCR and restriction digestion and transformed into host cell (“sBOND1”). The pBOND3 expression vector was transformed into the sBOND1 host cell by electroporation in a Bio-Rad Gene Pulser™.

The cell suspension was spread onto tryptophan dropout selection plates containing 6.8 g/L yeast nitrogen base without amino acids (Sigma Y0626), 1.9 g/L yeast synthetic dropout medium without tryptophan (Sigma Y1876), and 2% glucose (w/v).

### Cell Culture

Cells were grown in medium containing 6.8 g/L yeast nitrogen base without amino acids (Sigma Y0626), 1.9 g/L yeast dropout supplements without tryptophan (Sigma Y1876) and 20 g/L raffinose. The strain was cultured to an OD600 of 1, at which time profilin expression was induced by adding 20 g/L galactose. After induction, the culture was grown for an additional 18 hours. The final OD600 was around 8.

### Protein Analysis

Samples were taken at time of induction, after 5 hours, and at the end of the cultivation. Protein cell extracts were made as follows. The cells were lysed by a sodium hydroxide protocol (Kushnirov, 2000, Rapid and reliable protein extraction from yeast. Yeast, 16, 857-860). Cell pellets from 0.5 mL cell suspension were resuspended in 100 μL deionized water and 100 μL 0.2 N NaOH was added to each tube and then incubated at room temperature for 5 min. Subsequently, the cells were spun down for 1 min at 16000 g and resuspended in 40 μL sample buffer containing SDS (Laemmli, 1970, Cleavage of Structural Proteins during the Assembly of the Head of Bacteriophage T4. Nature, 227, 680-685).

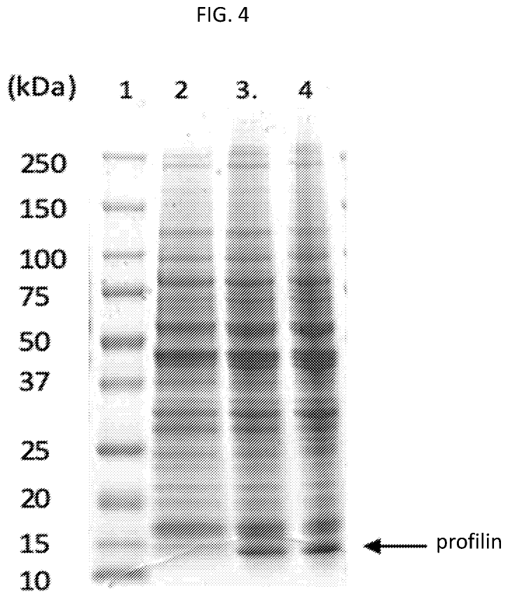

Equal amounts of total protein were loaded to each lane and analyzed by SDS-PAGE. Proteins were visualized by Coomassie staining, see FIG. 4.

### Results

FIG. 4 shows the results from the SDS-PAGE separation. The size of the profilin protein is 15 kDa. Lane 1 shows the molecular weight marker. Lane 2 shows protein expression before induction. Lane 3 shows protein expression 5 hours after induction by galactose. Lane 4 shows protein expression 18 hours after induction.

We observed increasing intensity of a 15 kDa protein band in lanes 3-4, where the S. cerevisiae host cell was induced to express the chicken profilin protein at increasing time durations. In contrast, a 15 kDa protein was not detected where there was no induction, lane 2. These results demonstrate that a chicken profilin protein can be produced in an S. cerevisiae host cell.

### 6.9.11. Example 11: The Expression of a Recombinant Chicken Profilin Protein Did not Severely Hinder the Growth of an Saccharomyces cerevisiae Host Cell

This study was conducted to determine if a recombinant S. cerevisiae host cell overexpressing a chicken profilin gene hinders the growth of the host cell.

### Methods

### Cell Culture

Cells were grown in flasks, two flasks for each strain, in either raffinose only or raffinose plus galactose, to induce induction of protein expression, yielding eight separate flasks (four for each strain). The medium also contained 6.8 g/L yeast nitrogen base without amino acids (Sigma Y0626) and 1.9 g/L yeast synthetic dropout medium without tryptophan (Sigma Y1876). The cultures were inoculated at an OD600 of about 0.2, and then grown for approximately 30 hours. Samples were taken every hour for the first 10 hours and then at various subsequent time points. The OD600 was measured at each time point. The OD600 values were graphed as averaged values from two flasks.

### Growth Analysis

Samples were taken every hour for the first 10 hours and then at various subsequent time points. The growth analysis was conducted as described in the Example 9.

Results

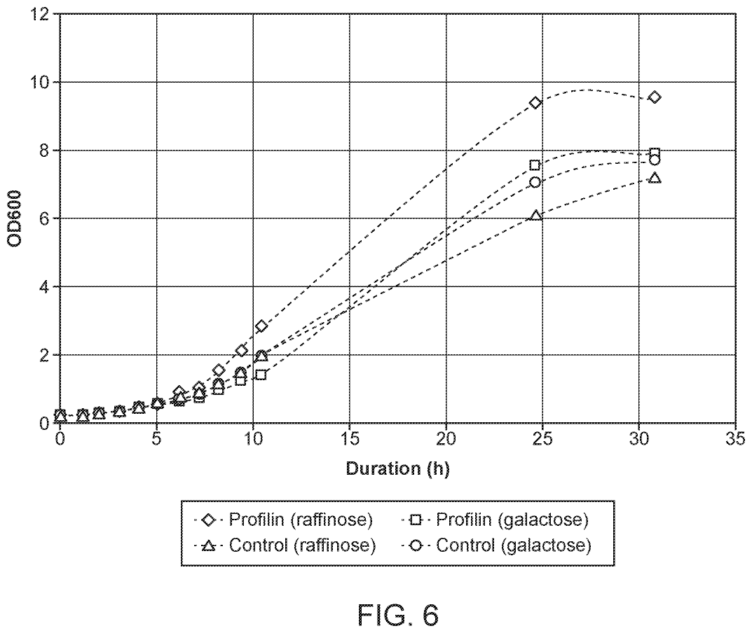

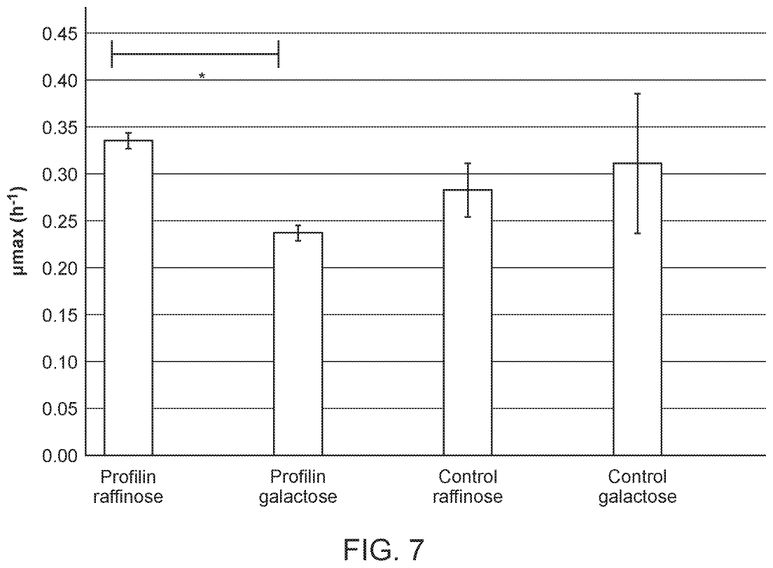

The results from the growth study are shown in FIG. 6 and FIG. 7. The growth curves are shown in FIG. 6. The maximum specific growth rates are shown in FIG. 7. We observed no significant differences between the final OD600 values or the maximum specific growth rates, between the strains expressing recombinant profilin and the empty control strains, using a 95% confidence statistical threshold (ANOVA). However, the maximum specific growth rate for the profilin strain was significantly higher for the uninduced culture compared to when induced with galactose (P value <0.05). The final OD600 values were also significantly higher for the uninduced culture. Still, the difference in final cell density was only around 20%. Thus, these results demonstrate that the expression of a chicken profilin-2 gene does not severely hinder the growth of a S. cerevisiae host cell.

### 6.9.12. Example 12: Recombinantly Expressed Profilin Protein can Increase the Amount of Essential Amino Acids

This study was conducted to determine if the recombinantly expressed profilin protein can increase the percentage of essential amino acids in a whole-cell extract.

### Methods

Identification of Profilin Gene from Chicken

The identification, cloning, and codon-optimization of the profilin gene were conducted as described in Example 10.

### Cell Culture

Profilin was expressed in several shake flask cultivations (about 10 L total) grown in 20 g/l raffinose medium (tryptophan dropout medium, as above) and induced by 20 g/l galactose at an OD600 around 1. After induction, the culture was grown for additional 22-24 hours. A control culture containing pBOND8 was prepared in the same host cell and ran in parallel.

### Amino Acid Analysis

The cells were concentrated by filtration using a 0.45 μm cellulose acetate membrane. The cells were dried at 65° C. for a minimum of 2 hours. The dried cells were submitted to Midwest laboratories for analysis.

### Results

Table 2 shows the amino acid analysis of S. cerevisiae expressing profilin compared to a control without profilin. Values reported as % (w/w).

TABLE 2
Crude protein and amino acid analysis of S. cerevisiae
expressing profilin compared to a control without profilin

| | | | Relative shift |
|---|---|---|---|
| | | | compared to |
| Analyte | Profilin | Control | control |
| Protein (crude) | 53.4% | 54.8% | |
| Aspartic acid | 5.16% | 4.15% | 24% |
| Threonine * | 1.63% | 1.38% | 18% |
| Serine | 2.55% | 2.07% | 23% |
| Glutamic acid | 5.85% | 5.41% | 8% |
| Proline | 1.87% | 1.55% | 21% |
| Glycine | 2.26% | 1.90% | 19% |
| Alanine | 3.04% | 3.37% | −10% |
| Cystine | 0.57% | 1.00% | −43% |
| Valine * | 3.00% | 2.82% | 6% |
| Methionine * | 0.88% | 0.71% | 24% |
| Isoleucine * | 2.49% | 2.11% | 18% |
| Leucine * | 3.73% | 3.40% | 10% |
| Tyrosine | 1.82% | 1.72% | 6% |
| Phenylalanine * | 2.25% | 1.87% | 20% |
| Lysine * | 3.92% | 3.62% | 8% |
| Histidine * | 1.16% | 1.23% | −6% |
| Arginine * | 2.76% | 2.69% | 3% |
| Tryptophan * | 0.63% | 0.54% | 17% |

* indicates that the amino acid is an essential amino acid for dogs.

These results show the recombinant host cell expressing profilin had an increase in all but one of the essential amino acids analyzed, as determined by % of dry weight. The only exception was histidine. See, the column labeled “Relative shift compared to control” in Table 2.

These results demonstrate that a recombinantly expressed profilin protein can increase the amount of essential amino acids in a whole-cell extract.

### 6.9.13. Example 13: Treat Composition Made with Recombinant Animal Protein

This study was conducted to determine if the recombinant animal protein powder can be used in a recipe with other ingredients to produce a food composition in the form of a dried pet treat.

### Methods

Identification of Profilin Gene from Chicken

The identification, cloning, and codon-optimization of the profilin gene were conducted as described in Example 10.

### Cell Culture

Cells were grown in flasks, in 13 L of 20 g/l raffinose medium, 6.8 g/L yeast nitrogen base without amino acids (Sigma Y0626), and 1.9 g/L yeast synthetic dropout medium without tryptophan (Sigma Y1876). When the culture reached an OD600 of 1 (i.e., exponential phase growth), expression of the profilin protein was induced by adding 20 g/l galactose. After induction, the culture was grown for another 22-24 hours, yielding a culture with an OD600 range of approximately 7-9.

The resulting cells were concentrated by filtration using a 0.45 μm cellulose acetate membrane. Next, the cells were dried at 70° C. for 1.5 hours. The dry weight yield was approximately 2.5 g/L.

Processing into a Treat

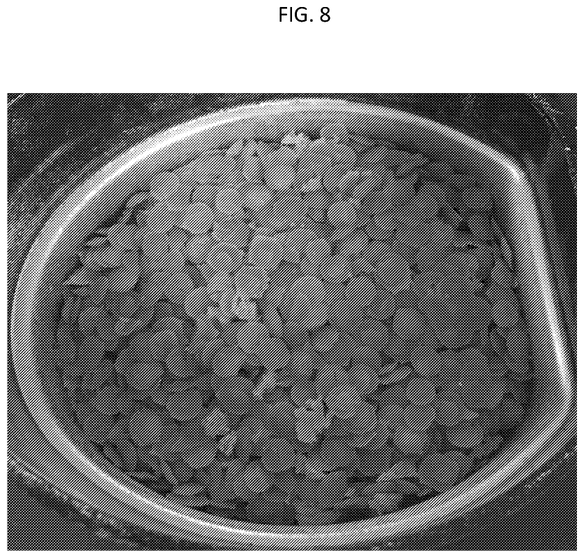

Whole-cells expressing the chicken profilin protein were pelleted and dried as described above. The dried pellets are shown in FIG. 8. Next, the pellets were ground until they became a fine powder. The profilin protein powder was then mixed with other ingredients, as outlined in Table 3, to make three different formulations. Each formulation contained different amounts of the profilin protein.

TABLE 3
Ingredients used for dog treats with recombinant chicken profilin protein

| | Ingredient (g) | | | Ingredient (wt %) | | |
|---|---|---|---|---|---|---|
| Ingredient | Treat #1 | Treat #2 | Treat #3 | Treat #1 | Treat #2 | Treat #3 |
| Red lentil flour | 5.5 | 5.5 | 5.5 | 9.2% | 8.4% | 9.9% |
| Sweet potato puree | 12.8 | 12.8 | 12.8 | 21.3% | 19.4% | 23.0% |
| Chickpea flour | 7.7 | 7.7 | 7.7 | 12.8% | 11.7% | 13.8% |
| Coconut oil | 5.1 | 5.1 | 5.1 | 8.5% | 7.8% | 9.2% |
| Oat flour | 5.1 | 5.1 | 5.1 | 8.5% | 7.8% | 9.2% |
| Quick oats | 2.6 | 2.6 | 2.6 | 4.3% | 3.9% | 4.6% |
| Pea protein | 5.1 | 5.1 | 5.1 | 8.5% | 7.8% | 9.2% |
| Cinnamon | 0.024 | 0.024 | 0.024 | 0.04% | 0.04% | 0.04% |
| Molasses | 4.4 | 4.4 | 4.4 | 7.3% | 6.6% | 7.8% |
| Water | 1.4 | 1.4 | 1.4 | 2.4% | 2.2% | 2.5% |
| S. cerevisiae containing | 10.3 (5.5) | 16.25 (8.7) | 6.0 (3.2) | 17.1% (9.1%) | 24.6% (13.2%) | 10.8% (5.8%) |
| chicken profilin (protein | | | | | | |
| contribution from | | | | | | |
| S. cerevisiae) | | | | | | |

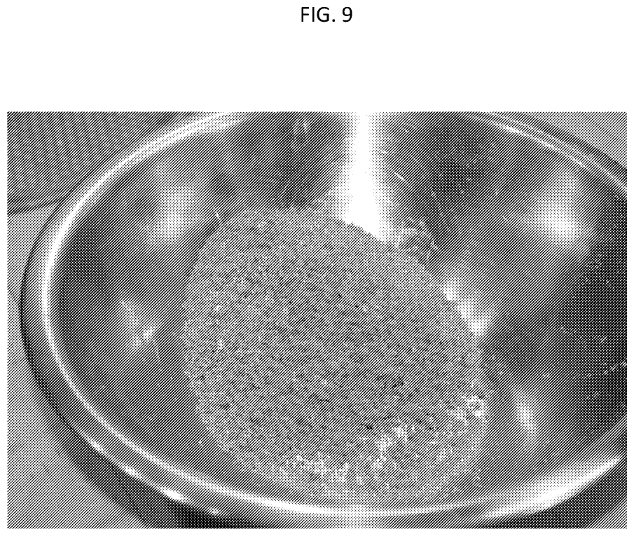

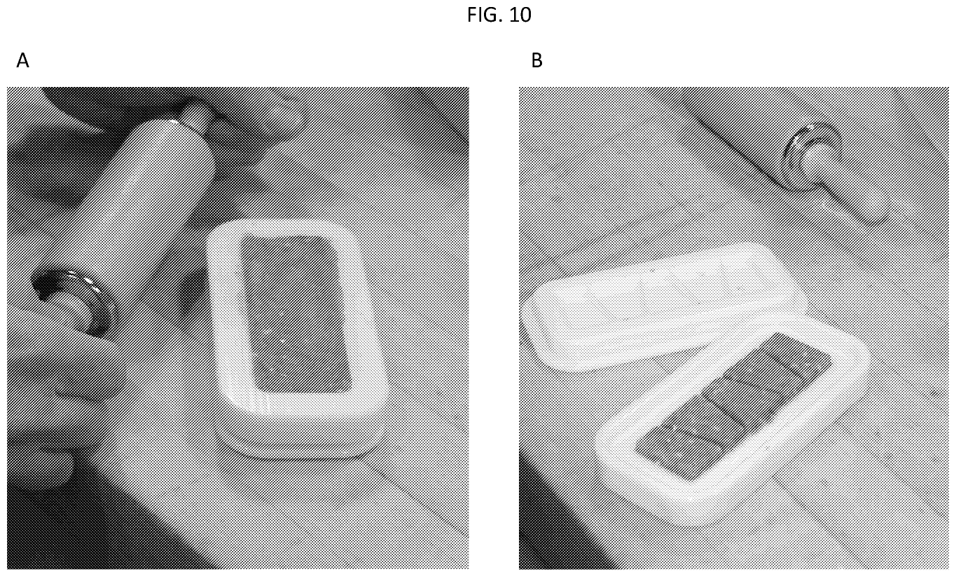

The treat was produced using the following method. The dry ingredients were mixed in a bowl. See FIG. 9. Next, the dry and wet ingredients were added to an electric mixer and mixed until the ingredients formed a dough-like consistency. Then the dough was compacted into the mold using a rolling pin. See FIG. 10A. The mold made a perforated pattern into the dough so that individual pieces can be broken off. See FIG. 10B. Finally, the treat was baked for 30 min at 250° F., and then dehydrated for 12 hours at 90° F.

### Results

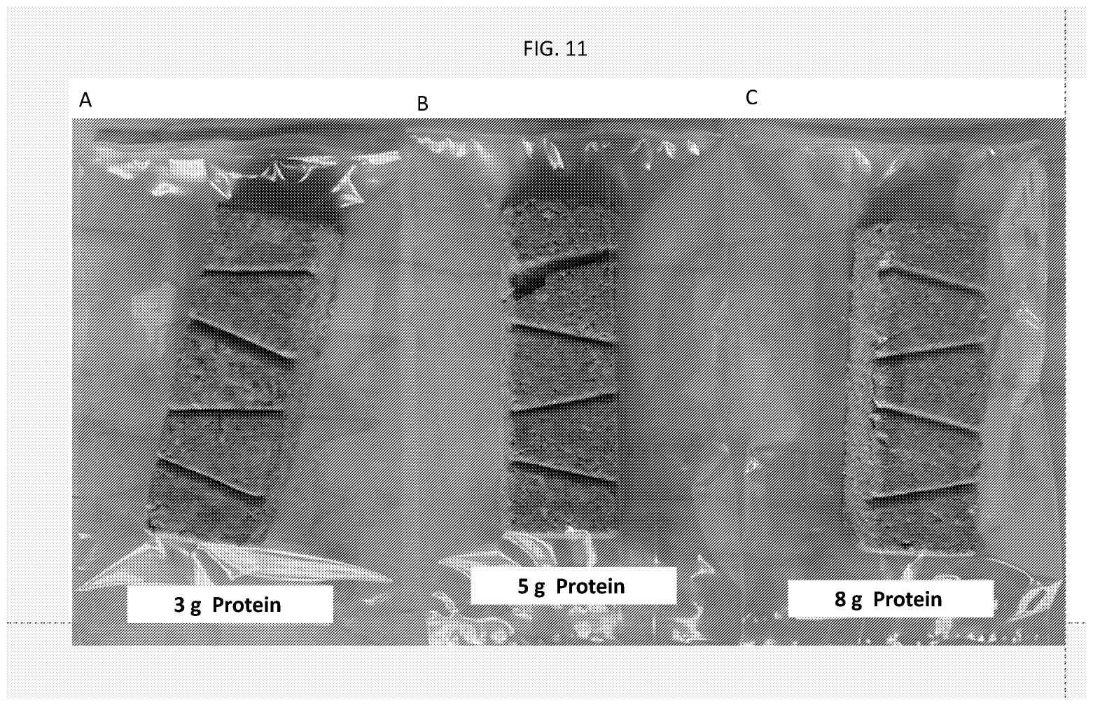

Three dog treats containing different amounts of the recombinant chicken profilin protein (3 grams, 5 grams, and 8 grams) were made. See FIGS. 11A-C. Taken together, the examples above demonstrate that the recombinant chicken profilin protein can be used to make treat composition that has a higher amount of essential amino acids.

### 6.9.14. Example 14: Production of Recombinant Coronin Protein from Chicken in an Saccharomyces cerevisiae Host Cell

This study was conducted to determine if an S. cerevisiae host cell could express a coronin protein from chicken.

Coronin has been classified as a side-binder and signaling protein. See Winder and Ayscough, J. Cell Science (2005) 118(4): 651-654. The particular coronin used here (coronin 6) is muscle-specific.

### Methods

Identification of the Coronin Gene Sequence from a Chicken Genome

The sequence of chicken coronin was obtained by searching Uniprot.org. The UniProt accession number was F1NXA5. The amino acid sequence was:

[SEQ ID NO: 5]
MSRRVVRQSKFRHVFGQPVKADQMYEDIRVSKVTWDSSFCAVNPKFVAI
IVEAGGGGAFMVLPLAKTGRVDKNHPLVTGHTAPVLDIDWCPHNDNVIA
SASEDTTVMVWQIPDYVPVRSITEPVVTLEGHSKRVGIICWHPTARNVL
LSAGCDNLVILWNVGTGEMLLALEDMHTDLIYNVGWNRNGSLLVTTCKD
KKVRVIDPRKQTVVAEITKPHDGARPIRAIFMADGKIFTTGFSKMSERQ
LGLWDLKNFEEPIALQEMDTSNGVLLPFYDPDTNIVYLCGKGDSSIRYF
EITDEAPYVHYLNTYSSKEPQRGMGFMPKRGLDVSKCEIARFFKLHERK
CEPIVMTVPRKSDLFQDDLYPDTPGPEPALEADEWLSGKDAEPILISLR
DGYVPVKNRELKVVKKNILDSKPPPGPRRSHSTSNTDISTPALDEVLEE
IRVLKETVQAQEKRISALEHKLCQFTNGTD

### Codon Optimization of Coronin

The amino acid sequence was codon optimized for expression in S. cerevisiae using ATUM's GeneGPS™ algorithm. The resulting gene sequence was:

[SEQ ID NO: 6]
ATGTCTCGTAGAGTTGTTAGACAATCCAAGTTCCGTCACGTGTTCGGCC
AACCAGTTAAGGCAGATCAGATGTACGAAGATATCAGAGTTTCAAAGGT
TACCTGGGACTCATCTTTTTGCGCTGTTAACCCAAAGTTCGTAGCAATA
ATTGTGGAAGCTGGCGGTGGGGGAGCATTTATGGTTTTACCACTAGCCA
AGACTGGTAGAGTCGACAAAAATCACCCTTTAGTCACTGGACATACAGC
ACCTGTATTAGATATTGACTGGTGTCCACATAACGACAATGTTATTGCA
AGTGCATCTGAGGATACAACTGTCATGGTATGGCAAATCCCAGACTACG
TTCCAGTAAGATCAATCACAGAACCAGTTGTCACGCTCGAGGGTCACTC
TAAGAGAGTTGGCATTATCTGTTGGCATCCTACAGCCAGAAATGTGTTG
TTGTCTGCCGGTTGCGATAACTTGGTAATTCTTTGGAACGTCGGTACAG
GCGAAATGTTGCTGGCGCTTGAAGATATGCACACTGACCTCATTTACAA
CGTCGGATGGAACAGAAACGGGTCGTTATTAGTCACCACATGTAAAGAT
AAAAAGGTAAGGGTTATCGACCCTAGAAAGCAAACAGTTGTTGCGGAAA
TCACAAAGCCACATGATGGTGCTAGACCAATTAGAGCTATATTCATGGC
CGATGGTAAGATTTTCACAACCGGATTCTCAAAAATGTCCGAGAGACAA
CTTGGGTTGTGGGATCTTAAAAACTTCGAGGAACCAATTGCTCTGCAGG
AAATGGATACTAGTAATGGTGTTTTGTTACCATTTTACGACCCAGACAC
AAACATCGTTTACCTCTGCGGCAAGGGTGATAGTAGCATCAGATATTTT
GAGATAACAGATGAAGCTCCTTACGTCCATTACTTGAATACTTACTCCT
CAAAGGAACCACAGAGAGGTATGGGATTCATGCCAAAGCGAGGACTAGA
TGTTTCTAAGTGTGAAATCGCTAGATTTTTCAAGTTACATGAGAGAAAA
TGCGAACCTATTGTGATGACAGTGCCTAGAAAATCTGATTTGTTCCAAG
ATGATCTATATCCAGATACTCCTGGCCCAGAACCAGCCCTTGAAGCTGA
TGAATGGTTATCTGGTAAAGATGCAGAGCCAATACTAATTTCTCTTAGA
GATGGGTACGTCCCAGTGAAAAACAGAGAGTTGAAAGTTGTTAAAAAAA
ATATTTTGGATAGCAAGCCTCCTCCAGGTCCTCGTAGATCTCACTCCAC
ATCAAACACCGATATATCAACACCAGCTTTGGATGAAGTTTTAGAGGAA
ATCCGGGTGTTGAAGGAAACTGTACAAGCACAAGAGAAGAGAATCTCAG
CACTGGAACATAAGCTATGTCAATTTACTAATGGTACCGACTAA

### Cloning of Coronin Gene

The gene was synthesized by ATUM and cloned into the pD1205 vector (ATUM), which is a 2-micron episomal vector that has a GAL1-promoter and the TRP1 selection marker gene. This expression vector was designated as (“pBOND2”).

The pBOND2 expression vector was transformed into chemically competent E. coli strain 5-alpha (New England Biolabs) by heat-shock transformation following the manufacturer's protocol. Selection for transformation was conducted on LB agar plates containing 25 μg/mL chloramphenicol. Colonies were patched on fresh LB agar plates with chloramphenicol and grown in liquid LB with 25 μg/mL chloramphenicol at 37° C. until saturation. The expression vector was purified using the Zyppy plasmid miniprep kit (Zymo Research) following the manufacturer's instructions. The gene insert was verified by PCR and restriction digestion. The expression vector was transformed into S. cerevisiae strain sBOND1 strain by electroporation using a Bio-Rad Gene Pulser™.

The cell suspension was spread onto selection plates comprising dropout tryptophan plates containing 6.8 g/L yeast nitrogen base without amino acids (Sigma Y0626), and 1.9 g/L yeast synthetic dropout medium without tryptophan (Sigma Y1876), and 2% glucose).

### Cell Culture

Cells were grown in a medium containing 6.8 g/L yeast nitrogen base without amino acids (Sigma Y0626), 1.9 g/L yeast dropout supplements without tryptophan (Sigma Y1876) and 20 g/L raffinose, until the culture reached an OD600 of 1, at which time coronin expression was induced by adding 20 g/L galactose (from a sterile filtered 40% (w/v) solution). After induction, cells were grown for an additional 18 hours. At the end of induction, the OD600 was around 8.

### Protein Analysis

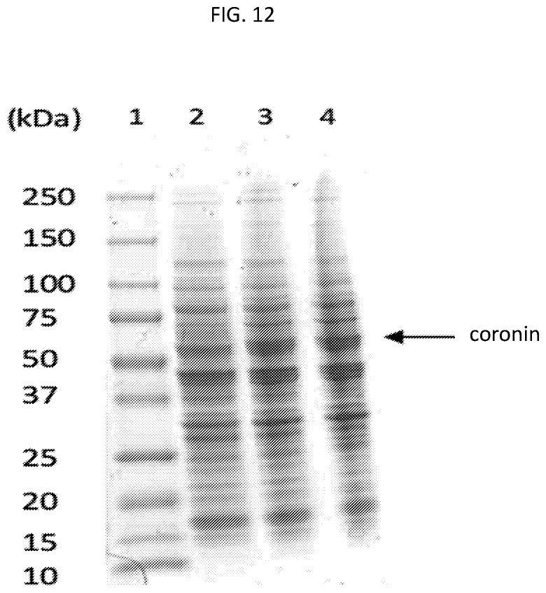

Samples were taken at the time of induction, after 5 hours after induction, and at the end of the cultivation. The cells were lysed as described in Example 10. Equal amounts of total protein were loaded onto each lane and analyzed by SDS-PAGE using a precast SDS-PAGE gel (MiniProtean TGX, 4-20% gradient, Bio-Rad). Proteins were visualized by Coomassie staining, see FIG. 12.

### Results

FIG. 12 shows the results of the SDS-PAGE analysis. The size of coronin is 53 kDa. Lane 1 shows the molecular weight marker. Lane 2 shows protein expression before induction. Lane 3 shows protein expression 5 hours after induction. Lane 4 shows protein expression 18 hours after induction.

Lanes 2-4 show an increasing amount of a 53 kDa protein, the expected size of codon optimized coronin protein. These results demonstrate that a chicken coronin protein can be produced in a S. cerevisiae host cell.

### 6.9.15. Example 15: Production of Recombinant Myozenin-1 Protein from Turkey in an Saccharomyces cerevisiae Host Cell

This study was conducted to determine if an S. cerevisiae host cell could express a myozenin-1 protein from turkey.

Myozenins function as calcineurin-interacting proteins that help tether calcineurin to the sarcomere of cardiac and skeletal muscle. They play an important role in modulation of calcineurin signaling. Myozenin 1 is predominantly expressed in fast-twitch skeletal muscle.

### Methods:

Identification of the Myozenin-1 Gene Sequence from a Turkey Genome

The sequence of turkey myozenin-1 was obtained by searching https://www.ncbi.nlm.nih.gov. The NCBI Reference number was XP_010712691.1. The amino acid sequence was:

[SEQ ID NO: 7]
MPLAGTPAPLKRKKPTKLIGKLTHEVMPQEVTKLNLGKKISIPRDVMLE
ELSLLTNKGSKMFKLRQLRVEKFIYENNPDAFSDNSVDHFQRFIPSGGH
YGEDAHGYGHGRMVGGVTAGQHGSSKQHYSTVPPRPGSKGGPGNSEGEH
EAEKSAGSAGGGHGTEKDGKSGGKKPLLKTYISPWERAMGISPEDKSQL
TIDLLSYSPKADFPHYKSFNRTAMPYGGYEKAAKRMTFKVPQFDICPLL
PESIVLYNQNFRNRPSFNRTPIPWMPSGESSEYHTDINVPRSGETEEL

### Codon Optimization of Myozenin-1

The amino acid sequence was codon optimized for expression in S. cerevisiae using ATUM's GeneGPS™ algorithm. The resulting gene sequence was:

[SEQ ID NO: 8]
ATGCCTTTAGCCGGAACCCCAGCACCATTGAAGAGAAAAAAGCCAACAA
AACTTATTGGTAAGCTGACACACGAAGTTATGCCACAGGAAGTTACCAA
GTTGAATCTAGGTAAAAAGATTTCTATCCCTAGAGATGTCATGTTGGAA
GAGTTATCGTTATTGACGAACAAAGGTTCCAAAATGTTCAAGTTGAGAC
AATTAAGAGTCGAGAAATTCATTTACGAAAACAATCCAGACGCATTCTC
CGATAACAGTGTTGATCATTTTCAACGTTTTATCCCATCTGGTGGACAT
TATGGTGAAGATGCCCATGGGTACGGTCATGGTCGTATGGTTGGGGGCG
TTACAGCCGGGCAACATGGTTCATCAAAGCAACATTACAGTACCGTGCC
TCCTCGACCTGGTTCTAAGGGTGGTCCAGGTAACTCTGAGGGTGAACAT
GCTGAAAAGTCAGCTGGGTCTGCTGGAGAGGGCGGCCACGGTACAGAAA
AGGATGGTAAGAGTGGTGGCAAAAAGCCTCTACTTAAGACTTACATCAG
CCCATGGGAGAGAGCGATGGGAATCTCACCAGAGGATAAGAGCCAGTTA
ACTATTGATCTTCTATCATATTCACCAAAGGCAGACTTCCCACACTACA
AATCTTTTAACAGAACAGCAATGCCATACGGCGGATACGAAAAAGCTGC
TAAGAGAATGACATTTAAGGTACCTCAATTCGATATCTGTCCACTGTTG
CCAGAATCCATAGTACTCTACAACCAAAATTTCAGAAACAGACCATCAT
TCAATAGAACTCCTATACCTTGGATGCCATCTGGCGAATCTTCCGAATA
CCACACTGACATTAACGTGCCAAGATCTGGAGAAACAGAGGAATTGTAA

### Cloning of Myozenin-1

The gene was synthesized by ATUM and cloned into the pD1211 (ATUM) vector, which is a yeast episomal plasmid, containing a 2-micron origin of replication, and the LEU2 selection marker. Expression of turkey myozenin-1 was driven by a yeast TEF1 promoter. This expression vector was designated as (“pBOND11”).

The pBOND11 expression vector was introduced into the host cell, S. cerevisiae ATCC® MYA-1108™ designated as (“sBOND28”) by transformation using Zymo Research™ Frozen-EZ Yeast Transformation II kit following the manufacturer's instructions. Transformants were selected using synthetic complete medium lacking leucine.

### Cell Culture

Cells were grown in flasks with selective media lacking leucine, containing 20 g/l glucose until the culture reached saturation, at which point the cells were collected by centrifugation.

### Protein Analysis

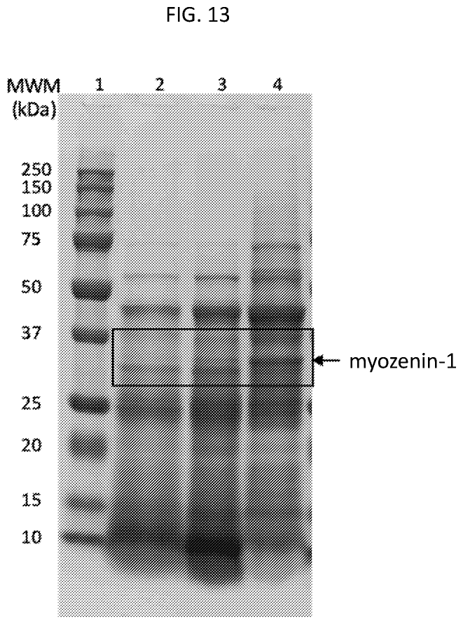

Cell pellets were weighed, and protein extracts were prepared using the Thermo Scientific™ YPER Yeast Protein Extraction Reagent according to manufacturer's instructions. Yeast extracts were quantitated using the Pierce™ BCA Protein Assay Kit according to manufacturer's guidelines. Equal amounts of total protein were loaded on each lane analyzed by SDS-PAGE. Proteins were visualized by Coomassie staining, see FIG. 13.

### Results

FIG. 13 shows a photograph of the SDS-PAGE gel after staining. The molecular size of myozenin-1 is 32 kDa. Lane 1 shows the molecular weight marker. Lane 2 shows the host cell (sBOND28). Lane 3 shows the host cell (sBOND28) with pBOND11 expressing myozenin-1 protein (clone 1). Lane 4 shows the host cell (sBOND28) with pBOND11 expressing myozenin-1 protein (clone 2).

We observed an increased expression of a 32 kDa protein with the clones expressing myozenin-1 protein (lanes 3-4), compared to the empty vector control strain (lane 2). These results demonstrate that an S. cerevisiae host cell could robustly produce a turkey myozenin-1 protein.

### 6.9.16. Example 16: Production of Recombinant Troponin C Protein from Pig in an Saccharomyces cerevisiae Host Cell

This study was conducted to determine if an S. cerevisiae host cell could express a troponin C protein from pig.

Troponin C is a protein that resides in the troponin complex on actin thin filaments of striated muscle and is responsible for binding calcium to activate muscle contraction.

### Methods:

Identification of the Troponin C Gene Sequence from a Pig Genome

The sequence of pig troponin C, skeletal muscle, was obtained by searching https://www.ncbi.nlm.nih.gov. The NCBI reference number NP_001001862.1. The amino acid sequence was:

[SEQ ID NO: 9]
MTDQQAEARSYLSEEMIAEFKAAFDMFDADGGGDISVKELGTVMRMLGQ
TPTKEELDAIIEEVDEDGSGTIDFEEFLVMMVRQMKEDAKGKSEEELAE
CFRIFDRNADGYIDAEELAEIFRASGEHVTDEELESLMKDGDKNNEGRI
DFDEFLKMMEGVQ

### Codon-Optimization of Troponin C

The amino acid sequence was codon-optimized for expression in S. cerevisiae using ATUM's GeneGPS™ algorithm. The resulting gene sequence was:

[SEQ ID NO: 10]
ATGACTGATCAACAAGCTGAAGCAAGATCTTACCTTAGTGAAGAGATGA
TAGCAGAGTTTAAGGCAGCGTTCGATATGTTCGATGCCGACGGTGGTGG
CGATATCTCTGTGAAGGAACTCGGTACAGTTATGAGAATGCTGGGGCAA
ACACCAACCAAGGAAGAGTTGGATGCAATCATCGAAGAGGTCGACGAAG
ATGGGTCAGGTACAATTGATTTTGAAGAGTTTTTGGTTATGATGGTAAG
ACAGATGAAAGAGGATGCTAAGGGTAAGTCAGAAGAGGAATTAGCTGAA
TGTTTTAGAATTTTCGATAGAAATGCTGATGGATACATTGACGCTGAGG
AACTAGCCGAAATTTTCCGTGCCTCTGGAGAACATGTCACTGATGAGGA
ATTGGAATCCTTAATGAAAGATGGCGACAAAAACAACGAGGGTAGAATC
GACTTCGACGAATTCCTTAAGATGATGGAAGGCGTTCAATAA

The gene was synthesized by ATUM and cloned into the pD1205 (ATUM) vector, which is a 2-micron episomal vector that has GAL1-promoter and the TRP1 gene to allow selection when transformed into a strain with tryptophan auxotrophy. The resulting expression vector was designated (“pBOND19”).

Transformation of pBOND19 into the S. cerevisiae host cell (“sBOND1”) was carried out using Zymo Research™ Frozen-EZ Yeast Transformation II kit following the manufacturer's instructions and selecting on synthetic complete media lacking tryptophan. An empty vector control (pBOND8) strain was prepared using the same host cell.

### Cell Culture

Cells were grown in flasks on selective media (lacking tryptophan) containing 2% (w/v) raffinose until the culture reached an OD600 of 1. Galactose was added to a final concentration of 2% (w/v) to induce expression of the protein. After induction, cultures were grown for another 24 hours with vigorous shaking.

### Protein Analysis

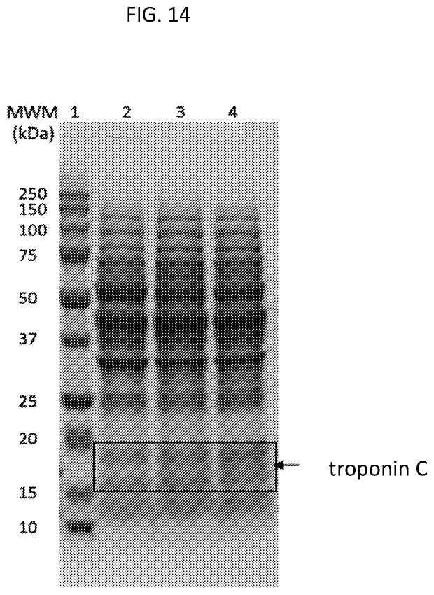

Cell pellets were weighed, and protein extracts were prepared using the Thermo Scientific™ YPER Yeast Protein Extraction Reagent according to manufacturer's instructions. Samples were quantitated using the Pierce™ BCA Protein Assay Kit according to the manufacturer's guidelines. Equal amounts of total protein were loaded onto each lane and analyzed by SDS-PAGE. Proteins were visualized by Coomassie staining, see FIG. 14.

### Results

FIG. 14 shows the stained SDS-PAGE gel. The molecular size of troponin C is 18 kDa. Lane 1 shows a molecular weight marker. Lane 2 shows the host cell (sBOND1) with an empty vector (pBOND8). Lane 3 shows the host cell (sBOND1) with pBOND19 expressing the troponin C protein (clone 1). Lane 4 shows the host cell (sBOND1) with pBOND19 expressing the troponin C protein (clone 2).

We observed an 18 kDa protein in lanes 3-4. Notably, there was no such 18 kDa protein observed in the empty control strain (lane 2). These results demonstrate that a S. cerevisiae host cell could produce a pig troponin C protein.

### 6.9.17. Example 17: Production of Recombinant Cofilin-2 from Chicken in a Komagataella phaffii Host Cell

This study was conducted to determine if a Komagataella phaffii (formerly known as Pichia pastoris) host cell could express a cofilin-2 protein from chicken.

### Methods:

Identification of the Cofilin-2 Gene Sequence from a Chicken Genome

The sequence of chicken cofilin-2 was obtained by searching https://www.ncbi.nlm.nih.gov. The NCBI reference number was NP_001004406.1. The amino acid sequence was [SEQ ID NO: 1].

### Codon Optimization of Cofilin-2

The amino acid sequence was codon optimized for expression in K. phaffii using ATUM's GeneGPS™ algorithm. The resulting gene sequence was:

[SEQ ID NO: 11]
ATGGCTTCTGGTGTGACTGTTAACGACGAAGTCATCAAGGTATTCAATG
ATATGAAAGTTAGAAAATCATCCACTCCAGAGGAAATCAAAAAGAGAAA
AAAAGCCGTTCTATTTTGCCTGTCGGACGACAAAAAGCAGATCATCGTT
GAGGAAGCCACACGTATTTTGGTCGGTGACATTGGTGACACAGTCGAAG
ATCCTTATACTGCTTTTGTTAAGCTGTTGCCCTTAAATGATTGTAGGTA
CGCTCTGTACGACGCAACTTACGAAACCAAAGAGTCCAAAAAAGAGGAT
TTGGTGTTCATCTTCTGGGCACCTGAAAGTGCTCCACTTAAGAGCAAGA
TGATTTATGCATCCTCTAAAGATGCTATTAAAAAAAAGTTTACAGGTAT
AAAGCATGAGTGGCAAGTGAACGGATTGGATGACATTAAAGATAGATCT
ACGTTGGGCGAAAAGCTTGGTGGAAATGTTGTAGTGTCATTAGAGGGAA
AGCCACTCTAA

### Cloning of Cofilin-2

The gene was synthesized by ATUM and cloned into the pD902 vector (ATUM), which is a yeast integrating plasmid that has a zeocin resistance gene for selection, and an AOX1 promoter. The resulting expression vector was designated as (“pBOND24”).

Komagataella phaffii (formerly Pichia pastoris) PPS-9016 was obtained from ATUM and designated as (“sBOND2”). The pBOND24 expression vector was linearized using the restriction enzyme PmeI and was introduced into the sBOND2 host cell by transformation using Zymo Research™ Frozen-EZ Yeast Transformation II kit following the manufacturer's instructions. Cells were allowed to recover overnight in non-selective media, and the following day they were plated on selective plates containing YPD (10 g/l yeast extract, 20 g/l peptone, 20 g/l glucose) with either 250 μg/ml or 1000 μg/ml zeocin.

### Cell Culture

The cells were grown in baffled flasks in BMGY plus zeocin media (10 g/l yeast extract, 20 g/l peptone, 13.4 g/L yeast nitrogen base (without amino acids), 100 mM potassium phosphate pH 6, 0.004 mg/L biotin, 1% (v/v) glycerol, and 500 μg/ml zeocin) until the culture reached an OD600 of 1. Methanol was added to a final concentration of 0.5% (v/v) to induce expression of the protein. After induction, cultures were grown for another 60 hours with vigorous shaking, and methanol was added every 24 hours to maintain and/or boost induction of protein expression.

### Protein Analysis

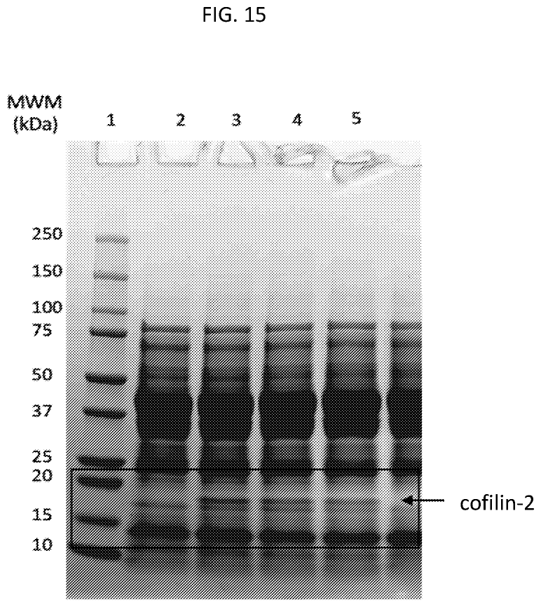

Cell cultures were collected by centrifugation and cell pellets were weighed. Protein extracts were prepared using the Thermo Scientific™ YPER Yeast Protein Extraction Reagent according to the manufacturer's instructions. Yeast extracts were quantitated using the Pierce™ BCA Protein Assay Kit according to the manufacturer's guidelines. Equal amounts of total protein were loaded to each lane and analyzed by SDS-PAGE. Proteins were visualized by Coomassie staining, see FIG. 15.

### Results

FIG. 15 shows the results of the SDS-PAGE gel. The molecular size of cofilin-2 is 19 kDa. Lane 1 shows the molecular weight marker. Lane 2 shows the host cell (sBOND2). Lane 3 shows the host cell (BOND2) with pBOND24 expressing the cofilin-2 protein (clone #2). Lane 4 shows the host cell (sBOND2) with pBOND24 expressing the cofilin-2 protein (clone #3). Lane 5 shows the host cell (sBOND2) with pBOND24 expressing the cofilin-2 protein (clone #5).

We observed a strong band at 19 kDa in the cofilin-2 expressing clones (lanes 3-5), while no such band was observed in the control lane (lane 2). These results demonstrate that a Komagataella phaffii host cell could robustly produce a chicken cofilin-2 protein.

### 6.9.18. Example 18: Production of Recombinant Profilin Protein from Chicken in a Komagataella phaffii Host Cell

This study was conducted to determine if a Komagataella phaffii host cell could express a profilin protein from chicken.

### Methods

Identification of Profilin Gene Sequence from Chicken

The sequence of chicken profilin was obtained by searching Uniprot.org. The UniProt accession number is Q5ZL50. The amino acid sequence was: [SEQ ID NO:34]

### Condon Optimization of Profilin

The amino acid sequence was codon optimized for K. phaffii using ATUM's GeneGPS™ algorithm. The resulting gene sequence was:

[SEQ ID NO: 12]
ATGGCCGGTTGGCAATCCTATGTAGACAATCTAATGTGTGACGGGTGTT
GCCAAGAGGCAGCAATTGTGGGTTACTGCGATGCTAAATACGTTTGGGC
AGCAACAGCCGGCGGAATCTTCCAATCAATAACCCCAGTGGAAATTGAT
ATGATTGTTGGAAAAGACCGTGAGGGATTTTTCACTAATGGTTTGACTC
TGGGTGCTAAAAAGTGTTCCGTTATCCGTGATAGCTTGTATGTCGATGG
TGACTGTACTATGGATATCAGGACAAAGTCGCAGGGTGGTGAACCTACG
TATAACGTAGCTGTCGGTAGAGCTGGAAGAGTGTTAGTCTTTGTTATGG
GTAAAGAGGGTGTTCATGGCGGTGGACTTAACAAAAAGGCTTACAGTAT
GGCTAAGTACTTGAGAGACTCTGGATTCTAA

### Cloning of Profilin

The gene was synthesized by ATUM and cloned into the pD902 vector, which is a yeast integrating plasmid that has a zeocin resistance gene to allow for the selection of transformants, and an AOX1 promoter. The resulting expression vector was designated (“pBOND25”).

Komagataella phaffii (formerly Pichia pastoris) PPS-9016 was obtained from ATUM and designated as (“sBOND2”). pBOND25 was linearized using the restriction enzyme, PmeI and was transformed into sBOND2 using the Zymo Research™ Frozen-EZ Yeast Transformation II kit following the manufacturer's instructions. Cells were allowed to recover overnight in non-selective media, and the following day they were plated on YPD agar plates containing 1000 μg/ml zeocin for selection.

### Cell Culture

Cells were grown in flasks in BMGY plus zeocin media (10 g/l yeast extract, 20 g/l (w/v) peptone, 13.4 g/l yeast nitrogen base (without amino acids), 100 mM potassium phosphate pH 6.0, 0.004 mg/l biotin, 1% (v/v) glycerol, and 500 μg/ml zeocin) until the culture reached a OD600 of 1. Methanol was added to a final concentration of 0.5% (v/v) to induce expression of the protein. After induction, cultures were grown for an additional 60 hours with vigorous shaking. Methanol was added every 24 hours after the first time to maintain and/or boost induction of protein expression.

### Protein Analysis

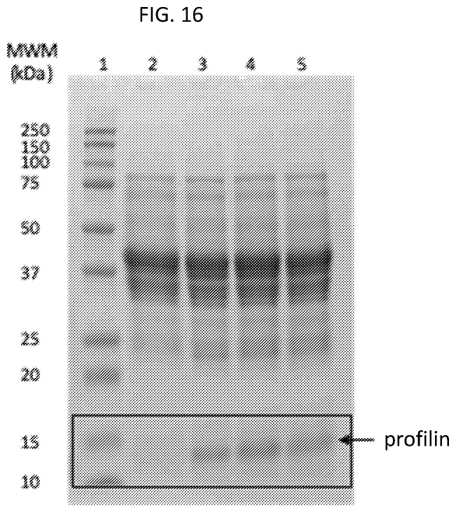

Cell cultures were collected by centrifugation and cell pellets were weighed. Protein extracts were prepared using the Thermo Scientific™ YPER Yeast Protein Extraction Reagent according to manufacturer's instructions. Yeast extracts were quantitated using the Pierce™ BCA Protein Assay Kit the according to manufacturer's guidelines. Equal amounts of total protein were loaded on each lane and analyzed by SDS-PAGE. Proteins were visualized by Coomassie staining, see FIG. 16.

### Results

FIG. 16 shows the results of the SDS-PAGE gel. The molecular size of profilin is 15 kDa. Lane 1 shows the molecular weight marker. Lane 2 shows the host cell (sBOND2). Lane 3 shows the host cell (sBOND2) with pBOND25 expressing the profilin protein (clone 1). Lane 4 shows the host cell (sBOND2) with pBOND25 expressing the profilin protein (clone 2). Lane 5 shows the host cell (sBOND2) with pBOND25 expressing the profilin protein (clone 3).

We observed a protein band at 15 kDa, in the profilin expressing clones (lanes 3-5), while no such band was observed in the empty control strain (lane 2). These results demonstrate that a Komagataella phaffii host cell could produce a chicken profilin protein.

### 6.9.19. Example 19: Production of Recombinant Profilin Protein from Chicken in a Kluyveromyces lactis Host Cell

This study was conducted to determine if a Kluyveromyces lactis host cell could express a profilin protein from chicken.

### Methods

Identification and Cloning of Profilin Gene from Chicken

The identification and cloning of the profilin gene were carried out as described in Example 10.

The profilin gene [SEQ ID NO: 4] was amplified from pBOND3 (origin and cloning described in Example 10) using the cloning primers oBOND20 and oBOND21 (Table 11). The resulting PCR fragment was digested with restriction enzymes HindIII and NdeI, gel purified, and then ligated with T4 DNA ligase into the integrating vector pKLAC2 (New England Biolabs). The vector was then linearized with the same restriction enzymes and dephosphorylated by Quick CIP (New England Biolabs). This generated the expression vector designated as (“pBOND22”).

The pBOND22 expression vector was transformed into 10-beta competent cells (New England Biolabs) by heat-shock transformation and the transformants were selected on LB agar plates containing 100 μg/mL carbenicillin. A few colonies were cultured in LB with 100 μg/mL carbenicillin and the expression vector was purified using the Zyppy plasmid miniprep kit (Zymo Research) by following the manufacturer's instructions. The gene insert was verified by restriction digestion.

The pBOND22 expression vector was linearized using the restriction enzyme SacII and desalted using the PCR and Cleanup kit (Monarch) and transformed into an K. lactis host cell GG799 (New England Biolabs) designated as (“sBOND68”) by chemical transformation. Two negative controls were prepared, an empty vector designated as (“pBOND27”), and a control gene expressing a maltose-binding protein designated as (“pBOND28”) transformed into the same host cell. Cells were grown on YCB agar plates containing 5 mM acetamide, for 4 days at 30° C. Several colonies were patched on new YCB agar plates containing 5 mM acetamide.

### Cell Culture and Protein Analysis

A few colonies were inoculated into YPGal medium (10 g/L yeast extract, 20 g/L peptone, and 20 g/l galactose) and allowed to grow at 30° C.

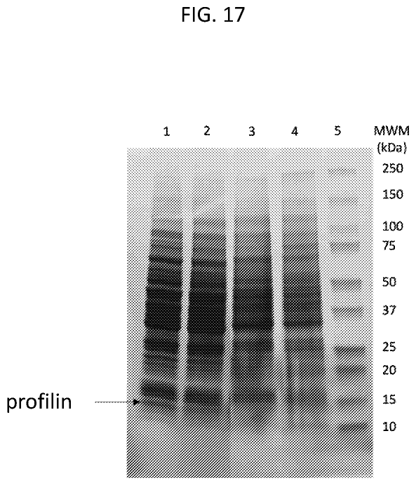

Samples were taken and analyzed after approximately 24 hours, when the OD600 range was about 25-30. Cell cultures were collected by centrifugation and cell pellets were weighed. The cells were lysed as described in Example 10. Equal amounts of total protein estimated by OD600 values loaded on each lane and analyzed by SDS-PAGE. Proteins were visualized by Coomassie staining, see FIG. 17.

### Results

FIG. 17 shows the SDS-PAGE gel. The molecular size of profilin is 15 kDa. Lane 1 shows the host cell (sBOND68) with pBOND22 expressing the profilin protein (clone 2). Lane 2 shows the host cell (sBOND68) with pBOND22 expressing the profilin protein (clone 3). Lane 3 shows the host cell (sBOND68) with pBOND27 empty vector. Lane 4 shows host cell (sBOND68) with pBOND28 expressing the control gene, maltose-binding protein. Lane 5 shows the molecular weight marker.

We observed a 15 kDa protein in the profilin expressing clones (lanes 1-2), while no such band was observed in the empty vector control or the control gene (lanes 3-4). These results demonstrate that a K. lactis host cell could produce a chicken profilin protein.

### 6.9.20. Example 20: Production of Recombinant Profilin Protein from Chicken in a Schizosaccharomyces pombe Host Cell

This study was conducted to determine if a Schizosaccharomyces pombe host cell could express a profilin protein from chicken.

Identification and Cloning of Profilin Gene from Chicken

The identification and cloning of the profilin gene were carried out as described in Example 10.

The profilin gene [SEQ ID NO: 4] was amplified from pBOND3 expression vector using primers oBOND5 and oBOND6 (see Table 11). The PCR fragment was digested with restriction enzymes XhoI and SmaI, gel purified, and then ligated into pBOND10 (REP4X [ATCC 87604]) vector which was cut with the same restriction enzymes. This placed the profilin gene in front of the S. pombe nmtl promoter and generated the expression vector designated as (“pBOND29”), which is a high copy plasmid.

The pBOND29 expression vector was introduced into an S. pombe strain, designated as (“sBOND3”) using the Zymo Research™ Frozen-EZ Yeast Transformation II kit following the manufacturer's instructions. An empty vector “pBOND10” strain was also generated using the same host cell.

### Cell Culture

Cell were grown in flasks in glucose selective media, lacking uracil and containing thiamine, to repress the expression of profilin, until the culture reached an OD600 of 1. Next, cells were transferred to media lacking thiamine, to induce expression of profilin. Subsequently, the cells were grown at 37° C. for an additionally 30 hours with vigorous shaking. After, cells were collected by centrifugation.

### Protein Analysis

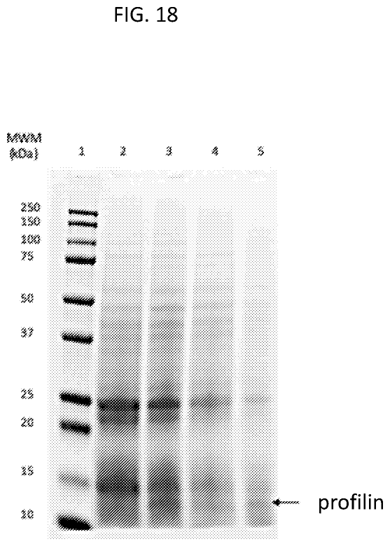

Protein extracts were prepared by treating cells with 0.3N NaOH for 15 minutes, and then boiling the cell pellet in SDS sample buffer for 5 minutes (Matsuo, Asakawa, Toda, & Katayama, 2006, A Rapid Method for Protein Extraction from Fission Yeast. Bioscience, Biotechnology, and Biochemistry, 70(8), 1992-1994). Equal amounts of total protein were loaded on each lane and analyzed by SDS-PAGE. Proteins were visualized by Coomassie staining, see FIG. 18.

### Results

FIG. 18 shows the results of the SDS-PAGE gel. Lane 1 shows the molecular weight marker. Lane 2 shows the host cell (sBOND3) with the vector pBOND10 empty vector. Lane 3 shows the host cell (sBOND3) with pBOND29 expressing the profilin protein (clone 2). Lane 4 shows the host cell (sBOND3) with pBOND29 expressing the profilin protein (clone 3). Lane 5 shows the host cell (sBOND3) with pBOND29 expressing the profilin protein (clone 5).

We observed a protein at 15 kDa, in the sBOND3 profilin expressing clones (lanes 3-5), while no such band was observed in the pBOND10 empty vector strain (lane 2). These results demonstrate that a S. pombe host cell could express a chicken profilin protein.

### 6.9.21. List of Strains, Expression Vectors, and Oligonucleotides

TABLE 9
Host Cell Strains

| | | Other | |
|---|---|---|---|
| Name | Species | designation | Genotype |
| sBOND1 | Saccharomyces | ATCC 208288 | MATalpha ura3-52 trp1 leu2- |
| | cerevisiae | | delta1 his 3-delta200 pep4::HIS3 |
| | | | prb1-delta1.6R can1 GAL |
| sBOND2 | Komagataella | Pichia pastoris | pep4Δ prb1Δ |
| | phaffii | PPS-9016 | |
| sBOND3 | Schizosaccharomyces | FY118/ATCC | h90 ura4-D18 |
| | pombe | 201401 | leu1-32 ade6-M216 |
| sBOND28 | Saccharomyces | ATCC MYA-1108 | MATa trp1-289 leu2-3 leu2-112 |
| | cerevisiae | CEN.PK2-1Ca | ura3-52 his3-delta1MATa |
| | | | trp1-289 leu2-3 leu2-112 |
| | | | ura3-52 his3-delta1 |
| sBOND68 | Kluyveromyces | GG799 | MATα |
| | lactis | | [pGKI1⁺] |

*MBP = Maltose binding protein

TABLE 10
Expression Vectors

| | | | Bacterial | Yeast | |
|---|---|---|---|---|---|
| Name | Other designation | Gene | marker | marker | Promoter |
| pBOND2 | pD1205-Chck_Coronin | Chicken | CmR | TRP1 | GAL1 |
| | | coronin | | | |
| pBOND3 | pD1205-Chck_Profilin | Chicken | CmR | TRP1 | GAL1 |
| | | profilin | | | |
| pBOND4 | pD1248-Chck_Cofilin-2 | Chicken | AmpR | URA3 | GAL1 |
| | | cofilin-2 | | | |
| pBOND8 | pRS424 | Empty | AmpR | TRP1 | GAL1 |
| | | Control | | | |
| pBOND10 | pREP4x | Empty | AmpR | ura4⁺ | NMT1 |
| | | Control | | | |
| pBOND11 | pD1211_Myozenin-1 | Turkey | KanR | LEU2 | TEF1 |
| | | myozenin1 | | | |
| pBOND19 | pD1205_Pig_TroponinC | Pig | CmR | TRP1 | GAL1 |
| | | troponinC | | | |
| pBOND21 | pRS424 P-GAP_Cofilin2 | Chicken | AmpR | TRP1 | GAL1 |
| | | cofilin-2 | | | |
| pBOND22 | pKLAC2_P-LAC4_Profilin | Chicken | AmpR | amdS | LAC4 |
| | | profilin | | | |
| pBOND24 | pD902_Chck_Cofilin-2 | Chicken | ZeocinR | ZeocinR | AOX1 |
| | | cofilin-2 | | | |
| pBOND25 | pD902_Chck_Profilin | Chicken | ZeocinR | ZeocinR | AOX1 |
| | | profilin | | | |
| pBOND27 | pKLAC2 | Empty | AmpR | amdS | LAC4 |
| | | Control | | | |
| pBOND28 | pKLAC1-malE | MBP* | AmpR | amdS | LAC4 |
| pBOND29 | pREP4x-Chck_Profilin-5 | Chicken | AmpR | ura4⁺ | NMT1 |
| | | profilin | | | |

TABLE 11
Oligonucleotide primers for cloning

| SEQ ID NO: | Name | Sequence of Oligonucleotide |
|---|---|---|
| SEQ ID NO: 13 | oBOND5 | 5′-ATAACTCGAGATGGCTGGCTGGCAATCTTATG-3′ |
| SEQ ID NO: 14 | oBOND6 | 5′-TCCCGGGTTAAAAACCGGAATCTCTCAAG-3′ |
| SEQ ID NO: 15 | oBOND11 | 5′-TATTCTCGAGACGGATTAGAAGCCGCCGAGC-3′ |
| SEQ ID NO: 16 | oBOND12 | 5′-TACAGAATTCTTTTGCGTGCAGGTGAGG-3′ |
| SEQ ID NO: 17 | oBOND20 | 5′-TATAAGCTTATGGCTGGCTGGCAATCTTATG |
| SEQ ID NO: 18 | oBOND21 | 5′-ATACCATATGTTAAAAACCGGAATCTCTCAAG |

### 7. SEQUENCES

TABLE 1
Sequences of Animal Proteins
Skeletal muscle tissue

| | Accession | | |
|---|---|---|---|
| SEQ ID | number | Common | |
| NO: | (database) | name/animal | Amino acid sequence |
| SEQ ID | A4IFM8 | Actin, alpha 1, | MCDEDETTALVCDNGSGLVKAGFA |
| NO: 19 | (UniProtKB) | skeletal | GDDAPRAVFPSIVGRPRHQGVMVG |
| | | muscle/Bos | MGQKDSYVGDEAQSKKGILTLKYPI |
| | | taurus | EHGIITNWDDMEKIWHHTFYNELRV |
| | | | APEEHPTLLTEAPLNPKANREKMTQI |
| | | | MFETFNVPAMYVAIQAVLSLYASGR |
| | | | TTGIVLDSGDGVTHNVPIYEGYALPH |
| | | | AIMRLDLAGRDLTDYLMKILTERGY |
| | | | SFVTTAEREIVRDIKEKLCYVALDFE |
| | | | NEMATAASSSSLEKSYELPDGQVITI |
| | | | GNERFRCPETLFQPSFIGMESAGIHET |
| | | | TYNSIMKCDIDIRKDLYANNVMSGG |
| | | | TTMYPGIADRMQKEITALAPSTMKIK |
| | | | IIAPPERKYSVWIGGSILASLSTFQQM |
| | | | WITKQEYDEAGPSIVHRKCF |
| SEQ ID | P68137 | Actin, alpha | MCDEDETTALVCDNGSGLVKAGFA |
| NO: 20 | (UniProtKB) | skeletal | GDDAPRAVFPSIVGRPRHQGVMVG |
| | | muscle/Sus | MGQKDSYVGDEAQSKRGILTLKYPI |
| | | scrofa | EHGIITNWDDMEKIWHHTFYNELRV |
| | | | APEEHPTLLTEAPLNPKANREKMTQI |
| | | | MFETFNVPAMYVAIQAVLSLYASGR |
| | | | TTGIVLDSGDGVTHNVPIYEGYALPH |
| | | | AIMRLDLAGRDLTDYLMKILTERGY |
| | | | SFVTTAEREIVRDIKEKLCYVALDFE |
| | | | NEMATAASSSSLEKSYELPDGQVITI |
| | | | GNERFRCPETLFQPSFIGMESAGIHET |
| | | | TYNSIMKCDIDIRKDLYANNVMSGG |
| | | | TTMYPGIADRMQKEITALAPSTMKIK |
| | | | IIAPPERKYSVWIGGSILASLSTFQQM |
| | | | WITKQEYDEAGPSIVHRKCF |
| SEQ ID | P68139 | Actin, alpha | MCDEDETTALVCDNGSGLVKAGFA |
| NO: 21 | (UniProtKB) | skeletal | GDDAPRAVFPSIVGRPRHQGVMVG |
| | | muscle/Gallus | MGQKDSYVGDEAQSKRGILTLKYPI |
| | | gallus | EHGIITNWDDMEKIWHHTFYNELRV |
| | | | APEEHPTLLTEAPLNPKANREKMTQI |
| | | | MFETFNVPAMYVAIQAVLSLYASGR |
| | | | TTGIVLDSGDGVTHNVPIYEGYALPH |
| | | | AIMRLDLAGRDLTDYLMKILTERGY |
| | | | SFVTTAEREIVRDIKEKLCYVALDFE |
| | | | NEMATAASSSSLEKSYELPDGQVITI |
| | | | GNERFRCPETLFQPSFIGMESAGIHET |
| | | | TYNSIMKCDIDIRKDLYANNVMSGG |
| | | | TTMYPGIADRMQKEITALAPSTMKIK |
| | | | IIAPPERKYSVWIGGSILASLSTFQQM |
| | | | WITKQEYDEAGPSIVHRKCF |
| SEQ ID | P68138 | Actin, alpha | MCDEDETTALVCDNGSGLVKAGFA |
| NO: 22 | (UniProtKB) | skeletal | GDDAPRAVFPSIVGRPRHQGVMVG |
| | | muscle/Bos | MGQKDSYVGDEAQSKRGILTLKYPI |
| | | taurus | EHGIITNWDDMEKIWHHTFYNELRV |
| | | | APEEHPTLLTEAPLNPKANREKMTQI |
| | | | MFETFNVPAMYVAIQAVLSLYASGR |
| | | | TTGIVLDSGDGVTHNVPIYEGYALPH |
| | | | AIMRLDLAGRDLTDYLMKILTERGY |
| | | | SFVTTAEREIVRDIKEKLCYVALDFE |
| | | | NEMATAASSSSLEKSYELPDGQVITI |
| | | | GNERFRCPETLFQPSFIGMESAGIHET |
| | | | TYNSIMKCDIDIRKDLYANNVMSGG |
| | | | TTMYPGIADRMQKEITALAPSTMKIK |
| | | | IIAPPERKYSVWIGGSILASLSTFQQM |
| | | | WITKQEYDEAGPSIVHRKCF |
| SEQ ID | P02609 | Myosin | MAPKKAKRRAAEGSSNVFSMFDQT |
| NO: 23 | (UniProtKB) | regulatory light | QIQEFKEAFTVIDQNRDGIIDKDDLRE |
| | | chain 2, skeletal | TFAAMGRLNVKNEELDAMIKEASGP |
| | | muscle | INFTVFLTMFGEKLKGADPEDVIMG |
| | | isoform/Gallu | AFKVLDPDGKGSIKKSFLEELLTTQC |
| | | gallus | DRFTPEEIKNMWAAFPPDVAGNVDY |
| | | | KNICYVITHGEDKEGE |
| SEQ ID | Q9PTY2 | Skeletal myosin | MSSDAEMAIFGEAAPYLRKSEKERIE |
| NO: 24 | (UniProtKB) | heavy | AQNKPFDAKTSVFVVHAKESYVKST |
| | | chain/Gallus | IQSKESGKVTVKTESGETLTVKEDQI |
| | | gallus | FSMNPPKYDKIEDMAMMTHLHEPA |
| | | | VLYNLKERYAAWMIYTYSGLFCVTV |
| | | | NPYKWLPVYNPEVVLAYRGKKRQE |
| | | | APPHIFSISDNAYQFMLTDRENQSILI |
| | | | TGESGAGKTVNTKRVIQYFATIAASG |
| | | | DKKKEEQPAGKMQGTLEDQIISANP |
| | | | LLEAFGNAKTVRNDNSSRFGKFIRIH |
| | | | FGATGKLASADIETYLLEKSRVTFQL |
| | | | KAERSYHIFYQIMSNKKPELIEMLLIT |
| | | | TNPYDYHYVSQGEITVPSINDQEELM |
| | | | ATDSAIDILGFTPDEKTAIYKLTGAV |
| | | | MHYGNLKFKQKQREEQAEPDGTEV |
| | | | ADKAAYLMGLNSADLLKALCYPRV |
| | | | KVGNEFVTKGQTVQQVYNSVGALA |
| | | | KAVFEKMFLWMVVRINQQLDTKQP |
| | | | RQYFIGVLDIAGFEIFDFNSLEQLCIN |
| | | | FTNEKLQQFFNHHMFVLEQEEYKKE |
| | | | GIEWEFIDFGMDLAACIELIEKPMGIF |
| | | | SILEEECMFPKATDTSFKNKLYDQHL |
| | | | GKSNNFQKPKPGKGKAEAHFSLVHY |
| | | | AGTVDYNISGWLDKNKDPLYETVV |
| | | | GLYQKSSLKTLALLFASAGGEAESG |
| | | | GGGKKGGKKKGSSFQTVSALFRENL |
| | | | NKLMTNLRSTHPHFVRCIIPNETKTP |
| | | | GAMEHELVLHQLRCNGVLEGIRICR |
| | | | KGFPSRILYADFKQRYKVLNASAIPE |
| | | | GQFIDSKKASEKLLGSIDVDHTQYKF |
| | | | GHTKVFFKAGLLGLLEEMRDEKLAQ |
| | | | LITRTQARCRGFLMRVEYRRMVERR |
| | | | ESIFCIQYNIRSFMNVKHWPWMKLFF |
| | | | KIKPLLKSAESEKEMANMKGEFEKT |
| | | | KEELAKSGAKRKDLEGKMVSLLQEK |
| | | | NDLQLQVQAEADALADAEERCDQLI |
| | | | KTKIQLEAKIKEVTERAEDEEEINAEL |
| | | | TAKKRKLEDECSELKKDIDDLELTLA |
| | | | KVEKEKHATENKVKNLTEEMAALD |
| | | | ENIAKLTKEKKAPQEAHQQTLDDLQ |
| | | | AEEDKVNTLTKAKTKLEQQVDDLEG |
| | | | SLEQEKKLRMDLERAKRKLEGDLKL |
| | | | AHDSIMDLENDKQQLDEKLKKKDFE |
| | | | ISQIQSKIEDEQALGMQLQKKIKELQ |
| | | | ARTEELEEEIEAERTSRAKAEKHRAD |
| | | | LSRELEEISERLEEAGGATAAQIDMN |
| | | | KKREAEFQKMRRDLEEATLQHEATA |
| | | | AALRKKHADSTAELGEQIDNLQRVK |
| | | | QKLEKEKSELKMEIDDLASNMESVS |
| | | | KAKASLEKTCRALEDQMSEIKTKEE |
| | | | EHQRMINDVNAQRARLQTESGEYSR |
| | | | QVEEKDALISQLSRGKQAFTQQIEEL |
| | | | KRHLEEEIKAKNALAHGLQSARHDC |
| | | | DLLREQYEEEQEAKGELQRALSKAN |
| | | | SEVAQWRTKYETDAIQRTEELEEAK |
| | | | KKLAQRLQDAEEHVEAVNSKCASLE |
| | | | KTKQRLQNEVEDLMIDVERANSACA |
| | | | ALDKKQKNFDKILSEWKQKYEETQA |
| | | | ELEASQKESRSLSTELFKMKNAYEES |
| | | | LDHLETLKRENKNLQQEISDLTEQIA |
| | | | EGGKAIHELEKVKKQIEQEKSELQAS |
| | | | LEEAEASLEHEEGKILRLQLELNQVK |
| | | | SEIDRKIAEKDEEIDQLKRNHLRIVES |
| | | | MQRTLDAEVRSRNEALRLKKKMEG |
| | | | DLNEMEIQLNHANRMAAEAQKNLR |
| | | | NTQGVLKDTQIHLDDALRSQEDLKE |
| | | | QVAMVERRANLLQAETEELRAALEQ |
| | | | TERSRKVAEQELLDASERVQLLHTQ |
| | | | NTSLINTKKKLESDISQIQSEMEDTIQ |
| | | | EARNAEEKAKKAITDAAMMAEELK |
| | | | KEQDTSAHLERMKKNLDQTVKDLQ |
| | | | HRLDEAEQLALKGGKKQIQKLEARV |
| | | | RELEGEVDAEQKRSAEAVKGVRKYE |
| | | | RRVKELTYQSEEDRKNVLRLQDLVD |
| | | | KLQMKVKSYKRQAEEAEELSNVNLS |
| | | | KFRKIQHELEEAEERADIAESQVNKL |
| | | | RVKSREFHKKIEEEEI |
| SEQ ID | A0A1S3L3X1 | Myosin heavy | MSTDAEMQVYGKAAIYLRKSEKER |
| NO: 25 | (UniProtKB) | chain, fast | MEAQAMPFDSKNSCYVTDKVELYL |
| | | skeletal muscle- | KGLVTARADGKCTVTVTKPDGTKEE |
| | | like | GKEFKDADIYEMNPPKYDKIEDMAM |
| | | isoform/Salmo | MTYLNEASVLYNLKERYAAWMIYT |
| | | salar | YSGLFCATVNPYKWLPVYDEEVVN |
| | | | AYRGKKRVEAPPHIFSVSDNAFQFM |
| | | | MIDKENQSVLITGESGAGKTVNTKR |
| | | | VIQYFATIAVSGGKKEADPNKMQGS |
| | | | LEDQIIAANPLLESYGNAKTVRNDNS |
| | | | SRFGKFIRIHFQAGKLAKADIETYLLE |
| | | | KSRVSFQLPDERGYHIFFQMMTGHK |
| | | | PELVELALLTTNPYDFPMCSQGQIAV |
| | | | ASINDNEELDATDEAITILGFTNEEKL |
| | | | GIYKLTGAVVHHGNLKFKQKQREEQ |
| | | | AEPDGTEVADKIAYLLGLNSAEMLK |
| | | | ALCYPRVKVGNEYVTKGQTVAQVN |
| | | | NSVSALAKSIYERMFLWMVIRINEM |
| | | | LDTKNPRQFYIGVLDIAGFEIFDYNS |
| | | | MEQLCINFTNEKLQQFFNHTMFVLE |
| | | | QEEYKKEGIVWAFIDFGMDLAACIEL |
| | | | IEKPLGIFSILEEECMFPKSSDTTFKDK |
| | | | LYAQHLGKTKAFEKPKPAKGKAEA |
| | | | HFSLVHYAGTVDYNITGWLEKNKDP |
| | | | LNDSVCQLYGKSGVKILAALYPPPPP |
| | | | EDKAKKGGKKKGGSMQTVSSQFRE |
| | | | NLHKLMTNLRSTHPHFVRCLIPNESK |
| | | | TPGLMENFLVIHQLRCNGVLEGIRIC |
| | | | RKGFPSRIIYADFKQRYKVLNASVIPE |
| | | | GQFMDNKKASEKLLGSIDVNHEDYK |
| | | | FGHTKVFFKAGLLGVLEEMRDEKLA |
| | | | TLVGMVQALSRGFLMRREFSKMME |
| | | | RRESIYAIQYNIRSFMNVKTWPWMK |
| | | | LYFKIKPLLQSAETEKELANMKENYE |
| | | | KMKTDLAKALSTKKQMEEKLVSLT |
| | | | QEKNDLALQVASEGESLNDAEERCE |
| | | | GLIKSKIQQEAKLKETTERLEDEEEIN |
| | | | AELTAKKRKLEDECSELKKDIDDLEL |
| | | | TLAKVEKEKHATENKVKNLTEEMAS |
| | | | MDESVAKLTKEKKALQEAHQQTLD |
| | | | DLQAEEDKVNTLTKAKTKLEQQVD |
| | | | DLEGSLEQEKKLRMDLERAKRKLEG |
| | | | DLKLAQESIMDLENDKQQADEKIKK |
| | | | KEFETTQLLSKIEDEQSLGAQLQKKI |
| | | | KELQARIEELEEEIEAERAARAKVEK |
| | | | QRADLSRELEEISERLEEAGGATAAQ |
| | | | IEMNKKREAEFQKLRRDLEESTLQHE |
| | | | ATAAALRKKQADSVAELGEQIDNLQ |
| | | | RVKQKLEKEKSEYKMEIDDLSSNME |
| | | | AVAKAKGNLEKMCRTLEDQLSELKT |
| | | | KNDENVRQVNDISGQRARLLTENGE |
| | | | FGRQLEEKEALVSQLTRGKQAFTQQ |
| | | | VEELKRLIEEEVKAKNALAHGVQSA |
| | | | RHDCDLLREQFEEEQEAKAELQRGM |
| | | | SKANSEVAQWRTKYETDAIQRTEEL |
| | | | EEAKKKLAQRLQEAEETIEATNSKC |
| | | | ASLEKTKQRLQGEVEDLMIDVERAN |
| | | | ALAANLDKKQRNFDKVLAEWKQKY |
| | | | EEGQAELEGAQKEARSMSTELFKMK |
| | | | NSYEEALDHLETLKRENKNLQQEISD |
| | | | LTEQIGETGKSIHELEKAKKTVETEK |
| | | | SEIQTALEEAEGTLEHEESKILRVQLE |
| | | | LNQIKGEVDRKIAEKDEEMEQIKRNS |
| | | | QRVVDSMQSTLDSEVRSRNDALRVK |
| | | | KKMEGDLNEMEIQLSHSNRQAAEAQ |
| | | | KQLRNVQGQLKDAQLHLDDAVRAA |
| | | | EDMKEQAAMVERRNGLMVAEIEEL |
| | | | RVALEQTERGRKVAETELVDASERV |
| | | | GLLHSQNTSLLNTKKKLETDLVQVQ |
| | | | GEVDDIVQEARNAEEKAKKAITDAA |
| | | | MMAEELKKEQDTSSHLERMKKNLE |
| | | | VTVKDLQHRLDEAENLAMKGGKKQ |
| | | | LQKLESRVRELETEVEAEQRRGVDA |
| | | | VKGVRKYERRVKELTYQTEEDKKN |
| | | | VNRLQDLVDKLQMKVKAYKRQAEE |
| | | | AEEAANQHMSKFRKVQHELEEAEER |
| | | | ADIAETQVNKLRAKTRDSGKGKEAA |
| | | | E |
| SEQ ID | A0A1S3QIZ8 | Myosin heavy | MSTDAEMQIYGKAAIYLRKSEKERM |
| NO: 26 | (UniProtKB) | chain, fast | EAQAAPFDSKNSCYVADKVELYLKG |
| | | skeletal muscle- | LITARADGKCTVTVTKPDGTKEEGK |
| | | like/Salmo salar | EFKDADIYEMNPPKYDKIEDMAMM |
| | | | TYLNEASVLYNLKERYAAWMIYTYS |
| | | | GLFCATVNPYKWLPVYDAEVVNAY |
| | | | RGKKRMEAPPHIFSVSDNAFQFMLID |
| | | | KENQSVLITGESGAGKTVNTKRVIQY |
| | | | FATIAVSGGEKKKEVDPSKMQGSLE |
| | | | DQIIAANPLLEAYGNAKTVRNDNSSR |
| | | | FGKFIRIHFQGGKLAKADIETYLLEKS |
| | | | RVSFQLPDERGYHIFFQMMTGHKPEI |
| | | | VEMALITTNPYDFPMCSQGQIAVASI |
| | | | DDKEELDATDDAITILGFTNDEKIGIY |
| | | | KLTGAVVHHGNLKFKQKQREEQAE |
| | | | PDGTEVADKIGYLLGLNSAEMLKAL |
| | | | CYPRVKVGNEYVTKGQTVPQVNNS |
| | | | VMALAKSIYERMFLWMVIRINEMLD |
| | | | TKNPRQFYIGVLDIAGFEIFDYNSME |
| | | | QLCINFTNEKLQQFFNHTMFVLEQEE |
| | | | YKKEGIVWAFIDFGMDLAACIELIEK |
| | | | PLGIFSILEEECMFPKSSDTTFKDKLY |
| | | | SQHLGKTQAFEKPKPAKGKAEAHFS |
| | | | LVHYAGTVDYNITGWLEKNKDPLN |
| | | | DSVCQLYGKSGVKILAALYPAAPPE |
| | | | DTTKKGGKKKGGSMQTVSSQFRENL |
| | | | HKLMTNLRSTHPHFVRCLIPNESKTP |
| | | | GLMENFLVIHQLRCNGVLEGIRICRK |
| | | | GFPSRIIYADFKQRYKVLNASVIPEG |
| | | | QFMDNKKASEKLLGSIDVNHEDYKF |
| | | | GHTKVSQILYFKIKPLLQSAETEKEL |
| | | | ANMKENYEKMTADLAKALSTKKQ |
| | | | MEEKLVALMQEKNDLALQVAS |
| | A0JNJ5 | Myosin light | MAPKKDVKKPAAAAAPAPAPAPAP |
| | (UniProtKB) | chain 1/3, | APAPAPPKEEKIDLSAIKIEFSKQQQD |
| | | skeletal muscle | EFKEAFLLFDRTGECKITLSQVGDVL |
| | | isoform/Bos | RALGTNPTNAEVKKVLGNPSNEEMN |
| | | taurus | AKKIEFEQFLPMLQAISNNKDQGTYE |
| | | | DFVEGLRVFDKEGNGTVMGAELRH |
| | | | VLATLGEKMKEEEVEALMAGQEDS |
| | | | NGCINYEAFVKHIMSN |
| SEQ ID | P02604 | Myosin light | MAPKKDVKKPAAAAAPAPAPAPAP |
| NO: 27 | (UniProtKB) | chain 1, skeletal | APAPAKPKEPAIDLKSIKIEFSKEQQD |
| | | muscle | DFKEAFLLFDRTGDAKITLSQVGDIV |
| | | isoform/Gallus | RALGQNPTNAEINKILGNPSKEEMNA |
| | | gallus | KKITFEEFLPMLQAAANNKDQGTFE |
| | | | DFVEGLRVFDKEGNGTVMGAELRH |
| | | | VLATLGEKMTEEEVEELMKGQEDSN |
| | | | GCINYEAFVKHIMSV |
| SEQ ID | P02605 | Myosin light | MSFSPDEINDFKEAFLLFDRTGDAKI |
| NO: 28 | (UniProtKB) | chain 3, skeletal | TLSQVGDIVRALGQNPTNAEINKILG |
| | | muscle/Gallus | NPSKEEMNAKKITFEEFLPMLQAAA |
| | | gallus | NNKDQGTFEDFVEGLRVFDKEGNGT |
| | | | VMGAELRHVLATLGEKMTEEEVEEL |
| | | | MKGQEDSNGCINYEAFVKHIMSV |
| SEQ ID | B5DGT2 | Myosin light | MADAAPAEASGASAFTADQIEDFKE |
| NO: 29 | (UniProtKB) | chain 3, skeletal | AFGLFDRVGDSMIGYNQVADVMRA |
| | | muscle/Salmo | LGQNPQNKEVAAILGKPSADDMAN |
| | | salar | KRLAFADFMPMMEKVDKIVKGTLD |
| | | | DYVEGLRVFDKEGNGTVSGAELRIV |
| | | | LGTLGEKMSEAEIDSLLIGQEDENGSI |
| | | | NYEAFVKHVLSV |
| SEQ ID | P13538 | Myosin heavy | MASPDAEMAAFGEAAPYLRKSEKER |
| NO: 30 | (UniProtKB) | chain, skeletal | IEAQNKPFDAKSSVFVVHPKESFVKG |
| | | muscle, adult/ | TIQSKEGGKVTVKTEGGETLTVKED |
| | | Gallus gallus | QVFSMNPPKYDKIEDMAMMTHLHE |
| | | | PAVLYNLKERYAAWMIYTYSGLFCV |
| | | | TVNPYKWLPVYNPEVVLAYRGKKR |
| | | | QEAPPHIFSISDNAYQFMLTDRENQSI |
| | | | LITGESGAGKTVNTKRVIQYFATIAA |
| | | | SGEKKKEEQSGKMQGTLEDQIISANP |
| | | | LLEAFGNAKTVRNDNSSRFGKFIRIH |
| | | | FGATGKLASADIETYLLEKSRVTFQL |
| | | | PAERSYHIFYQIMSNKKPELIDMLLIT |
| | | | TNPYDYHYVSQGEITVPSIDDQEELM |
| | | | ATDSAIDILGFSADEKTAIYKLTGAV |
| | | | MHYGNLKFKQKQREEQAEPDGTEV |
| | | | ADKAAYLMGLNSAELLKALCYPRV |
| | | | KVGNEFVTKGQTVSQVHNSVGALA |
| | | | KAVYEKMFLWMVIRINQQLDTKQPR |
| | | | QYFIGVLDIAGFEIFDFNSFEQLCINFT |
| | | | NEKLQQFFNHHMFVLEQEEYKKEGI |
| | | | EWEFIDFGMDLAACIELIEKPMGIFSI |
| | | | LEEECMFPKATDTSFKNKLYDQHLG |
| | | | KSNNFQKPKPAKGKAEAHFSLVHYA |
| | | | GTVDYNISGWLEKNKDPLNETVIGL |
| | | | YQKSSVKTLALLFATYGGEAEGGGG |
| | | | KKGGKKKGSSFQTVSALFRENLNKL |
| | | | MANLRSTHPHFVRCIIPNETKTPGAM |
| | | | EHELVLHQLRCNGVLEGIRICRKGFP |
| | | | SRVLYADFKQRYRVLNASAIPEGQF |
| | | | MDSKKASEKLLGSIDVDHTQYRFGH |
| | | | TKVFFKAGLLGLLEEMRDDKLAEIIT |
| | | | RTQARCRGFLMRVEYRRMVERRESI |
| | | | FCIQYNVRSFMNVKHWPWMKLFFKI |
| | | | KPLLKSAESEKEMANMKEEFEKTKE |
| | | | ELAKSEAKRKELEEKMVVLLQEKND |
| | | | LQLQVQAEADSLADAEERCDQLIKT |
| | | | KIQLEAKIKEVTERAEDEEEINAELTA |
| | | | KKRKLEDECSELKKDIDDLELTLAK |
| | | | VEKEKHATENKVKNLTEEMAVLDE |
| | | | TIAKLTKEKKALQEAHQQTLDDLQV |
| | | | EEDKVNTLTKAKTKLEQQVDDLEGS |
| | | | LEQEKKLRMDLERAKRKLEGDLKLA |
| | | | HDSIMDLENDKQQLDEKLKKKDFEI |
| | | | SQIQSKIEDEQALGMQLQKKIKELQA |
| | | | RIEELEEEIEAERTSRAKAEKHRADLS |
| | | | RELEEISERLEEAGGATAAQIEMNKK |
| | | | REAEFQKMRRDLEEATLQHEATAAA |
| | | | LRKKHADSTAELGEQIDNLQRVKQK |
| | | | LEKEKSELKMEIDDLASNMESVSKA |
| | | | KANLEKMCRTLEDQLSEIKTKEEQN |
| | | | QRMINDLNTQRARLQTETGEYSRQA |
| | | | EEKDALISQLSRGKQGFTQQIEELKR |
| | | | HLEEEIKAKNALAHALQSARHDCEL |
| | | | LREQYEEEQEAKGELQRALSKANSE |
| | | | VAQWRTKYETDAIQRTEELEEAKKK |
| | | | LAQRLQDAEEHVEAVNAKCASLEKT |
| | | | KQRLQNEVEDLMVDVERSNAACAA |
| | | | LDKKQKNFDKILAEWKQKYEETQTE |
| | | | LEASQKESRSLSTELFKMKNAYEESL |
| | | | DHLETLKRENKNLQQEIADLTEQIAE |
| | | | GGKAVHELEKVKKHVEQEKSELQAS |
| | | | LEEAEASLEHEEGKILRLQLELNQIKS |
| | | | EIDRKIAEKDEEIDQLKRNHLRIVES |
| | | | MQSTLDAEIRSRNEALRLKKKMEGD |
| | | | LNEMEIQLSHANRMAAEAQKNLRNT |
| | | | QGTLKDTQIHLDDALRTQEDLKEQV |
| | | | AMVERRANLLQAEVEELRGALEQTE |
| | | | RSRKVAEQELLDATERVQLLHTQNT |
| | | | SLINTKKKLETDIVQIQSEMEDTIQEA |
| | | | RNAEEKAKKAITDAAMMAEELKKE |
| | | | QDTSAHLERMKKNMDQTVKDLHVR |
| | | | LDEAEQLALKGGKKQLQKLEARVRE |
| | | | LEGEVDSEQKRSAEAVKGVRKYERR |
| | | | VKELTYQCEEDRKNILRLQDLVDKL |
| | | | QMKVKSYKRQAEEAEELSNVNLSKF |
| | | | RKIQHELEEAEERADIAESQVNKLRV |
| | | | KSREIHGKKIEEEE |
| SEQ ID | Q8AXY6 | Muscle, skeletal | MRDLLVVPLGHVLTLAALSLAETLQ |
| NO: 31 | (UniProtKB) | receptor tyrosine | KAPFISTPLETVDALVEDVPKFVCVV |
| | | protein | ESYPEPEITWTRNSIPIRLFDTRYSIQR |
| | | kinase/Gallus | NGQLLTILSVEDSDDGVYCCTADNG |
| | | gallus | VGAAAQSCGALQVKMRPKITRPPVN |
| | | | VEIIEGLKAVLPCTTMGNPKPSVSWI |
| | | | KGETVVKENARIAVLDSGNLRIHNV |
| | | | QREDAGQYRCVAKNSLGSAYSKPAT |
| | | | VVVEVFARILKAPESQNITFGSMVTL |
| | | | RCTAAGAPVPTVTWLENGKAVSAGS |
| | | | IAESVKDRVVDSRLQVYVTRPGLFTC |
| | | | LATNKHSKTFGAAKAAATISVSEWS |
| | | | KLYKGDAGYCSTYRGEVCSAILSRN |
| | | | ALVFFNSSYADPEETQELLVHTAWT |
| | | | ELKTVSSFCQPAAESLLCNYIFQECK |
| | | | PSGVGPAPKPICRENCLAVKDLYCFK |
| | | | EWLSMEENSQRGIYKPGLMLLALPE |
| | | | CNRLPSLHQDPSACTHIPFFDFKKENI |
| | | | TRTCYSGNGQFYQGWANVTASGIPC |
| | | | QKWSDQAPHLHRRTPQVFPELSDAE |
| | | | NYCRNPGGENERPWCYTKDPSVTW |
| | | | EYCSVSPCGDASLSLGTRKPNGETQN |
| | | | LPPPPSYSPTYSMNVIILIISSFALIVIL |
| | | | GIITLVCCRRRKQWKNKKRESETPTL |
| | | | TTLPSELLLDRLHPNPMYQRMPLLLN |
| | | | PKLLSLEYPRNNIEYVRDIGEGAFGR |
| | | | VFQARAPGLLPYEPFTMVAVKMLKE |
| | | | EASADMQADFQREAALMAEFDNPNI |
| | | | VKLLGVCAVGKPMCLLFEYMAYGD |
| | | | LNEYLRDRSPRNLCSLVQGGLEARA |
| | | | CLLNPLALCCTSQLCIAKQVAAGMA |
| | | | YLSERKFVHRDLATRNCLVGENMV |
| | | | VKIADFGLSRNMYSADYYKANEND |
| | | | AIPIRWMPPESIFYNRYTTESDVWAY |
| | | | GVVLWEIFSYGMQPYYGMAHEEVIY |
| | | | YVRDGNILSCPDNCPLELYNLMRLC |
| | | | WSKLPADRPSFASIHRILERMYERAV |
| | | | ASPQV |
| SEQ ID | P02588 | Troponin C, | MASMTDQQAEARAFLSEEMIAEFKA |
| NO: 32 | (UniProtKB) | skeletal | AFDMFDADGGGDISTKELGTVMRM |
| | | muscle/Gallus | LGQNPTKEELDAIIEEVDEDGSGTIDF |
| | | gallus | EEFLVMMVRQMKEDAKGKSEEELA |
| | | | NCFRIFDKNADGFIDIEELGEILRATG |
| | | | EHVTEEDIEDLMKDSDKNNDGRIDF |
| | | | DEFLKMMEGVQ |
| SEQ ID | P02587 | Troponin C, | TDQQAEARSYLSEEMIAEFKAAFDM |
| NO: 33 | (UniProtKB) | skeletal | FDADGGGDISVKELGTVMRMLGQTP |
| | | muscle/Sus | TKEELDAIIEEVDEDGSGTIDFEEFLV |
| | | scrofa | MMVRQMKEDAKGKSEEELAECFRIF |
| | | | DRNMDGYIDAEELAEIFRASGEHVT |
| | | | DEEIESIMKDGDKNNDGRIDFDEFLK |
| | | | MMEGVQ |
| SEQ ID | P68246 | Troponin 1, fast | MSDEEKKRRAATARRQHLKSAMLQ |
| NO: 34 | (UniProtKB) | skeletal | LAVTEIEKEAAAKEVEKQNYLAEHC |
| | | muscle/Gallus | PPLSLPGSMQELQELCKKLHAKIDSV |
| | | gallus | DEERYDTEVKLQKTNKELEDLSQKL |
| | | | FDLRGKFKRPPLRRVRMSADAMLRA |
| | | | LLGSKHKVNMDLRANLKQVKKEDT |
| | | | EKEKDLRDVGDWRKNIEEKSGMEG |
| | | | RKKMFEAGES |
| SEQ ID | P68247 | Troponin 1, fast | MSDEEKKRRAATARRQHLKSAMLQ |
| NO: 35 | (UniProtKB) | skeletal | LAVTEIEKEAAAKEVEKQNYLAEHC |
| | | muscle/Cotumix | PPLSLPGSMQE |
| | | japonica | LQELCKKLHAKIDSVDEERYDTEVK |
| | | | LQKTNKELEDLSQKLFDLRGKFKRPP |
| | | | LRRVRMSAD |
| | | | AMLRALLGSKHKVNMDLRANLKQV |
| | | | KKEDTEKEKDLRDVGDWRKNIEEKS |
| | | | GMEGRKKMFEA |
| | | | GES |
| SEQ ID | Q8MKI3 | Troponin T, fast | MSDEEVEHVEEEYEEEEEAQEEAPPP |
| NO: 36 | (UniProtKB) | skeletal | PAEVPEVHEEVHEVHEPEEVQEEEKP |
| | | muscle/Bos | RPRLTAPKIPEGEKVDFDDIQKKRQN |
| | | taurus | KDLMELQALIDSHFEARKKEEEELV |
| | | | ALKERIEKRRAERAEQQRIRAEKERE |
| | | | RQNRLAEEKARREEEDAKRRAEDDL |
| | | | KKKKALSSMGANYSSYLAKADQKR |
| | | | GKKQTAREMKKKVLAERRKPLNIDH |
| | | | LSEDKLRDKAKELWDTLYQLETDKF |
| | | | EYGEKLKRQKYDITNLRSRIDQAQK |
| | | | HSKKAGTAPKGKVGGRWK |
| SEQ ID | Q75ZZ6 | Troponin T, | MSDAEEQEYEEEQPEEEEAAEEEEAP |
| NO: 37 | (UniProtKB) | slow skeletal | EEPEPVAEREEERPKPSRPVVPPLIPP |
| | | muscle/Sus | KIPEGERVDFDDIHRKRMEKDLLELQ |
| | | scrofa | TLIDVHFEQRKKEEEELVALKERIER |
| | | | RRAERAEQQRFRTEKERERQAKLAE |
| | | | EKMRKEEEEAKKRAEDDAKKKKVL |
| | | | SNMGAHFGGYLVKAEQKRGKRQTG |
| | | | REMKQRILSERKKPLNIDHMGEDQL |
| | | | REKAQELSDWIHQLESEKFDLMAKL |
| | | | KQQKYEINVLYNRISHAQKFRKGAG |
| | | | KGRVGGRWK |
| SEQ ID | NP_990105.1 | Tropomodulin- | MTSYRQELEKYRDIDEDKILQELSAE |
| NO: 38 | (NCBI) | 4/Gallus gallus | ELEQLDTELLEMDPENVLLPAGLRQ |
| | | | RDQTQKSPTGPLDREALLQHLEKQA |
| | | | LEAKEREDLVPFTGEKKGKPFVPKNP |
| | | | TREIPREEQITLEPELEEALANATEAE |
| | | | MCDIAAILGMYTLMSNKQYYDAICS |
| | | | GTITNTEGINSVVKPDKYKPVPDEPP |
| | | | NPTNVEETLRQIQANDSALEDVNLN |
| | | | NIKDIPISTLKAICEAMKTNTHVKKLS |
| | | | LVATRSNDPVATAVAEMLAENKTLQ |
| | | | SLNIESNFITSAGMMSVIKAMYQNST |
| | | | LSELKVDNQCQRLGNTVEMEMATM |
| | | | LEQCPSVVRFGYHFTQQGPRARAAI |
| | | | AITRNNELRRKQKKT |
| SEQ ID | P68139 | Alpha-actin- | MCDEDETTALVCDNGSGLVKAGFA |
| NO: 39 | (UniProtKB) | 1/Gallus gallus | GDDAPRAVFPSIVGRPRHQGVMVG |
| | | | MGQKDSYVGDEAQSKRGILTLKYPI |
| | | | EHGIITNWDDMEKIWHHTFYNELRV |
| | | | APEEHPTLLTEAPLNPKANREKMTQI |
| | | | MFETFNVPAMYVAIQAVLSLYASGR |
| | | | TTGIVLDSGDGVTHNVPIYEGYALPH |
| | | | AIMRLDLAGRDLTDYLMKILTERGY |
| | | | SFVTTAEREIVRDIKEKLCYVALDFE |
| | | | NEMATAASSSSEEKSYELPDGQVITI |
| | | | GNERFRCPETLFQPSFIGMESAGIHET |
| | | | TYNSIMKCDIDIRKDLYANNVMSGG |
| | | | TTMYPGIADRMQKEITALAPSTMKIK |
| | | | IIAPPERKYSVWIGGSILASLSTFQQM |
| | | | WITKQEYDEAGPSIVHRKCF |
| SEQ ID | P20111 | Alpha-actinin- | MNSMNQIETNMQYTYNYEEDEYMT |
| NO: 40 | (UniProtKB) | 2/Gallus gallus | QEEEWDRDLLLDPAWEKQQRKTFT |
| | | | AWCNSHLRKAGTQIENIEEDFRNGL |
| | | | KLMLLLEVISGERLPKPDRGKMRFH |
| | | | KIANVNKALDYIASKGVKLVSIGAEE |
| | | | IVDGNVKMTLGMIWTIILRFAIQDISV |
| | | | EETSAKEGLLLWCQRKTAPYRNVNI |
| | | | QNFHLSWKDGLGLCALIHRHRPDLI |
| | | | DYSKLNKDDPIGNINLAMEIAEKHLD |
| | | | IPKMLDAEDIVNTPKPDERAIMTYVS |
| | | | CFYHAFAGAEQAETAANRICKVLAV |
| | | | NQENERLMEEYERLASELLEWIRRTI |
| | | | PWLENRTPEKTMQAMQKKLEDFRD |
| | | | YRRKHKPPKVQEKCQLEINFNTLQT |
| | | | KLRISNRPAFMPSEGKMVSDIAGAW |
| | | | QRLEQAEKGYEEWLLNEIRRLERLE |
| | | | HLAEKFRQKASTHEQWAYGKEQILL |
| | | | QKDYESASLTEVRAMLRKHEAFESD |
| | | | LAAHQDRVEQIAAIAQELNELDYHD |
| | | | AASVNDRCQKICDQWDSLGTLTQKR |
| | | | REALERTEKLLETIDQLHLEFAKRAA |
| | | | PFNNWMEGAMEDLQDMFIVHSIEEI |
| | | | QSLISAHDQFKATLPEADGERQAILSI |
| | | | QNEVEKVIQSYSMRISASNPYSTVTV |
| | | | EEIRTKWEKVKQLVPQRDQSLQEEL |
| | | | ARQHANERLRRQFAAQANVIGPWIQ |
| | | | TKMEEIARSSIEMTGPLEDQMNQLK |
| | | | QYEQNIINYKHNIDKLEGDHQLIQEA |
| | | | LVFDNKHTNYTMEHIRVGWELLLTT |
| | | | IARTINEVETQILTRDAKGITQEQMN |
| | | | DFRASFNHFDRRKNGLMDHDDFRA |
| | | | CLISMGYDLGEAEFARIMSLVDPNG |
| | | | QGTVTFQSFIDFMTRETADTDTAEQV |
| | | | IASFRILASDKPYILADELRRELPPEQ |
| | | | AQYCIKRMPQYTGPGSVPGALDYTS |
| | | | FSSALYGESDL |
| SEQ ID | P20111-2 | Alpha-actinin- | MNSMNQIETNMQYTYNYEEDEYMT |
| NO: 41 | (UniProtKB) | 2/Gallus gallus | QEEEWDRDLLLDPAWEKQQRKTFT |
| | | | AWCNSHLRKAGTQIENIEEDFRNGL |
| | | | KLMLLLEVISGERLPKPDRGKMRFH |
| | | | KIANVNKALDYIASKGVKLVSIGAEE |
| | | | IVDGNVKMTLGMIWTIILRFAIQDISV |
| | | | EETSAKEGLLLWCQRKTAPYRNVNI |
| | | | QNFHLSWKDGLGLCALIHRHRPDLI |
| | | | DYSKLNKDDPIGNINLAMEIAEKHLD |
| | | | IPKMLDAEDIVNTPKPDERAIMTYVS |
| | | | CFYHAFAGAEQAETAANRICKVLAV |
| | | | NQENERLMEEYERLASELLEWIRRTI |
| | | | PWLENRTPEKTMQAMQKKLEDFRD |
| | | | YRRKHKPPKVQEKCQLEINFNTLQT |
| | | | KLRISNRPAFMPSEGKMVSDIAGAW |
| | | | QRLEQAEKGYEEWLLNEIRRLERLE |
| | | | HLAEKFRQKASTHEQWAYGKEQILL |
| | | | QKDYESASLTEVRAMLRKHEAFESD |
| | | | LAAHQDRVEQIAAIAQELNELDYHD |
| | | | AASVNDRCQKICDQWDSLGTLTQKR |
| | | | REALERTEKLLETIDQLHLEFAKRAA |
| | | | PFNNWMEGAMEDLQDMFIVHSIEEI |
| | | | QSLISAHDQFKATLPEADGERQAILSI |
| | | | QNEVEKVIQSYSMRISASNPYSTVTV |
| | | | EEIRTKWEKVKQLVPQRDQSLQEEL |
| | | | ARQHANERLRRQFAAQANVIGPWIQ |
| | | | TKMEEIARSSIEMTGPLEDQMNQLK |
| | | | QYEQNIINYKHNIDKLEGDHQLIQEA |
| | | | LVFDNKHTNYTMEHIRVGWELLLTT |
| | | | IARTINEVETQILTRDAKGITQEQMN |
| | | | DFRASFNHFDRRKNGLMDHDDFRA |
| | | | CLISMGYDLDESDNLHSDEFKACLIS |
| | | | LGEVGNDLQGEAEFARIMSLVDPNG |
| | | | QGTVTFQSFIDFMTRETADTDTAEQV |
| | | | IASFRILASDKPYILADELRRELPPEQ |
| | | | AQYCIKRMPQYTGPGSVPGALDYTS |
| | | | FSSALYGESDL |
| SEQ ID | P13127 | F-actin-capping | MADFEDRVSDEEKVRIAAKFITHAPP |
| NO: 42 | (UniProtKB) | protein subunit | GEFNEVFNDVRLLLNNDNLLREGAA |
| | | alpha-1/Gallus | HAFAQYNMDQFTPVKIEGYDDQVLI |
| | | gallus | TEHGDLGNGRFLDPRNKISFKFDHLR |
| | | | KEASDPQPEDTESALKQWRDACDSA |
| | | | LRAYVKDHYPNGFCTVYGKSIDGQQ |
| | | | TIIACIESHQFQPKNFWNGRWRSEWK |
| | | | FTITPPTAQVAAVLKIQVHYYEDGNV |
| | | | QLVSHKDIQDSVQVSSDVQTAKEFIK |
| | | | IIENAENEYQTAISENYQTMSDTTFK |
| | | | ALRRQLPVTRTKIDWNKILSYKIGKE |
| | | | MQNA |
| SEQ ID | P28497 | F-actin-capping | MADLEEQLSDEEKVRIAAKFIIHAPP |
| NO: 43 | (UniProtKB) | protein subunit | GEFNEVFNDVRLLLNNDNLLREGAA |
| | | alpha-2/Gallus | HAFAQYNLDQFTPVKIDGYDEQVLIT |
| | | gallus | EHGDLGNGKFLDPKNKISFKFDHLR |
| | | | KEATDPRPHEVENAIESWRNSVETA |
| | | | MKAYVKEHYPNGVCTVYGKTIDGQ |
| | | | QTIIACIESHQFQAKNFWNGRWRSE |
| | | | WKFTISPSTTQVAGILKIQVHYYEDG |
| | | | NVQLVSHKDIQDSLTVSNEAQTAKE |
| | | | FIKIVEAAENEYQTAISENYQTMSDT |
| | | | TFKALRRQLPVTRTKIDWNKILSYKI |
| | | | GKEMQNA |
| SEQ ID | A0M8U0 | Capping protein | MADLEEQLSDEEKVRIAAKFIIHAPP |
| NO: 44 | (UniProtKB) | (Actin filament) | GEFNEVFNDVRLLLNNDNLLREGAA |
| | | muscle Z-line, | HAFAQYNLDQFTPVKIDGYDEQVLIT |
| | | alpha 2/Gallus | EHGDLGNGKFLDPKNKISFKFDHLR |
| | | gallus | KEATDPRPHEVENAIESWRNSVETA |
| | | | MKAYVKEHYPNGVCTVYGKTIDGQ |
| | | | QTIIACIESHQFQAKNFWNGRWRSE |
| | | | WKFTISPSTTQVAGILKIQVHYYEDG |
| | | | NVQLVSHKDIQDSLTVSNEAQTAKE |
| | | | FIKIVEAAENEYQTAISENYQTMSDT |
| | | | TFKALRRQLPVTRTKIDWNKILSYKI |
| | | | GKEMQNA |
| SEQ ID | NP_001265047.1 | F-actin-capping | MSVGQGLCESEKVSLICGLMRQSPP |
| NO: 45 | (NCBI) | protein subunit | GEFRQVVQDLCDLLQDDELVKQQA |
| | | alpha-3/Gallus | ARAGARHNKNNFTPVLVNGNTVLLT |
| | | gallus | QYNDLGGNRFFYPQDKFSFEFDHLS |
| | | | GVTSKTHLHRVMLDEGELWRGALH |
| | | | KGLNAYVNYHFPVGNCCVFKKSLG |
| | | | KRQMLVACIEAHQYQPSKHWNSLW |
| | | | KSDWTFSLTPVMTRVTGIFLLQLHYF |
| | | | RNANLHVTISKSVSESLHVIDRNQFV |
| | | | TDFVKFVKTEDNKIHNAILENIQALS |
| | | | EHTWRKNLRRRLPITRTFMNWNELL |
| | | | NNQHLKTGVSRKEVPP |
| SEQ ID | P02565 | Myosin-1B/ | MATDADMAIFGEAAPYLRKSEKERI |
| NO: 46 | (UniProtKB) | Gallus gallus | EAQNKPFDAKSSVFWHAKESYVKS |
| | | | TIQSKESGKVTVKTEGGETLTVKEDQ |
| | | | IFSMNPPKYDKIEDMAMMTHLHEPA |
| | | | VLYNLKERYAAWMIYTYSGLFCVTV |
| | | | NPYKWLPVYNPEVVLAYRGKKRQE |
| | | | APPHIFSISDNAYQFMLTDRENQSILI |
| | | | TGESGAGKTVNTKRVIQYFATIAASG |
| | | | DKKKEEQPAGKMQGTLEDQIISANP |
| | | | LLEAFGNAKTVRNDNSSRFGKFIRIH |
| | | | FGATGKLASADIETYLLEKSRVTFQL |
| | | | KAERSYHIFYQIMSNKKPELIEMLLIT |
| | | | TNPYDYQYVSQGEITVPSINDQEELM |
| | | | ATDSAIDILGFTPDEKTAIYKLTGAV |
| | | | MHYGNLKFKQKQREEQAEPGGTEV |
| | | | ADKAAYLMGLNSADLLKALCYPRV |
| | | | KVGNEYVTKGQTVQQVYNSVGALA |
| | | | KSVFEKMFLWMVVRINQQLDTKQP |
| | | | RQYFIGVLDIAGFEIFDFNSLEQLCIN |
| | | | FTNEKLQQFFNHHMFVLEQEEYKKE |
| | | | GIEWEFIDFGMDLAACIELIEKPMGIF |
| | | | SILEEECMFPKATDTSFKNKLYDQHL |
| | | | GKSNNFQKPKPGKGKAEAHFSLVHY |
| | | | AGTVDYNITGWLEKNKDPLNETVVG |
| | | | LYQKSSLKTLALLFASVGGAEAESG |
| | | | AGGKKGGKKKGSSFQTVSALFRENL |
| | | | NKLMSNLRSTHPHFVRCLIPNETKTP |
| | | | GAMEHELVLHQLRCNGVLEGIRICR |
| | | | KGFPIRILYADFKQRYKVLNASAIPE |
| | | | GQFIDSKKASEKLLGSIDVDHTQYKF |
| | | | GHTKVFFKAGLLGLLEEMRDEKLAQ |
| | | | LITRTQARCRGFLMRVEFKKMMERR |
| | | | ESIFCIQYNVRAFMNVKHWPWMKLF |
| | | | FKIKPLLKSAESEKEMANMKEEFEKT |
| | | | KEELAKSEAKRKELEEKMVSLLQEK |
| | | | NDLQLQVQAEADGLADAEERCDQLI |
| | | | KTKIQLEAKIKELTERAEDEEEMNAE |
| | | | LTAKKRKLEDECSELKKDIDDLELTL |
| | | | AKVEKEKHATENKVKNLTEEMAAL |
| | | | DETIAKLTKEKKALQEAHQQTLDDL |
| | | | QAEEDKVNTLTKAKTKLEQQVDDLE |
| | | | GSLEQEKKLRMDLERAKRKLEGDLK |
| | | | MTQESTMDLENDKQQLDEKLKKKD |
| | | | FEISQIQSKIEDEQALGMQLQKKIKEL |
| | | | QARIEELEEEIEAERTSRAKAEKHRA |
| | | | DLSRELEEISERLEEAGGATAAQIDM |
| | | | NKKREAEFQKMRRDLEEATLQHEAT |
| | | | AAALRKKHADSTADVGEQIDNLQRV |
| | | | KQKLEKEKSELKMEIDDLASNMESV |
| | | | SKAKANLEKMCRSLEDQLSEIKTKEE |
| | | | EQQRTINDISAQKARLQTESGEYSRQ |
| | | | VEEKDALISQLSRGKQAFTQQIEELK |
| | | | RHLEEEIKAKKCPAHALQSARHDCD |
| | | | LLREQYEEEQEAKGELQRALSKANS |
| | | | EVAQWRTKYETDAIQRTEELEEAKK |
| | | | KLAQRLQDAEEHVEAVNSKCASLEK |
| | | | TKQRLQNEVEDLMIDVERSNAACAA |
| | | | LDKKQKNFDKILSEWKQKYEETQAE |
| | | | LEASQKESRSLSTELFKMKNAYEESL |
| | | | DHLETLKRENKNLQQEISDLTEQIAE |
| | | | GGKAIHELEKVKKQIEQEKSELQTAL |
| | | | EEAEASLEHEEGKILRVQLELNQVKS |
| | | | DIDRKIAEKDEEIDQLKRNHLRVVDS |
| | | | MQSTLDAEIRSRNEALRLKKKMEGD |
| | | | LNEIEIQLSHANRQAAEAQKNLRNTQ |
| | | | GVLKDTQIHLDDALRSQEDLKEQVA |
| | | | MVERRANLLQAEIEELRAALEQTERS |
| | | | RKVAEQELLDASERVQLLHTQNTSLI |
| | | | NTKKKLESDISQIQSEMEDTIQEARN |
| | | | AEEKAKKAITDAAMMAEELKKEQD |
| | | | TSAHLERMKKNLDQTVKDLQHRLD |
| | | | EAEQLALKGGKKQIQKLEARVRELE |
| | | | GEVDAEQKRSAEAVKGVRKYERRV |
| | | | KELTYQSEEDRKNVLRLQDLVDKLQ |
| | | | MKVKSYKRQAEEAEELSNVNLSKFR |
| | | | KIQHELEEAEERADIAESQVNKLRAK |
| | | | SREIGKKAESEE |
| SEQ ID | Q9TV63 | Myosin-2/Sus | MSSDQEMAIFGEAAPYLRKSEKERIE |
| NO: 47 | (UniProtKB) | scrofa | AQNRPFDAKTSVFVAEPKESFVKGTI |
| | | | QSREGGKVTVKTEAGATLTVKEDQV |
| | | | FPMNPPKFDKIEDMAMMTHLHEPGV |
| | | | LYNLKERYAAWMIYTYSGLFCVTVN |
| | | | PYKWLPVYNPEVVTAYRGKKRQEA |
| | | | PPHIFSISDNAYQFMLTDRENQSILIT |
| | | | GESGAGKTVNTKRVIQYFATIAVTGE |
| | | | KKKEEPTSGKMQGTLEDQIISANPLL |
| | | | EAFGNAKTVRNDNSSRFGKFIRIHFG |
| | | | TTGKLASADIETYLLEKSRVTFQLKA |
| | | | ERSYHIFYQITSNRKPELIEMLLITTNP |
| | | | YDYPFISQGEISVASIDDQEELIATDS |
| | | | AIDILGFTNEEKVSIYKLTGAVMHYG |
| | | | NLKFKQKQREEQAEPDGTEVADKA |
| | | | AYLQSLNSADLLKALCYPRVKVGNE |
| | | | YVTKGQTVEQVTNAVGALAKAVYE |
| | | | KMFLWMVTRINQQLDTKQPRQYFIG |
| | | | VLDIAGFEIFDFNSLEQLCINFTNEKL |
| | | | QQFFNHHMFVLEQEEYKREGIEWTFI |
| | | | DFGMDLAACIELIEKPMGIFSILEEEC |
| | | | MFPKATDTSFKNKLYEQHLGKSANF |
| | | | QKPKPAKGKVEAHFSLIHYAGTVDY |
| | | | NITGWLDKNKDPLNDTVVGLYQKS |
| | | | ALKTLAFLFSGAQTGEAEAGGTKKG |
| | | | GKKKGSSFQTVSALFRENLNKLMTN |
| | | | LRSTHPHFVRCIIPNETKTPGAMEHE |
| | | | LVLHQLRCNGVLEGIRICRKGFPSRIL |
| | | | YADFKQRYKVLNASAIPEGQYIDSK |
| | | | KASEKLLASIDIDHTQYKFGHTKVFF |
| | | | KAGLLGLLEEMRDDKLAQLITRTQA |
| | | | RCRGFLARVEYQKMVERRESIFCIQY |
| | | | NIRAFMNVKHWPWMKLFFKIKPLLK |
| | | | SAESEKEMATMKEEFQKTKDELAKS |
| | | | EAKRKELEEKMVTLLKEKNDLQLQV |
| | | | QAEAEGLADAEERCDQLIKTKIQLEA |
| | | | KIKEVTERAEDEEEINAELTAKKRKL |
| | | | EDECSELKKDIDDLELTLAKVEKEKH |
| | | | ATENKVKNLTEEMAGLDETIAKLTK |
| | | | EKKALQEAHQQTLDDLQAEEDKVN |
| | | | TLTKAKTKLEQQVDDLEGSLEQEKK |
| | | | LRMDLERAKRKLEGDLKLAQESIMD |
| | | | IENEKQQLDEKLKKKEFEISNLQSKIE |
| | | | DEQALAIQLQKKIKELQARIEELEEEI |
| | | | EAERASRAKAEKQRSDLSRELEEISE |
| | | | RLEEAGGATSAQIEMNKKREAEFQK |
| | | | MRRDLEEATLQHEATAAALRKKHA |
| | | | DSVAELGEQIDNLQRVKQKLEKEKS |
| | | | EMKMEIDDLASNMETVSKAKGNLE |
| | | | KMCRTLEDQLSELKSKEEEQQRLIND |
| | | | LTAQRGRLQTESGEFSRQLDEKEAL |
| | | | VSQLSRGKQAYTQQIEELKRQLEEEI |
| | | | KAKNALAHALQSSRHDCDLLREQYE |
| | | | EEQESKAELQRALSKANTEVAQWRT |
| | | | KYETDAIQRTEELEEAKKKLAQRLQ |
| | | | AAEEHVEAVNAKCASLEKTKQRLQ |
| | | | NEVEDLMLDVERTNAACAALDKKQ |
| | | | RNFDKILAEWKQKYEETHAELEASQ |
| | | | KEARSLGTELFKMKNAYEESLDQLE |
| | | | TLKRENKNLQQEISDLTEQIAEGGKR |
| | | | IHELEKIKKQVEQEKSEIQAALEEAE |
| | | | ASLEHEEGKILRIQLELNQVKSEVDR |
| | | | KIAEKDEEIDQLKRNHVRVVESMQS |
| | | | MLDAEIRSRNDAIRLKKKMEGDLNE |
| | | | MEIQLNHANRMAAEALRNYRNTQGI |
| | | | LKDTQIHLDDALRGQEDLKEQLAMV |
| | | | ERRANLLQAEIEELRATLEQTERSRK |
| | | | VAEQELLDASERVQLLHTQNTSLINT |
| | | | KKKLETDISQMQGEMEDILQEARNA |
| | | | EEKAKKAITDAAMMAEELKKEQDTS |
| | | | AHLERMKKNMEQTVKDLQHRLDEA |
| | | | EQLALKGGKKQIQKLEARVRELEGE |
| | | | VESEQKRNAEAVKGLRKHERRVKEL |
| | | | TYQTEEDRKNILRLQDLVDKLQAKV |
| | | | KSYKRQAEEAEEQSNTNLSKFRKLQ |
| | | | HELEEAEERADIAESQVNKLRVKSRE |
| | | | VHTKVISEE |
| SEQ ID | Q9TV62 | Myosin-4/Sus | MSSDQEMAIFGEAAPYLRKSEKERIE |
| NO: 48 | (UniProtKB) | scrofa | AQNKPFDAKTSVFVAEPKESFVKGT |
| | | | VQSREGGKVTVKTEAGATLTVKEDQ |
| | | | VFPMNPPKFDKIEDMAMMTHLHEPA |
| | | | VLYNLKERYAAWMIYTYSGLFCVTV |
| | | | NPYKWLPVYNAEVVTAYRGKKRQE |
| | | | APPHIFSISDNAYQFMLTDRENQSILI |
| | | | TGESGAGKTVNTKRVIQYFATIAVTG |
| | | | EKKKEEPTPGKMQGTLEDQIISANPL |
| | | | LEAFGNAKTVRNDNSSRFGKFIRIHF |
| | | | GTTGKLASADIETYLLEKSRVTFQLK |
| | | | AERSYHIFYQIMSNKKPELIEMLLITT |
| | | | NPYDYAFVSQGEITVPSIDDQEELMA |
| | | | TDSAIEILGFTSDERVSIYKLTGAVM |
| | | | HYGNLKFKQKQREEQAEPDGTEVA |
| | | | DKAAYLQGLNSADLLKALCYPRVK |
| | | | VGNEFVTKGQTVQQVYNAVGALAK |
| | | | AVYDKMFLWMVTRINQQLDTKQPR |
| | | | QYFIGVLDIAGFEIFDFNSLEQLCINFT |
| | | | NEKLQQFFNHHMFVLEQEEYKKEGI |
| | | | EWEFIDFGMDLAACIELIEKPMGIFSI |
| | | | LEEECMFPKATDTSFKNKLYEQHLG |
| | | | KSNNFQKPKPAKGKAEAHFSLIHYA |
| | | | GTVDYNITGWLDKNKDPLNETVVGL |
| | | | YQKSSVKTLAFLFAERQSSEEGGTKK |
| | | | GGKKKGSSFQTVSALFRENLNKLMT |
| | | | NLRSTHPHFVRCIIPNETKTPGAMEH |
| | | | ELVLHQLRCNGVLEGIRICRKGFPSRI |
| | | | LYADFKQRYKVLNASAIPEGQFIDSK |
| | | | KASEKLLGSIDIDHTQYKFGHTKVFF |
| | | | KAGLLGTLEEMRDEKLAQLITRTQA |
| | | | MCRGFLMRVEFRKMMERRESIFCIQ |
| | | | YNIRAFMNVKHWPWMKLYFKIKPL |
| | | | LKSAETEKEMANMKEEFEKTKEDLA |
| | | | KSEAKRKELEEKMVALMQEKNDLQ |
| | | | LQVQAEADGLADAEERCDQLIKTKI |
| | | | QLEAKIKEVTERAEDEEEINAELTAK |
| | | | KRKLEDECSELKKDIDDLELTLAKVE |
| | | | KEKHATENKVKNLTEEMAGLDENIA |
| | | | KLTKEKKALQEAHQQTLDDLQAEED |
| | | | KVNTLTKAKTKLEQQVDDLEGSLEQ |
| | | | EKKLRMDLERAKRKLEGDLKLAQES |
| | | | TMDIENDKQQLDEKLKKKEFEMSNL |
| | | | QSKIEDEQALAMQLQKKIKELQART |
| | | | EELEEEIEAERASRAKAEKQRSDLSR |
| | | | ELEEISERLEEAGGATSAQIEMNKKR |
| | | | EAEFQKMRRDLEEATLQHEATAAAL |
| | | | RKKHADSVAELGEQIDNLQRVKQKL |
| | | | EKEKSELKMEIDDLASNMETVSKAK |
| | | | GNLEKMCRTLEDQLSEVKTKEEEHQ |
| | | | RLINELSAQKARLQTESGEFSRQLDE |
| | | | KEALVSQLSRGKQAFTQQIEELKRQL |
| | | | EEETKAKSALAHAVQSSRHDCDLLR |
| | | | EQYEEEQEAKAELQRAMSKANSEVA |
| | | | QWRTKYETDAIQRTEELEEAKKKLA |
| | | | QRLQDAEEHVEAVNAKCASLEKTK |
| | | | QRLQNEVEDLMLDVERSNAACAAL |
| | | | DKKQRNFDKILAEWKHKYEETQAEL |
| | | | EASQKESRSLSTELFKVKNAYEESLD |
| | | | QLETLKRENKNLQQEISDLTEQIAEG |
| | | | GKHIHELEKVKKQIEQEKSELQAALE |
| | | | EAEASLEHEEGKILRIQLELNQVKSEI |
| | | | DRKIAEKDEEIDQMKRNHIRVVESM |
| | | | QSTLDAEIRSRNDALRIKKKMEGDL |
| | | | NEMEIQLNHANRQATEAIRNLRNTQ |
| | | | GVLKDTQLHLDDAIRGQDDLKEQLA |
| | | | MVERRANLMQAEIEELRASLEQTER |
| | | | SRRVAEQELLDASERVQLLHTQNTS |
| | | | LINTKKKLETDISQIQGEMEDIVQEA |
| | | | RNAEEKAKKAITDAAMMAEELKKE |
| | | | QDTSAHLERMKKNMEQTVKDLQHR |
| | | | LDEAEQLALKGGKKQIQKLEARVRE |
| | | | LENEVENEQKRNVEAVKGLRKHERR |
| | | | VKELTYQTEEDRKNVLRLQDLVDKL |
| | | | QSKVKAYKRQAEEAEEQSNVNLSKF |
| | | | RKLQHELEEAEERADIAESQVNKLR |
| | | | VKSREVHTKVISEE |
| SEQ ID | P02565 | Myosin-1B/ | MATDADMAIFGEAAPYLRKSEKERI |
| NO: 49 | (UniProtKB) | Gallus gallus | EAQNKPFDAKSSVFWHAKESYVKS |
| | | | TIQSKESGhKVTVKTEGGETLTVKED |
| | | | QIFSMNPPKYDKIEDMAMMTHLHEP |
| | | | AVLYNLKERYAAWMIYTYSGLFCVT |
| | | | VNPYKWLPVYNPEVVLAYRGKKRQ |
| | | | EAPPHIFSISDNAYQFMLTDRENQSIL |
| | | | ITGESGAGKTVNTKRVIQYFATIAAS |
| | | | GDKKKEEQPAGKMQGTLEDQIISAN |
| | | | PLLEAFGNAKTVRNDNSSRFGKFIRI |
| | | | HFGATGKLASADIETYLLEKSRVTFQ |
| | | | LKAERSYHIFYQIMSNKKPELIEMLLI |
| | | | TTNPYDYQYVSQGEITVPSINDQEEL |
| | | | MATDSAIDILGFTPDEKTAIYKLTGA |
| | | | VMHYGNLKFKQKQREEQAEPGGTE |
| | | | VADKAAYLMGLNSADLLKALCYPR |
| | | | VKVGNEYVTKGQTVQQVYNSVGAL |
| | | | AKSVFEKMFLWMVVRINQQLDTKQ |
| | | | PRQYFIGVLDIAGFEIFDFNSLEQLCI |
| | | | NFTNEKLQQFFNHHMFVLEQEEYKK |
| | | | EGIEWEFIDFGMDLAACIELIEKPMGI |
| | | | FSILEEECMFPKATDTSFKNKLYDQH |
| | | | LGKSNNFQKPKPGKGKAEAHFSLVH |
| | | | YAGTVDYNITGWLEKNKDPLNETVV |
| | | | GLYQKSSLKTLALLFASVGGAEAES |
| | | | GAGGKKGGKKKGSSFQTVSALFREN |
| | | | LNKLMSNLRSTHPHFVRCLIPNETKT |
| | | | PGAMEHELVLHQLRCNGVLEGIRICR |
| | | | KGFPIRILYADFKQRYKVLNASAIPE |
| | | | GQFIDSKKASEKLLGSIDVDHTQYKF |
| | | | GHTKVFFKAGLLGLLEEMRDEKLAQ |
| | | | LITRTQARCRGFLMRVEFKKMMERR |
| | | | ESIFCIQYNVRAFMNVKHWPWMKLF |
| | | | FKIKPLLKSAESEKEMANMKEEFEKT |
| | | | KEELAKSEAKRKELEEKMVSLLQEK |
| | | | NDLQLQVQAEADGLADAEERCDQLI |
| | | | KTKIQLEAKIKELTERAEDEEEMNAE |
| | | | LTAKKRKLEDECSELKKDIDDLELTL |
| | | | AKVEKEKHATENKVKNLTEEMAAL |
| | | | DETIAKLTKEKKALQEAHQQTLDDL |
| | | | QAEEDKVNTLTKAKTKLEQQVDDLE |
| | | | GSLEQEKKLRMDLERAKRKLEGDLK |
| | | | MTQESTMDLENDKQQLDEKLKKKD |
| | | | FEISQIQSKIEDEQALGMQLQKKIKEL |
| | | | QARIEELEEEIEAERTSRAKAEKHRA |
| | | | DLSRELEEISERLEEAGGATAAQIDM |
| | | | NKKREAEFQKMRRDLEEATLQHEAT |
| | | | AAALRKKHADSTADVGEQIDNLQRV |
| | | | KQKLEKEKSELKMEIDDLASNMESV |
| | | | SKAKANLEKMCRSLEDQLSEIKTKEE |
| | | | EQQRTINDISAQKARLQTESGEYSRQ |
| | | | VEEKDALISQLSRGKQAFTQQIEELK |
| | | | RHLEEEIKAKKCPAHALQSARHDCD |
| | | | LLREQYEEEQEAKGELQRALSKANS |
| | | | EVAQWRTKYETDAIQRTEELEEAKK |
| | | | KLAQRLQDAEEHVEAVNSKCASLEK |
| | | | TKQRLQNEVEDLMIDVERSNAACAA |
| | | | LDKKQKNFDKILSEWKQKYEETQAE |
| | | | LEASQKESRSLSTELFKMKNAYEESL |
| | | | DHLETLKRENKNLQQEISDLTEQIAE |
| | | | GGKAIHELEKVKKQIEQEKSELQTAL |
| | | | EEAEASLEHEEGKILRVQLELNQVKS |
| | | | DIDRKIAEKDEEIDQLKRNHLRVVDS |
| | | | MQSTLDAEIRSRNEALRLKKKMEGD |
| | | | LNEIEIQLSHANRQAAEAQKNLRNTQ |
| | | | GVLKDTQIHLDDALRSQEDLKEQVA |
| | | | MVERRANLLQAEIEELRAALEQTERS |
| | | | RKVAEQELLDASERVQLLHTQNTSLI |
| | | | NTKKKLESDISQIQSEMEDTIQEARN |
| | | | AEEKAKKAITDAAMMAEELKKEQD |
| | | | TSAHLERMKKNLDQTVKDLQHRLD |
| | | | EAEQLALKGGKKQIQKLEARVRELE |
| | | | GEVDAEQKRSAEAVKGVRKYERRV |
| | | | KELTYQSEEDRKNVLRLQDLVDKLQ |
| | | | MKVKSYKRQAEEAEELSNVNLSKFR |
| | | | KIQHELEEAEERADIAESQVNKLRAK |
| | | | SREIGKKAESEE |
| SEQ ID | Q9TV61 | Myosin-1/Sus | MSSDQEMAIFGEAAPYLRKSEKERIE |
| NO: 50 | (UniProtKB) | scrofa | AQNKPFDAKTSVFVAEPKESFVKGT |
| | | | VQSREGGKVTVKTEAGATLTVKEDQ |
| | | | VFPMNPPKFDKIEDMAMMTHLHEPA |
| | | | VLYNLKERYAAWMIYTYSGLFCVTV |
| | | | NPYKWLPVYNAEVVTAYRGKKRQE |
| | | | APPHIFSISDNAYQFMLTDRENQSILI |
| | | | TGESGAGKTVNTKRVIQYFATIAVTG |
| | | | EKKKEEPTSGKMQGTLEDQIISANPL |
| | | | LEAFGNAKTVRNDNSSRFGKFIRIHF |
| | | | GTTGKLASADIETYLLEKSRVTFQLK |
| | | | AERSYHIFYQIMSNKKPELIEMLLITT |
| | | | NPYDYAFVSQGEITVPSIDDQEELMA |
| | | | TDSAIEILGFTSDERVSIYKLTGAVM |
| | | | HYGNLKFKQKQREEQAEPDGTEVA |
| | | | DKAAYLQGLNSADLLKALCYPRVK |
| | | | VGNEFVTKGQTVQQVYNAVGALAK |
| | | | AVYDKMFLWMVTRINQQLDTKQPR |
| | | | QYFIGVLDIAGFEIFDFNSLEQLCINFT |
| | | | NEKLQQFFNHHMFVLEQEEYKKEGI |
| | | | EWEFIDFGMDLAACIELIEKPMGIFSI |
| | | | LEEECMFPKATDTSFKNKLYEQHLG |
| | | | KSNNFQKPKPAKGKVEAHFSLIHYA |
| | | | GTVDYNITGWLDKNKDPLNETVVGL |
| | | | YQKSSVKTLAFLFTGAAGADAEAGG |
| | | | GKKGGKKKGSSFQTVSALFRENLNK |
| | | | LMTNLRSTHPHFVRCIIPNETKTPGA |
| | | | MEHELVLHQLRCNGVLEGIRICRKGF |
| | | | PSRILYADFKQRYKVLNASAIPEGQFI |
| | | | DSKKASEKLLGSIDIDHTQYKFGHTK |
| | | | VFFKAGLLGLLEEMRDEKLAQLITRT |
| | | | QARCRGFLARVEYQKMVERRESIFCI |
| | | | QYNIRAFMNVKHWPWMKLYFKIKP |
| | | | LLKSAETEKEMANMKEEFEKTKESL |
| | | | AKAEAKRKELEEKMVALMQEKNDL |
| | | | QLQVQAEADSLADAEERCDQLIKTKI |
| | | | QLEAKIKEVTERAEDEEEINAELTAK |
| | | | KRKLEDECSELKKDIDDLELTLAKVE |
| | | | KEKHATENKVKNLTEEMAGLDETIA |
| | | | KLTKEKKALQEAHQQTLDDLQAEED |
| | | | KVNTLTKAKTKLEQQVDDLEGSLEQ |
| | | | EKKLRMDLERAKRKLEGDLKLAQES |
| | | | TMDIENDKQQLDEKLKKKEFEMSNL |
| | | | QSKIEDEQALAMQLQKKIKELQARIE |
| | | | ELEEEIEAERASRAKAEKQRSDLSRE |
| | | | LEEISERLEEAGGATSAQIEMNKKRE |
| | | | AEFQKMRRDLEEATLQHEATAATLR |
| | | | KKHADSVAELGEQIDNLQRVKQKLE |
| | | | KEKSEMKMEIDDLASNMETVSKAK |
| | | | GNLEKMCRTLEDQLSELKTKEEEQQ |
| | | | RLINDLTAQRARLQTESGEYSRQLDE |
| | | | KDTLVSQLSRGKQAFTQQIEELKRQL |
| | | | EEEIKAKSALAHAVQSSRHDCDLLRE |
| | | | QYEEEQEAKAELQRAMSKANSEVA |
| | | | QWRTKYETDAIQRTEELEEAKKKLA |
| | | | QRLQDAEEHVEAVNAKCASLEKTK |
| | | | QRLQNEVEDLMIDVERSNAACAALD |
| | | | KKQRNFDKILAEWKQKYEETHAELE |
| | | | ASQKESRSLSTELFKVKNAYEESLDQ |
| | | | LETLKRENKNLQQEISDLTEQIAEGG |
| | | | KRIHELEKIKKQVEQEKSEIQAALEE |
| | | | AEASLEHEEGKILRIQLELNQVKSEV |
| | | | DRKIAEKDEEIDQLKRNHVRVVESM |
| | | | QSMLDAEIRSRNDAIRLKKKMEGDL |
| | | | NEMEIQLNHANRMAAEALRNYRNT |
| | | | QGILKDTQIHLDDALRSQEDLKEQLA |
| | | | MVERRANLLQAEIEELRATLEQTERS |
| | | | RKVAEQELLDASERVQLLHTQNTSLI |
| | | | NTKKKLETDISQIQGEMEDIIQEARN |
| | | | AEEKAKKAITDAAMMAEELKKEQD |
| | | | TSAHLERMKKNLEQTVKDLQHRLDE |
| | | | AEQLALKGGKKQIQKLEARVRELEG |
| | | | EVESEQKRNVETVKGLRKHERRVKE |
| | | | LTYQTEEDRKNILRLQDLVDKLQAK |
| | | | VKSYKRQAEEAEEQSNVNLSKFRKL |
| | | | QHELEEAEERADIAESQVNKLRVKS |
| | | | REVHTKIISEE |
| SEQ ID | Q9DGM4 | Fast myosin | MASSDAEMAAFGEAAPYHRKSEKE |
| NO: 51 | (UniProtKB) | heavy chain | RIEAQNKPFDAKSSVFVAHPKESFVK |
| | | isoform 3/Gallus | GTIQSRETGKVTVKTEGGETLTVKED |
| | | gallus | QVFSMNPPKYDKIEDMAMMTHLHE |
| | | | PAVLYNLKERYAAWMIYTYSGLFCV |
| | | | TVNPYKWLPVYNPEVVLAYRGKKR |
| | | | QEAPPHIFSISDNAYQFMLTDRENQSI |
| | | | LITGESGAGKTVNTKRVIQYFATIAA |
| | | | SGEKKKEEQSGKMQGTLEDQIISANP |
| | | | LLEAFGNAETVRNDNSSRFGKFIRIH |
| | | | FGATGKLASADIETYLLEKSRVTFQL |
| | | | KAERSYHIFYQIMSNKKPELIDMLLIT |
| | | | TNPYDYHFVSQGEITVPSIDDQEELM |
| | | | ATDSAIDILGFTADEKTAISKLTGAV |
| | | | MHYGNLKFKQKQREEQAEPDGTEV |
| | | | ADKAAYLMGLNSADLLKALCYPRV |
| | | | KVGNEYVTKGQTVQQVHNAVGALA |
| | | | KAVYEKMFLWMVVRINQQLDTKQP |
| | | | RQYFIGVLDIAGFEIFDFNSFEQLCINF |
| | | | TNEKLQQFFNHHMFVLEQEEYKKEG |
| | | | IEWTFIDFGMDLAACIELIEKPMGIFSI |
| | | | LEEECMFPKATDTSFKNKLYDQHLG |
| | | | KSSNFQKPKPAKGKAEAHFSLVHYA |
| | | | GTVDYNITGWLEKNKDPLNETVIGL |
| | | | YQKSSVKTLALLFATYGGADAEAGG |
| | | | GGKKGGKKKGSSFQTVSALFRENLN |
| | | | KLMTNLRSTHPHFVRCIIPNETKTPG |
| | | | AMEHELVLHQLRCNGVLEGIRICRK |
| | | | GFPSRVLYADFKQRYKVLNASAIPEG |
| | | | QFIDSKKASEKLLSSIDVDHTQYKFG |
| | | | HTKVFFKAGLLGLLEEMRDEKLAQL |
| | | | ITRTQARSRGFLMRVEYQRMVERRE |
| | | | SIFCIQYNVRSFMNVKHWPWMKLFF |
| | | | KIKPLLKSAESEKEMANMKEEFEKT |
| | | | KEELAKSEAKRKELEEKMVKLVQEK |
| | | | NDLQLQVQAEADSLADAEERCDQLI |
| | | | KTKIQLEAKIKEVTERAEDEEEINAEL |
| | | | TAKKRKLEDECSELKKDMDDLELTL |
| | | | AKVEKEKHATENKVKNLTEEMAAL |
| | | | DETIVKLTKEKKALQEAHQQTLDDL |
| | | | QAEEDKVNTLTKAKTKLEQQVDDLE |
| | | | GSLEQEKKLRMDLERAKRKLEGDLK |
| | | | LAHDSIMDLENDKQQLDEKLKKKDF |
| | | | EISQIQSKIEDEQALGMQLQKKIKEL |
| | | | QARIEELEEEIEAERTSRAKAEKHRA |
| | | | DLSRELEEISERLEEAGGATAAQIDM |
| | | | NKKREAEFQKMRRDLEEATLQHEAT |
| | | | AAALRKKHADSTAELGEQIDNLQRV |
| | | | KQKLEKEKSELKMEIDDLASNMESV |
| | | | SKAKANLEKMCRTLEDQLSEIKTKE |
| | | | EEHQRMINDLNTQRARLQTEAGEYS |
| | | | RQVEEKDALISQLSRGKQAFTQQIEE |
| | | | LKRHLEEEIKAKNALAHALQSARHD |
| | | | CDLLREQYEEEQEAKGELQRALSKA |
| | | | NSEVAQWRTKYETDAIQRTEELEEA |
| | | | KKKLAQRLQDAEEHVEAVNAKCAS |
| | | | LEKTKQRLQNEVEDLMIDVERANAA |
| | | | CAALDKKQKNFDKILAEWKQKYEE |
| | | | TQAELEASQKESRSLSTELFKMKNA |
| | | | YEESLDHLQTLKRENKNLQQEISDLT |
| | | | EQIAEGGKAIHELEKVKKQIEQEKSEI |
| | | | QAALEEAEASLEHEEGKILRLQLELN |
| | | | QVKSEIDRKIAEKDEEIDQLKRNHLRI |
| | | | VESLQSSLDAEIRSRNEALRLKKKME |
| | | | GDLNEMEIQLSHANRVAAEAQKNLR |
| | | | NTQAVLKDTQIHLDDALRTQEVLKE |
| | | | QVAMVERRANLLQAEIEELRAALEQ |
| | | | TERSRKVAEQELMDASERVQLLHTQ |
| | | | NTSLINTKKKLETDIAQIQSEMEDTIQ |
| | | | EARNTEEKAKKAITDAAMMAEELK |
| | | | KEQDTSAHLERMKKNLDQTVKDLQ |
| | | | HRLDEAEQLALKGGKKQIQKLEARV |
| | | | RELEGEVDAEQKRSAEAVKGVRKYE |
| | | | RRVKELTYQSEEDLKNILRLQDLVD |
| | | | KLQMKVKSYKRQAEEAEELSNVNLS |
| | | | KFRKIQHELEEAEERADIAESQVNKL |
| | | | RVKSREFHSKKIEEEE |
| SEQ ID | P13538 | Myosin heavy | MASPDAEMAAFGEAAPYLRKSEKER |
| NO: 52 | (UniProtKB) | chain, skeletal | IEAQNKPFDAKSSVFVVHPKESFVKG |
| | | muscle, | TIQSKEGGKVTVKTEGGETLTVKED |
| | | adult/Gallus | QVFSMNPPKYDKIEDMAMMTHLHE |
| | | gallus | PAVLYNLKERYAAWMIYTYSGLFCV |
| | | | TVNPYKWLPVYNPEVVLAYRGKKR |
| | | | QEAPPHIFSISDNAYQFMLTDRENQSI |
| | | | LITGESGAGKTVNTKRVIQYFATIAA |
| | | | SGEKKKEEQSGKMQGTLEDQIISANP |
| | | | LLEAFGNAKTVRNDNSSRFGKFIRIH |
| | | | FGATGKLASADIETYLLEKSRVTFQL |
| | | | PAERSYHIFYQIMSNKKPELIDMLLIT |
| | | | TNPYDYHYVSQGEITVPSIDDQEELM |
| | | | ATDSAIDILGFSADEKTAIYKLTGAV |
| | | | MHYGNLKFKQKQREEQAEPDGTEV |
| | | | ADKAAYLMGLNSAELLKALCYPRV |
| | | | KVGNEFVTKGQTVSQVHNSVGALA |
| | | | KAVYEKMFLWMVIRINQQLDTKQPR |
| | | | QYFIGVLDIAGFEIFDFNSFEQLCINFT |
| | | | NEKLQQFFNHHMFVLEQEEYKKEGI |
| | | | EWEFIDFGMDLAACIELIEKPMGIFSI |
| | | | LEEECMFPKATDTSFKNKLYDQHLG |
| | | | KSNNFQKPKPAKGKAEAHFSLVHYA |
| | | | GTVDYNISGWLEKNKDPLNETVIGL |
| | | | YQKSSVKTLALLFATYGGEAEGGGG |
| | | | KKGGKKKGSSFQTVSALFRENLNKL |
| | | | MANLRSTHPHFVRCIIPNETKTPGAM |
| | | | EHELVLHQLRCNGVLEGIRICRKGFP |
| | | | SRVLYADFKQRYRVLNASAIPEGQF |
| | | | MDSKKASEKLLGSIDVDHTQYRFGH |
| | | | TKVFFKAGLLGLLEEMRDDKLAEIIT |
| | | | RTQARCRGFLMRVEYRRMVERRESI |
| | | | FCIQYNVRSFMNVKHWPWMKLFFKI |
| | | | KPLLKSAESEKEMANMKEEFEKTKE |
| | | | ELAKSEAKRKELEEKMVVLLQEKND |
| | | | LQLQVQAEADSLADAEERCDQLIKT |
| | | | KIQLEAKIKEVTERAEDEEEINAELTA |
| | | | KKRKLEDECSELKKDIDDLELTLAK |
| | | | VEKEKHATENKVKNLTEEMAVLDE |
| | | | TIAKLTKEKKALQEAHQQTLDDLQV |
| | | | EEDKVNTLTKAKTKLEQQVDDLEGS |
| | | | LEQEKKLRMDLERAKRKLEGDLKLA |
| | | | HDSIMDLENDKQQLDEKLKKKDFEI |
| | | | SQIQSKIEDEQALGMQLQKKIKELQA |
| | | | RIEELEEEIEAERTSRAKAEKHRADLS |
| | | | RELEEISERLEEAGGATAAQIEMNKK |
| | | | REAEFQKMRRDLEEATLQHEATAAA |
| | | | LRKKHADSTAELGEQIDNLQRVKQK |
| | | | LEKEKSELKMEIDDLASNMESVSKA |
| | | | KANLEKMCRTLEDQLSEIKTKEEQN |
| | | | QRMINDLNTQRARLQTETGEYSRQA |
| | | | EEKDALISQLSRGKQGFTQQIEELKR |
| | | | HLEEEIKAKNALAHALQSARHDCEL |
| | | | LREQYEEEQEAKGELQRALSKANSE |
| | | | VAQWRTKYETDAIQRTEELEEAKKK |
| | | | LAQRLQDAEEHVEAVNAKCASLEKT |
| | | | KQRLQNEVEDLMVDVERSNAACAA |
| | | | LDKKQKNFDKILAEWKQKYEETQTE |
| | | | LEASQKESRSLSTELFKMKNAYEESL |
| | | | DHLETLKRENKNLQQEIADLTEQIAE |
| | | | GGKAVHELEKVKKHVEQEKSELQAS |
| | | | LEEAEASLEHEEGKILRLQLELNQIKS |
| | | | EIDRKIAEKDEEIDQLKRNHLRIVES |
| | | | MQSTLDAEIRSRNEALRLKKKMEGD |
| | | | LNEMEIQLSHANRMAAEAQKNLRNT |
| | | | QGTLKDTQIHLDDALRTQEDLKEQV |
| | | | AMVERRANLLQAEVEELRGALEQTE |
| | | | RSRKVAEQELLDATERVQLLHTQNT |
| | | | SLINTKKKLETDIVQIQSEMEDTIQEA |
| | | | RNAEEKAKKAITDAAMMAEELKKE |
| | | | QDTSAHLERMKKNMDQTVKDLHVR |
| | | | LDEAEQLALKGGKKQLQKLEARVRE |
| | | | LEGEVDSEQKRSAEAVKGVRKYERR |
| | | | VKELTYQCEEDRKNILRLQDLVDKL |
| | | | QMKVKSYKRQAEEAEELSNVNLSKF |
| | | | RKIQHELEEAEERADIAESQVNKLRV |
| | | | KSREIHGKKIEEEE |
| SEQ ID | P16419 | Myosin-binding | MPEPSKAAPKKEAKKKEEKKEEKKE |
| NO: 53 | (UniProtKB) | protein C, fast- | APPPQEHKDEAPDDVHPPETPDPEGL |
| | | type/Gallus | FLSKPQNVMVESGRDVTVSARVAGA |
| | | gallus | ALPCAPAVKWFKGKWAELGDKSAR |
| | | | CRLRHSVDDDKVHTFELTITKVAMG |
| | | | DRGDYRCEVTAKEQKDSCSFSIDVE |
| | | | APRSSEGNVLQAFKRTGEGKDDTAG |
| | | | ELDFSGLLKKREVQVEEKKKKKDED |
| | | | DQFPPEIWELLKGVTKKSEYERIAFQ |
| | | | YGITDLRGMLKRLKKVHVEPKKSEA |
| | | | FIRKLDPAYQVDKGNKIKLVVELSDP |
| | | | DLPLKWYKNGQLLKPSTKYVFENVG |
| | | | LKRILTIHKCSLADDAAYECRVNDEK |
| | | | CFTEVFVKEPPVTVVRGLEDQQVVV |
| | | | GDRVVLEAEVSEEGAQVMWLKDGV |
| | | | DVTRDDAFKYRFKKDGKKHFLIINE |
| | | | AELSDSAHYKIMTNGGESEAELSVEE |
| | | | KQLEVLQDMADLTVKASEQAVFKC |
| | | | EVSDEKVTGRWFRNGVEVKPSKRIHI |
| | | | SHNGRFHKLVIDDVRPEDEGDYTFIP |
| | | | DGYALSLSAKLNFLEIKVEYVPKQEP |
| | | | PKIHLDCSGKAAENTIVVVAGNKVR |
| | | | LDVPISGEPAPTVTWKRGDQLFTATE |
| | | | GRVHIDSQADLSSFVIESAERSDEGR |
| | | | YCITVTNPVGEDSATLHVRVVDVPD |
| | | | PPQSVRVTSVGEDWAVLSWEAPPFD |
| | | | GGMPITGYLMERKKKGSMRWMKLN |
| | | | FEVFPDTTYESTKMIEGVFYEMRVFA |
| | | | VNAIGVSQPSLNTQPFMPIAPTSEPTH |
| | | | VVLEDVTDTTATIKWRPPERIGAGG |
| | | | VDGYLVEWCREGSNEWVAANTELV |
| | | | ERCGLTARGLPTGERLLFRVISVNMA |
| | | | GKSPPATMAQPVTIREIVERPKIRLPR |
| | | | HLRQTYIRRVGEQVNLVIPFQGKPRP |
| | | | QVTWSREGGALPAEVQTRTSDVDSV |
| | | | FFIRSAARPLSGNYEMRVRIDNMEDC |
| | | | ATLRLRVVERPGPPQAVRVMEVWG |
| | | | SNALLQWEPPKDDGNAEISGYTVQK |
| | | | ADTRTMEWFTVLEHSRPTRCTVSEL |
| | | | VMGNEYRFRVYSENVCGTSQEPATS |
| | | | HNTARIAKEGLTLKMVPYKERDLRA |
| | | | APQFLTPLVDRSVVAGYTVTLNCAV |
| | | | RGHPKPKVTWLKNSVEIGADPKFLS |
| | | | RHGLGVLSLLIRRPGPFDGGTYGCRA |
| | | | VNEMGEATTECRLDVRVPQ |
| SEQ ID | E1BNV1 | Myosin binding | MPEAKPAAKKAPAGKDAAAKPAPK |
| NO: 54 | (UniProtKB) | protein C, fast | EATPQEAPAAPPTEAPPEDQSPTAEE |
| | | type/Bos taurus | PTGVFLKKPDSVSVETGKDTVIVAKL |
| | | | NGKELPAKPAVKWFKGKWLELGSK |
| | | | SGARFSFKESHDAASNVYTIELHITK |
| | | | VVLGDRGDYRIEVKAKDFCDSCAFN |
| | | | IDVEAPRQDSAGQSLESFKRSGEAKS |
| | | | DTAGELDFSGLLKKRQVVEEEKKKK |
| | | | KDDDDLGIPPEIWELLKGAKKSEYER |
| | | | IAFQYGITDLRGMLKRLKKAKVEVK |
| | | | KSAAFTKKLDPAYQVDRGNKIKLVV |
| | | | EISDPDLPLKWFKNGQEIKPSSKYVF |
| | | | ENVGKKRILTINKCTLADDAAYEVV |
| | | | VKDEKCFTELFVKVEPPVLIVTPLED |
| | | | QQVFVGDRVEMAVEVSEEGAQVM |
| | | | WLKDGVELTREDSFKARYRLKKDG |
| | | | KRHILIYSEVTMEDKGHYQVMTNGG |
| | | | QCEAELIVEEKQLEVLQDIADLTVKA |
| | | | SEQAVFKCEVSDEKVTGKWYKNGV |
| | | | EVRPSKRITISHTGRFHKLVIDDVRPE |
| | | | DEGDYTFVPDGYALSLSAKLKFLEIK |
| | | | VEYVPKQEPPKIHLDCSGQTSENAIV |
| | | | VVAGNKLRMDVSITGEPRPVATWM |
| | | | KEDEVFTGTEGRVRIEQRGDISSFVIE |
| | | | SAERGDEGRYTIKVTNPVGEDVASIL |
| | | | LKVVDVPDPPEAVRVTSVGEDWAV |
| | | | LVWEPPKYDGGRPVTGYLLERKKK |
| | | | GSQRWMKLNFEVFTETTYESTNMIE |
| | | | GILYEMRVFAAPLIQQLKTLTPQPHA |
| | | | PFSPTSEPQHLTVEDVTDTTTTLKWR |
| | | | PPDKIGAGGIDGYLIEYCVEGSDEWI |
| | | | PANTEPVERCGFTVKNLPTGAKITFR |
| | | | VVGVNIAGRSQPATLAQPVTIREIVE |
| | | | QPKIRLPRHLRQTYVRKVGEHLNLVI |
| | | | PFQVRRAKGGAPIDTSHVHVRTSDF |
| | | | DTVFFVRQAARSDSGEYELTVQIEN |
| | | | MKDTATVCIRVVEKAGPPQNVMVK |
| | | | EVWGTNALVEWQPPKDDGNSEITGY |
| | | | FVQKADKKTMEWFTVYERNRHTSC |
| | | | TVSDLIMGNEYYFRVYSENVCGLSD |
| | | | LPGVSKNTARIVKTGMTLKLPEYKE |
| | | | HDFRTPPKFLTPLPDRVVVAGYAAA |
| | | | LNCAVRGHPKPKVVWMKNKMEIRE |
| | | | DPKFLMTNQQGVLTLNIRRPSPFDSG |
| | | | TYSCRAVNELGEALAECKLEVRAPR |
| SEQ ID | Q05623 | Myosin-binding | MTGKTAPAAAKKAPAAKKAPAPAS |
| NO: 55 | (UniProtKB) | protein H/Gallus | KKAPEPAPKEKPAPTPKEGHAPTPKE |
| | | gallus | EHAPPPKEEHAPPPKEEHAPAPAAET |
| | | | PPAPEHPPDAEQPAAPAAEHAPTPTH |
| | | | EAAPAHEEGPPPAAPAEAPAPEPEPE |
| | | | KPKEEPPSVPLSLAVEEVTENSVTLT |
| | | | WKAPEHTGKSSLDGYVVEICKDGST |
| | | | DWTAVNKEPFLSTRYKIHDLASGEK |
| | | | VHVRVKAISASGTSDPATLEQPVLIR |
| | | | EITDLPRIRLPRQLRQVYVRHVGEAV |
| | | | NLLIPFQGKPQPQVTWTKDNQPLDTS |
| | | | RVNIRNTDKDTIFFIRTAQRSDSGKY |
| | | | QLSVRINGAEDKAILDIRVIERPGPPQ |
| | | | NLKLVDVWGFNVALEWSPPADNGN |
| | | | SEIKGYTVQKSDKKSGKWFTVLERC |
| | | | TRTSCTISDLIIGNTYSFRVFSENACG |
| | | | MSETAAVAAGVAHIKKTVYQPQKIP |
| | | | ERDMMEPPKFTQPLTDRATTRGYST |
| | | | HLFCSVRGFPQPKIIWMKNKMEIRED |
| | | | PKYIAMIEQGVCSLEIRKPSPFDAGV |
| | | | YTCKAVNPLGEASVDCKLDVKMPK |
| SEQ ID | G3X6W9 | Myosin binding | MTEKATSEAPACGLEETTSESAHVPL |
| NO: 56 | (UniProtKB) | protein H/Bos | TEPSGDTAAPQAPGGEQAPRGQQAS |
| | | taurus | DPQESARQPPDPAASAAPAGPAATD |
| | | | PALPREDVPSAPLLLAVEDVSDSSVT |
| | | | VSWEPPERLGRLGLQGYVLELRREG |
| | | | ALDWVPVNARPMMVTQQTLRNLAV |
| | | | GDKFFVRVAAVSSAGAGPPAVLERLI |
| | | | HIQETIEAPKIRVPRHLRQTYIRQVGE |
| | | | SINLQIPFQGNPKPQALWTHNGHALD |
| | | | SQRVSVRTGDQDSILFIRSAQRSDSG |
| | | | CYELTVQLKDLEAKAAINILVIEKPG |
| | | | PPRSIRLLDVWNCNATLEWTPPQDT |
| | | | GNTELLGYTVQKADKKTGQWFTVL |
| | | | ERCHPTSCTVSDLIVGNSYSFRVFSE |
| | | | NLCGLSASAAVTKELAHIVKTDIVAK |
| | | | PKSFVERDFSEAPSFTQPLADHTSTPG |
| | | | YSTQLSCSVRASPKPKIIWMKNKMDI |
| | | | QGDPKYRALSEQGVCTLEIRKPSPFD |
| | | | SGVYTCKAINVLGEASVDCRLEVKA |
| | | | SATH |
| SEQ ID | A6BM71 | Connectin | MTTKAPTFTQPLQSVVALEGSAATFE |
| NO: 57 | (UniProtKB) | (Fragment)/Gallus | AHISGFPVPEVSWYRDGQVLSAATLP |
| | | gallus | GVQISFSDGRAKLVIPSVTEANSGRY |
| | | | TIQATNGSGQATSTAELLVTAGTAPP |
| | | | NFSQRLQSMTARQGSQVRLDVRVTG |
| | | | IPTPVVKFYRDGVEIQSSPDFQILQEG |
| | | | DLYSLIIAEAYPEDSGTYSVNATNNV |
| | | | GRATSTAELLIQGEEEAVPAKKTKTI |
| | | | VSTAQISQTRQARIEKKIETHFDARSL |
| | | | TSVEMVIEGAAAQQLPHKAPPRMPP |
| | | | RPTSKSPTPPVITAKAQMARQQSPSP |
| | | | VRQSPSPVRHVRAPTPSPVRSVSPAG |
| | | | RISTSPIRPVKSPSPIRKAQVVTPGAE |
| | | | VLPPWRQEGYSATAEAQMKETRVST |
| | | | SATEIRTEERWEGRYGLQEQVTISGA |
| | | | AAGEVAAGAKEVRKEPEKTPVPTVII |
| | | | ATDKAKEQERISTAREEISARHEQVH |
| | | | VSHEQIEAGKRAEAVATVVAAVDQ |
| | | | ARVRSPWETEQVDETYVKKKTLEYG |
| | | | YKEHAVKDHEAQAEHHVATKEVKT |
| | | | VYVPPEKHIPAAEKKEVHVSTEIKRE |
| | | | TEAKIEKTIHIEHPRPRTASPHFTVSKI |
| | | | AVPKPDHTYEVSIAGSAMATLEKELS |
| | | | ATSAAQKITKPVKPPQLKPHEVKIKP |
| | | | ESAPPQFPFTEAAETYKAHYDVETK |
| | | | KEVDVSIKGEAVREDHLLLRKESEA |
| | | | KVTETARVPVPAEIPVTPPTLVWGLK |
| | | | NKTVTEGESVTLECHISGHPQPTVTW |
| | | | YREDYKIESSMDFQITFKAGLARLVI |
| | | | REAFAEDSGRFTCTATNKAGSVSTSC |
| | | | HLHVKVSEETETRETISEKVVTEEKS |
| | | | YVETKDVVMEDVSAAAEEVSGEPVP |
| | | | PFFIRKPVVHKLIEGGSIIFECQVGGN |
| | | | PKPHVLWKKGGVPLTTGYRYKVSY |
| | | | KRETGECKLEISMTFADDAGEYTIVI |
| | | | RNKFGEASATVSLLEEADYEAYIKSQ |
| | | | QEMMYQTQVTAYVQEPKVAEVAPPI |
| | | | SYGDFDKEYEKEQALIRKKMAKDTV |
| | | | MVRTFVEDEEFHISSFEERLIKEIELRI |
| | | | IKTTLDELLEEDGEEMMIDISESEAIG |
| | | | AGFDLRLKNYRTFEGTGVTFHCKTT |
| | | | GYPLPKIAWYKDGKRIRHGERYHME |
| | | | VLQDGSASLRLPVVLPEDEGIYTVFA |
| | | | SNMKGNAICSAKLYVEPVAPTATPG |
| | | | YMPGPEVMRRYRSISPRSPSRSPARS |
| | | | SPSCSPARRLDETDEGQLERLYKPVF |
| | | | VLKPTSVKCSQGQTARFDLKVVGRP |
| | | | MPETYWFHNGQQVVNDYTHKIVIKE |
| | | | DGTQSLIIVPAMPEDSGEWAVIAQNR |
| | | | AGKASVSVTLSVEAKEDLVRPRFVE |
| | | | RLRNVSVKEGSRLHMAVKATGNPNP |
| | | | DIVWLKNSDIIVPHKYPRIRIEGTKGA |
| | | | AALNIESTARQDAAWYTATAINKAG |
| | | | RDTTRCKVNVEVEHAEPEPERRLIIP |
| | | | KGTYKAKEIAAPELEPLHLRYGQEQ |
| | | | WEEGDLYDKEKQQKPFFKKKLTSLR |
| | | | LKQFGPAHFECRLTPIGDPTMVVEW |
| | | | LHDGKPLEAANRLRMINEFGYCSLD |
| | | | YGVAYSRDSGVITCRATNKYGTDHT |
| | | | SATLIVKDEKSLVEESQLPEGRRGMQ |
| | | | RIEELERMAHEGALPAVAVDQKEKQ |
| | | | KPELVLVPEPARVLEGETARFRCRVT |
| | | | GYPLPKVNWYLNSQLIRKSKRFRLR |
| | | | YDGIHYLDIVDCKSYDTGEVKVTAE |
| | | | NPEGFIEHKVKLEIQQREDFRSVLRR |
| | | | APEPRHEPVVTEPGKLLFEVQKIDKP |
| | | | AEATTKEVVKLKRAERITHEKLSEES |
| | | | EELRSKFKRRTEEGYYEAITAVELKS |
| | | | RKKDESYEEMLKKTKEELLHWTKEI |
| | | | PEEEKKALPPEGKITIPTFKPEKVELS |
| | | | PSMEAPKIFERIQSQTVAQGTDAHFR |
| | | | VRVVGKPDPECQWFRNGVQIERTDR |
| | | | IYWYWPEDNVCELVIRDVTADDSAS |
| | | | IMVKAVNIAGETSSHAFLLVQAKQLI |
| | | | SFIQNLQDVVAKERDSMATFECETSE |
| | | | PFIKVKWFKNGIEIHSGEKYRMHSDR |
| | | | KAHFLSVLAVEMSDADDYSCALVED |
| | | | ESVKTTAKLIVEGAVVEFIKELEDVE |
| | | | VPESFTGELECEVSPEDIEGKWYHGD |
| | | | VELSSNHKYVLASRRGRRILTIKDVN |
| | | | KDDQGEYSFVVDGKRTHCKLKMKP |
| | | | RPMTILQGLTDQKVCEGDIVQLEVK |
| | | | VSVENVEGVWMKDGHEIQSSDRIHI |
| | | | VLDKQSHMLLIEDATQEDSGTYSFSI |
| | | | PGLELSTTGQVTVYSVEIIVPLKDVH |
| | | | VVEGTKAILECKVSAPDVTSSKWYL |
| | | | NDHQIKPDERVQAVCKGTKQRLVIT |
| | | | RTHASDEGHYKLMVGKVETSCNVT |
| | | | VEEIEIIRGLHDITCTETQNVSFEVELS |
| | | | HSGIDVIWHFKGQEIKAGPKYKIEAR |
| | | | GKIYKLTVVKMMKDDEGEYVFYAG |
| | | | GKKTSGKLIVAGGAISKPLADLTVAE |
| | | | SQRAVFECEVANPESEGQWLKNGKP |
| | | | LPMTDQYRAETDGVKRRLNIPAAKM |
| | | | DDMGEYSYEIASSKTSAKLHVEAVKI |
| | | | KKTLKNLTVTETQEAVFSVELSHPD |
| | | | VKGALWIKNGVELESNDKYEISVKG |
| | | | TVHTLKIKHCVVTDESVYSFKLGKIG |
| | | | ANARLHVETVKIIKKPKDVTALENA |
| | | | VVSFELSVSHDTVPVRWFHKNVELK |
| | | | QSDKYKMISQRKVHKLMLHNISPAD |
| | | | AGEYTAFVGQLECKAKLFVETIHITK |
| | | | TMKSIEIPETKTASFQCEVSHFNVPSV |
| | | | WLKNGVEIEMSEKFKIVVQGKLHQL |
| | | | NIMNTSSEDSAEYTFVCGNDRVSATL |
| | | | TVKPILITSMLEDINAEEKDTITFEVT |
| | | | VNYEGISYKWLKNGVEIKSTDKCQIR |
| | | | TKKLTHSLSIRNVHFGDAAEYSFVAG |
| | | | KAASSATLYVEARHIEFRKHIKDIKV |
| | | | VEKKRAIFECEISEPDVQVQWMKDG |
| | | | QELQIGDRMKIQREKYVHRLIIPSTK |
| | | | MSDAGQYTVVAGGNTSSANLIVEGR |
| | | | DVRIRSIRKEIQVIERQRAEIEFEVNE |
| | | | DDIEPQWYKDGIEINFHYEERYSYVV |
| | | | ERRIHRMSIFETTYSDAGEYTFVAGR |
| | | | NRSSVVLYVNAPEPPQIIQELQPTTVE |
| | | | SGKPARFCAIISGKPQPKVSWYKDDQ |
| | | | QLSPGFKCKFLHDAQEYTLLLIETFPE |
| | | | DSAVYTCEAKNDYGVATTSASLSVE |
| | | | IPEVVSPELEVPVYPPAVIVPLRDAVT |
| | | | SEGQSARFQCRVTGTDLKVSWYSKD |
| | | | REIKPSRFFRMTQFEDTYQLEIAEAYP |
| | | | EDEGTYTFVASNSVGQVTSTAILKLE |
| | | | APEKIMYEKLEEEIEMEVKVAPILRR |
| | | | RLEPLEVAVNHVAKFTCEVETTPNV |
| | | | KFQWYKAGREIYDGDKYSIRSSNYL |
| | | | STLEIPRPQVVDCGEYSCKASNQHGS |
| | | | VSSTAFLTVTEPPRFIKKLDSSRLVKQ |
| | | | HDSTRYECKVGGSPEIKVTWYKGET |
| | | | EIHPSEKYSMSFVDSVAVLEMHNLS |
| | | | VEDSGDYSCEAQNPAGSASTSTSLK |
| | | | VKAPPAFTKKPHPVQTLKGSDVHLE |
| | | | CELQGTPPFQISWYKDKREIRSSKKY |
| | | | KVMSENYLASIHILNVDTADVGEYH |
| | | | CKAVNDVGSDSCIGSVTLRAPPTFVK |
| | | | KLSDVTVVVGETIELQAAVEGAQPIS |
| | | | VLWLKDKGEIIRESENLWISYSENVA |
| | | | SLKIGNAEPTNAGKYICQIKNDAGFQ |
| | | | ECFAKLTVLEPAVIVEKPGPVKVTAG |
| | | | DSCTLECTVDGTPELTARWFKDGNE |
| | | | LSTDHKYKISFFNKVSGLKILNAGLE |
| | | | DSGEYTFEVKNSVGKSSCTASLQVS |
| | | | DRIMPPSFTRKLKETYGQLGSSAVLE |
| | | | CKVYGSPPILVSWFHDGQEITSGDKY |
| | | | QATLTDNTCSLKVNGLQESDMGTYS |
| | | | CTATNVAGSDECSAFLSVREPPSFVK |
| | | | KPEPFNVLSGENITFTSIVKGSPPLEV |
| | | | KWFRGSIELAPGHKCNITLQDSVAEL |
| | | | ELFDVQPLQSGDYTCQVSNEAGKISC |
| | | | TTHLFVKEPAKFVMKVNDLSVEKGK |
| | | | NLILECTYTGTPPISVTWKKNGVILK |
| | | | HSEKCSITTTETSAILEIPNSKLEDQG |
| | | | QYSCHIENDSGQDNCHGAITILEPPYF |
| | | | VTPLEPVQVTVGDSASLQCQVAGTP |
| | | | EMIVSWYKGDTKLRGTATVKMHFK |
| | | | NQVATLVFSQVDSDDSGEYICKVEN |
| | | | TVGEATSSSLLTVQERKLPPSFTRKL |
| | | | RDVHETVGLPVTFDCGIAGSEPIEVS |
| | | | WFKDNVRVKEDYNVHTSFIDNVAIL |
| | | | QILKTDKSLMGQYTCTASNAIGTASS |
| | | | SGKLVLTEGKTPPFFDTPITPVDGIIG |
| | | | ESADFECHISGTQPIRVTWAKDNQEI |
| | | | RTGGNYQISYVENTAHLTILRVDRG |
| | | | DSGKYTCYASNEVGKDSCTAQLNV |
| | | | KERKTPPTFTRKLSEAVEETEGNELK |
| | | | LEGRVAGSQPLTVSWYKNNQEVHSS |
| | | | PHCEISFKNNTLLLHIKSVGQSDAGL |
| | | | YTCKVSNEAGSVLCTSSVVIREPKKP |
| | | | PVFDQPLQPAATEEGDTLQLSCHVR |
| | | | GSEPIRIQWLKAGREIRASERCSFSFA |
| | | | NGVALLELAAVTKSDSGEYVCKASN |
| | | | VAGTDTCRSKVTVKEKAALVSAAK |
| | | | KADIEGKLYFVSEPQSIKVMEKTVAT |
| | | | FIAKVGGDPIPNVKWMKGKWRQLN |
| | | | QGGRIIIQQRGDEAKLEIKDTTKTDS |
| | | | GLYKCVAFNQHGEIERSVNLQVEER |
| | | | KQEVVEEDVRGKLKRIPTKKKEDEE |
| | | | QTIDILELLKNVDPKEYEKYARMYGI |
| | | | TDFRGLLQAFELLKQTREEESHRLEI |
| | | | ELTEKAQKEDQEFEELVAFIQQRLTQ |
| | | | TEPVTLIRDIENQTVLTDEDAIFECEI |
| | | | KINYPEIKLSWYKGTQKLDSSDKYKI |
| | | | KIEGDRHILKIKNCQLEDQGNYRIVC |
| | | | GPHIASARLTVIEPAVERHLHDTTFK |
| | | | EGNTCTLSCQFSIPNAKSQWYRNGRP |
| | | | IKIGGRYSTQVSDKVHKLIIKDVRTE |
| | | | DQGQYTCKLDNLETTADLTIEAEPIQ |
| | | | FTKSIQNIVVSEHQSATFECEVSFDDA |
| | | | VVTWYKGPTELTESPKYSFRSEGRC |
| | | | HYMTIHNVTAEDEGVYSVIARLEPR |
| | | | GEARSTAELYLVTKEIKLELKPPDVP |
| | | | DAKVAVPPQKPAEAAPIPILLPLIPTP |
| | | | EEKKPAEKKVPVKKVSKKVVKKGP |
| | | | KEIPPPEVPEILPEKPKEVKITSMARR |
| | | | EEIHEEKMEIYEKPKKVYEEWEEDY |
| | | | GEDHDYYFKEEGYDEGEEEWEETY |
| | | | DKREVAYEEEQIIHEEVVEVPKKPVP |
| | | | ERKPPAITAEKKEKKEVTVHKVPDV |
| | | | PKKIPEEKVPITVPKKPEPTPAKEPEV |
| | | | HKIIEEKIKVSEPKKPEVPPAKVPEVP |
| | | | KKVVKEEVSVEVPKKPEPPPAKVPE |
| | | | VPKKVVPEEKVHVEVPKKAEPPPPK |
| | | | VLRKKPEEEEPKPEKEPKPEVKPVPT |
| | | | PVEKIKKPEVPEVPKKKTEIPVIKKEE |
| | | | KHVPEPPKEPKPAAISVPGPEPKPAV |
| | | | ALKSPKPAAEPAPAITTAPVTTPVVG |
| | | | KKAEAKPTKEETPKGISPVKAKKTPS |
| | | | PADAERRKLRPGSGGEKPPEEPPFTY |
| | | | QLKAVPLKFVKEMKDIVLKEAESVG |
| | | | SSAIFEVLISPSTAITSWMKDGSNIRES |
| | | | PKHKFIADGKDRKLHIIDVQLSDAGE |
| | | | YTCVLRLGNKEKTSTAKLIVEELPVR |
| | | | FVKTLEEEVTVVKGQPLYLTCELNK |
| | | | ERSVVWRRDGKIIKDKPGKFALGIIG |
| | | | LSHSLTITDSDDSDAGTYTVTVEDSE |
| | | | LSCSSCVKVVEVIRDWLVKPIRDQH |
| | | | VKPKGTATFTCDIVKDTPNIKWFKG |
| | | | DEEIPAEPTDKTEILKEGNKIFLKIKN |
| | | | AGPADIGEYSVEVEGRRYPAKLTLG |
| | | | EREVELLKPLEDVTVYEKETANFDTE |
| | | | ISEEDIPGEWKLKGEILRPSPTCEIKAE |
| | | | GGKRFLTLHKVKLEQAGEVLYQALN |
| | | | AVTTAILTVKEIELDFAVPLKDVTVP |
| | | | EKRQARFECVLTREANVIWSKGTDIL |
| | | | KIGEKFDIIADGKKHILVINDSQFDDE |
| | | | GEYTAEVEGKKSTARLFVEGVRLKFI |
| | | | TPLQDQTVKEGETAYFQFELSHEGM |
| | | | LVKWYKNDKRLHTSRTVFITSEGKV |
| | | | HKLEMREVTLDDISEIKAVVKDMNT |
| | | | QANLKVLEADPYFTVKLQDYSALEK |
| | | | DDITLQCELSKDVPVKWYKDGEELV |
| | | | ASSRISIKTDGLRRILTIKKATEGDKG |
| | | | VYECSCGTDKTNCNIGVEPRLIKVER |
| | | | PLYGVEVFVGETARFEIEISEPDVHPI |
| | | | WKLKGETLTPSPECEIIEDGKKHILIL |
| | | | HQGRLDMTGEVSFQAANAKSAANL |
| | | | KVKELPLIFITPLSDVKVFEKDEAKFE |
| | | | CEVSREPKTFRWLKGTQEITPDERFEI |
| | | | ISDGTKHALIIKSVAFDDEAKYMFEA |
| | | | EDKRTSAKLIIEGIRLKFITPLKDVTK |
| | | | KERETAVFTVELSHENIPVVWFKND |
| | | | QRLHTSKVVSMTDDGKFHTLTIKDL |
| | | | TIDDTSQIRVEAMGKSSEAKLTVLEG |
| | | | DPYFTGKLQDYTAVEKDEVILQCEIS |
| | | | KADAPVKWMKDGKPITPSKNVVIKA |
| | | | DGKKRILILKKALKKDIGQYTCDCGT |
| | | | DQTSANLNIEDRDIEIIRPLYSVEVIET |
| | | | ETARFDIEISEEGVHGNWKLKGEPLT |
| | | | ESPDCEIKEEGKKHFLTLYNVRLDQA |
| | | | GGVDFQAANAKSGAHLRVKPRVIGL |
| | | | LRPLKDVTVTAGESATFDCELSYEGI |
| | | | PVEWYLQGKKLEPSDKVVTRAEGR |
| | | | AHTLILRDVKLTDAGEVSLTAKDFRT |
| | | | QANLFVKEPPVEFTKPLEDQTVEEEA |
| | | | TAILECEVSRENAKVKWFKNGEEIH |
| | | | KTKKYDIISEGRVRKLIIHGCTLDDA |
| | | | KTYTCDAKDFKTSCFLNVEPLRVEFL |
| | | | RPLTDLEVKEKRICSVLNVKFLAQGV |
| | | | KVKWFKDGIEIEKAEYDIISKGAERIL |
| | | | VISKCLFDDEAEYACEAKTARTSGLL |
| | | | TVIEEEAVFTKNLHDLEVNENETVRL |
| | | | ICEISKPNAEVTWFKGDEEVPDGGRF |
| | | | EYISDGRKRILVIRNAHPEDAGKYTC |
| | | | KLPSSSTTGKLTVHELAAEFLTRPQN |
| | | | LEVLEGDKAEFACSVSKEAITVQWL |
| | | | WGDTVLEPGDKYDIISDGKKRTLVV |
| | | | KDSVVGDAGKYTVMVGEAKATARL |
| | | | TVIEKTQDYNSLKDQEVNEGQEIIFN |
| | | | CEVNKEGAKEKWYKNGEAIFDSAK |
| | | | YIIVQKDLVYTLRIRDTQLKDQATYS |
| | | | ISLSNHRGEHAESSAALTVLEEGLRIV |
| | | | EPLEDIETMEKKTVTFWCKVNRLNA |
| | | | TLKWTKNGEEITFNKRILYKIDKYKH |
| | | | SLIIKDCGFKDEGEYTVTAGQDKSVA |
| | | | ELLITEAPADFIEHLQDQTVTEFDDA |
| | | | VFTCQLSKEKASVKWYRNGREIKEG |
| | | | KKYQFEKDGNLHRLIIKDCRLDDECE |
| | | | YSCGVDDRKSRARLFVEEIPVEIIRPP |
| | | | QDVYEAPGADVIFMAELNKDGVEV |
| | | | KWLRNNMIIIQGDKHQMMSEGKVH |
| | | | RLQVCEIKPRDQGEYRFIAKDKEARA |
| | | | KLELAAAPRIKTGDQNLVVDVGNPL |
| | | | TMTVPYDAYPRAEAEWLKGEESLPT |
| | | | TTVDTTTDCTTFKIYEAKKSDKGRY |
| | | | KVVLRNKHAQAEAFINVEVIDVPGP |
| | | | VRNLEVTEIYDGEVGLAWQEPESDG |
| | | | GSKIIGYVVERRDIKRKTWIVVTDHA |
| | | | ENCEYTVTGLQKGGVEYLFRVSARN |
| | | | RVGTGEPVETERPVEAKSKFEVPGPP |
| | | | QNVEVTDVNRFGRTLTWEAPEYDG |
| | | | GSPITGYVIELRNRASIKWEPTMTTG |
| | | | ADELSAVLTDVVENEEYFFRVRAQN |
| | | | MVGVGKPSHPTRAVKITDPIERPSGN |
| | | | INLDHSDQTKTSVQLTWEPPLEDGGS |
| | | | PILGYIIERKEEGTDKWIRCNPKLVPA |
| | | | CAFKVTGLKAGSSYYYRVSAENAAG |
| | | | VSDPAEAIGPLTADDPFVDLQWPLSA |
| | | | FKDGLEVIVPEPIKIRVPITGYPIPTAT |
| | | | WSFGDQVLEEGGRVTMKTTSTFAEL |
| | | | VITPSERPDKGIYTLTLENPVSSVSGEI |
| | | | DVNVLARPSAPKELKVVDVSRSTVQ |
| | | | LSWEPPEDDGGSPVIDYIIEKREVSRK |
| | | | TWIKVMDHVIDQEFSVPDLIQGKEYL |
| | | | FRVCACNKCGPGEPAYIDEPINMSAP |
| | | | ATVPDPPENVKWRNPTSKGIFLTWEP |
| | | | PKYDGGARIKGYLVEKCQRGTDKW |
| | | | EICGEPVIETKMEVTKLKEGEWYAY |
| | | | RVKALNRIGASKPSKPTDDIQAIDAK |
| | | | EAPEIFLDVKLLAGLTVKAGTKIELP |
| | | | AKITGKPEPQITWTKAEKLLRPDDRI |
| | | | TIETTPNHSTVTITDSKRSDSGTYIIEA |
| | | | VNSSGRATAVVEVNVLDKPGPPAAF |
| | | | DVSEITNESCLLTWNPPRDDGGSKIT |
| | | | NYVLEKRATDSEIWHKLSSTI |
| SEQ ID | A6BLM7 | Titin isoform | EGEEEWEETYDKREVAYEEEQIIHEE |
| NO: 58 | (UniProtKB) | Ch12 | VVEVPKKPVPERKPPAITAEKKEKKE |
| | | (Fragment)/Gallus | VTVHKVVKKPVDEKVEITTQRVAEE |
| | | gallus | KLKQAEVTKKIPSAKPTPMIIEEKVM |
| | | | KKEAHKEVEEEEEREVISEELETHHV |
| | | | EVPDVPKKIPEEKVPITVPKKPEPTPA |
| | | | KEPEVHKIIEEKIKVSEPKKPEVPPAK |
| | | | VPEVPKKVVKEEVSVEVPKKPEPPPA |
| | | | KVPEVPKKVVPEEKVHVEVPKKAEP |
| | | | PPPKVLRKKPEEEEPKPEKEPKPEVK |
| | | | PVPTPVEKIKKPEVPEVPKKKTEIPVI |
| | | | KKEEKHVPEPPKEPKPAAISVPGPEP |
| | | | KPAVALKSPKPAAEPAPAITTAPVTT |
| | | | PWGKKAEAKPTKEETPKGISPVKA |
| | | | KKTPSPADAERRKLRPGSGGEKPPEE |
| | | | PPFTYQLKAVPLKFVKEMKDIVLKE |
| | | | AESVGSSAIFEVLISPSTAITSWMKDG |
| | | | SNIRESPKHKFIADGKDRKLHIIDVQL |
| | | | SDAGEYTCVLRLGNKEKTSTAKLIVE |
| | | | ELPVRFVKTLEEEVTVVKGQPLYLTC |
| | | | ELNKERSVVWRRDGKIIK |
| SEQ ID | A6BLM8 | Titin isoform | SDKYKIKIEGDRHILKIKNCQLEDQG |
| NO: 59 | (UniProtKB) | b11 | NYRIVCGPHIASARLTVIAEPIQFTKSI |
| | | (Fragment)/Gallus | QNIVVSEHQSATFECEVSFDDAVVT |
| | | gallus | WYKGPTELTESPKYSFRSEGRCHYM |
| | | | TIHNVTAEDEGVYSVIARLEPRGEAR |
| | | | STAELYLVTKEIKLELKPPDVPDAKV |
| | | | AVPPQKPAEAAPIPILLPLIPTPEEKKP |
| | | | AEKKVPVKKVSKKVVKKGPKEIPPP |
| | | | EVPEILPEKPKEVKITSMARREEIHEE |
| | | | KMEIYEKPKKVYEEWEEDYGEDHD |
| | | | YYFKEEGYDEGEEEWEETYDKREVA |
| | | | YEEEQIIHEEVVEVPKKPVPERKPPAI |
| | | | TAEKKEKKEVTVHKVPDVPKKIPEE |
| | | | KVPITVPKKPEPTPAKEPEVHKIIEEKI |
| | | | KVSEPKKPEVPPAKVPEVPKKVVKE |
| | | | EVSVEVPKKPEPPPAKVPEVPKKVVP |
| | | | EEKVHVEVPKKAEPPPPKVLRKKPEE |
| | | | EEPKPEKEPKPEVKPVPTPVEKIKKPE |
| | | | VPEVPKKKTEIPVIKKEEKHVPEPPKE |
| | | | PKPAAISVPGPEPKPAVALKSPKPAA |
| | | | EPAPAITTAPVTTPVVGKKAEAKPTK |
| | | | EETPKGISPVKAKKTPSPADAERRKL |
| | | | RPGSGGEKPPEEPPFTYQLKAVPLKF |
| | | | VKEMKDIVLKEAESVGSSAIFEVLISP |
| | | | STAITSWMKDGSNIRESPKHKFIADG |
| | | | KDRKLHIIDVQLSDAGEYTCVLRLG |
| | | | NKEKTSTAKLIVEELPVRFVKTLEEE |
| | | | VTVVKGQPLYLTCELNKERSVVWRR |
| | | | DGKIIKDKPGKFALGIIGLSHSLTITDS |
| | | | DDSDAGTYTVTVEDSELSCSSCVKV |
| | | | VEVIRDWLVKPIRDQHVKPKGTATF |
| | | | TCDIVKDTPNIKWFKGDEEIPAEPTD |
| | | | KTEILKEGNKIFLKIKNAGPADIGEYS |
| | | | VEVEGRRYPAKLTLGEREVELLKPLE |
| | | | DVTVYEKETANFDTEISEEDIPGEWK |
| | | | LKGEILRPSPTCEIKAEGGKRFLTLHK |
| | | | VKLEQAGEVLYQALNAVTTAILTVK |
| | | | EIELDFAVPLKDVTVPEKRQARFEC |
| SEQ ID | P79757 | Connectin/titin | KSRLRRRREREITEITSEEEEEIEMVQ |
| NO: 60 | (UniProtKB) | (Fragment)/Gallus | HVHREFSPPSRLLRRRRSLSPTYIELM |
| | | gallus | RPVSELIRPRSRPPEESERRSPTPERTR |
| | | | PRSPSPVSTERSLSRFERMARFDIFSR |
| | | | YESMKSALKTQKTMERKYEVLTQQP |
| | | | FTLDHAPRITLRMRSHRVPCGHNTRF |
| | | | ILNVQSKPTADVKWYHNGIELQESS |
| | | | KIHFSNTSGVLTLEILDCHIDDSGTYR |
| | | | AVCTNYKGECSDYATLDVTGGDYT |
| | | | TYSSQRRDEEVPRSILPDLTRTEAYA |
| | | | VSSFKKATAAEASSSVREVKSEVSAT |
| | | | RESLLSYEHHASSEEKITASEEKSLEE |
| | | | RTVHKAFKSTLPATILTKPRSITVSEG |
| | | | ETARFSCDVDGEPAPTITWVRAGQPI |
| | | | VSSRRFQITRTQYKSTFEISLVQIADE |
| | | | GSYTVVVENSEGRQEAHFTLTVQRK |
| | | | RIPEKAITSPPRIKSPEPRVKSPEPVKS |
| | | | PKRVKSPEPISTPSKAKSPPGDKTAPV |
| | | | EKVQLPTASPPKIKEQLKAETLGDKV |
| | | | KLSCAVESSVLSIREVAWYKDGKKL |
| | | | KEDHHFKFHYAADGTYELKIHNLTE |
| | | | SDKGEYTCEIMGEGGISKTNFQFTGQ |
| | | | VFKNIHSQVQSVSETPKSVEKGDKVL |
| | | | AVSTQKKSSAATEEKAAIEEVIKKSI |
| | | | VTEDVKQLQAEIRASSTQMTVSEGQ |
| | | | KVTLKANIPGASEVKWVLNGMELR |
| | | | NSDDYRYGISGSNHTLTIKKASNKDE |
| | | | GILTCEGKTDEGTIKCQYVLTFSKEPS |
| | | | NEPAFITQPKSQNVNEGQDVLFTCEV |
| | | | SGDPSPEVEWLRNNQPIAVSSHMRA |
| | | | TRSKNTYSLEIRNAAVSDTGKYTVK |
| | | | AKNYHGQCSATASLTVFPLIEEPPKE |
| | | | VVLKTSGDASMHESFSSQSFQMAAS |
| | | | KQEASFSSFSSSSMTETKFASMSAKS |
| | | | MSSMKESFVEMSSSSIMGKSSMAQL |
| | | | ESSTSKMLKSGVRGVPPKIEALPSDIS |
| | | | IDEGKVLTLSCAFSGEPAPEITWYCR |
| | | | GRKITSQDQQGRFHIETSEDLTTLIIM |
| | | | DVQKNDGGIYTLNLGNEFGTDSATV |
| | | | NINIRS |
| SEQ ID | Q98918 | Connectin/titin | MTTKAPTFTQPLQSVVALEGSAATFE |
| NO: 61 | (UniProtKB) | (Fragment)/Gallus | AHISGFPVPEVSWYRDGQVLSAATLP |
| | | gallus | GVQISFSDGRAKLVIPSVTEANSGRY |
| | | | TIQATNGSGQATSTAELLVTAGTAPP |
| | | | NFSQRLQSMTARQGSQVRLDVRVTG |
| | | | IPTPVVKFYRDGVEIQSSPDFQILQEG |
| | | | DLYSLIIAEAYPEDSGTYSVNATNNV |
| | | | GRATSTAELLIQGEEEAVPAKKTKTI |
| | | | VSTAQISQTRQARIEKKIETHFDARSL |
| | | | TSVEMVIEGAAAQQLPHKAPPRMPP |
| | | | RPTSKSPTPPVITAKAQMARQQSPSP |
| | | | VRQSPSPVRHVRAPTPSPVRSVSPAG |
| | | | RISTSPIRPVKSPSPIRKAQVVTPGAE |
| | | | VLPPWRQEGYSATAEAQMKETRVST |
| | | | SATEIRTEERWEGRYGLQEQVTISGA |
| | | | AAGEVAAGAKEVRKEPEKTPVPTVII |
| | | | ATDK |
| | | | AKEQERISTAREEISARHEQVHVSHE |
| | | | QIEAGKRAEAVATVVAAVDQARVR |
| | | | SPWETEQVDETYVKKKTLEYGYKEH |
| | | | AVKDHEAQAEHHVATKEVKTVYVP |
| | | | PEKHIPAAEKKEVHVSTEIKRETEAKI |
| | | | EKTIHIEHPRPRTASPHFTVSKIAVPK |
| | | | PDHTYEVSIAGSAMATLEKELSATSA |
| | | | AQKITKPVKPPQLKPHEVKIKPESAPP |
| | | | QFPFTEAAETYKAHYDVETKKEVDV |
| | | | SIKGEAVREDHLLLRKESEAKVTETA |
| | | | RVPVPAEIPVTPPTLVWGLKNKTVTE |
| | | | GESVTLECHISGHPQPTVTWYREDY |
| | | | KIESSMDFQITFKAGLARLVIREAFAE |
| | | | DSGRFTCTATNKAGSVSTSCHLHVK |
| | | | VSEETETRETISEKVVTEEKSYVETK |
| | | | DVVMEDVSAAAEEVSGEPVPPFFIRK |
| | | | PVVHKLIE |
| | | | GGSIIFECQVGGNPKPHVLWKKGGV |
| | | | PLTTGYRYKVSYKRETGECKLEISMT |
| | | | FADDAGEYTIVIRNKFGEASATVSLL |
| | | | EEADYEAYIKSQQEMMYQTQVTAY |
| | | | VQEPKVAEVAPPISYGDFDKEYEKE |
| | | | QALIRKKMAKDTVMVRTFVEDEEFH |
| | | | ISSFEERLIKEIELRIIKTTLDELLEEDG |
| | | | EEMMIDISESEAIGAGFDLRLKNYRT |
| | | | FEGTGVTFHCKTTGYPLPKIAWYKD |
| | | | GKRIRHGERYHMEVLQDGSASLRLP |
| | | | VVLPEDEGIYTVFASNMKGNAICSA |
| | | | KLYVEPVAPTATPGYMPGPEVMRRY |
| | | | RSISPRSPSRSPARSSPSCSPARRLDET |
| | | | DEGQLERLYKPVFVLKPTSVKCSQG |
| | | | QTARFDLKVVGRPMPETYWFHNGQ |
| | | | QVVNDYTHKIVIKEDGTQSLIIVPAM |
| | | | PEDSGEWAVIA |
| | | | QNRAGKASVSVTLSVEAKEDLVRPR |
| | | | FVERLRNVSVKEGSRLHMAVKATG |
| | | | NPNPDIVWLKNSDIIVPHKYPRIRIEG |
| | | | TKGAAALNIESTARQDAAWYTATAI |
| | | | NKAGRDTTRCKVNVEVEHAEPEPER |
| | | | RLIIPKGTYKAKEIAAPELEPLHLRYG |
| | | | QEQWEEGDLYDKEKQQKPFFKKKL |
| | | | TSLRLKQFGPAHFECRLTPIGDPTMV |
| | | | VEWLHDGKPLEAANRLRMINEFGYC |
| | | | SLDYGVAYSRDSGVITCRATNKYGT |
| | | | DHTSATLIVKDEKSLVEESQLPEGRR |
| | | | GMQRIEELERMAHEGALPAVAVDQ |
| | | | KEKQKPELVLVPEPARVLEGETARFR |
| | | | CRVTGYPLPKVNWYLNSQLIRKSKR |
| | | | FRLRYDGIHYLDIVDCKSYDTGEVK |
| | | | VTAENPEGFIEHKVKLEIQQREDFRS |
| | | | VLRRAPEPRHEPVVT |
| | | | EPGKLLFEVQKIDKPAEATTKEVVKL |
| | | | KRAERITHEKLSEESEELRSKFKRRTE |
| | | | EGYYEAITAVELKSRKKDESYEEML |
| | | | KKTKEELLHWTKEIPEEEKKALPPEG |
| | | | KITIPTFKPEKVELSPSMEAPKIFERIQ |
| | | | SQTVAQGTDAHFRVRVVGKPDPECQ |
| | | | WFRNGVQIERTDRIYWYWPEDNVCE |
| | | | LVIRDVTADDSASIMVKAVNIAGETS |
| | | | SHAFLLVQAKQLISFIQNLQDVVAKE |
| | | | RDSMATFECETSEPFIKVKWFKNGIEI |
| | | | HSGEKYRMHSDRKAHFLSVLAVEM |
| | | | SDADDYSCALVEDESVKTTAKLIVE |
| | | | GAVVEFIKELEDVEVPESFTGELECE |
| | | | VSPEDIEGKWYHGDVELSSNHKYVL |
| | | | ASRRGRRILTIKDVNKDDQGEYSFVV |
| | | | DGKRTHCKLKMKPRPMTILQGLTDQ |
| | | | KVCEGDIV |
| | | | QLEVKVSVENVEGVWMKDGHEIQS |
| | | | SDRIHIVLDKQSHMLLIEDATQEDSG |
| | | | TYSFSIPGLELSTTGQVTVYSVEIIVPL |
| | | | KDVHVVEGTKAILECKVSAPDVTSS |
| | | | KWYLNDHQIKPDERVQAVCKGTKQ |
| | | | RLVITRTHASDEGHYKLMVGKVETS |
| | | | CNVTVEEIEIIRGLHDITCTETQNVSF |
| | | | EVELSHSGIDVIWHFKGQEIKAGPKY |
| | | | KIEARGKIYKLTVVKMMKDDEGEY |
| | | | VFYAGGKKTSGKLIVAGGAISKPLA |
| | | | DLTVAESQRAVFECEVANPESEGQW |
| | | | LKNGKPLPMTDQYRAETDGVKRRL |
| | | | NIPAAKMDDMGEYSYEIASSKTSAK |
| | | | LHVEAVKIKKTLKNLTVTETQEAVFS |
| | | | VELSHPDVKGALWIKNGVELESNDK |
| | | | YEISVKGTVHTLKIKHCVVTDESVYS |
| | | | FKLGKIGANARLHVE |
| | | | TVKIIKKPKDVTALENAVVSFELSVS |
| | | | HDTVPVRWFHKNVELKQSDKYKMI |
| | | | SQRKVHKLMLHNISPADAGEYTAFV |
| | | | GQLECKAKLFVETIHITKTMKSIEIPE |
| | | | TKTASFQCEVSHFNVPSVWLKNGVE |
| | | | IEMSEKFKIVVQGKLHQLNIMNTSSE |
| | | | DSAEYTFVCGNDRVSATLTVKPILIT |
| | | | SMLEDINAEEKDTITFEVTVNYEGIS |
| | | | YKWLKNGVEIKSTDKCQIRTKKLTH |
| | | | SLSIRNVHFGDAAEYSFVAGKAASSA |
| | | | TLYVEARHIEFRKHIKDIKVVEKKRA |
| | | | IFECEISEPDVQVQWMKDGQELQIGD |
| | | | RMKIQREKYVHRLIIPSTKMSDAGQY |
| | | | TVVAGGNTSSANLIVEGRDVRIRSIR |
| | | | KEIQVIERQRAEIEFEVNEDDIEPQWY |
| | | | KDGIEINFHYEERYSYVVERRIHRMSI |
| | | | FETTYS |
| | | | DAGEYTFVAGRNRSSVVLYVNAPEP |
| | | | PQIIQELQPTTVESGKPARFCAIISGKP |
| | | | QPKVSWYKDDQQLSPGFKCKFLHD |
| | | | AQEYTLLLIETFPEDSAVYTCEAKND |
| | | | YGVATTSASLSVEIPEVVSPELEVPV |
| | | | YPPAVIVPLRDAVTSEGQSARFQCRV |
| | | | TGTDLKVSWYSKDREIKPSRFFRMT |
| | | | QFEDTYQLEIAEAYPEDEGTYTFVAS |
| | | | NSVGQVTSTAILKLEAPEKIMYEKLE |
| | | | EEIEMEVKVAPILRRRLEPLEVAVNH |
| | | | VAKFTCEVETTPNVKFQWYKAGREI |
| | | | YDGDKYSIRSSNYLSTLEIPRPQVVD |
| | | | CGEYSCKASNQHGSVSSTAFLTVTEP |
| | | | PRFIKKLDSSRLVKQHDSTRYECKVG |
| | | | GSPEIKVTWYKGETEIHPSEKYSMSF |
| | | | VDSVAVLEMHNLSVEDSGDYSCEAQ |
| | | | NPAGSAST |
| | | | STSLKVKAPPAFTKKPHPVQTLKGSD |
| | | | VHLECELQGTPPFQISWYKDKREIRS |
| | | | SKKYKVMSENYLASIHILNVDTADV |
| | | | GEYHCKAVNDVGSDSCIGSVTLRAP |
| | | | PTFVKKLSDVTVVVGETIELQAAVE |
| | | | GAQPISVLWLKDKGEIIRESENLWIS |
| | | | YSENVASLKIGNAEPTNAGKYICQIK |
| | | | NDAGFQECFAKLTVLEPAVIVEKPGP |
| | | | VKVTAGDSCTLECTVDGTPELTARW |
| | | | FKDGNELSTDHKYKISFFNKVSGLKI |
| | | | LNAGLEDSGEYTFEVKNSVGKSSCT |
| | | | ASLQVSDRIMPPSFTRKLKETYGQLG |
| | | | SSAVLECKVYGSPPILVSWFHDGQEI |
| | | | TSGDKYQATLTDNTCSLKVNGLQES |
| | | | DMGTYSCTATNVAGSDECSAFLSVR |
| | | | EPPSFVKKPEPFNVLSGENITFTSIVK |
| | | | GSPPLEVKWF |
| | | | RGSIELAPGHKCNITLQDSVAELELF |
| | | | DVQPLQSGDYTCQVSNEAGKISCTT |
| | | | HLFVKEPAKFVMKVNDLSVEKGKN |
| | | | LILECTYTGTPPISVTWKKNGVILKHS |
| | | | EKCSITTTETSAILEIPNSKLEDQGQY |
| | | | SCHIENDSGQDNCHGAITILEPPYFVT |
| | | | PLEPVQVTVGDSASLQCQVAGTPEM |
| | | | IVSWYKGDTKLRGTATVKMHFKNQ |
| | | | VATLVFSQVDSDDSGEYICKVENTV |
| | | | GEATSSSLLTVQERKLPPSFTRKLRD |
| | | | VHETVGLPVTFDCGIAGSEPIEVSWF |
| | | | KDNVRVKEDYNVHTSFIDNVAILQIL |
| | | | KTDKSLMGQYTCTASNAIGTASSSG |
| | | | KLVLTEGKTPPFFDTPITPVDGIIGES |
| | | | ADFECHISGTQPIRVTWAKDNQ |
| SEQ ID | Q07784 | Titin | RESDHRAWTPVSYTVTRQNAVVQG |
| NO: 62 | (UniProtKB) | (Fragment)/Gallus | LTEGKAYFFRIAAENIIGMGPFTETTK |
| | | gallus | ELIIRDPITVPDRPEDLEVKAVTKNSV |
| | | | TLTWNPPKYNGGSDITMYVLESRLIG |
| | | | KEKFHRVTKEKMLDRKYTVEGLKE |
| | | | GDTYEYRVSACNIVGQGKPSFCTKPI |
| | | | TCKDEIAPPTLDLDFRDKLIVRVGEA |
| | | | FSLTGRYSGKPTPKITWLRDDIVLKE |
| | | | DDRTKIKTTPTTLCLGILKSVREDSG |
| | | | KYCVTVENSTGSRKGFCQVTVVDRP |
| | | | SPPVGPVIFDEVHKDHMIVSWKPPLD |
| | | | DGGSEITNYIIEKKDTHRDLWMPVTS |
| | | | ATVKTTCKIPKLLEGREYQVRIYAEN |
| | | | LYGISDPLISDEMKAKDRFSVPDAPE |
| | | | QPVIKDVTKDSAVVVWNKPYDGGK |
| | | | PITNYIVEKKETMATRWVRVTKDPIF |
| | | | PSTQFKVPDLLEGCQYEFRVSAENEI |
| | | | GVGNPSPPSKPIFARDPIVKPSPPVNP |
| | | | EAVDKTKNSVDLTWQPPRHDGNGKI |
| | | | IGYLVEYQKVGDEEWKKANLTPDSC |
| | | | PETKYKVTGLTEGLTYKFRVMAVNA |
| | | | AGESEPAYVPDPVEVKDRLEPPELIL |
| | | | DANMAREQHVRAGDTLRLSAVIKG |
| | | | VPFPKVSWKKEDKEVSPKADIEVTG |
| | | | VGSKLEIRNTVHEDGGIYSLTVENPA |
| | | | GSKTVSVKVLVLDKPGPPRNLQVSD |
| | | | VRSDSCYLTWKEPEDDGGSVITNYV |
| | | | VERKDVASAQWVSVSSSSKKRSHM |
| | | | AKYLMEGTQYLFRVAAENLFGRGPY |
| | | | VETLKPIKAMDPLHPPGPPKNLHHID |
| | | | VDKTEVSLVWNRPDRDGGAEITGYL |
| | | | VEYQEDGADEWTKFQTVPMLDCVV |
| | | | TGL |
| SEQ ID | Q90720 | Titin | VTTKQTVISRTREGIVTSQEQRHISRE |
| NO: 63 | (UniProtKB) | (Fragment)/Gallus | KVRKEPEKTPVPTVIIVTAKVKEQERI |
| | | gallus | STAREEISARHEQVHVSHEQIRREDV |
| | | | KPSVPKVVITTDKPKAPVLISQSKEGI |
| | | | ATKKEHVSISHEKIKKEAKKTTAVLK |
| | | | IIAPVTVSRTREEITAKPEQMHLAYD |
| | | | QIEAGKRAEAVATVVAAVDQARVR |
| | | | SPWETEQVDETYVKKKTLEYGYKEH |
| | | | AVKAHEAQAEHHVATKEVKTVYVP |
| | | | PEKHIPAAEKKEVHVSTEIKRETEAKI |
| | | | EKTNHIEHPRPRTASPHFTVSKIAVPK |
| | | | PDHTYEVSIAGSAMATLEKELSATSA |
| | | | EQKITKPVKPPQLKPHEVKIKPESAPP |
| | | | PFPFTEAAETYKAHYDVETKKEVDV |
| | | | SIKGGAVREDHLLLRKESEAKVTETA |
| | | | RVPVPAEIPVTPTHLVWGLKNKTVT |
| | | | EGESVTLECHISGHPQPTVTWYRDD |
| | | | YKIESSMDFQITFKAGLARLVIREAF |
| | | | AEASGRFTCTATNKAGSVSTSCHLH |
| | | | VKVSEETETRETISEKVVTEEKSYVE |
| | | | TQDVVMEDASPPPEEVSGEPVPPFFI |
| | | | RKPVVHTLIVGGSIIFEFHVGGNPKPH |
| | | | VLLKKGGVPLTTGYRYKVSYKRETG |
| SEQ ID | F1N757 | Titin/Bos taurus | MATQAPTFTQPLQSVVVLEGSTATFE |
| NO: 64 | (UniProtKB) | | AHISGFPVPEVSWFRDGQVISTSTLPG |
| | | | VQISFSDGRAKLTIPAVTKANSGRYS |
| | | | LRATNGSGQATSTAELLVTAETAPPN |
| | | | FTQRLQSMTVRQGSQVRLQVRVTGI |
| | | | PTPVVKFYRDGAEIQSSLDFQISQEGE |
| | | | LYSLLIAEAYPEDSGTYSVNATNSVG |
| | | | RATSTAELLVQGEEVVPAKRTKTIVS |
| | | | TAQISETRQTRIEKKIEAHFDARSIAA |
| | | | VEMVIDGAAGQQLPHKTPPRIPPKPK |
| | | | SRSPTPPSIAAKAQLARQQSPSPIRHS |
| | | | PSPVRHVRAPTPSPVRSVSPAGRISTS |
| | | | PIRSVKSPLLVRKAQTPTMPPGPEVPP |
| | | | PWKQEGYVASSEAEMRETTVTSATQ |
| | | | IRTEERWEGRYGIQEQVTISGAAGAA |
| | | | ASTTFAAGAVAAGAAEVKQEADKS |
| | | | AAVATVVAAVDMARVREPVISAVE |
| | | | QTAQRTTTTAVHIQPAHEQIRKEAEK |
| | | | TAVTKVVVAADKAKEQEVKARTRE |
| | | | VITTKQEQVHVTHEQIRKETEKAFVP |
| | | | KVVISAAKAKEKETKITGEITTKQEQ |
| | | | KQITQETIIQKAETAAVSMVVVETAK |
| | | | PTTLETILGAQEEIVTQQDQMHITHE |
| | | | KILKETRKTVVPKVIVATPKVKEQDV |
| | | | VSRTREGITTKREQVQVTHEKMRKE |
| | | | AEKTALSTIAVATAKAKEQETILRTR |
| | | | EEMATRQEQIQVTHGKVGVGKKAE |
| | | | AVATVVAAVDQARVREPREPRHVEE |
| | | | SYAQQTTLEYGYKEHISATKVAEQPP |
| | | | RPASEPQVVPKAVKPGVIHAPSETHI |
| | | | ATTDQMGMHISSQIKKTTDLTSERLV |
| | | | HVDKRPRTASPHFTVSKISVPKTEHG |
| | | | YEASIAGSAIATLQKELSATPSAQKV |
| | | | TKSVKAPTVKPAEARVRAEPTPSPQF |
| | | | PFAETPETFKSQAGIEVKKEVGVSITG |
| | | | VAVREERFEALRERETKVTETARVP |
| | | | APVEIPVTPPTLVSGLKNVTVIEGESV |
| | | | TLECHISGYPSPKVTWYREDYQIESSI |
| | | | DFQITFQSGIARLLIREAFAEDSGRFT |
| | | | CTAVNEAGTVSTSCYLAVQVSEEFE |
| | | | KETTAVAEKITTEEKRFVESRDVVM |
| | | | TDTSITEEHAGPGEPAAPFFITKPVVQ |
| | | | KLVEGGSIVFECQVGGNPKPHVYWK |
| | | | KSGVPLTSGYRYKVSYNKQTGECRL |
| | | | VISMTFADDAGEYTIVIRNKHGETSA |
| | | | SASLLEEADYESLMKSQQEMLYQTQ |
| | | | VTAFVQEPKVGEIAPGYVYSEYEKE |
| | | | YEKEQALIRKKMAKDTVMVRTFVE |
| | | | DQEFHISSFEERLIKEIEYRIIKTTLEEL |
| | | | LEEDGEEKMAVDISESEAIEAGFDLR |
| | | | VKNYRILEGMGVTFHCKMSGYPLPK |
| | | | IAWYKDGKRIKHGERYHMDFLQDG |
| | | | RASLRIPVVLPEDEGIYTAFASNMKG |
| | | | NAICSGKLYVEPAAPLSAPTYIPTPEP |
| | | | VSRIRSISPRSVSRSPIRMSPARMSPA |
| | | | RMSPGRRLEETDESQLERLYKPVFVL |
| | | | KPTSFKCVEGQTARFDLKVVGRPMP |
| | | | ETFWFHDGQQIVNDYTHKVVVKED |
| | | | GTQSLLIVPATPSDSGEWTVVAQNR |
| | | | AGKSSISVILTVEAVEHQVKPVFVEK |
| | | | LRNLNIKEGSRLEMKVRATGNPNPDI |
| | | | VWLKNSEIIVPHKYPKIRIEGTKGEA |
| | | | ALKIDSTVSQDSAWYTATAINKAGR |
| | | | DTTRCKVNVEVEFAEPEPERRLIIPRG |
| | | | TYRAKEIAAPELEPLHLRYGQEQWE |
| | | | EGDLYDKEKQQKPFFKKKLTSLRLK |
| | | | RFGPAHFECRLTPIGDPTMVVEWLH |
| | | | DGKPLEAANRLRMINEFGYCSLDYA |
| | | | VAYSRDSGVITCRATNKYGTDHTSA |
| | | | TLIVKDEKSLVEESQLPEGRKGLQRI |
| | | | EELERMAHEGALTGVTTDQKEKQKP |
| | | | DIVLFPEPVRVLEGETARFRCRVTGY |
| | | | PQPKVNWYLNGQLIRKSKRFRVRYD |
| | | | GIHYLDIVDCKSYDTGEVKVTAENPE |
| | | | GVIDHKVKLEIQQREDFRSVLRRAPE |
| | | | PKPEFHVHEPGKLQFEVQKVDRPVD |
| | | | TTETKEIVRLKRAERITHEKVSEESEE |
| | | | LRSKFKRRTEEGYYEAITAVELKSRK |
| | | | KDESYEELLRKTKEELLHWTKELTE |
| | | | EEKKALAEEGKITIPTFKPDKIELSPS |
| | | | MEAPKIFERIQSQTVGQGSDAHFRVR |
| | | | VVGKPDPECEWYKNGVKIERSDRIY |
| | | | WYWPEDNVCELVIRDVTAEDSASIM |
| | | | VKAINIAGETSSHAFLLVQAKQLITFT |
| | | | QELQDVVAKEKDTMATFECETSEPF |
| | | | VKVKWYKDGVEIHTGDKYRMHSDR |
| | | | KVHFLSVLMIDTSDAEDYSCVLVED |
| | | | ENVKTTAKLIVEGAVVEFVKELQDIE |
| | | | VPESFSGELECIITPENIEGKWYHKGV |
| | | | ELKSNGKYTITSRRGRQNLTVKDVT |
| | | | KEDQGEYSFVVDGKKTTCKLKMKP |
| | | | RPIAILQGLSDQKVCEGDIVQLEVKV |
| | | | SLENVEGVWMKDGEEVQPGDRVHI |
| | | | VIDKQSHMLLIEDMTKEDAGHYSFTI |
| | | | PSLGLSTHGRVSVYSVDVITPLKDVN |
| | | | VLEGTKAVLECKVSVPDVTSVKWYL |
| | | | NDEQIKPDDRVHAIVKGTKQRLVINR |
| | | | THASDEGPYKLVVGRVETSCDLSVE |
| | | | KIKIIRGLRDLTCTETQNVVLEVELSH |
| | | | SGIDVLWNFKDKEIKPSSKYKIEAHG |
| | | | KIYKLTVLNMMKDDEGEYTFYAGE |
| | | | NRTSGKLTVAGGAISKPLTDQTVAES |
| | | | QEAVFECEVANPDSEGEWLKDGKHL |
| | | | PLSKTIRSESDGRKRRLIIAATKLDDI |
| | | | GEYTYKVATSKTSAKLKVEAVKIKK |
| | | | TLKNLTVTETQDAVFTVELTHPNVK |
| | | | GVQWIRNGVVLESSDKYTVSVKGTV |
| | | | YSLRIKNCAVADESVYGFKLGKLGA |
| | | | SARLHVETVKIIKKPKDVTALENATV |
| | | | SFEVSVSHDTVPVKWFHKNVEIKPSD |
| | | | KHRLVSERKVHKLMLQHISPSDAGE |
| | | | YTAVVGQLECKAKLFVETLHITKTM |
| | | | KNIEVPETKTASFECEVSHFNVPSMW |
| | | | LKNGVEIEMSEKFKIVVQGKLHQLII |
| | | | MNTSTEDSAEYTFVCGNDQVSATLK |
| | | | VTPIMITSMLKDINAEEKDTITFEVTV |
| | | | NYEGISYKWLKNGVEIKSTDRCQMR |
| | | | TKKLTHSLNIRNVHFGDAAEYTFVA |
| | | | GKATSTATLYVEARHIEFRKHIKDIK |
| | | | VLEKKRAMFECEVSEPDITVQWMKD |
| | | | GQELQLVDRIKIQKEKYVHRLLIPST |
| | | | RMSDAGKYTVVAGGNMSSANLFVE |
| | | | GRDVRIRSIKREVQVIEKQRAVVEFE |
| | | | VNEDDVDAHWYKDGIEINFQVQERH |
| | | | QYVVERRIHRMFISETRHSDAGEYTF |
| | | | VAGRNRSSVTLYVNAPEPPQILQELQ |
| | | | PITVQSGKPARFCAVISGRPQPKISWY |
| | | | KEEQLLSTGFKCKFLHDGQEYTLLLI |
| | | | EAFPEDAAVYTCEARNDYGVATTSA |
| | | | SLSVEVPEVVSPDQEMPVYPPAIVSP |
| | | | LQDAVTSEGRPAHFQCRVSGTDLKV |
| | | | SWYSKDKKIKPSRFFKMTQFEDTYQ |
| | | | LEVAEAFPEDEGIYTFVASNAVGQVS |
| | | | STATLRLEAPEAMLHEQIEMEMKEFS |
| | | | ISLLSGKEERPQTSSWDLQLFDINETL |
| | | | EPLSEPSVYSPKFDSKKEGNAPVFIKE |
| | | | VSNAEISIGDVAKLFVTVTGIPTPQIQ |
| | | | WFFNGAMLTPSADYKFVFDGNDHSL |
| | | | IILFTKLEDEGEYTCIASNEYGQAMC |
| | | | SAYLKINSEAEGHEEPESAEKSLEKL |
| | | | QGPCPPYFLKELKPVHCVQGLPAIFE |
| | | | YVVLGEPAPTVLWFKENKQLCTNVY |
| | | | YTIIHNPDGSGTFIVNDPQKEDGGLY |
| | | | VCKAENVWGGSTCAAELFVLLEDTD |
| | | | MTDATCKAAPTPEAPEGFPHTSVKD |
| | | | PAVETFDSEQEVVTFVKDAILKASLI |
| | | | AEEKQQFSYEHVVKTNELSSQVTLK |
| | | | AEQLQSTVILEHDMPTPESTRELLSIS |
| | | | GTIHVQPIKELPPSLQLQIAQSQDTLP |
| | | | REDTLMCQEPESQVVLSDTEKIFPSF |
| | | | MSIEEISLSTVESLQTLLAEPEESYPQP |
| | | | LIKETPAHSYPTSVAEEVLLSKDKTV |
| | | | SAVDRGQRETSQKQESQNALFLNQN |
| | | | LAEGHAQSLQGPDVTVSRVSSEHTC |
| | | | TESEILMESVDQLDSAGQDFAARIEE |
| | | | GQSLRFPLACEEKQVLLKEEHSDTLA |
| | | | LPLNQTTEYKKEPMAVNGVQEVQGS |
| | | | DFLSKESLLPGIPEEQRLNLKTQIRSA |
| | | | LQAAVASEQLSLFSEWLRNIEQVEV |
| | | | KAIDFTQEPNCIMCTYLITSVKSLTEE |
| | | | VTVTIKGIDPQMADLKTELKEALCSI |
| | | | CEEMNILTAEEPGIEKGATVVLQEDV |
| | | | SFSSSQKLEAITEPEAESKYLVSKEEIS |
| | | | YFKVESQVKAVGTTTASAAVSDEKQ |
| | | | DELPKPSEEKEKVSEGGTEEVGTVEI |
| | | | QEAEDEEDRKLGEDGPDVQTPLVDT |
| | | | IAEEGDIISLTSCITNAKEVNWYFESK |
| | | | LVPSDEKFKCLQDQNTYTLVIDKVN |
| | | | REEHQGEYTCEALNDNGKATTSAKL |
| | | | TVVKRAAPVIRRRIEPLEVALGHLAK |
| | | | FTCEIHSAPNVRFQWFKAGREIYESD |
| | | | KCSIRSANYVTTLEILRTQVVDCGEY |
| | | | TCKASNEYGSVSCTATLTVTEAYPPT |
| | | | FFSRPKSVTTFVGKAAKFLCTVTGTP |
| | | | VIETIWQKDGMALSPSPTCKISDVDN |
| | | | KHILELSNLTVHDKGVYSCKASNKF |
| | | | GADTCQAELDIIDKPHFIKELEPVQSA |
| | | | INKKIHLEGQVDEDRKVTITWSKNG |
| | | | QKLPPGKDYKIYFEDKIASLEIPLAKL |
| | | | KDSGTYVCTASNEAGSSSTSATVTIR |
| | | | EPPSFVKKVDPSYLMLPGESARLHCR |
| | | | LKGSPVIQVTWFKNNKELTESNTIRM |
| | | | SFVNSEAVLDITDVKVEDSGTYSCEA |
| | | | VNDVGSDSCSAEIVVKEPPSFIKTLEP |
| | | | ADIVRGTNALLQCEIAGTGPFEISWF |
| | | | KDKKQIRSSKKYRLFSQKSTVSLEIFS |
| | | | FNSADVGDYECVVANEVGKCGCVA |
| | | | THLLKVEPPTFVKKVDDFTALAGQT |
| | | | VTLQAAVRGSEPISVTWMKGQEIIKE |
| | | | DGKIKMSFANGVAVLIIPDVQISFGD |
| | | | KYTCLAENEAGSQTSVGELIVKEPAK |
| | | | IIERAELIQVTAGDPATLEYTVAGTPE |
| | | | LKPKWYKDGRPLVASKKYRISFKNN |
| | | | VAQLKFYSAELHDSGQYTFEISNEVG |
| | | | SSSCETTFTVLDRDIAPFFTKPLRNVD |
| | | | SVVSGTCRLDCKIAGSLPMRVSWFK |
| | | | DGKEVTSSDRYRIAFVEGTASLEISR |
| | | | VDMSDAGNFTCRATNSVGSKDCSG |
| | | | ALIVQEPPSFVTKPASKDILPGSAVFL |
| | | | KSTFQGSTPFTIRWFKGDKELVSGGN |
| | | | CYINKEALESSLELYSVKTTDSGTYT |
| | | | CKVSNVAGAVECSANLFVKEPATFV |
| | | | ERLELSQLLKKGDTTQLACKVTGTPP |
| | | | IKITWFANDREIKESSKHKMSFVEST |
| | | | AVLRLTDIAVEDSGEYMCEAQNEAG |
| | | | SDHCSSILIVKESPYFTKEFKPIEVLKE |
| | | | YDVMLLAEVAGTPPFEITWFKDNTT |
| | | | LRSGRKYKTFIQDRLVSLQILKFVAA |
| | | | DVGKYQCRVTNEVGSSTCSARVTLR |
| | | | EPPTFVKKIESTSSLRGGTAAFQATL |
| | | | KGSLPITVTWLKDNEEITEDDNIRMT |
| | | | FENNVASLYLSGIEVKHDGKYVCQA |
| | | | KNDAGIQRCFAVLSVKEPATIIEEAV |
| | | | SIDVTQGDPATLQVKFSGTKEITAKW |
| | | | FKDGQELTLGQKYKISVTDTVSILKII |
| | | | STEKRDSGEYTFEVQNDVGRSSCKA |
| | | | SINVLDLIIPPSFTKKLKKMDSIKGSSI |
| | | | DLECIVAGSHPISIQWFKDDQEITASE |
| | | | KYKFSFHDNTAFLEISQLEGSDSGTY |
| | | | TCSATNKAGHNQCSGHLTVKEPPYF |
| | | | LEKPQSQDVNPNTRVQLKALVGGTA |
| | | | PMTIKWFKDNKELHSGAARSVWKD |
| | | | DTSTILELFSAKAADSGTYICQLSND |
| | | | VGTATTKASLFVKEPPQFIKKPSPVL |
| | | | VLRNGQSTTFECQITGTPEIRVSWYL |
| | | | DGNEITAVEKHGISFIDGLATFQISGA |
| | | | RVENSGTYVCEAQNDAGTASCSIEL |
| | | | KVKEPPTFIRELKPVEVVKDSDVELE |
| | | | CEVMGTSPFQVTWLRNNKEIRSSKK |
| | | | YTLTDRVSVFNLNINRCDPSDTGDY |
| | | | QCIVSNEGGSCSCSARVSLKEPPSFIK |
| | | | KIENITTVLKSSATFQSTVAGSPPISIT |
| | | | WLKDDQILDEDDNVHISFVNNVATL |
| | | | QIRSVDNGHSGRYTCQAKNESGVER |
| | | | CYAFLLVQEPAQIIEKAKSVDVTERD |
| | | | PVTLECVVAGTPELKVKWLKDGKQI |
| | | | VPSRYFSMSFENNVASFRIQSVMKQ |
| | | | DSGEYTFKVENDFGSSSCDAYLRVL |
| | | | DQNIPPSFTKKLTKMDKVLGSSIHME |
| | | | CKVSGSLPISAQWFKDGKEISTSAKY |
| | | | RLVCHENTVSLDVNNLELEDTANYT |
| | | | CRVSNVAGDAACSGILTVKEPPSFLV |
| | | | KPERQQAIPDSTVEFKAVLKGTPPFKI |
| | | | KWFKDDVELASGPKCFIGLEGSTSFL |
| | | | NLYSVDASKTGQYTCQVTNDVGSDS |
| | | | CTTTLLVTEPPKFVKKLEATKIVKAG |
| | | | DSARLECKITGSPDIRVVWYRNEHEL |
| | | | PASDKYRMAFIDSVAVLQMNYLGTE |
| | | | DTGDFICEAQNPAGSTSCSTKVIVKE |
| | | | PPVFSSFPPVVETLKNAEVSIECELSG |
| | | | TPPFEVVWYKDKRQLRSSKKYKIAS |
| | | | KNFHASIHILNVDTLDIGEYHCKAQN |
| | | | EVGSDTCICIVKLKEPPRFVSKLNSLT |
| | | | VVAGEPAELQASIEGTQPISVQWLKE |
| | | | KEEVVRESENIRITFVENVATLQFSK |
| | | | AEPANAGKYICQIKNDGGMRENMA |
| | | | TLSFISEPAVIVEKAGSMTVTVGETC |
| | | | TLECKVAGTPELSVEWYKDGKLLTS |
| | | | SQKHKFSFYNKISSLKILSVEKQDAG |
| | | | TYTFQVQNNVGKSSCTAVVDVSDR |
| | | | MVPPSFTRRLKDTIGVLGTSCILECK |
| | | | VAGSSPISVAWFHGKTKIVSGAKYQ |
| | | | TTFSDNVCTLQLNSLDSSDMGSYTC |
| | | | VAANVAGSDECRAVLAVQEPPSFVK |
| | | | EPEPLEVLPGKNITFTSVIRGTPPFKV |
| | | | GWFRGARELVKGDRCNIYFEDTVAE |
| | | | LELFNVDISQSGEYTCVVSNNAGQTS |
| | | | CTTRLFVKEPAVFVKKLSDHSVEPG |
| | | | KSIILESTYKGTLPISVTWKKDGFNIT |
| | | | PSEKCSIVTTEKTCILEILNSTKRDAG |
| | | | RYSCEIENEAGRDVCEALVSTLEPPY |
| | | | FVTELEPLEASVGDSVSLQCQVAGSP |
| | | | EITVSWYKGDTKLRPTPEYRTYFTNN |
| | | | VATLVFNKVNINDSGEYTCKAENSIG |
| | | | TASSKTVFRIQERQLPPSFARQLKDIE |
| | | | QTVGLPVTLTCRLNGSAPIQVSWYR |
| | | | DGVLLRDDENLQTSFVDNVATLKIL |
| | | | QTDLSHSGQYSCSASNPLGTASSSAR |
| | | | LTAREPKKSPFFDIKPVSIDIIAGESAD |
| | | | FECHVTGAQPMRITWSKDNKEIRPG |
| | | | GNYTITCVGNTPHLRILKVGKGDSG |
| | | | QYTCQATNDVGKDMCSAQLSVKEP |
| | | | PKFVKKLEASKVAKQGESIHLECKIS |
| | | | GSPEIKVSWFRNDSELHESWKYNMS |
| | | | FVDSVAVLTINEASAEDSGDYICEAH |
| | | | NGVGDASCSTALTIKAPPVFTQKPSPI |
| | | | GALKGSDVVFQCEISGTPPFEVVWV |
| | | | KDRKQVRSSKKFKITSKNFDASLHIL |
| | | | NLEAADVGEYYCKATNEVGSDTCV |
| | | | CTLKFKVEPPRFVKKLSDTSTLVGDA |
| | | | VELRAVVEGFQPISVVWLKDKGDVI |
| | | | RESENTRISFVDNVATLQLGSPEASD |
| | | | SGKYVCQIKNDAGMRECSAILTVLEP |
| | | | ARIIEKPEPMTVTTGNAFALECVVTG |
| | | | TPELSAKWFKDGREIAADSKHHITFV |
| | | | NKVASLKIPCAEMSDKGLYSFEVKN |
| | | | SVGKSTCTVSVHVSDRIVAPSFIRKL |
| | | | KDINAIVGASVVLECRVSGSAPISVG |
| | | | WFQDGNEIVSGPKCQSSFSENVCTLN |
| | | | LSFLEPSDTGTYTCVAANVAGSDECS |
| | | | AVLTIQEPPSFEQTPDSVEVLPGTSLT |
| | | | FTSVIRGTPPFKVKWFKGSRELVSGE |
| | | | SCSISLEDFVTELELFEVEPLQSGDYS |
| | | | CLVTNDAGSASCTAHLFVKEPATFV |
| | | | KRLADFSVETGSPIVLEATFTGTPPIS |
| | | | VSWMKNEFALTQSQNCSITMTEKSTI |
| | | | LEILDSTIEDYAQYSCLIENEAGQDIC |
| | | | DAVVSVLEPPYFIEPLEHVEAVIGEPI |
| | | | TLQCKVDGTPEIRISWYKEHTKLRSA |
| | | | PAYKMQFKNNVASLVINKVDHSDV |
| | | | GEYTCKAENIVGAVASSSVLVIKERK |
| | | | LPPSFARKLKDVQETLGFPVAFECRI |
| | | | NGSEPLQVSWYKDGVLLKDDANLQ |
| | | | TSFVHNVATLQILQTDQSHVGQYNC |
| | | | SASNPLGTASSSAKLVLLEHEVPPFF |
| | | | DLKPVSVDLALGESGSFKCHVTGTA |
| | | | PMKITWAKDNREIRPGGNYKMTLVE |
| | | | NTATLTVLKIGKGDAGQYTCYASNV |
| | | | AGKDSCSAHLGVQASIEPPRFIKKLE |
| | | | PSRIVKQDESTRYECKIGGSPEIKVL |
| | | | WYKGETEIQESSKFRMSFVDSVAVL |
| | | | EMHGLSVEDSGDYTCEACNAAGSAS |
| | | | STTSLKVKALEPPIFRKKPHPVETLK |
| | | | GADVHLECELQGTPPFQVSWHKDKR |
| | | | ELRSGKKYKIMSENFLTSIHILSVDAA |
| | | | DVGEYQCKATNDVGSDTCVGSITLK |
| | | | APPRFVKKPSDVSTIVGEEVQLQATV |
| | | | EGAEPISVVWFKDKGEIVRESDNIWI |
| | | | SYSENTATLQFSRAETANAGKYTCQI |
| | | | KNDAGMQECSATVSILEPAAIVEKPE |
| | | | SITVTTGDTCTLECSVTGTPELTTKW |
| | | | FKDGKELTSDNKYKISFFNKVSGLKII |
| | | | NVAPSDSGVYSFEVQNPVGKDSCTA |
| | | | SVQVSDSDRIVPPSFTRRLKETNGLS |
| | | | GSSVVMECKVYGSPPISVSWFHERN |
| | | | EISSGRKYQTTLTDNTCALTVNMLEE |
| | | | SDAGNYTCIATNVAGSDECSAPLTV |
| | | | REPPSFVQKPDPMDVLTGTNVTFTSI |
| | | | VKGTPPFSVSWFKGSTELVPGDTCN |
| | | | VSLEDSVAELELFDVDTSQSGEYTCI |
| | | | VTNEAGKVSCTTHLYVKAPARFVKK |
| | | | LNDYSIEKGKPLILEGTYTGTPPISVT |
| | | | WKKNGINVTPSQRCNITTTERSAILEI |
| | | | PSSTVEDAGQYNCYIENTSGKDSCSA |
| | | | QILILEPPYFVRQLEPVKVTVGDSASL |
| | | | QCQLGGTPEIAVSWFKGDTKLRPTA |
| | | | TYKMHFRNNIATLVFNQVDSNDSGE |
| | | | YICRAENSVGEVSSSTFLTVQEQKLP |
| | | | PSFSRQLRDVQETVGLPVVFECAVSG |
| | | | SEPISVSWFKDGRPVKDSPNIQASFL |
| | | | DNVATLNIFQTDRSFAGQYSCTATNP |
| | | | IGSASSSARLILTEGKNPPFFDIPLAPV |
| | | | DAVVGESADFECHVTGTQPIKVAWA |
| | | | KDNREIRSGGNYQISYLENSAHLTIL |
| | | | KVDKGDSGQYTCYAVNEVGKDSCT |
| | | | AQLNIKERLIPPSFTKKLSETVEETEG |
| | | | NSFKLEGRVAGSQPISVAWYKNNIEI |
| | | | HPTSNCEITFKNNTLLLQIKRAGMDD |
| | | | AGLYTCKVSNDAGSALCTSSIVIKEP |
| | | | KKPPVFDQHLTPVTASEGEFVQLSCH |
| | | | VWGSEPIRIQWLKAGREIKPSDRCSF |
| | | | SFANGTAVLELKDVTKADAGDYVC |
| | | | KASNVAGSDTSKSKVTIKDKPAAVP |
| | | | AAKKAAVDGKLFFVSEPQSIRVVEK |
| | | | TTATFIAKVGGDPIPNVKWTKGKWR |
| | | | QLNQGGRILIHQKGDEAKLEIRDTTK |
| | | | TDSGLYRCVAFNKHGEIESNVNLQV |
| | | | DERKKQEKIEGDLRAMLKKTPVLKK |
| | | | GAGEEEEIDIMELLKNVDPKEYEKY |
| | | | ARMYGITDFRGLLQAFELLKQTEGE |
| | | | ETHRLEIEEIEKSEKDEKEFEELVSFIQ |
| | | | QRLSQTEPVTLIKDIENQTVLKDNDA |
| | | | VFEIDIKINYPEIKLSWYKGTEKLEPS |
| | | | DKFEISIDGDRHTLRVKNCQLKDQG |
| | | | NYRLVCGPHIASAKLTVIATEPAWER |
| | | | HLQDVTLKEGQTCTMTCQFSVPNVR |
| | | | SEWFRNGRILKPQGRYKTEVEHKVH |
| | | | KLTIADVRAEDQGRYTCKHEDLETS |
| | | | AELRIEAEPIQFTKRIQNIVVSEHQSA |
| | | | TFECEVSFDDAIVTWYKGPTELTESQ |
| | | | KYNFRNDGRCHYMTIHNVTPDDEG |
| | | | VYSVIARLEPRGEARSTAELYLTTKEI |
| | | | KLEMKPPDIPDSRVPIPTMPIRAIPPEE |
| | | | IPPTAVPPIPLLLPLPEEKKPPEKRVEV |
| | | | TKKAVKKDAKKVVAKPKEEAPPPK |
| | | | VIEVPKKPPRPTALIPAEAPEIIDVSSK |
| | | | AEEVKITTITRKKEVQKEKEAVYERK |
| | | | QAVYEEKKVYIESLEEPYDELEVETY |
| | | | TEPYEEPYYEEPEEDYEETQVEAKRE |
| | | | VHEEWEEDFEEGQEYYEREEGYDEG |
| | | | EEEWEETYQEREVVQVQKEVYEESR |
| | | | ERKIPAKVPEKKELPPPKVVKKPVVE |
| | | | KIEKTSRRMEEEKVQVTKVPEVSKKI |
| | | | VPQKPSRTPVQEEIIEVKVPAVHTKK |
| | | | MVISEEKMFFAAHTEEEEEVSVTVPE |
| | | | VQKKTVTEEKVHVAVSKKIEPPPKV |
| | | | PEPPKKPEEVVPVPVPKKVEPPPAKV |
| | | | PEVPKKPVPEEKKPVPVPKKEPAAPP |
| | | | KVPEVPKKPVPEEKVPVPIAKKKEAP |
| | | | PAKVPEVQKRVVTEEKITIVTEREESP |
| | | | PPAVPEIPKKKLPEEKRPVPRKEEEVP |
| | | | PPKVPAVPKKPVPEEKVPVPVPVAK |
| | | | KAPPPRAEVSKKMVMEEKRFAAEEK |
| | | | LSVTVPQRVEVMRHEVSAEKEWRYS |
| | | | EEEERVSVSVYREEEREEEEVEVTEY |
| | | | EVMEEPEEYIVEEEEHFISEEVEAEPA |
| | | | EESFQVPEKKIIPKPKIPAKVEEPPPA |
| | | | KVPEAPKKIVPEKKIPAAVPKKEKVP |
| | | | PTKVPEEPKKPVPEKRAPPKVAKIEE |
| | | | PPPTKVTERHMQITQEEKVHVAVTK |
| | | | KVEPPRPKVPEEPKRAVPEEKFPKLK |
| | | | PRKEEEPPAKVTELRKRAVKEEKVSI |
| | | | EVAKREPPAAKEVTVTAEKEWAYT |
| | | | KEEEAVSVEREEEYEEYEEYDYKEFE |
| | | | EYEPTEEYDQYEEYEEREFEHYEEYK |
| | | | EHEEYVTEPEKPVPVKPIQEEPVPTKP |
| | | | KAPPAKVPKKVIPEEKVPVPIPKKLK |
| | | | PPPPKAREEAKKVVEEKIQISVIKREK |
| | | | EQVTEPAAKVPMKPKRVVTEEKVPV |
| | | | PKKEVAAPVRVPEVPKKEEPEEIAFE |
| | | | EEVVTHEEEYYEEEEEEEEYIHEEEEI |
| | | | ITEEEVVPVKVFKVPEVPKKPVPEEK |
| | | | KPVPVPKKKEAPPAKVPEIPKKPEEK |
| | | | VPVPVPKKEKAPPAKVPEVPKKPVPE |
| | | | EKVPVPEEKVPVPVPKKVEAPPAKV |
| | | | PEVPKKPVPEKKVPVPAPKKVEAPPA |
| | | | KVPEVPKKVLPEEKKPTPIRKKVEPP |
| | | | PPKVPKKREPVPVPIPAALLREEKVV |
| | | | HKEEIVVEEEEVLPEEEEISPEEEVPL |
| | | | EEEEEEEEVLPEEEEVPPEEEEVPPEE |
| | | | EEVPPEEEEFVPEEEVVPEKEEVLPEI |
| | | | KPKVPVPARVPEVKKKVPEKKVIIPK |
| | | | KEEVPPAKVPEVPKKVEEKRIIVPEEE |
| | | | EVPPVEVFEEPEEPIPEEEIPEEAPIVE |
| | | | EVEEEAPPRVPEVVKKAVPEAPTPVP |
| | | | KKVEVPPAKVPKKVPEEKPPVPVQK |
| | | | KEAPPAKVPEVPKKVPEKKVPVPKK |
| | | | EVVPPAKGRAVLEEKVSVAFHKEEV |
| | | | VEERTELEIVEAQEEALEEEFHEVEE |
| | | | YFEEEEFHEVEEFLKVEKYRAEEEVH |
| | | | RAEEVHRVIEVLEAEEEEAYEKPKAP |
| | | | PKGPEISEKVIPPKKPPTKVVPRKEPP |
| | | | AKVPEVPKKIVVEEKVHVPEEPKVA |
| | | | PAKVPEAPKEVVPEKKVPAAPPKKP |
| | | | EVPPVTVPEAPKEVVPEKKVPVVPPK |
| | | | KPEVPPAKVPEVPKAAVPEKKVPEAI |
| | | | PPKPESPPPAVPEAPKEVVPEKKVPV |
| | | | MPPKKPEVPPAKVPEAPKEVVPEKK |
| | | | VPVMPPKKPEVPPAKVPEVPKAAVP |
| | | | EKKVPEAIPPKPESPPPAVYEEPEEIA |
| | | | PEEPPVEVVEEPEPVPAPPPKVTVPPK |
| | | | KPVPEKKPPAVVAKKPEPPPAKVPEV |
| | | | PKEVVPEKKVPPVVPKKPEVPPPKVP |
| | | | EVPKEVVPEKKVAVPKKPEVPPAKV |
| | | | PEVPKKPVLEEKPAIPIPEKVESPPPEE |
| | | | EEEPVPVVEEEEPVPVVEEEEPVPVV |
| | | | EEVEPEAPPPAVPEEPKKIVPEKKVP |
| | | | VIKKPEPPPPKEPEPEKKVIEKPKLKP |
| | | | RPPAPPPAPPKEDVKEKIFQLKAVSK |
| | | | KKVPEKPAVPEKVELTPLKVAGGEK |
| | | | KVRKLLPEPKPQPKEEVILKSVLRKR |
| | | | PEEEEPKVEPKKVEKIKKPAVPEPPP |
| | | | KAVEEVEAPPAVTKKERKIPEPTKVP |
| | | | EIKPPIPLPAPEPKPKPEAEVKIIKPPP |
| | | | VEPPPTPIAAPVTVPVVGKKAEAKAP |
| | | | KEEAAKPKGPIKGVAKKTPSPIEAER |
| | | | KKLRPGSGGEKPPDEAPFTYQLKAV |
| | | | PLKFVKEIKDIVLTEAESVGSSAIFEC |
| | | | LVSPSTAVTSWMKDGSNIRESPKHRF |
| | | | IADGKDRKLHIIDVQLSDAGEYTCVL |
| | | | RLGNKEKTSTAKLIVEELPVRFVKTL |
| | | | EEEVTVVKGQPLYLSCELNKERDVV |
| | | | WRKDGKIVVEKPGKIVPGVIGLLRAL |
| | | | TINDADDSDAGTYTVTVENANNLEC |
| | | | SSCVKVVEVIRDWLVKPIRDQHVKP |
| | | | KGTAVFTCVIAKDTPNIKWYKGYDE |
| | | | IPWEPTDKTEIIRDGNHIHLKVKNAM |
| | | | PEDIDEYAVEIEGKRYPAKLTLGERE |
| | | | VELLKPIEDVTIYEKESASFDAEISEV |
| | | | DVPGQWKLKGELLRPSPTCEIKAED |
| | | | GKRFLTLHNVKLDQAGEVLYQALN |
| | | | AVTTAILTVKEIELDFAVPLKDVTVP |
| | | | EKRQARFECVLTREANVIWSKGPDII |
| | | | KSSDKFDIITDGKKHILVINNSQFDDE |
| | | | GVYTAEVEGKKTSARLFVTGIRLKFI |
| | | | SPLEDQTVKEGETATFVCELSHEKM |
| | | | HVVWFKNDAKLHTSRTVLISSEGKT |
| | | | HKLEMREVTMDDISQIKAQVKDLSS |
| | | | TANLKVIEADPYFTVKLHDKTGVEK |
| | | | DEIVLRCEVSKDVPVKWFKDGEELL |
| | | | PSPKHSIKADGLRRILKIKKADLKDK |
| | | | GEYVCDCGTDTTKANVTVEARLIKV |
| | | | EKPLYGVEVFVGETARFEIELSEPDV |
| | | | HGQWKLKGEPLTASPDCEIIEDGKK |
| | | | HILVLYNCRLDMTGKVSFQAANAKS |
| | | | AANLKVKELPLIFITPLSDVKVFEKD |
| | | | EAKFECEVSREPKTYRWLKGTQEITG |
| | | | DDRIELIKDGTKHSLVIKSAAFEDEA |
| | | | KYIFEAEDQHTSGKLIIEGIRLKFLTPL |
| | | | KDVTAKERESAVFTVELSHDNIRVR |
| | | | WFKNDQRLHTSKFVSMDDEGKTHSI |
| | | | TFRNLSIDDTSQIKVEAMGLSSEAKL |
| | | | TVLEGDPYFTGKLQDYTGVEKEEVIL |
| | | | QCEISKADAPVKWFKDGQEIKPSKN |
| | | | AVIKADGKKRMLILKKPLKSDIGQYT |
| | | | CDCGTDKTSGKLNIEDREIKLVRPLY |
| | | | SVEVMETETARFETEISEDDIHANWK |
| | | | LKGEALLQTPECEIKEEGKTHFLILH |
| | | | NCRLDQTGGVDFQAANVKSSAHLR |
| | | | VKPRVIGLLRPLKDITVTAGETATFD |
| | | | CELSYEDIPVEWYLRGKKLEPSDKV |
| | | | VMRSEGRVHTLTLRDVKLEDAGEV |
| | | | QLVAKDFKTQAKLFVKEPPVEFTKP |
| | | | LEDQTVEEEATAVLECEVSRENAKV |
| | | | KWFKNGTEILKSKKYEIVADGRVRK |
| | | | LIIHGCTPEDIKTYTCDAKDFKTSCNL |
| | | | NVVPPHVEFLRPLTDLRVKEKEMAR |
| | | | FECEISRENAKVHWFKDGAEIKKGK |
| | | | KYDIISKGAVRILVINKCLLDDEAEYS |
| | | | CEVRTARTSGMLTVLEEEAVFTKNL |
| | | | ANIEVSETDTVKLVCEVSKPGAEVT |
| | | | WYKGDEEIIETGRYEILTDGRKRILII |
| | | | QNAHLEDAGNYNCRLPSSRTDGKVK |
| | | | VHELAAEFISKPQNLEILEGEKAEFV |
| | | | CSISKENFEVQWKRDDKTLESGDKY |
| | | | DVIADGKKRVLVVKDATLQDMGTY |
| | | | VVMVGAARAAAHLTVIEKLRIIVPLK |
| | | | DTRVKEQQEVVFNCEVNTEGAKAK |
| | | | WFRNDEAIFDSSKYIILQKDLVYTLRI |
| | | | RNAQLDDQANYNVSLTNHRGENVK |
| | | | SAANLIVEEEDLRIIEPLKDIETMEKK |
| | | | SVTFWCKVNRLNVTLKWTKNGEEV |
| | | | AFDNRVLYRIDKYKHSLIIKDCGFPD |
| | | | EGEYVVTAGQDKSVAELLIIEAPTEF |
| | | | VEHLEDQTVTEFDDAVFSCQLSREK |
| | | | ANVKWYRNGREIKEGKKYKFEKDG |
| | | | SIHRLIIKDCRLDDECEYACGVDDRK |
| | | | SRARLFVEEIPVEIIRPPQDILEAPGAD |
| | | | VVFLAELNKDKVEVQWLKNNMIIVQ |
| | | | GDKHQMMSEGKIHRLQICDIKPRDQ |
| | | | GEYRFIAKDKEARAKLELAAAPKIKT |
| | | | ADQDLVVDVGQPLTMVVPYDAFPK |
| | | | AEAEWFKENEPLSSKTVDTTAEQTSF |
| | | | RILKAKKEDKGRYKVVLQNKHGKA |
| | | | EGFINLKVIDVPGPVRNLEVTETFDG |
| | | | EVSLAWEEPLTDGGSKIIGYVVERRD |
| | | | IKRKTWVLATDRADSCEFTVTGLQK |
| | | | GGVEYLFRVSARNRVGTGEPVETDS |
| | | | PVEARSKYDVPGPPLNVTVTDVNRF |
| | | | GVSLTWEPPEYDGGAEITNYVIELRD |
| | | | KTSIRWETAMTVRAEDLSATVTDVV |
| | | | EGQEYSFRVRAQNRIGVGKPSAATPF |
| | | | IKVADPIERPSPPVNLNASDQTQSSV |
| | | | QLTWEPPLKDGGSPILGYIIERCEEGK |
| | | | DNWIRCNKKLVPELTYKVTGLQKGN |
| | | | KYLYRVSAENEAGVSDPSEILGPLTA |
| | | | DDADAEPTMDLSAFKDGLEVIVPNPI |
| | | | KILVPSTGYPRPTATWSFGDKVLEAG |
| | | | DRVKMKTLSAYAELVISPSERPDKGI |
| | | | YTLKLENRAKAISGEIDVNVIARPSA |
| | | | PRELKFGDITKDSVHLTWEPPEDDGG |
| | | | SPLTGYVIEKREVSRKTWTKVIDSVT |
| | | | DLEFTVPDLLQGKEYLFKVCACNKC |
| | | | GPGEPAYVDEPVNMSAPATVPDPPE |
| | | | NVKWRDRTAKSIFLTWDPPKHDGGS |
| | | | RIKGYIVEKCPRGSDRWVACGEPVA |
| | | | DTKMEVTGLEEGQWYAYRVKALNR |
| | | | LGASKPSKPTEEIQAIDTQEAPEIFLD |
| | | | VKLLAGITVKAGTKIELPATVTGKPE |
| | | | PKITWTKADMLLKQDKRITIENVPKK |
| | | | STVTIMDSKRSDTGRYIIEAVNVCGR |
| | | | ATAVVEVNVLDKPGPPAAFDITDVT |
| | | | NESCLLTWNPPRDDGGSKITNYVVE |
| | | | RRATDSDMWHKLSSTVKDTKFKAT |
| | | | KLTPNKEYIFRVAAENMYGVGEPVQ |
| | | | ATPITAKFQFDPPGPPTRLEPSDITKD |
| | | | AVTLTWCEPDDDGGSPITGYWVERL |
| | | | DPESDKWVRCNKMPIKDTTYRVKGL |
| | | | TNKKKYRFRVLAENLAGPGKPSKST |
| | | | EPILIKDAIDPPWPPGKPVVKDIGRTS |
| | | | LMLNWTKPEHDGGAKIDSYVIEMLK |
| | | | TGTEDWVRVAEGVPTTQHLLPGLME |
| | | | GQEYSFRVRAVNKAGESEPSEPSDPV |
| | | | LCREKLYPPSPPRWLEVINITKNTAD |
| | | | LKWTVPEKDGGSPITNYIVEKRDVR |
| | | | RKGWQTVDTTVKDTKCTVTPLTEGS |
| | | | LYVFRVAAENAIGQSDYCEVEDSVL |
| | | | AKDTFTTPGPPYALAVVDVTKRHVD |
| | | | LKWEPPKNDGGRPIQRYVIEKKEKL |
| | | | GTRWVKAGKTSGPDCHFRVTDVIEG |
| | | | TEVQFQVRAENEAGVGHPSEPTEILK |
| | | | IEDPTSPPSPPLDLHVTDAGRKHIAIA |
| | | | WKPPEKNGGSPIIGYHVEMCPVGTE |
| | | | KWMRVNSRPIKDLKFKVEEGVVPDK |
| | | | EYVLRVRAVNAVGVSEPSEISENVV |
| | | | AKDPDCRPTIDLETHDIVVIEGEKLSI |
| | | | PVPFRAVPVPTVSWHKDGKEVKASD |
| | | | RLTMKNDHISAHLEVPKSVRADAGI |
| | | | YTVTLENKLGSATASINVKVIGLPGP |
| | | | CRDLKASDVTKSSCKLTWEPPEYDG |
| | | | GSPILHYVLERREAGRRTYIPVMSGE |
| | | | NKLSWTVKDLIPNGEYFFRVKAVNK |
| | | | IGGGEYIELKNPVIAQDPKQPPDPPV |
| | | | DVEVHNPTAEAMTITWKPPLYDGGS |
| | | | KIMGYIIEKIAKGEERWKRCNEHLVP |
| | | | VLTYTAKGLEEGKEYQFRVRAENAA |
| | | | GISEPSRATPPTKAVDPIDAPKVIMKT |
| | | | SLEVRRGDEIALDAIISGSPYPTITWL |
| | | | KDESIITPEEIKKRVEPLVRRRRGEVQ |
| | | | EEQPFVLPLTQRLSIDNSQKGESQLR |
| | | | VRDSVRPDHGLFMIKAENDHGIAKA |
| | | | PCTVCVLDIPGPPINFVFEDMRKTSV |
| | | | LCKWEPPLDDGGSEILNYTLEKKDK |
| | | | TKPDSEWMVVTSTLRHCKYSVTKLI |
| | | | EGKEYLFRVRAENRFGPGPPCVSKPF |
| | | | MAKDPFEPPDAPDKPIVEDVTSNSML |
| | | | VTWNEPKDNGSPILGYWLEKREVNS |
| | | | THWSRVNRNLLNSLKTKVDGLLEGL |
| | | | TYVFRVCAENAAGPGKFSPPSDPKT |
| | | | AQDPISPPGPPVPRVTDTSSTTIELAW |
| | | | EPPAFNGGGEIVGYYVYKQLVGTNE |
| | | | WSRCTEKMIKPREYTVREIREGADY |
| | | | KLRVTAVNVAGEGPPGETEPVTVAE |
| | | | PQAEPPTVELDVSVKGGIQIMAGKTL |
| | | | RIPAVVTGRPVPTKVWTIEEGELDKD |
| | | | RVEIENVGTKSELIIKNALRKDHGRY |
| | | | VITATNSCGSKFAAARVEVFDVPGPV |
| | | | LDLKPVVTNRKMCLLNWSDPEDDG |
| | | | GSEITGFIIERKDAKMHTWRQPIETER |
| | | | SKCDITGLLEGQEYMFRVIAKNKFGC |
| | | | GPPVEIGPILAVDPLGPPTSPERLTYT |
| | | | ERTKSTITLDWKEPRSNGGCPIQGYII |
| | | | EKRRHDKPDFERVNKHLCPTTSFLVE |
| | | | DLDEHQMYEFRVKAVNEIGESEPSLP |
| | | | LNVVIQDDEVPPTIKLRLSVRGDTIK |
| | | | VKAGEPVNIPADVTGLPMPKIEWSK |
| | | | NETVIEKPTDALKITKEEVSRSEAKTE |
| | | | LSIPKATREDKGTYTVTASNRLGSVF |
| | | | RNVHVEVYDRPSPPRNLAVTDIKAES |
| | | | CYLTWDAPLDNGGSEITHYIIDKRDA |
| | | | SRKRAEWEEVTNSAVERRYGIWKLI |
| | | | PNGQYEFRVRAVNKYGISDECKSDK |
| | | | VVIQDPYRTPGPPGKPKVLERTKGS |
| | | | MLVSWTPPLDNGGSPITGYWLEKRE |
| | | | EGGAYWSRVSRAPITKVGLKGVEFN |
| | | | VPRLIEGVKYQFRAMAINAAGVGPP |
| | | | SEPSDPEVAGDPIYPPGAPSRPEVKD |
| | | | KTKSSITEAWKPPAKDGGSPIKGYIV |
| | | | EMQEEGTTDWKSVNEPDKLLPTCEC |
| | | | VVPNLKELKKYRFRVKAVNEAGESE |
| | | | PSDTTGEIPATDIQEVPEVFIDIGAQD |
| | | | CLICQAGTQIRIPAVIKGRPTPKSSWE |
| | | | FDGKAKKAMKDGVHDIPEDAQLET |
| | | | AENSSVIIIPECKRSHSGKYSITAKNK |
| | | | AGQKTANCRVKVMDVPGPPKDLKV |
| | | | SDITRGSCRLSWKMPDDDGGDRIKG |
| | | | YVIEKRTIDGKAWTKVNPNCVSTSF |
| | | | VVPDLIAGQEYFFRVRAENRFGVGA |
| | | | PAETIQRTTARDPIYPPDPPIKLKIGLV |
| | | | TKNTVHLSWKPPKNDGGSPVTHYIV |
| | | | ECLAWDPTGTKKEAWRQCNKRDVE |
| | | | ELEFTVEDLIEGGEYEFRVKAVNAA |
| | | | GVSKPSATVGPVIVKDQTCPPAIELK |
| | | | EFMEVEEGTDVNIVAKIKGVPFPTLT |
| | | | WFKAPPKKPDNKEPIVYDTHVNKLV |
| | | | VDDTCTLVIPQSRRSDTALYTITAVN |
| | | | NLGTASKEMRLNVLGRPGPPVGPIKF |
| | | | ESISADQMTLSWLPPIDDGGSKITNY |
| | | | VIEKREANRKTWVRVSSEPKECTYTI |
| | | | PKLLEGHEYVFRIMAQNKYGIGEPLD |
| | | | SEPETARNLFSVPGAPDKPTVSSVTR |
| | | | NSMTVNWEEPEYDGGSPVTGYWLE |
| | | | MKDTTTKRWKRVNRDPIKAMTLGV |
| | | | SYKVTGLIEGSDYQFRVYAINAAGV |
| | | | GPASLPSDPATARDPIAPPGPPFPKVT |
| | | | DWTKSSADLEWSPPLKDGGSRVTGY |
| | | | IVEYKEEGKEEWEKAKDKEVRGTKL |
| | | | VVTGLKEGAFYKFRVRAVNIAGIGEP |
| | | | GEVTDAIEMKDRLELPDLQLDASVR |
| | | | DRIVVHAGGVIRIIAYVSGKPPPTVT |
| | | | WNMNERALPQEATIETTAISSSMVIK |
| | | | NCQRSHQGVYSLLAKNAAGERKKTI |
| | | | IVDVLDVPGPVGIPFLAHNLTNDSCK |
| | | | LTWFSPEDDGGSPITNYVIEKREADH |
| | | | RAWTPVTYTVTRHNATVQGLIQGKA |
| | | | YFFRIAAENSIGMGPFVETTDALVIR |
| | | | DPITVPERPEDLEVKEVTKESVTLTW |
| | | | NPPKYDGGSDIINYVLESRLIGTEKFH |
| | | | KVTSDNLMSRKYTVKGLKEGDTYE |
| | | | YRVSAVNIVGQGKPSFCTKPITCKDE |
| | | | LAPPTLDLDFRDKLTIRVGEAFALTG |
| | | | RYSGKPKPKVSWFKDEADVLEDDRT |
| | | | HIKTTPTTLALEKLKAKRSDSGKYSV |
| | | | VVENSTGSRKGVCQVNVVDRPGPPV |
| | | | GPVIFDEVTKDYMVISWKPPLDDGG |
| | | | SEITNYIIEKKEVGKDVWMPVTSASA |
| | | | KTTCKVSKLLEGKDYIFRIHAENLYG |
| | | | ISDPLVSDSMKAKDRFRVPDAPDQPI |
| | | | VTEVTKDSALVSWNPPHDGGKPITN |
| | | | YILEKRETMSKIWARVTKEPIHPYTK |
| | | | FRVPDLLEGCHYEFRVSAENEIGIGD |
| | | | PSPPSKPVFAKDPIAKPSPPVNPEAID |
| | | | TTCNSVDLTWQPPRRDGGSKILGYIV |
| | | | EYQKVGDEEWKRANHTPESCPETNY |
| | | | KVTGLRDGQSYKFRVIAVNAAGESD |
| | | | PAHVPEPVLVKDRLEPPELILDANMA |
| | | | REQHIRVGDTLRLSAIIKGVPFPKVT |
| | | | WKKEDRAAPTKARIDVTPVGSKLEI |
| | | | RNAAHEDGGIYSLTVENPAGSKTVS |
| | | | VKVLVLDKPGPPRDLEVSEIRKDSCY |
| | | | LTWKEPLDDGGSVITNYVVERKDVA |
| | | | STEWSPLSTTSKKKSHLAKHLNEGN |
| | | | QYVFRVAAENQYGRGPFVETPKPIK |
| | | | AVDPLHPPGPPKNLHHVDVDKTEVS |
| | | | LVWNKPDRDGGSPITGYLVEYQEEG |
| | | | TPDWIKFKTVTNLACVVTGLQQGKT |
| | | | YRFRVKAENIVGLGLPDTTIPIECQEK |
| | | | LVPPSVELDVKLIEGLVVKAGTTVRF |
| | | | PAIIRGVPIPTAKWTTDGNEIKTDEHY |
| | | | TVETDNFSSVLTIKNCLRKDTGEYQI |
| | | | TVSNAAGTKTVAVHLTVLDVPGPPT |
| | | | GPINILEVTPEYMTISWLPPKDDGGSP |
| | | | VINYIVEKKDTKKDTWGVVSSGSSK |
| | | | TKLKVPHLQKGYEYVFRVKAENKIG |
| | | | IGPPLDSVPTVAKHKFSPPSPPGKPVV |
| | | | TDITENAATVAWTLPKSDGGSPITGY |
| | | | YLERREVTGKWVRVNKTPLVDLKFR |
| | | | VTGLYEGNTYEFRVFAENLAGLSGP |
| | | | SPSSDPIKACRPIKPPGPPINPKLKDKT |
| | | | RESADLVWTKPLSDGGSPILGYVVE |
| | | | CQKAGTTQWDRINKDELIRQCAFRV |
| | | | PGLIEGNEYRFRIKAANIVGEGEPREL |
| | | | AESVIAKDILHPPEVELDVTCRDVITV |
| | | | RVGQTIRILARVKGRPEPDITWSKEG |
| | | | KALVRDKRVNIIHELPRVELQIKEAV |
| | | | RADHGKYIISAKNSSGHAQGFAIVNV |
| | | | LDRPGPCQNLKVTNVTKENCTISWE |
| | | | NPLDNGGSEITNFIVEYRKPNQKGWS |
| | | | IVASDVTKRLIKANLLANNEYYFRVC |
| | | | AENKVGVGPTIETKTPILAINPIDRPG |
| | | | EPENLHIADKGKTFVYLKWRRPDYD |
| | | | GGSPNLSYHVERRLKGAADWERVH |
| | | | KGSIKETHYMVDKCVENQIYEFRVQ |
| | | | TKNEGGESDWVKTEEVVVKEDLQK |
| | | | PVLDLKLSGVLTVKAGDTIRLEAGV |
| | | | RGKPFPEVSWTKDKDATDLTRSPRV |
| | | | KIDTSGDSSKFSLTKAKRSDGGKYV |
| | | | VTATNTAGSFVAFATVNVLDKPGPIR |
| | | | NLKITDVCSDRCSLRWDPPEDDGGC |
| | | | EIQNYILEKCESKRMVWSTFSSTILTP |
| | | | GTTVTRLIEGNEYIFRVRAENKIGTGP |
| | | | PTETKPVIAKTKYDKPGRPDPPEVTK |
| | | | VSKEEMTVVWAPPEYDGGKSITGYY |
| | | | LEKKEKHSTRWVPVNKSAIPERRLK |
| | | | VQNLLPGHEYQFRVKAENEVGIGEP |
| | | | SLPSRPVVAKDPIEPPGPPTNLKVVD |
| | | | TTKSSITLGWGKPVYDGGAPIIGYVV |
| | | | EMRPKKADMSPDEGWKRCNAAAQL |
| | | | VRMEFTVTSLDENQEYEFRVCAQNQ |
| | | | VGIGRPAELKDAIKPKEILEPPEIDLD |
| | | | ASMRKLVTVRAGCPIRLFAIVRGRPA |
| | | | PKVTWRKVGIDNVVRKGQVDLVDT |
| | | | MAFLVIPNSTRDDSGKYSLTLVNPAG |
| | | | EKAVFVNVRVLDTPGPVSDLKVSDV |
| | | | TKTSCHISWAPPENDGGSQVTHYIVE |
| | | | KREAERKTWATVTPEVKKTSFHVTN |
| | | | LVPGTEYFFRVTAVNEYGPGVPTDV |
| | | | PKPVLATDPLSEPDPPRKLEVTEMTK |
| | | | NSAVLAWLPPLRDGGAKIDGYIVSY |
| | | | REEEQPADRWTEYSVVKDLSLVVTG |
| | | | LKEGKKYKFRVAARNAVGVSLPREA |
| | | | EGVYEAKEQLLPPKILMPEQITIKAG |
| | | | KKLRIEAHVYGKPNPVCKWKKGED |
| | | | DVVTSSHLAVHKAENSSVLIIKDVTR |
| | | | KDSGYYSLTAENSSGTDTQKIKVIVM |
| | | | DAPGPPQPPFDISDIDADACSLSWHIP |
| | | | LEDGGSNITNYIVEKCDVSRGDWVT |
| | | | ALASVTKTSCRVGKLIPGQEYIFRVR |
| | | | AENRFGISEPLTSPKMVAKFPFGVPS |
| | | | EPKNARVTKVNKDCIFVAWDRPDSD |
| | | | GGSPITGYLIERKERNSLLWVKANDT |
| | | | PVRSTEYPCAGLVEGLEYSFRIYALN |
| | | | KAGSSPPSKPTEYVTARTPVDPPGKP |
| | | | EVIDVSKSTVSLVWARPKHDGGSKII |
| | | | GYFVEACKLPGDQWIRCNTSPHQIPQ |
| | | | EEFTVTGLEENAQYQFRAIAKTAVNI |
| | | | SQPSEPSDPVTIMAESVPPRIELSVAM |
| | | | KSLLTVKAGTNVCLDATVFGKPKPT |
| | | | VSWKKDGTLLKPSEGIKMAMKRNL |
| | | | CTLELFSVTRKDSGDYTITAENPSGS |
| | | | KSATIKLKVLDKPGPPASVKINKMYS |
| | | | DRAMLSWEPPLEDGGSEITNYIVDKR |
| | | | ETSRPNWAQVSATVPITSCSVEKLIE |
| | | | GHEYQFRICAENKYGVGDPILTEPAI |
| | | | AKNPYDPPGRCDPPVISNVTKDHMT |
| | | | VSWKPPADDGGSPITGYLVEKRETH |
| | | | AVNWTKVNRKPVIERTIKATGLQEG |
| | | | TEYEFRVIAINKAGPGKPSDASKAVY |
| | | | AQDPLYPPGPPAFPKVYDTTRSSVSL |
| | | | TWGKPAYDGGSPIIGYLVEVKRADS |
| | | | DNWVRCNLPQKLQKTRFEVTGLME |
| | | | NTEYQFRVYAVNKIGYSDPSDVPDK |
| | | | HCPKDILIPPEGELDADLRKTLILRAG |
| | | | VTMRLYVPVKGRPPPKITWSKPNVN |
| | | | LRERVGLDIKSTDFDTFLRCEKVNKY |
| | | | DAGKYILTLENSCGKKEYTIVVKVL |
| | | | DTPGPPVNVTVKEISKDSAYITWDPPI |
| | | | IDGGSPIINYVVEKRDAERKSWSTVT |
| | | | TECSKTSFRVSNLEEGKSYFFRVFAE |
| | | | NEYGIGDPGETRDAVKASETPGPVV |
| | | | DLKVTSVTKSSCSIGWKKPRSDGGSR |
| | | | IIGYVVDFLTEENKWQRVMKSLSLQ |
| | | | YTTKDLTEGKEYTFRVSAENENGEG |
| | | | TPSEITAVAKDDVVAPDLDLKDLPDL |
| | | | CYLAKENSNFRLKIPIKGKPVPSVSW |
| | | | KKGEDPLATDTRVSVESSAVNTTLV |
| | | | VYDCRKSDAGKYTITLKNVAGTKEG |
| | | | TLTIKVVGKPGIPTGPIKFEEVTAEAV |
| | | | TLKWGPPKDDGGSEITNYILEKRDSV |
| | | | NNKWVTCASAVQKTTFRVMRLHEG |
| | | | MEYTFRVSAENKYGVGEGLKSEPIV |
| | | | ARHPFDVPDAPPPPNIVDVRHDSVSL |
| | | | TWTDPRKTGGSPITGYHIEFKERNSL |
| | | | LWKRANKTPIRMKDFKVTGLTEGLE |
| | | | YEFRVMAINLAGVGKPSLPSEPVVAL |
| | | | DPIDPPGKPEVISVTRNSVTLVWTEP |
| | | | KYDGGHKLTGYIVEKRELPSKSWTK |
| | | | ANHVNVPDCAFTVTDLVEGGKYEFR |
| | | | VRAKNTAGAISAPSESTDTIICKDEYE |
| | | | APTIVLDPTIKDGLTVKAGDTIILSAIS |
| | | | ILGKPLPKSSWSKAGKDIRPSDIVQIT |
| | | | STPTSSMIAVKYATRKDAGEYTITAT |
| | | | NPFGTKSEHVKVTVLDVPGPPGPIEIS |
| | | | NVSAEKATLTWTPPLEDGGSPIKSYV |
| | | | LEKRETSRLLWTVVAEDIQSCRHVA |
| | | | TKLIQGNEYVFRVSAVNQYGKGEPV |
| | | | QSEPVKMVDRFGPPGPPGKPEVSNV |
| | | | TKNTATVSWKRPVDDGGSEITGYHV |
| | | | ERREKKSLRWVRATKTPVSDLRCKV |
| | | | TGLQEGNTYEFRVSAENKAGIGPPSD |
| | | | ASNPVLMKDVAYPPGPPSNARVTDT |
| | | | TKKSASLAWGKPHYDGGLEITGYVV |
| | | | EHQKVGDEGWIKDTTGTALRITEFV |
| | | | VPDLETKEKYNFRISAINDAGVGEPA |
| | | | VIPNVEIVEREMAPDFELDAELRRTL |
| | | | VVRAGLSIRIFVPIKGRPAPEVTWTK |
| | | | DNINLKNRANIENTESFTLLIIPECNR |
| | | | YDTGKFVMTIENPAGKKSGFVNVRV |
| | | | LDTPGPVLNLRPTDITKESVTLHWDL |
| | | | PLIDGGSRITNYIVEKREATRKSYSTV |
| | | | TTKCHKCTYKVTGLSEGCEYFFRVM |
| | | | AENEYGIGEPAETTEPVRASEAPSPP |
| | | | DSLNIMDITKSTVSLAWPKPKHDGGS |
| | | | KITGYVIEAQRKGSDQWTHITTVKGL |
| | | | ECVVKNLTEGEEYTFQVMAVNSAG |
| | | | RSAPRESRPVIVKEQTMLPELDLRGI |
| | | | YQKLVIAKAGDNIKVEIPVLGRPKPT |
| | | | VTWKKGDQILKQTQRVNFENTATST |
| | | | ILNINECVRSDSGPYPLTARNIVGEVG |
| | | | DVITIQVHDIPGPPTGPIKFDEVSSDF |
| | | | VTFSWEPPENDGGVPISNYVVEMRQ |
| | | | TDSTTWVELATTVIRTTYKATRLTTG |
| | | | VEYQFRVKAQNRYGVGPSITSAPVV |
| | | | ANYPFKVPGPPGTPQVAAVTKDSITI |
| | | | SWHEPLSDGGSPILGYHVERKERNGI |
| | | | LWQTVSKALVPGNIFKSSGLTDGIAY |
| | | | EFRVIAENMAGKGKPSKPSEPILALD |
| | | | PIDPPGKPVPLNITRHTVTLKWAKPE |
| | | | YTGGFKITSYIVEKRDLPNGRWLKA |
| | | | NFSNILENEFTVSGLTEDAAYEFRVIA |
| | | | KNAAGAISPPSEPSDAITCRDDIEAPR |
| | | | IMVDVKFKDTITLKAGEAFKLEADV |
| | | | SGRPPPTMEWTKDGKELENTAKLEI |
| | | | KIADFSTNLVNKDSLRRDGGAYTLT |
| | | | ATNPGGFAKHIFHVKVLDRPGPPEGP |
| | | | LAVSEVTSEKCVLSWLPPLDDGGAKI |
| | | | EYYVVQKRETSRLAWTNVASEVQV |
| | | | TKLKVTKLLKSNEYIFRVMAVNKYG |
| | | | VGEPLDSEPVLAVDPYGPPDPPKNPE |
| | | | VTSITKDSMVVCWGHPDSDGGSEIIN |
| | | | YIVERRDKAGQRWVKCNKKTLTDL |
| | | | RYKVSGLTEGHEYEFRVMAENRAGI |
| | | | SAPSATSPFYKACDAVFKPGPPGNPR |
| | | | VLDTSRSSISIAWNKPIYDGGSEITGY |
| | | | MVEIALPEEDEWKIVTPPSGLKATSY |
| | | | TITNLTENQEYKIRIYAMNSEGIGEPA |
| | | | LVPGTPKAEDRMLPPEIELDAELRKV |
| | | | VTIRACCTLRLFVPIKGRPAPEVKWT |
| | | | REHGESLDKASIESTSSYTLLIIGNVN |
| | | | RFDSGKYILTIENSSGSKSAFVNVRV |
| | | | LDTPGPPQDLKVKEVTKTSVTLTWE |
| | | | PPLIDGGSKINNYIVEKRESTRKAYST |
| | | | VTTNCHKTSWKVDQLQEGCSYYFR |
| | | | VLAENEYGIGLPAETAESVKASERPL |
| | | | PPGKITLVDVTRNSVSLSWEKPEHDG |
| | | | GSRILGYIVEMQSKGSDKWVTCATV |
| | | | KVTEATITGLIQGEEYSFRVSAQNEK |
| | | | GISDPRQLSVPVIAKDLVIPPAFKLLF |
| | | | TTFTVLAGEDLKVDVPFTGRPTPAVT |
| | | | WHKDDVPLKQTTRVNAESTENNSLL |
| | | | TIKEACREDVGHYIVKLTNSAGEATE |
| | | | TLNVIVLDKPGPPTGPVKMEEVTADS |
| | | | VTFSWGPPKYDGGSSINNYIVEKRDT |
| | | | STTTWQIVSATVARTTIKACRLKTGC |
| | | | EYQFRIAAENRYGKSTYLTSEPVVAQ |
| | | | YPFKVPGPPGTPFVTLSSKDSMEVH |
| | | | WNEPVSDGGSKVIGYHLERKERNSIL |
| | | | WVKLNKTPIPQTKFKTTGLDEGIEYE |
| | | | FRVSAENIVGIGKPSKVSESYVARDP |
| | | | CDPPGRPEPIIVTRNSVTLQWKKPAY |
| | | | DGGSKITGYIVEKKELPDGRWMKAS |
| | | | FTNVIDTQFEVTGLVEDHRYEFRVIA |
| | | | RNAAGVFSEPSESTGAITARDEVEPP |
| | | | QIRMDPKYKDTIVVHAGELFKIDADI |
| | | | HGKPIPTTQWIKGDHELSNTARLEIK |
| | | | STDFATSLSVKDAIRIDSGSYILKAKN |
| | | | VAGEKSVTVNVKVLDRPGPPEGPIVI |
| | | | SGVTAEKCTLAWKPPLQDGGSDIINY |
| | | | IVERRETSRLVWTVVDANVQTLGCK |
| | | | VTKLLEGNEYIFRVMAVNKYGVGEP |
| | | | LESEPVTAKNPFVVPDAPKAPEITAV |
| | | | TKDSMIVVWERPAFDGGSEILGYVL |
| | | | EKRDKEGIRWTRCHKRLIGELRLRVT |
| | | | GLLENHNYEFRVSAENAAGLSEPSPP |
| | | | SAYQKACDPIYKPGPPNNPRVTDITR |
| | | | SSVFLSWGKPIYDGGCEIQGYIVEKC |
| | | | DTSVGEWTMCTPPTGINKTNIEVEKL |
| | | | LEKHEYNFRICAVNKAGVGEHADVP |
| | | | GPVIVEEKLEAPDIDLDLELRKILNIR |
| | | | AGGSLRLFVPIRGRPTPEVKWGKVD |
| | | | GEIRDAAIIDSTSSFTSLVLDNVNRYD |
| | | | SGKYTLTLENSSGTKSAFVTVRVLDT |
| | | | PSPPVNLKVTEITKDSVSITWEPPLLD |
| | | | GGSKIKNYIVEKREATRKSYAAVVT |
| | | | NCHKNSWKIDQLQEGCSYYFRVTAE |
| | | | NEYGIGLPAHTDDPVKVAEVPQPPG |
| | | | KITVDDVTRNSVSLSWTKPEHDGGS |
| | | | KIIQYIVEMQAKHSEKWSECARVKSL |
| | | | EAVITNLTQGEEYLFRVVAVNEKGR |
| | | | SDPRSLAVPIVAKDLVIEPDVRPAFNS |
| | | | YSVQVGQDLKIEVPISGRPKPTITWT |
| | | | KDDLPLKQTTRINVTDSLDLTVLSIK |
| | | | ETHKDDGGHYGITVANVVGQKTASI |
| | | | EIITLDKPDPPKGPVKFDEVSAESITLS |
| | | | WEPPLYTGGCQITNYVVQKRDTTTT |
| | | | VWEWSATVARTTLKVTKLKTGTEY |
| | | | QFRIFAENRYGQSFALESEPIVAQYP |
| | | | YKEPGPPGTPFVTAVSKDSMVVQWH |
| | | | EPINNGGSPVIGYHLEKKERNSILWT |
| | | | KVNKTIIHDTQFKAVNLEEGIEYEFR |
| | | | VYAENIVGIGKASKNSECYVARDPC |
| | | | DPPGTPEAIIVKRHEITLQWTKPAYD |
| | | | GGSVITGYIVEKRDLPEGRWMKASF |
| | | | TNVIETQFTVSGLTEDQRYEFRVIAK |
| | | | NAAGAVSKPSDSTGPITARDEVELPR |
| | | | ISMDPKFRDTIVVNAGETFRLEADVH |
| | | | GKPLPTIEWLRGDKEIEESARYEIKNT |
| | | | DFKALLIVKDAIRIDGGQYILRASNV |
| | | | AGSKSFPVNVKVLDRPGPPEGPVQV |
| | | | TGVTAEKCTLTWSPPLQDGGSDISHY |
| | | | VVEKRETSRLAWTVVASEVVTNSLK |
| | | | VTKLLEGNEYIFRIMAVNKYGAGEP |
| | | | LESAPVLMKNPFVLPGPPKSLEVTNI |
| | | | AKDSMTVCWNRPDSDGGSEIIGYIVE |
| | | | KRDRSGIRWIKCNKRRITDLRLRVTG |
| | | | LTEDHEYEFRVSAENAAGVGEPSPA |
| | | | TLYYKACDPVFKPGPPINAHVVDTT |
| | | | KNSITLAWGKPIYDGGSEILGYVVEI |
| | | | CKADEEEWQIVTPQTGLKATRFEISK |
| | | | LTEHQEYKVRVCALNKVGLGEATA |
| | | | VPGTVKPEDKLEAPELDLDSELRKGI |
| | | | VVRAGGSVRIHIPFKGRPTPEITWSRE |
| | | | EGEFTDKVQIEKAANYTQLSIDNCDR |
| | | | NDAGKYVLKLENSSGSKSAFVTVKV |
| | | | LDTPGPPQNLAVKEVRKDSVLLVWD |
| | | | PPLIDGGAKVKNYVIDKRESTRKAY |
| | | | ANVSNKCSKTSLKVENLTEGAIYYFR |
| | | | VMAENEFGIGVPVETVDAVKASEPP |
| | | | SPPGKVTLTDVSQTSASLMWEKPEH |
| | | | DGGSRILGYVVEMQPKGTEKWSVV |
| | | | AESKVCSAWTGLSSGQEYHFRVKA |
| | | | YNEKGQSDPRVLGVPVIAKDLTIQPS |
| | | | FKLPFNTYSVQAGEDLKIEIPVIGRPR |
| | | | PEISWVKDGEPLKQTTRVNVEKTAT |
| | | | STILHIKESNKDDFGKYTVTATNSAG |
| | | | TATENLSIIILEKPGPPVGPVRFDEVS |
| | | | ADFVVISWEPPAYTGGCQISNYIVEK |
| | | | RDTTTTTWHMVSATVARTTIKITKL |
| | | | KMGSEYQFRIFAENRYGKSAPLDSKP |
| | | | VIVQYPFKEPGPPGTPFVTSVSKDQM |
| | | | LVQWHEPVNDGGSKVIGYHLEQKE |
| | | | KNSILWVKLNKTPIQDTKFKTTGLDE |
| | | | GLEYEFRVSAENIVGIGKASKVSECF |
| | | | VARDPCDPPGRPEAIVITRNNVTLKW |
| | | | KKPAYDGGSKITGYIVEKKDLPDGR |
| | | | WMKASFTNVLDTEFTVSGLVEDQRY |
| | | | EFRVIARNAAGNFSEPSESTGAITAR |
| | | | DEIDAPNASLDPKYKDVIVVHAGETF |
| | | | VLEADIRGKPIPDIVWSKDGRELEET |
| | | | AARMEIKSTIQRTTLVVKDCIRSDGG |
| | | | QYILKLSNVGGTKTIPITVKVLDRPGP |
| | | | PEGPLKVSGVTAEKCYLAWNPPLQD |
| | | | GGASISHYIIEKRETSRLSWTQVSTEV |
| | | | QALNYKVTKLLPGNEYIFRVMAVNK |
| | | | YGTGDPLESEPVIARNPYKPPGPPSPP |
| | | | EVSAITKDSMVVTWARPVDDGGAEI |
| | | | EGYILEKRDKEGVRWTKCNKKTLTD |
| | | | LRFRVTGLTEGHSYEFRVAAENAAG |
| | | | VGEPSEPSVFSRACDALYPPGPPSNP |
| | | | KVTDTSRSSVSLAWNKPIYDGGAPV |
| | | | KGYVVEVKEATADEWTTCTPPTGLQ |
| | | | GKQFTVTELKENTEYNFRICAINSEG |
| | | | VGEPATIPGSVAAKERQEPPEIELDA |
| | | | DLRKVVILRASATLRLFVTIKGRPEPE |
| | | | VKWEKAEGVLTDRAQIEVTSSYTML |
| | | | VIDNVTRFDSGRYNLTLENNSGSKTA |
| | | | FVNVRVLDSPSAPVNLTVREVKKDS |
| | | | VILAWEPPLIDGGAKITNYIVEKRETT |
| | | | RKAYATVTNNCTKNSFKIENLQEGC |
| | | | SYYFRVLASNEYGIGLPAETTEPVKA |
| | | | SEPPLPPGRVTLVDVTRNTATIKWEK |
| | | | PESDGGSKITGYVVEMQTKGSEKWS |
| | | | ICTQVKTLEATISGLTAGEEYIFRVAA |
| | | | INEKGKSDPRQLGVPVIARDIEIKPSV |
| | | | ELPFNTFNVKARDQLKIDIPFKGRPQ |
| | | | ATVSWKKDGQTLKETTRVNVSSSKT |
| | | | VTSLIIKEASREDVGTYELCVSNSAG |
| | | | SVTVPITVIVLDKPGPPGPIHIDEVSC |
| | | | DNITISWNPPEYDGGCQISNYIVEKRE |
| | | | TTSTTWHVVSQAVARTSIKIVRLVTG |
| | | | SEYQFRVCAENRFGKSSYSESSAVVA |
| | | | EYPFSPPGPPGTPKVVHATKSTMLVT |
| | | | WQVPVNDGGSRVLGYHLEYKERSSI |
| | | | LWSKANKTLIADTQMKVSGLDEGL |
| | | | MYEYHVYAENIAGIGKCSKSCEPVP |
| | | | ARDPCDPPGQPEVTNITRKSVSLKWS |
| | | | KPHYDGGAKITGYIIERRELPDGRWL |
| | | | KCNFTNVPETYFEVTELTEDQRYEFR |
| | | | VFARNAADSISEPSESTGPITVKDDVE |
| | | | APRIMMDVKFRDVIVVKAGEVLKIN |
| | | | ADVAGRPLPIISWAKDGVEIEERART |
| | | | EIVSTDYTTLLTVKDCVRRDSGQYV |
| | | | LTVKNVAGTRSMAVNCKVLDKPGP |
| | | | PAGPLEITGLTAEKCSLSWGPPQEDG |
| | | | GAAIDYYIVEKRETSRLAWTICEGEL |
| | | | RTTFCKVTKLLKGNEYIFRVTGVNK |
| | | | YGVGEPLESMAVKALDPFTVPSPPTS |
| | | | LEITSVTKEFMTLCWSRPESDGGSEIS |
| | | | GYIVERREKNSLRWVRVNKKPVYDL |
| | | | RVKSTGLREGCEYEYRVYAENAAGL |
| | | | SLPSESSPLIRAEDPVFLPSPPSKPNV |
| | | | MDSGKTNITIGWVKPLFDGGAPITGY |
| | | | TVEFKKSDETDWQTAIQNLRGTEYT |
| | | | VSGLTTGAEYVFRVRSINKVGASDPS |
| | | | DSSDPQIAKEREEEPVFDLDTEMRKT |
| | | | LIVKAGASFTMTVPFRGRPVPSVSWS |
| | | | KPDTDLRTRAYIDSTESRTSLTIENAN |
| | | | RNDSGKYTLMIQNVLNAASLTLVVK |
| | | | VLDSPGPPANITVHDVTKESAVLSW |
| | | | DVPENDGGAPVKNYHIEKREASKKA |
| | | | WVSVTNNCNRLSYKITNLQEGAIYY |
| | | | FRVSGENEFGVGVPAETKEGVKITEK |
| | | | PSPPEKLGVTSVSRDSVSLAWLKPEH |
| | | | DGGSRIVHYVIEALEKGQTTWMRCA |
| | | | VVKSTHHVVSGLRQNSEYFFRVFAE |
| | | | NQAGLSDPRELLLPVLIKEQLEPPEID |
| | | | MKNFPSHTVYVRAGSNLKVDIPISGK |
| | | | PLPKVTLSRDGVPLKATMRFNTEITA |
| | | | ENLTINLKESVAADAGRYEITAANSS |
| | | | GTTKAFINIVVLDRPGPPTGPVVISDI |
| | | | TEDSVTLKWEPPKYDGGSQVTNYIV |
| | | | LKRETSTAVWTEVSATVARTMIKVM |
| | | | KLTTGEEYQFRIKAENRFGISDHIDSA |
| | | | CVVVKLPYTTPGPPSTPWVTNVTRES |
| | | | ITVGWHEPVSNGGSPVTGYHLEMKD |
| | | | RNSILWQKANKMVIRTTHFKVTTISA |
| | | | GLIYEFRVYAENAAGIGKPSHPSEPV |
| | | | LAIDACEPPRNVRITDISKNSVNLSW |
| | | | QQPAFDGGSKITGYMVERRDLPDGR |
| | | | WAKASFTNVTETHFTVSGLTQNAQY |
| | | | EFRVFARNAVGSISNPSEVVGPITCID |
| | | | SYGGPVIDLPLEYTEVVKYRAGTSV |
| | | | KLRAGISGKPEPTIEWYKDDKELQTN |
| | | | ALVCVENTTDLASILIKDATRLNSGT |
| | | | YELKLRNALGSASATIRLQILDKPGP |
| | | | PGGPIEFKTVTAEKITLLWQPPADDG |
| | | | GAKITHYIVEKRETSRVVWSMVSEN |
| | | | LEECIIKTTKIIKGNEYIFRVRAVNKY |
| | | | GIGDALESHPVIARNAFVTPGPPSVPE |
| | | | VTKINKNSMTVVWSRPVADGGSDIS |
| | | | GYILEKRDKKSLGWFKVLKETIRDTR |
| | | | QKVTGLTEHSDYQYRVCAVNAAGQ |
| | | | GPFSEPSDFYKAADPIDPPGPPAKIRI |
| | | | ADSTKSSITLGWSKPVYDGGSAVTG |
| | | | YVVAMRQGEEEEWTVVSTKGEVRT |
| | | | TEYVVSNLSPGVNYYFQVSAVNCAG |
| | | | QGEPIAMTEPVQAKDILEEPEIDLDV |
| | | | AFRTSIIAKAGEDVHALVPFKGRPPP |
| | | | TVTWRKDEKNLGSDARYSIQNTDSS |
| | | | SILTIPQVTRHDTGKYILTIENGVGQP |
| | | | KSSTVSVKVLDTPAACQNLQVKHVS |
| | | | QGTVTLFWDPPLIDGGSPIINYIIEKK |
| | | | DATKRTWSSVSHKCSSTSFKVTDLSE |
| | | | KTPFFFRVFAVNEIGIGEPCETTEPVK |
| | | | AAEVPAPIRDLSVKDSTKTSVTLSWT |
| | | | KPDFDGGSVITDYIVERKGKGEQTW |
| | | | SHAGISKTCEIEVGKLKELSELEFRVS |
| | | | ARNEKGLSDSVSIGPVTVKELVIPPE |
| | | | VDLSEIPGAQVAVRIGHNVHLELPYK |
| | | | GKPKPSISWLKDGLPLKESELVRLGK |
| | | | TENKLTLSIKNAKKENGGKYTIILDN |
| | | | AVCRNTYPITVITLGPPSKPKGPIRFD |
| | | | EIKADSVIMSWDVPDDDGGGEITCYS |
| | | | IEKREASQTNWKMVCSSVARTTFKV |
| | | | PNLVKDAEYQFRVRAENRYGVSQPL |
| | | | VSNIILAKHQFRIPGPPGKPVTYNVTS |
| | | | DGMSLTWDAPVYDGGSEVTGYHVE |
| | | | KKERNSILWQRINMSPISGREYRATG |
| | | | LMEGLDYQFRVFAENSAGLSSPSDPS |
| | | | KFTLAVSPVDPPGTPDYIDVTRETITL |
| | | | KWNPPLRDGGSKIVAYSIEKRQGSD |
| | | | RWTRCNFTDVSECQYTVTGLSPGDR |
| | | | YEFRIIARNAVGTISPPSQSSGVIMTR |
| | | | DENVAPTVEFGPEYFDGITIKSGESLR |
| | | | IKALVQGRPVPRVTWFKDGVEIEKK |
| | | | MNMEITDVLGSTSLFVRDATRDHRG |
| | | | VYRVEAKNASGSTKAEITVRVQDTP |
| | | | GKPVGPIRFTNITGEKMTLWWDAPH |
| | | | NDGCAPVSHYLIEKRETSRLAWALIE |
| | | | DNCEALSYTATKLITGNEYQFRISAV |
| | | | NKFGVSRPLDSDPVVAQIQYTIPDAP |
| | | | GTPEPSNVTGNSITLTWARPESDGGS |
| | | | EIQQYILERREKKSTRWVKVISKRPIC |
| | | | ETRFKVTGLTEGNEYEFHVMAENAA |
| | | | GVGPASGVSRLIKCREPVNPPGAPTV |
| | | | VKVTDTSKTTVSLEWSKPVFDGGLEI |
| | | | IGYIIEMCKADLGDWHKVNVEACVK |
| | | | TRYTVTDLQAGEEYKFRVSAINAAG |
| | | | KGDSCEVTGTIKAVDRLSAPELDIDA |
| | | | NFKQTHVVRAGASIRLFIAYQGRPTP |
| | | | TAVWSKPDSNLSIRADIHTTDSFSTLT |
| | | | VENCNRTDAGKYTLTVENNSGSKSI |
| | | | TFTVKVLDSPGPPGPITFKDVTRGSA |
| | | | TLMWDAPLLDGGARIHHYVVEKRE |
| | | | ASRRSWQVVSEKCIRQILRVSDLLEG |
| | | | VPYYFRVSAENEYGVGEPYEMPEPM |
| | | | VATEQPAPPRRLDIVDTSKSSVVLAW |
| | | | LKPDHDGGSRITGYLLEMRQKGSDF |
| | | | WVEAGHTKQMTFTVERLVENTEYEF |
| | | | RVKAKNDAGYSEPREAFSSVIIKEPQI |
| | | | EPTADLTGITNQLITCKAGSTFTIDVPI |
| | | | SGRPAPKVTWKLEEMRLKETDRVSI |
| | | | TTTKDRTTLSVKDSMRGDSGRYFLT |
| | | | LENTAGVKTFTITVVVIGRPGPVTGPI |
| | | | EVSSISAESCVLSWAEPQDNGGTDIT |
| | | | NYIVEKRESGTTAWQLVNSSVKRTQI |
| | | | KVTHLTKYMEYSFRVSSENRFGVSK |
| | | | PVESAPIVAEHPFVPPSAPTRPEVYYV |
| | | | SANAMSIRWEEPYHDGGSKVIGYWV |
| | | | EKKERNTILWVKENKVPCLECNYKV |
| | | | TGLVEGLEYQFRTYALNAAGVSKAS |
| | | | EASRPVMAQNPVDAPGRPEVTDVTR |
| | | | STVSLIWSAPVYDGGSKVVGYIIERK |
| | | | PVSEVGDGRWLKCNYTIVSDNFFTV |
| | | | TALSEGDTYEFRVLAKNAAGVISKG |
| | | | SESTGPITCRDEYAPPKAELDARLQG |
| | | | DLVTIRAGSDLVLDAAVGGKPEPKII |
| | | | WTKGDKELDMCEKISLQYTGKRAT |
| | | | AVIKFCDRSDSGKYTLTVKNASGTK |
| | | | AVSVLVKVLDSPGPCGKLTVSRVTE |
| | | | EKCTLAWSLPQEDGGAEITHYIVERR |
| | | | ETSRLNWVIVEGECPTLSHVVTRLIK |
| | | | NNEYIFRVRAVNKYGPGVPVESEPIV |
| | | | ARNSFTIPSQPGIPEEVGAGKEHIIIQ |
| | | | WTKPESDGGNDISNYLVDKREKKSL |
| | | | RWTRVNKDHVVYDTRLKVTGLMEG |
| | | | CDYQFRVTAVNAAGNSEPSEASNFIS |
| | | | CREPSYTPGPPSAPRVVDTTKHSISLA |
| | | | WTKPMYDGGTDIIGYVLEMQEKDT |
| | | | DQWYRVHTNATIRNNEFTVTDLKM |
| | | | GQKYSFRVAAVNVKGMSEYSESTAE |
| | | | IEPVERLEIPDLELADDLKKTVTIRAG |
| | | | ASLRLMVSVSGRPPPVITWSKKGIDL |
| | | | ASRAIIDTTESYSLLIVDKVNRYDAG |
| | | | KYTIEAENQSGKKSATVLVKVYDTP |
| | | | GPCPSVKVKEVSRDSVTITWEVPTID |
| | | | GGAPVNNYIIEKREAAMRAFKTVTT |
| | | | KCSKTLYRISGLIEGAMYYFRVLPEN |
| | | | IYGIGEPCETSDAVLVSEVPLVPTKLE |
| | | | VVDVTKSTVTLAWEKPLYDGGSRLT |
| | | | GYVLEACRAGTERWMKVVTLKPTV |
| | | | LEHTVISLNEGEQYLFRVRAQNEKG |
| | | | VSEPREIVTAVTVQDLRVLPTIDLST |
| | | | MPQKTIHVPAGRPVELVIPIAGRPPPA |
| | | | ASWFFAGSKLRESERVTVETHTKVA |
| | | | KLTIRETTIKDTGDYVLELKNVTGTT |
| | | | SETIKVIVLVDKPGPPSGPIKVDEIDA |
| | | | TSVTISWGPPELDGGAPLSGYVVEQR |
| | | | DAHRPGWLPVSESVTRSTFKFTRLIE |
| | | | GNEYVFRVAATNRFGIGSYLQSEVIE |
| | | | CRSSINIPGPPETLQIFDVSREGMTLT |
| | | | WYPPEDDGGSQVTGYIVERKEVRAD |
| | | | RWVRVNKVPVTMTRYRSTGLIEGLE |
| | | | YEHRVTAVNVRGTGKPSRPSKPTVA |
| | | | MDPIAPPGKPQNPRVTDTTRTSVSLA |
| | | | WSVPEDEGGSKVTGYLIEMQKVDQ |
| | | | HEWTKCNTTPTKIREYTLTHLPQGAE |
| | | | YRFRVLACNAGGPGEPAEVPGTVKV |
| | | | TEMLEYPDYELDERYQEGILVRQGG |
| | | | VIRLTIPIKGKPFPICKWTKEGQDISK |
| | | | RAMIATSETHTELVIKEADRDDSGTY |
| | | | DLILENKCGKKAVYIKVRVIGNPNTP |
| | | | EGPLEYDDIQARSVRVSWRPPADDG |
| | | | GADILGYILERREVPKSAWYTIDSRV |
| | | | RGTSLVVKGLKENVEYHFRVSAENQ |
| | | | FGISKPLKSEEPVIPKTPLNPPEPPSNP |
| | | | PEILDVTKSSVSLSWSRPKDDGGSRV |
| | | | TGYYIERKETSTDKWVRHNKTQITTT |
| | | | MYTVTGLVPDAEYQFRIIAQNDVGL |
| | | | SETSPASEPVVCKDPFDKPSQPGELEI |
| | | | LSISKDSVTLQWEKPECDGGKEILGY |
| | | | WVEYRQSGDSAWKKSSKDRIKDRQ |
| | | | FTIGGLLEATEYEFRVFAENETGLSRP |
| | | | RRTAMSVKTKLTSGEAPGVRKEMK |
| | | | DVTTKLGEAAQLSCQIVGRPLPDIKW |
| | | | FRFGKELVQSRKYKMSSDGRTHTLT |
| | | | VMTEEQEDEGVYTCVATNEVGEVES |
| | | | SSKLLLQATPQFHPGYPLKEKYYGA |
| | | | VGSTLRIHVMYIGRPVPAITWFRGQK |
| | | | LLQNSENITIENTEHYTHLVMKNVQR |
| | | | KTHAGKYKVQLSNVLGTVDTMLDV |
| | | | EIQDKPDKPTGPIVIEALLKNSVVISW |
| | | | KPPADDGGSWITNYVVEKCEAKEGA |
| | | | EWQLVSSAISVTTCRIVNLTENAGYY |
| | | | FRVSAQNMFGISEPLEVSSVVIIKSPF |
| | | | EKPGAPGKPTITAVTKDSCVVAWKP |
| | | | PASDGGAKIRNYYLEKREKKQNKWI |
| | | | AVTTDEIRETVFSVQNLIEGLEYEFR |
| | | | VKCENLGGESEWSEISEPVTPKSDVPI |
| | | | QAPHFKEELRNLNVRYQSNATLVCK |
| | | | VTGHPKPIVKWYRQGKEIIADGLKY |
| | | | RIQEFKGGYHQLIIASVTDDDATVYQ |
| | | | VRATNQGGSVSGTASLEVEDTVPAK |
| | | | IHLPKNLEGMGAVHALRGEVISIKIPF |
| | | | SGKPDPVITWQKGQDLIDNNGHYQV |
| | | | IVTRSFTSLVFPNGVERKDAGFYVVC |
| | | | AKNRFGIDQKTVELDVADVPDPPRG |
| | | | VKVSDVSRDSVNLTWTEPASDGGSK |
| | | | VTNYIVEKCATTAERWIRVGQARET |
| | | | RYTVINLFGKTSYQFRIIAENKFGLSK |
| | | | PSEPSEPTVTKEDKTRAMNYDEEVD |
| | | | ETREVSMTKASHSSTKELYEKYMIA |
| | | | EDLGRGQFGIVHRCVETSSKKTYMA |
| | | | KFVKVKGTDQVLVKKEISILNIARHR |
| | | | NMLYLHESFESMEELVMIFEFISGLDI |
| | | | FERINTSAFELNEREIVSYVRQVCEAL |
| | | | EFLHSHNIGHFDIRPDNIIYQTRRSSTI |
| | | | KIIEFGQARQLKPGDNFRLQFTAPEY |
| | | | YAPEVHQHDVVSTATDMWSLGTLV |
| | | | YVLLSGINPFLAETNQQIIENIMNAEY |
| | | | TFDEEAFQEISLEAMDFVDRLLVKER |
| | | | KSRMTASEALQHPWLKQKTERVSTK |
| | | | VIRTLKHRRYYHTLVKKDLNMVVSA |
| | | | ARISCGGAIRSQKGVSIAKVKVASIEI |
| | | | GPVSGQIMHAVGEEGGYVKYVCRIE |
| | | | NYDQSTQVTWYFGVRQLENSEKYEI |
| | | | TYEDGVATMYVKDITKFDDGTYRC |
| | | | KVVNDYGEDSSYAELFVKGVREVY |
| | | | DYYCRRTVKKLKRRTDAMRLLERPP |
| | | | EFTLPLYNKTAYVGENVRFGVTITVH |
| | | | PEPRVTWYKSGQKIKPGDDDRKYIFE |
| | | | SDKGLYQLTINSVTMDDDAEYTVVA |
| | | | RNKYGEDSCKAKLTVTPHPPPSDTTL |
| | | | RPMFKRLLANAECQEGQSVCFEIRVS |
| | | | GIPPPTLKWEKDGRPLSLGPNIEIIHE |
| | | | GLDYYALHIRDTLPEDTGYYRVTAT |
| | | | NTAGSSSCQAHLQVERLRYVKQEFK |
| | | | TKEEHERHVQRQIDKTLRMAEILSGT |
| | | | EAVPLTQVAQEALREAAILYKPAVST |
| | | | KTVKGEYRLETEEKKEERKLRMPYE |
| | | | VPEPRKYKQTTIEEDQHIKQFVPMSD |
| | | | MKWYKKIRDQFEMPGKIDRVVQKR |
| | | | PKRIRLSRWEQFYVMPLPRITDQYRP |
| | | | KWRIPKLSQDDLEMVRPARRRTPSP |
| | | | DYYYYYRPRRRSLGDISDEELLLPID |
| | | | DYLAMKRTEEERLRLEEELELGFSAS |
| | | | PPSRSPPRFELSSLRYSSPQAHVKVEE |
| | | | ARRDFRYSTYHIPTKEETSTSYAELR |
| | | | ERHARASHRQAHQRQRIMAEREDHE |
| | | | LLRPATTTQRLLEYKSELDHLAKEA |
| | | | KSRKKSRRQREVTEITEIEEEYEISKH |
| | | | AQRETSSSVSRLLRRRRSLSPTYIELM |
| | | | RPVSELIRPRPRPAEEYEDEEEEEEED |
| | | | VERRSPTPERTRPRSPSPVSSERSLSR |
| | | | FERSARFDIFSRYESMKAALKTQKTS |
| | | | ERKYEVLSQQPFTLDHAPRITLRMRS |
| | | | HRVPCGQNTRFILNVQSKPTAEVKW |
| | | | YHNGVELQESSKIHYTNTSGVLTLEI |
| | | | LDCHIDDSGTYRAVCTNYKGEASDY |
| | | | ATLDVTGGDYTTYASQRRDEQVPRS |
| | | | VFPELTRTEAYAVSSFKKTSEMEASS |
| | | | SVREMKSLMTETRESLSSYEHHASA |
| | | | EMKSAAIEEKSLEEKSTHRKIKTTLA |
| | | | ARILTKPRSITVYEGESARFSCDTDGE |
| | | | PVPTVTWLRGGQVISTSARHQVTTS |
| | | | KYKSTFEISSVQASDEGNYSVVVENS |
| | | | DGRQEAQFTLTVQKARVTEKAVTSP |
| | | | PRVKSPEPRVKSPEGAKSPKRVKSPE |
| | | | PVTPHPKAVPPTETKPTPTEKVQQKP |
| | | | VAAPPKIIQSLKAEASKDIAKLTCVV |
| | | | ESSALCAKEVTWYKDGRRLKENGHF |
| | | | QFHYSADGTYELKILNLAESDRGEY |
| | | | VCEVSGEGGTSKTNFQFVGQAFKSIH |
| | | | EQVSSISETNKKAAPKTAEPTETKKT |
| | | | EPKAPAPISTKPVIVTGLQDTTVSSDS |
| | | | VAKFAVKVTGEPQPTVIWTKDGKAI |
| | | | SQGGKYKLSEDKGAFFLEIHKTDTSD |
| | | | SGLYTCTITNSAGSVSSSCKLTIKVVE |
| | | | DTEIQKVSAQKTSEITSQKKASVQEEI |
| | | | SKKALISEEIKTSEVKSHEKLALKEEA |
| | | | STVLISEKVKKSEAASLEKSVVHEEIT |
| | | | KTSQASEEIRTHAEIKAFSTQMSITAG |
| | | | QKVTLKANIAGATDVKWVLNGVEL |
| | | | SNSDEYRYGISGSDQTLTIRQAGHKD |
| | | | EGILTCIGKTSQGIIKCQYDLTLSKEL |
| | | | SDAPAFISQPRSQNVNEGQNVLFSCEI |
| | | | SGEPSPEIEWFKNNLPISISSNISVSRS |
| | | | RNVYSLEIRNASVSDSGKYTIKAKNF |
| | | | HGQCSATASLTVLPLVEEPPREVVLR |
| | | | TSGDTSLQGSFSSQSVQMSASKQEAS |
| | | | FSSFSSSSASSMTEMKFASMSAQSMS |
| | | | SMQESFVEMSSSSFMGKSSMTQLESS |
| | | | TSRMLKAGLRGIPPKIEALPSDISIDE |
| | | | GKVLTVACAFTGEPTPEITWSRGGRT |
| | | | IHDQEQRGRFMENTDDL |
| SEQ ID | NP_990445.1 | Troponin T, | MSEAEEEYEEEQPEEEEEAAAAAEEE |
| NO: 65 | (NCBI) | slow skeletal | EEEEAEASKPHEEPEEERPRPRPVVP |
| | | muscle/Gallus | QLAPPKIPEGERVDFDDIHRKRMEKD |
| | | gallus | LLELQTLIDAHFEQRRREENELVALM |
| | | | ERIERRRAERNEQLRSRTEKERERQA |
| | | | RLAEEKLRKEEEEAKKRAEDDAKKK |
| | | | KVLSNMPHFGGYLAKAEQRRGKRQ |
| | | | TGREMKLRILAERKKPLHIEHMREDE |
| | | | LRAKAKELHDWIQQLESEKFDLMEK |
| | | | LRRQKYEINVLYNRISHAQKFKKVV |
| | | | GKGRVGGRWK |
| SEQ ID | Q98916 | Slow muscle | MSEAEEEYEEEQPEEEEEAAAAAEEE |
| NO: 66 | (UniProtKB) | troponin | EEEEAEASKPHEEPEEERPRPRPVVP |
| | | T/Gallus gallus | QLAPPKIPEGERVDFDDIHRKRMEKD |
| | | | LLELQTLIDAHFEQRRREENELVALM |
| | | | ERIERRRAERNEQLRSRTEKERERQA |
| | | | RLAEEKLRKEEEEAKKRAEDDAKKK |
| | | | KVLSNMPHFGGYLAKAEQRRGKRQ |
| | | | TGREMKLRILAERKKPLHIEHMREDE |
| | | | LRAKAKELHDWIQQLESEKFDLMEK |
| | | | LRRQKYEINVLYNRISHAQKFKKVV |
| | | | GKGRVGGRWK |
| SEQ ID | XP_419242.3 | Troponin 1, slow | MPEPRERKSKITASRKLLLKSLMLAK |
| NO: 67 | (NCBI) | skeletal | AKEEWEQEIVDKQSEKERYLSERITP |
| | | muscle/Gallus | LHTSGLSLSQLQDLCRELHEKVEIVD |
| | | gallus | EERYDIEAKCNHNTREIKDLKLKVLD |
| | | | LRGKFKRPPLRRVRVSADAMLRALL |
| | | | GSKHKVSMDLRANLKSVKKEDTEK |
| | | | ERPVEVGDWRKNVEAMSGMEGRKK |
| | | | MFDAAKSPTGQ |
| SEQ ID | F1NUT9 | Troponin 11, | LPIHTSTVCLCPQSLMLAKAKEEWE |
| NO: 68 | (UniProtKB) | slow skeletal | QEIVDKQSEKERYLSERITPLHTSGLS |
| | | type/Gallus | LSQLQDLCRELHEKVEIVDEERYDIE |
| | | gallus | AKCNHNTREIKDLKLKVLDLRGKFK |
| | | | RPPLRRVRVSADAMLRALLGSKHKV |
| | | | SMDLRANLKSVKKEDTEKERPVEVG |
| | | | DWRKNVEAMSGMEGRKKMFDAAK |
| | | | SPTGQ |
| SEQ ID | NP_001001862.1 | Troponin C, | MTDQQAEARSYLSEEMIAEFKAAFD |
| NO: 69 | (NCBI) | skeletal | MFDADGGGDISVKELGTVMRMLGQ |
| | | muscle/Sus | TPTKEELDAIIEEVDEDGSGTIDFEEF |
| | | scrofa | LVMMVRQMKEDAKGKSEEELAECF |
| | | | RIFDRNADGYIDAEELAEIFRASGEH |
| | | | VTDEELESLMKDGDKNNEGRIDFDE |
| | | | FLKMMEGVQ |
| SEQ ID | B5DH00 | Fast myotomal | MSDTEEVEAQKPQFKVPKIPDGDKV |
| NO: 70 | (UniProtKB) | muscle troponin- | DFDDIQKKRQNKDLIELQALIDAHFE |
| | | T-1/Salmo salar | HRKKEEEELIALKERIEKRRAERAEQ |
| | | | NRIRSEKEKERAARREEERLKREEAD |
| | | | AKKKADEDAKKKSALSSMGSNYSS |
| | | | HLQKADSKRGGKKETEREKKKKILA |
| | | | GRRKALNIDHLNEEKLKEKAKELHE |
| | | | WMQTLESEKFDHIERLKRQKYEVTT |
| | | | LRKRVEELSKFSKKGKTVRRK |
| SEQ ID | O57559 | Troponin T | MSDTEEVEHGEAHEAEEVHEEAHHE |
| NO: 71 | (UniProtKB) | variant TnTx7- | EAHHEEAHHEEAHHAEAHHAEAHH |
| | | e16/Gallus | EEAHAHAEEVHEPAPPPEEKPRIKLT |
| | | gallus | APKIPEGEKVDFDDIQKKRQNKDLIE |
| | | | LQALIDSHFEARRKEEEELVALKERI |
| | | | EKRRAERAEQQRIRAEKEKERQARL |
| | | | AEEKARREEEDAKRKAEDDLKKKK |
| | | | ALSSMGASYSSYLAKADQKRGKKQ |
| | | | TARETKKKVLAERRKPLNIDHLNED |
| | | | KLRDKAKELWDWLYQLQTEKYDFA |
| | | | EQIKRKKYEILTLRCRLQELSKFSKK |
| | | | AGAKGKVGGRWK |
| SEQ ID | A0A287BD71 | Myotubularin | MASAPTSKYNSHSLENESIKRTSRDG |
| NO: 72 | (UniProtKB) | 1/Sus scrofa | VNRDMGEAVPRLPGETPITDKEVIYI |
| | | | CPFNGPIKGRVYITNYRLYLRSLETD |
| | | | SALILDVPLGVISRIEKMGGATSRGE |
| | | | NSYGLDITCKDMRNLRFALKQEGHS |
| | | | RRDMFEILTRYAFPLAHSLPMFAFLN |
| | | | EEKFNVDGWTVYNPVEEYRRQGLP |
| | | | NHHWRITFINKCYELCDTYPALLVVP |
| | | | YRAADEDLRRVATFRSRNRIPVLSWI |
| | | | HPENKTVIVRCSQPLVGMSGKRNKD |
| | | | DEKYLDVIRETNRQVNKLTIYDARP |
| | | | NVNAVANKATGGGYESDDAYHNAE |
| | | | LFFLDIHNIHVMRESLKKVKDIVYPN |
| | | | VEESHWLSSLESTHWLEHIKLVLTGA |
| | | | IQVADRVSSGKSSVVVHCSDGWDRT |
| | | | AQLTSLAMLMLDSFYRSIEGFEILVQ |
| | | | KEWISFGHKFASRIGHGDKNHADAD |
| | | | RSPIFLQFIDCVWQMSKQFPTAFEFN |
| | | | EHFLITILDHLYSCRFGTFLYNCESAR |
| | | | ERQKVTERTVSLWSLINSNKDKFKN |
| | | | PFYTKEINRVLYPVASMRHLELWVN |
| | | | YYIRWNPRIKQQQPNPVEQRYMELL |
| | | | ALRDEYIKRLEELQLANSAKLSEPAA |
| | | | APSSPSQMVSHVQTHF |
| SEQ ID | Q4PS85 | Myozenin-1/Sus | MPLSGTPAPNKKRKSSKLIMELTGG |
| NO: 73 | (UniProtKB) | scrofa | GQESSGLNLGKKISVPRDVMLEELSL |
| | | | LTNRGSKMFKLRQMRVEKFIYENHP |
| | | | DVFSDSSMDHFQKFLPTVGGQLGTA |
| | | | GQGFSYSKGSSGGQAGGSSSAGQYG |
| | | | SGQQHHHQGSGSGSGGAGGPGSQTG |
| | | | RGGDAGTTGVGETGTGDQAGGEGK |
| | | | HITVFKTYISPWEKAMGVDPHQKVE |
| | | | LGIDLLAYGAKAELPQYKSFNRTAM |
| | | | PYGGYEKASKRMTFQMPKFDLGPLL |
| | | | SEPLVLYNQNLSNRPSFNRTPIPWLSS |
| | | | GEPVDYNVDIGIPLDGETEEL |
| SEQ ID | XP_010712691.1 | myozenin-1 | MPLAGTPAPLKRKKPTKLIGKLTHEV |
| NO: 74 | (NCBI) | isoform | MPQEVTKLNLGKKISIPRDVMLEELS |
| | | X1/Meleagris | LLTNKGSKMFKLRQLRVEKFIYENN |
| | | gallopavo | PDAFSDNSVDHFQRFIPSGGHYGEDA |
| | | | HGYGHGRMVGGVTAGQHGSSKQH |
| | | | YSTVPPRPGSKGGPGNSEGEHAEKSA |
| | | | GSAGEGGHGTEKDGKSGGKKPLLKT |
| | | | YISPWERAMGISPEDKSQLTIDLLSYS |
| | | | PKADFP |
| | | | HYKSFNRTAMPYGGYEKAAKRMTF |
| | | | KVPQFDICPLLPESIVLYNQNFRNRPS |
| | | | FNRTPIPWMPSGESSEYHTDINVPRS |
| | | | GETEEL |
| SEQ ID | Q0III9 | Alpha-actinin- | MMMVLQPEGLGTGEGPFAGGRGGG |
| NO: 75 | (UniProtKB) | 3/Bos taurus | EYMEQEEDWDRDLLLDPAWEKQQR |
| | | | KTFTAWCNSHLRKAGTQIENIEEDFR |
| | | | NGLKLMLLLEVISGERLPRPDKGKM |
| | | | RFHKIANVNKALDFIASKGVKLVSIG |
| | | | AEEIVDGNLKMTLGMIWTIILRFAIQ |
| | | | DISVEETSAKEGLLLWCQRKTAPYR |
| | | | NVNVQNFHTSWKDGLALCALIHRHR |
| | | | PDLIDYAKLRKDDPIGNLNTAFEVAE |
| | | | KYLDIPKMLDAEDIVNTPKPDEKAIM |
| | | | TYVSCFYHAFAGAEQAETAANRICK |
| | | | VLAVNQENEKLMEEYEKLASELLEW |
| | | | IRRTVPWLENRVGEPSMSAMQRKLE |
| | | | DFRDYRRLHKPPRVQEKCQLEINFNT |
| | | | LQTKLRLSHRPAFMPSEGKLVSDIAN |
| | | | AWRGLEQAEKGYEDWLLSEIRRLQR |
| | | | LQHLAEKFQQKASLH |
| | | | EAWTRGKEDMLSQRDYETASLQEV |
| | | | RALLRRHEAFESDLAAHQDRVEHIA |
| | | | ALAQELNELDYHEAASVNSRCQAIC |
| | | | DQWDNLGTLTQKRRDALERMEKLL |
| | | | ETIDQLQLEFARRAAPFNNWLDGAV |
| | | | EDLQDVWLVHSVEETQSLVTAHDQF |
| | | | KATLPEADRERGAILGIQGEIQKICQT |
| | | | YGLRPSSTNPYITLTPQDINTKWDTV |
| | | | RKLVPSRDQMLQEELTRQQVNERLR |
| | | | RQFAAQANAIGPWIQGKVEEVGRLA |
| | | | AGMAGSLEEQMAGLRQQEQNIINYK |
| | | | SNIDRLEGDHQLLQESLVFDNKHTV |
| | | | YSMEHIRVGWEQLLTSIARTINEVEN |
| | | | QVLTRDAKGLSQEQLNEFRASFNHF |
| | | | DRKRNGMMEPDDFRACLISMGYDL |
| | | | GEVEFARIMTMVDPNAAGVVTFQAF |
| | | | IDFMTRETAETDTAEQVVA |
| | | | SFKILAGDKNYITAEELRRELPAEQA |
| | | | EYCIRRMAPYKGAGAPAGALDYVAF |
| | | | SSALYGESDL |
| SEQ ID | G3X7I1 | Alpha-actinin- | MMMVLQPEGLGTGEGPFAGGRGGG |
| NO: 76 | (UniProtKB) | 3/Bos taurus | EYMEQEEDWDRDLLLDPAWEKQQR |
| | | | KTFTAWCNSHLRKAGTQIENIEEDFR |
| | | | NGLKLMLLLEVISGERLPRPDKGKM |
| | | | RFHKIANVNKALDFIASKGVKLVSIG |
| | | | AEGEEVAGGGRWGWGRGTTICTSQ |
| | | | EQLIYQVETSAKEGLLLWCQRKTAP |
| | | | YRNVNVQNFHTSWKDGLALCALIHR |
| | | | HRPDLIDYAKLRKDDPIGNLNTAFEV |
| | | | AEKYLDIPKMLDAEDIVNTPKPDEK |
| | | | AIMTYVSCFYHAFAGAEQAETAANR |
| | | | ICKVLAVNQENEKLMEEYEKLASEL |
| | | | LEWIRRTVPWLENRVGEPSMSAMQR |
| | | | KLEDFRDYRRLHKPPRVQEKCQLEIN |
| | | | FNTLQTKLRLSHRPAFMPSEGKLVSD |
| | | | IANAWRGLEQAEKGYEDWLLSEIRR |
| | | | LQRLQHLAEKFQQKASLH |
| | | | EAWTRGKEDMLSQRDYETASLQEV |
| | | | RALLRRHEAFESDLAAHQDRVEHIA |
| | | | ALAQELNELDYHEAASVNSRCQAIC |
| | | | DQWDNLGTLTQKRRDALERMEKLL |
| | | | ETIDQLQLEFARRAAPFNNWLDGAV |
| | | | EDLQDVWLVHSVEETQSLVTAHDQF |
| | | | KATLPEADRERGAILGIQGEIQKICQT |
| | | | YGLRPSSTNPYITLTPQDINTKWDTV |
| | | | RKLVPSRDQMLQEELTRQQVNERLR |
| | | | RQFAAQANAIGPWIQGKVEEVGRLA |
| | | | AGMAGSLEEQMAGLRQQEQNIINYK |
| | | | SNIDRLEGDHQLLQESLVFDNKHTV |
| | | | YSMEHIRVGWEQLLTSIARTINEVEN |
| | | | QVLTRDAKGLSQEQLNEFRASFNHF |
| | | | DRKRNGMMEPDDFRACLISMGYDL |
| | | | GEVEFARIMTMVDPNAAGVVTFQAF |
| | | | IDFMTRETAETDTAEQVVA |
| | | | SFKILAGDKNYITAEELRRELPAEQA |
| | | | EYCIRRMAPYKGAGAPAGALDYVAF |
| | | | SSALYGESDL |
| SEQ ID | Q3ZC55 | Alpha-actinin- | MNQIEPGVQYNYVYEDDEYMIQEEE |
| NO: 77 | (UniProtKB) | 2/Bos taurus | WDRDLLLDPAWEKQQRKTFTAWCN |
| | | | SHLRKAGTQIENIEEDFRNGLKLMLL |
| | | | LEVISGERLPKPDRGKMRFHKIANVN |
| | | | KALDYIASKGVKLVSIGAEEIVDGNV |
| | | | KMTLGMIWTIILRFAIQDISVEETSAK |
| | | | EGLLLWCQRKTAPYRNVNIQNFHTS |
| | | | WKDGLGLCALIHRHRPDLIDYSKLN |
| | | | KDDPIGNINLAMEIAEKHLDIPKMLD |
| | | | AEDIVNTPKPDERAIMTYVSCFYHAF |
| | | | AGAEQAETAANRICKVLAVNQENER |
| | | | LMEEYERLASELLEWIRRTIPWLENR |
| | | | TPEKTMQAMQKKLEDFRDYRRKHK |
| | | | PPKVQEKCQLEINFNTLQTKLRISNRP |
| | | | AFMPSEGKMVSDIAGAWQRLEQAE |
| | | | KGYEEWLLNEIRRLERVEHLAEKFR |
| | | | QKASTHETWAYGK |
| | | | EQILLQKDYESSTLTEVRALLRKHEA |
| | | | FESDLAAHQDRVEQIAAIAQELNELD |
| | | | YHDAVNVNDRCQKICDQWDRLGTL |
| | | | TQKRREALERTEKLLETIDQLHLEFA |
| | | | KRAAPFNNWMEGAMEDLQDMFIVH |
| | | | SIEEIQSLITAHEQFKATLPEADGERQ |
| | | | SILAIQNEVEKVIQSYSIRISSSNPYST |
| | | | VTVDEIRSKWDKVKQLVPIRDQSLQ |
| | | | EELARQHANERLRRQFAAQANAIGP |
| | | | WIQNKMEEIARSSIQITGALEDQMNQ |
| | | | LKQYEHNIINYKNNIDKLEGDHQLIQ |
| | | | EALVFDNKHTNYTMEHIRVGWELLL |
| | | | TTIARTINEVETQILTRDAKGITQEQM |
| | | | NEFRASFNHFDRRKNGLMDHEDFRA |
| | | | CLISMGYDLGEAEFARIMTLVDPNG |
| | | | QGTVTFQSFIDFMTRETADTDTAEQV |
| | | | IASFRILAS |
| | | | DKPYILAEELRRELPPDQAQYCIKRM |
| | | | PAYSGPGSVPGALDYTAFSSALYGES |
| | | | DL |
| SEQ ID | A0A1S3L6D5 | SALSA M- | MATKVMHFNQKKHVSHVSHHSSHT |
| NO: 78 | (UniProtKB) | protein, striated | TKHVVKEQSSRKSSSKSVNQVQHTH |
| | | muscle isoform | ATEAMAEPAYTVPAFRQRSADEIEE |
| | | X3/Salmo salar | YQRVSSHVGKGLASIQKELHRMRVV |
| | | | TKTQVENIAIKREVQEMMHKKMTLS |
| | | | TETDKAPDFMVALRPHTVWEKTPVK |
| | | | LFCTVDGHPRPIVKWYKGGKPVDPL |
| | | | SAPGKYKIESKYGVHSLIISRCMVSD |
| | | | TAEYSAVATNSHGSTTSKASVIVKRP |
| | | | AGGAYGSCQLFGQVPHLTVIHHSKL |
| | | | EITMLDRFGVSFGTEGGSISLVCTMV |
| | | | VVPDLPNVPPLAQWYRDDKLLKAG |
| | | | KLAEIKVGGGAATLTLPHLAKDDEG |
| | | | LYTLRMWTKDGTTEHSAYLFVKDA |
| | | | APSVAGAPGAPISVKAFDINSDYVLV |
| | | | AWKPPNTTNEAAITGYFVDKRESGS |
| | | | ATWSQCNDAPVQICKYPVH |
| | | | GLNVGHAYHFRVRAVNSAGISRPSR |
| | | | ESDKVTALDPAERERLQVIKLDGKH |
| | | | EVVIKDDDLEGVPSAPGQVVATRNT |
| | | | KSSVFVQWDPPKHPNHLMGYYIDAS |
| | | | LVGSKTWAPCNHKPYKNTRFVVHG |
| | | | LTPGETYVFRVQAVNVYGLSDESQE |
| | | | STPIAVEPALTTPSAPHGITMLSCDGA |
| | | | SMIIAWNSPKHRGGSKINAYYVDKR |
| | | | DADSLAWKEVNSAPTNTRTYTVDGL |
| | | | TEGTFYEFKVQAGNLAGVGVPSAPS |
| | | | TPLKCEAWTSAVPGPAYDLAFREVR |
| | | | GDSLVILWKAPVYTGASAVTGYIVE |
| | | | MAKKGHHYHPLNEEAIGHCYLQVT |
| | | | GLEAGAEYTFKVKAVNAEGVGKAS |
| | | | QTSEPVFAKALPGTQEIQCGVDEDTG |
| | | | DIFLSFEACEISETSNFAWSKNYKEIS |
| | | | DCTRVSIETKGKHSKLTF |
| | | | VNPDVSDLGAFSVHVTDTDGVSASH |
| | | | TVTEDALNTMLELSYNIRHPIVPLKH |
| | | | QLNYEVLEKGHVRFWLQCLKMSPA |
| | | | VNYRFIVNDKEVTSSDSHKISHDVNT |
| | | | GVIQMTVDHFSKASEATYTVQIHDG |
| | | | RAKNQSSLVLVGDVFKAALKEAEFQ |
| | | | RSEHIRKQGPHFAEYLSYHVDDDCT |
| | | | VLLVCKVANVKKETVFHWFKDEEEI |
| | | | VPETPPNVMSGSVALPISLFSRKDQG |
| | | | HYKAKLSDDRGKDTSVFDISGQVFD |
| | | | DIINAIAHIAGSSASELVLQCTPEGIRL |
| | | | QCYMKYYTEEIRTAWLHKDSKISSSE |
| | | | KMRIAGTAEMAWMQIREPSEKEKG |
| | | | HYSIQIMDATKSHTRTFDLSGQAYTD |
| | | | AYEEYLRLKAAAFAEKNRGRVVGG |
| | | | LPDVVTIMEQKTLSLTCTVWGDPSPE |
| | | | VTWFKNENEVVSTE |
| | | | HAKITLEANKFASLTITAVTSEDSGK |
| | | | YSINVRNKYGGEFVEITVSVYKQGES |
| | | | PPEPKMGGRGATPAPLASKTPAKTPT |
| | | | PAQTPTPSLKSPTPSLKSPTPSLRSPA |
| | | | KSPTPTPKSPTPPRRVKSPSPARSLKS |
| | | | PTPPRK |
| SEQ ID | P19352 | Tropomyosin | MEAIKKKMQMLKLDKENAIDRAEQ |
| NO: 79 | (UniProtKB) | beta | AEADKKQAEDRCKQLEEEQQGLQK |
| | | chain/Gallus | KLKGTEDEVEKYSESVKEAQEKLEQ |
| | | gallus | AEKKATDAEAEVASLNRRIQLVEEE |
| | | | LDRAQERLATALQKLEEAEKAADES |
| | | | ERGMKVIENRAMKDEEKMELQEMQ |
| | | | LKEAKHIAEEADRKYEEVARKLVVL |
| | | | EGELERSEERAEVAESKCGDLEEELK |
| | | | IVTNNLKSLEAQADKYSTKEDKYEE |
| | | | EIKLLGEKLKEAETRAEFAERSVAKL |
| | | | EKTIDDLEDEVYAQKMKYKAISEEL |
| | | | DNALNDITSL |
| SEQ ID | Q3ZC09 | Beta- | MAMQKIFAREILDSRGNPTVEVDLH |
| NO: 80 | (UniProtKB) | enolase/Bos | TAKGRFRAAVPSGASTGIYEALELRD |
| | | taurus | GDKSRYLGKGVLKAVEHINKTLGPA |
| | | | LLEKKLSVVDQEKVDKFMIELDGTE |
| | | | NKSKFGANAILGVSLAVCKAGAAEK |
| | | | GVPLYRHIADLAGNPELILPVPAFNVI |
| | | | NGGSHAGNKLAMQEFMILPVGASSF |
| | | | REAMRIGAEVYHHLKGVIKAKYGK |
| | | | DATNVGDEGGFAPNILENNEALELL |
| | | | KTAIQAAGYPDKVVIGMDVAASEFY |
| | | | RNGKYDLDFKSPDDPARHISGEKLG |
| | | | ELYKNFIKNYPVVSIEDPFDQDDWAT |
| | | | WTSFLSGVNIQIVGDDLTVTNPKRIA |
| | | | QAVEKKACNCLLLKVNQIGSVTESIQ |
| | | | ACKLAQSNGWGVMVSHRSGETEDT |
| | | | FIADLVVGLCTGQIKTGAPCRSERLA |
| | | | KYNQLMRIEEALGDKAVFAGRKFRN |
| | | | PKAK |
| SEQ ID | Q1AG05 | Calsarcin 3/Sus | MIPKEQKGPVVTAMGDLTEPAPLLD |
| NO: 81 | (UniProtKB) | scrofa | LGKKLSVPQDLMVEELSLPNNRGSL |
| | | | LFQERQRRVQKFTFEFAGSQRASAA |
| | | | GTAQGTVTATATPGRAANSPEGQNY |
| | | | SSELHVSLESPGGPEDVQPAAPRAVS |
| | | | APRPSALAPGYAEPLKGIPPEKFNHT |
| | | | AIPKGYRCPWQEFISYRDYLGEGRSH |
| | | | TPSLADYRNFNKTPVPFGGLLAGETL |
| | | | PRAGTPSVPELSSGLELLRLRPSFNRV |
| | | | AQGWVRNLPESEEL |
| SEQ ID | Q1AG02 | Calsarcin | MPLSGTPAPNKKRKSSKLIMELTGG |
| NO: 82 | (UniProtKB) | 2/Oryctolagus | GQESSGLNLGKKISVPRDVMLEELSL |
| | | cuniculus | LTNRGSKMFKLRQMRVEKFIYENHP |
| | | | DVFSDSSMDHFQKFLPTVGGQLGTA |
| | | | GQGVSYSKGSGGGQAGGSGSAGQY |
| | | | GSDHQHHHGSGSGAGGAGGPGGQA |
| | | | GKGGAAGAGGVGETGSGDQTGGDG |
| | | | KHITVFKTYISPWEQPWGVDPQQKV |
| | | | ELGIDLLAYGAKAELPKYKSFNRTA |
| | | | MPYGGYEKASKRMTFQMPKFDLGP |
| | | | LLSEPLVLYNQNLSNRPSFNRTPIPW |
| | | | LSSGEHADYHVDIGIPLDGETEEL |
| SEQ ID | A6QLT4 | Myotubularin | MASAPTSKYNSHSLENESIKRTSRDG |
| NO: 83 | (UniProtKB) | OS = Bos taurus | VNRDVGETLPRLPGEIRITDKEVIYIC |
| | | | PFNGPIKGRVYITNYRLYLRSLETDS |
| | | | ALILDVPLGVISRIEKMGGATSRGEN |
| | | | SYGLDITCKDLRNLRFALKQEGHSRR |
| | | | DMFEILTRYAFPLAHSLPIFAFLNEEK |
| | | | FNVDGWTVYNPVEEYRRQGLPNHH |
| | | | WRITFINKCYKLCDTYPALLVVPYRA |
| | | | SDEDLRRVATFRSRNRIPVLSWIHPE |
| | | | NKTVIVRCSQPLVGMSGKRNKEDER |
| | | | YLDVIRETNRQVNKLTIYDARPNVN |
| | | | AVANKATGGGYESDDVYHNAELFF |
| | | | LDIHNIHVMRESLKKVKDIVYPNVEE |
| | | | SHWLSSLESTHWLEHIKLVLTGAIQV |
| | | | ADRVSSGKSSVVVHCSDGWDRTAQ |
| | | | LTSLAMLMLDSFYRSIEGFEILVQKE |
| | | | WISFGHKFASRIGHGDKNHADADRS |
| | | | PIFLQFIDCVWQMSKQFPTAFEFNER |
| | | | FLITILDHLYSCRFGTFLYNCESAREK |
| | | | QKVTERTVSLWSLINSNKDKFKNPF |
| | | | YTKEINRVLYPVASMRHLELWVNYY |
| | | | IRWNPRIKQQQPNPVEQRYMELLAL |
| | | | RDEYIKRLDELQLANSAKLSDPSASP |
| | | | SSPSQMMPHVQTHF |
| SEQ ID | Q7YS81 | Myogenin/Bos | MELYETSPYFYQEPHFYDGENYLPV |
| NO: 84 | (UniProtKB) | taurus | HLQGFEPPGYERAELSLSPEARVPLE |
| | | | DKGLGPAEHCPGQCLPWACKVCKR |
| | | | KSVSVDRRRAATLREKRRLKKVNEA |
| | | | FEALKRSTLLNPNQRLPKVEILRSAIQ |
| | | | YIERLQALLSSLNQEERDLRYRGGGG |
| | | | PQAAVPSECSSHSASCSPQWGSALEF |
| | | | GPNPGDHLLPADPTDAHNLHSLTSIV |
| | | | DSITVEDVAAAFPDETIPN |
| SEQ ID | P49812 | Myogenin/Sus | MELYETSPYFYQEPHFYDGENYLPV |
| NO: 85 | (UniProtKB) | scrofa | HLQGFEPPGYERTELSLSPEARVPLE |
| | | | DKGLGTPEHCPGQCLPWACKVCKR |
| | | | KSVSVDRRRAATLREKRRLKKVNEA |
| | | | FEALKRSTLLNPNQRLPKVEILRSAIQ |
| | | | YIERLQALLSSLNQEERDLRYRGGGG |
| | | | PQPGVPSECSSHSASCSPEWGSALEF |
| | | | GPNPGDHLLTADPTDAHNLHSLTSIV |
| | | | DSITVEDVAVAFPDETMPN |
| SEQ ID | P31696-5 | Agrin Isoform | MGGSGAAATLALGLALGLALGGWA |
| NO: 86 | (UniProtKB) | 5/Gallus gallus | NCPERELQRREEEANVVLTGTVEEIM |
| | | | NVDPVHHTYSCKVRVWRYLKGKDI |
| | | | VTHEILLDGGNKVVIGGFGDPLICDN |
| | | | QVSTGDTRIFFVNPAPQYMWPAHRN |
| | | | ELMLNSSLMRITLRNLEEVEHCVEEH |
| | | | RKLLADKPNSYFTQTPPTPRDACRG |
| | | | MLCGFGAVCERSPTDPSQASCVCKK |
| | | | TACPVVVAPVCGSDYSTYSNECELE |
| | | | KAQCNQQRRIKVISKGPCGSKDPCAE |
| | | | VTCSFGSTCVRSADGQTAGCVCPAS |
| | | | CSGVAESIVCGSDGKDYRSECDLNK |
| | | | HACDKQENVFKKFDGACDPCKGILN |
| | | | DMNRVCRVNPRTRRVELLSRPENCP |
| | | | SKREPVCGDDGVTYASECVMGRTG |
| | | | AIRGLEIQKVRSGQCQHQDKCKDEC |
| | | | KFNAVCLKRWHARCSCDRITCDGTY |
| | | | RPVCARDSRTYSNDCERQKAECHQK |
| | | | AAIPVKHSGPCDLGTPSPCLSVECTF |
| | | | GATCVVKNREPVCECQQVCQGRYD |
| | | | PVCGSDNRTYGNPCELNAMACVLK |
| | | | REIRVKHKGPCDRCGKCQFGAICEAE |
| | | | TGRCVCPTECVPSSQPVCGTDGNTY |
| | | | GSECELHVRACTQQKNILVAAQGDC |
| | | | KSCGTTVCSFGSTCVGGQCVCPRCE |
| | | | QQPLAQVCGTDGLTYDNRCELRAAS |
| | | | CQQQKSIEVAKMGPCEDECGSGGSG |
| | | | SGDGSECEQDRCRHYGGWWDEDAE |
| | | | DDRCVCDFTCLAVPRSPVCGSDDVT |
| | | | YANECELKKTRCEKRQNLYVTSQGA |
| | | | CRALTTTPPPLPVVHCSQTIYGCCPD |
| | | | NMTLALGVGAAGCPSTCQCNPYGSY |
| | | | GGTCDPATGQCSCKPGVGGLKCDRC |
| | | | EPGFWNFRGIVTDSKSGCTPCNCDPV |
| | | | GSVRDDCEQMTGLCSCKTGITGMKC |
| | | | NQCPNGSKMGMAGCEKDPSAPKSC |
| | | | EEMSCEFGATCVEVNGFAHCECPSPL |
| | | | CSEANMTKVCGSDGVTYGDQCQLK |
| | | | TIACRQGQLITVKHVGQCHESITHTS |
| | | | HTMPPTPLPTLPLDKLIVPPPLQLTTQ |
| | | | APEPTELATTSLLMEASPTTRSHPTTR |
| | | | RVTTTRPVTTPWMTHGVLKTTVRPL |
| | | | STSPVVLATTQPPYAESGSAEGSGDQ |
| | | | EMSISGDQESSGAGSAGEEEVEESQV |
| | | | TPTPAIERATCYNTPLGCCSDGKTAA |
| | | | ADAEGSNCPATKVFQGVLILEEVEG |
| | | | QELFYTPEMADPKSELFGETARSIES |
| | | | ALDELFRNSDVKNDFKSIRVRDLGQS |
| | | | SAVRVIVESHFDPATSYTAADVQAA |
| | | | SLKQIRASKKRTILVKKPQQEHVKFM |
| | | | DFDWIPRIFTTTITTTTATTMAPATTR |
| | | | RHTTASAATTAHILRQDTVGHPSAK |
| | | | LAAPASTRRPTSTLPTTARRKPTRQP |
| | | | PSTTKKPSRPCDSHPCLHGGTCEDDG |
| | | | REFTCRCPAGKGGAVCEKPIRYFIPSF |
| | | | GGKSYLAFKMMKAYHTVRIAMEFR |
| | | | ATELSGLLLYNGQNRGKDFISLALVG |
| | | | GFVELRFNTGSGTGVITSKVRVEPGK |
| | | | WHQLVVNRNRRSGMLAVDGEHVSG |
| | | | ESPTGTDGLNLDTDLFVGGAPEDQM |
| | | | AVVAERTAATVGLKGSIRLLDVNNQ |
| | | | MYDLREKGSDVLYGSGVGECGNDP |
| | | | CHPNPCHHGASCHVKEAEMFHCECL |
| | | | HSYTGPTCADERNPCDPTPCHISATC |
| | | | LVLPEGGAMCACPMGREGEFCERVT |
| | | | EQDHTMPFLPEFNGFSYLELNGLQTL |
| | | | FLTCRQMSMEVVFLAKSPSGMIFYN |
| | | | GQKTDGKGDFVSLALHDGYLEYRY |
| | | | DLGKGAAVLRSKEPVPLNTWISVLL |
| | | | ERSGRKGVMRINNGERVMGESPVPH |
| | | | AFLNLKEPFYVGGAPDFSKLARAAAI |
| | | | STSFYGAVQRISIKGVPLLKEQHIRSA |
| | | | VEISTFRAHPCTQKPNPCQNGGTCSP |
| | | | RLESYECACQRGFSGAHCEKVIIEKA |
| | | | AGDAEAIAFDGRTYMEYHNAVTKSE |
| | | | KALQSNHFELSIKTEATQGLILWSGK |
| | | | GLERSDYIALAIVDGFVQMMYDLGS |
| | | | KPVVLRSTVPINTNHWTHIKAYRVQ |
| | | | REGSLQVGNEAPITGSSPLGATQLDT |
| | | | DGALWLGGMERLSVAHKLPKAYST |
| | | | GFIGCIRDVIVDRQELHLVEDALNNP |
| | | | TILHCSAK |
| SEQ ID | NP_001004406.1 | Cofilin-2/Gallus | MASGVTVNDEVIKVFNDMKVRKSST |
| NO: 87 | (NCBI) | gallus | PEEIKKRKKAVLFCLSDDKKQIIVEE |
| | | | ATRILVGDIGDTVEDPYTAFVKLLPL |
| | | | NDCRYALYDATYETKESKKEDLVFI |
| | | | FWAPESAPLKSKMIYASSKDAIKKKF |
| | | | TGIKHEWQVNGLDDIKDRSTLGEKL |
| | | | GGNVVVSLEGKPL |
| SEQ ID | P21566 | Cofilin-2/Gallus | MASGVTVNDEVIKVFNDMKVRKSST |
| NO: 88 | (UniProtKB) | gallus | PEEIKKRKKAVLFCLSDDKKQIIVEE |
| | | | AKQILVGDIGDTVEDPYTAFVKLLPL |
| | | | NDCRYALYDATYETKESKKEDLVFI |
| | | | FWAPESAPLKSKMIYASSKDAIKKKF |
| | | | TGIKHEWQVNGLDDIKDRSTLGEKL |
| | | | GGNVVVSLEGKPL |
| SEQ ID | Q679P3 | PDZ and LIM | MESYKVMLNGPAPWGFRLQGGKDF |
| NO: 89 | (UniProtKB) | domain protein | SMPLSISRLTPGGKAAQAGVGVGDW |
| | | 7/Gallus gallus | VLYIDGESTGTMTHIEAQNRIRACGD |
| | | | RLCLTLSRAQNHLGKPQKDSLPCSEP |
| | | | PKYNFAPSTALNKTARPFGASSPPNP |
| | | | RPGLVTKPVTYVPLAPACTPQHNGQ |
| | | | VSVPDPSPGAAMKTEPGLAPRTPAA |
| | | | TPGPTSRPPWAVDPSFAERYAPDKTS |
| | | | TVLSKHSQPATPTPMQNRSSIVQAAQ |
| | | | QAPESPGRTPLCYKCNKIIRGRYLVA |
| | | | LGHYYHPEEFTCCQCRKVLDEGGFF |
| | | | EEKGSIFCPKCYDTRYAPSCAKCKKK |
| | | | ITGEVMHALKMTWHVQCFTCAACK |
| | | | TPIRNRAFYMEEGQPYCERDYEKMF |
| | | | GTKCRGCDFKIDAGDRFLEALGFSW |
| | | | HDTCFVCAICQTNLEGKTFYSKKDK |
| | | | PLCKSHAFSHV |
| SEQ ID | F1NQD9 | Radixin/Gallus | MPKPINVRVTTMDAELEFAIQPNTTG |
| NO: 90 | (UniProtKB) | gallus | KQLFDQVVKTVGLREVWFFGLQYV |
| | | | DSKGYSTWLKLNKKVTQQDVRKEN |
| | | | PLQFKFRAKFFPEDVSEELIQEITQRL |
| | | | FFLQVKEAILNDEIYCPPETAVLLASY |
| | | | AVQSKYGDYNKEIHKLGYLANDRLL |
| | | | PQRVLEQHKLTKEQWEERIQNWHEE |
| | | | HRGMLREDSMMEYLKIAQDLEMYG |
| | | | VNYFEIKNKKGTELWLGVDALGLNI |
| | | | YEHDDKLTPKIGFPWSEIRNISFNDK |
| | | | KFVIKPIDKKAPDFVFYAPRLRINKRI |
| | | | LALCMGNHELYMRRRKPDTIEVQQ |
| | | | MKAQAREEKHQKQLERAQLENEKK |
| | | | KREIAEKEKERIEREKEELMERLRQIE |
| | | | EQTMKAQKELEEQTRRALELDQERK |
| | | | RAKEEAERLEKERRAAEEAKAALAK |
| | | | QAADQMKNQEQLAAELAEFTAKIAL |
| | | | LEEAKKKKEEEASEWQHKAFAAQE |
| | | | DLEKTKEELKSVMSAPPPPPPPPVIPP |
| | | | TENEHDEHDENNAEASAELSSDGVM |
| | | | NHRSEEERVTETQKNERVKKQLQAL |
| | | | SSELAQARDETKKTQNDVLHAENVK |
| | | | AGRDKYKTLRQIRQGNTKQRIDEFE |
| | | | AM |
| SEQ ID | XP_025008315.1 | nebulin isoform | MEEEEYEEVVEYYIEETIVEEGEPYE |
| NO: 91 | (NCBI) | X22/Gallus | VVTEITDSTSTEFTGPTTITRTIEYEKT |
| | | gallus | SGEGAATPVRKKTIRTKMDTSKFLTP |
| | | | YLQHSNKMKDLFSENKYKEKFNKER |
| | | | GKPYASTIDTPEIRRIKKVQEQLSEVK |
| | | | YRMAGEAARTICHVDEKAWDIEHA |
| | | | KKVSQQVSKVLYKQNWEENKDKYL |
| | | | LPPDAPELVNAIKNTAMFSKKLYTED |
| | | | WEGDKTLFYPYNDSPELRRVAQAQK |
| | | | ALSDIVYKKGHDERKSKYTSLPDPPD |
| | | | VEQAKKVTRQLSDIIYHDDYKNKIK |
| | | | GKWSQTPCYDVVIAKMNAENLSMK |
| | | | KYQEDFENVKDQIYFMQTETPEYEA |
| | | | NKRVSDNVSKIKYRADYEKNKAIAD |
| | | | YNVLPATENPLLRQLKTAGDVLSDK |
| | | | LYKEAYERSKGTSMNYCETPKFQTD |
| | | | NALKNFSDVKYKDAYQKNILGHYL |
| | | | GSFEDPHQIHCMKVEAMKSDKNYK |
| | | | ADYEEEKTKCYFPQTITQEYEAIKKL |
| | | | EQCKDHTYKKHPDQIKFTPVTDSPV |
| | | | QKQAEINSKQLSDKLYRSSGEEVKH |
| | | | KYTLPPDVPQFIQARYNAANVSDAY |
| | | | YKQDYHDLIAKGNNVSLDAIPITRAK |
| | | | ASRNIASDYKYKEAYEKAKGQQVGF |
| | | | KSLQDDPKLVHYMHVAKIQSDREYK |
| | | | KDYEKSKTNYHTPPDTFSIQAAKKSQ |
| | | | DVASTAHYKNLIHHYTYLPDAMDVE |
| | | | LAKNMMQIQSDNVYKQDYNSWFKG |
| | | | IGWSPLGSLDVEKAKKAGDALNEKK |
| | | | YRQHPDTIKFTSVPDSMTMVLAQHN |
| | | | TKQLSDVAYKQEGEKVKHKYKLDP |
| | | | DVPQFIQARVNAFNLSDANYKADW |
| | | | KKTIAKGYDLKPDAIPIIAAKASRNIA |
| | | | SDYKYKESYEKDKGRQVGYRSLQD |
| | | | DPKLVHYMHVAKMQSDREYKKDYE |
| | | | VTKTKYHTPLDMFSVTAAKKAQEA |
| | | | VTNTGYKQLIHHYTLLPDSVNLELSR |
| | | | NMMQLQSDNMYKADFNNWLRGVG |
| | | | WLPIQSLEVEKAKKASEILSEKKYRQ |
| | | | HPDKLKYSIPLDAMEQVLAKQNAKT |
| | | | MNKRLYTDKWNKEKTSIHVMPDTP |
| | | | EILQSRVNQITMSNKLYKAGWEEDK |
| | | | KKGYDMRPDAIPIKAAKTSQDIASDY |
| | | | KYKLAHEKAKGKHIGFRSLEDDPKL |
| | | | VHFMQVAKMQSDREYKKDYEKAKT |
| | | | NFHTPVDMLSVVAAKKAQEVATNA |
| | | | NYKNLIHVYNVLPDAMSLELAKNM |
| | | | MQIQSNNQYRAEYDESMKGVGWMP |
| | | | LGSLEAEKNKKAMEILSEKKYRQHP |
| | | | DKLKYSVPVDSMNMALALHNAKIM |
| | | | DEHQYKQAWEEDKKKVHMTPDIPQ |
| | | | FALAKANAFNISDKMYRHSFEEARK |
| | | | KGYDLRSDAIPIKAAKASRDIASDYK |
| | | | YKLGYEQDKGKLVGFRSLQDDPKLV |
| | | | HYMQVAKMQSDREYKKAYETSKTH |
| | | | YQTPSDALSIMAAKEAQDRVTNANY |
| | | | KRLIHHYMLLPDAMSFELYRNMNQI |
| | | | QSNNEYKQDYNEWFKGIGWSPAGSL |
| | | | DVEKSKKATEIASDQKYRQHPSIFPF |
| | | | TKQIDAMDMVLAKHNADIMNKHAY |
| | | | TQAWEKDKTQVHVMPDTPDILQAK |
| | | | QNKANYSQKQYKLDWQEMIKKGYD |
| | | | LTPEAISVKAAKASRDIASDYKYKEG |
| | | | YRKQQGHHIGFRSLQDDPKMMWSM |
| | | | QVAKMQSEREYKKDFEKWKTKFNM |
| | | | PVDMLGFLLAKKCQELVSDIDYKHM |
| | | | LHRWTCLPDQNDVTQAKRVYELQS |
| | | | DNLYKSDLQWLRGIGWSPLGSLESE |
| | | | KNKKASEILSEKKYRQHPDTIKFTSIP |
| | | | DAMNIILAKSNAKNRSDILYREAWD |
| | | | KDKTQVHIMPDTPEILLAKSNLINTS |
| | | | DKHYKLGYEELRRKGYDLPPDAIPL |
| | | | KSAKASRDIASEYQYKTAYRKQLGH |
| | | | HVGARNIEDDPKMMWSMHVAKIQS |
| | | | DREYKKAFEKTKTHFSSPVDMLGIV |
| | | | LAKKCQELVSDVDYKHLLHRWTCL |
| | | | PDQNDVVQARKVYDLQSDNVYKSD |
| | | | LQWLRGIGWSPLGSLDEEKNKRASM |
| | | | ILSDKKYRQHPDTIKFTSLPDSMPMV |
| | | | LAKHNSEIMNHRSYIAAWEKDKTSI |
| | | | HIMPDTPGILLAQQNKVNYSEKMYR |
| | | | LAMEEDKKKGYDLRADAIPIKAAKA |
| | | | SRDIASDYKYKEGYRKQLGHHIGAR |
| | | | NIEDDPKMMWSMHVAKVQSDREYK |
| | | | KAFEKTKTHFSSPVDMLGIVLAKKC |
| | | | QELVSDVDYKHLLHRWTCLPDQND |
| | | | VVQARKVYDLQSDNVYKSDLQWLR |
| | | | GIGWSPLGSLDEEKNKRASMILSDKK |
| | | | YRQHPDTIKFTSLPDSMPMVLAKHN |
| | | | SEIMNHRSYIAAWEKDKTSIHIMPDT |
| | | | PGILLAQQNKVNYSEKMYRLAMEED |
| | | | KKKGYDLRADAIPIKAAKASRDIASD |
| | | | YKYKEGYRKQLGHHIGARNIEDDPK |
| | | | MMWSMHVAKVQSDREYKKAFEKT |
| | | | KTHFSSPVDMLGIVLAKKCQELVSD |
| | | | VDYKHLLHRWTCLPDQNDVVQARK |
| | | | VYDLQSDNVYKSDLQWLRGIGWSPL |
| | | | GSLDEEKNKRASMILSDKKYRQHPD |
| | | | TIKFTSLPDSMPMVLAKHNSEIMNHR |
| | | | SYIAAWEKDKTSIHIMPDTPGILLAQ |
| | | | QNKVNYSEKMYRLAMEEDKKKGY |
| | | | DLRADAIPIKAAKASRDIASDYKYKE |
| | | | GYRKQLGHHIGARNIEDDPKMMWS |
| | | | MHVAKVQSDREYKKAFEKTKTHFSS |
| | | | PVDMLGIVLAKKCQELVSDVDYKHL |
| | | | LHRWTCLPDQNDVVQARKVYDLQS |
| | | | DNVYKSDLQWLRGIGWSPLGSLDEE |
| | | | KNKRASMILSDKKYRQHPDTIKFTSL |
| | | | PDSMPMVLAKHNSEIMNHRSYIAAW |
| | | | EKDKTSIHIMPDTPGILLAQQNKVNY |
| | | | SEKMYRLAMEEDKKKGYDLRADAI |
| | | | PIKAAKASRDIASDYKYKEGYRKQL |
| | | | GHHIGARNIEDDPKMMWSMHVAKI |
| | | | QSDREYKKAFEKTKTHFSSPVDMLGI |
| | | | VLAKKCQELVSDVDYRHYLHQWIC |
| | | | LPDQNDVIHARKAYDLQSDNFYKSD |
| | | | LEWMRGIGWVPIGSLDIEKAKRAGQI |
| | | | LSDKVYRQPPDTIKFTSVTDSLEMTL |
| | | | AKHNAEMMNKRLYTEAWDKDKTQI |
| | | | HIMPDTPEITLAKQNMHNYSEKLYK |
| | | | QAMEEAKKKGYDLRSDAIPIQAAKA |
| | | | SRQIASDYKYKEGYRKQLGHHIGAR |
| | | | NIEDDPKMMWSMHVAKIQSDREYK |
| | | | KAFEKTKTHFSSPVDMLGIVLAKKC |
| | | | QELVSDVDYRHYLHQWICLPDQND |
| | | | VIHARKAYDLQSDNFYKSDLEWMR |
| | | | GIGWVPIGSLDVEKAKRAGQILSDKV |
| | | | YRQPPDTIKFTSVTDSLEMTLAKHNA |
| | | | ETMNKRLYTEAWNKDKTTIHVMPD |
| | | | TPEILLAKQNQAHYSQKMYKLALEE |
| | | | SKKKGHDLRFDAIPIQAAKASREIAS |
| | | | DYKYKEGYRKQLGHHIGARNIEDDP |
| | | | KMMWSMHVAKIQSDREYKKAFEKT |
| | | | KTHFSSPVDMLGIVLAKKCQELVSD |
| | | | VDYRHYLHQWICLPDQNDVIHARKA |
| | | | YDLQSDAVYKSDLEWLKGIGWVPIG |
| | | | SLDVEKAKKAGEILSDRKYRQPADQI |
| | | | KFTSVTDSLAMMLAKHNAEIMNKRL |
| | | | YTEAWDADKTSIHVMPDTPTILLAK |
| | | | ANAANVSHKHYVQAWEDAKKKGY |
| | | | DMRADAIPIRSAKASRDIASDYKYKE |
| | | | AHEKQKGHYIGCRTAKEDPKLSWA |
| | | | ARAMLLQNDRIYRKAYNDSKAHIH |
| | | | MPVDAMSLQAAKECQTLVSDVDYR |
| | | | HYLHQWTCLPDQNDVMHARKAYD |
| | | | LQSDNVYKSDLEWLRGIGWLTEGSV |
| | | | DVIKAKKAQEILSDRLYRTQPDKMK |
| | | | FTSITDTPDVVQAKINAMQLSNHLYR |
| | | | EVWDKDKTQISIPSDTPELLQSKLNA |
| | | | LNISNKHYQKAWDEAKAKNYDLRA |
| | | | DAIPIKHAKASRDIASEYKYKEAHEK |
| | | | QKGHYIGCRTAKEDPKLSWAARAM |
| | | | LLQNDRIYRKAYNDSKAHIHMPVDA |
| | | | MSLQAAKECQTLVSDVDYRHYLHQ |
| | | | WTCLPDQNDVMHARKAYDLQSDNV |
| | | | YKSDLEWLRGIGWLTEGSVDVVKA |
| | | | KKAQEILSDRLYRTQPDKMKFTSVT |
| | | | DAPDVVQAKINAMQLSNRLYREAW |
| | | | DKDKTQISIPSDTPEMLQSKVNALNIS |
| | | | NKHYQKAWDEAKAKNYDLRADAIP |
| | | | IKHAKASRDIASEYKYKEAHEKQKG |
| | | | HYIGCRTAKEDPKLSWAARAMLLQ |
| | | | NDRIYRKAYNDSKAHIHMPVDAMSL |
| | | | QAAKECQTLVSDVDYRHYLHQWTC |
| | | | LPDHNDVVHARKAYDLQSDAVYKS |
| | | | DLEWLRGIGWLPNDSPGVQRVKHA |
| | | | QDLLSDKVYRTPIDSVKYTSVVDSPD |
| | | | ILLAKMNAEQLSIPKYKEAWEKDKT |
| | | | MIHIMPDTPEITLARSNAHNYSQKLY |
| | | | KEAWDEVKMSYDLRADAIPIKAAKA |
| | | | SREIASDYKYKLDHEKQKGHYVGVP |
| | | | NAKADTKIRFALGIGKVQSELEYKK |
| | | | HFAKWKTQCHLPVDMLSIQSAKHG |
| | | | QSLVSDVDYRHYLHQWICLPDQNDV |
| | | | IHARKAYDLQSDAVYKSDLEWLRGI |
| | | | GWLPNDSLGINHVKHAGDLLNERKY |
| | | | RTKAETLHFTPVADRVDYVTAKKSG |
| | | | EILSDIKYHKDWNEVKSNYTLTDTPQ |
| | | | LDMAREAARILNQSLYKESWEKEKA |
| | | | TGYLLPPDTVQIRHAKHSNDVQSEL |
| | | | KYKADYVKQRGHYVGVASMRDDP |
| | | | KLVWFEHAGEIQNDRLYKSNYHKTK |
| | | | SKIHIPADIMSVVAAKECQALVSDVD |
| | | | YRHYLHQWTCHPDQNDCIQARKAY |
| | | | DLQSDNIYKSDLEWLRGCGWIPLGS |
| | | | VEHKKVKHAQELINKRAYTKDAIEN |
| | | | FSKYTSVVDTPDIVLAKINSVNQSDL |
| | | | KYKETFNLEKGQYIGSDDTPELNHA |
| | | | RDMSLLYSDKLYKRDWEVCKPIGYT |
| | | | LDAKYIPLVGAKHANYVNSELKYKE |
| | | | IYEKLKGHYLAGKDIGDFPSVVHSLA |
| | | | FQKIRSALAYRKNYEDTKTRVHIPSD |
| | | | MMNHVLAKKCQYILSDLEYRTYLH |
| | | | HWNCSPEEHDVIQARRAQEILSDVV |
| | | | YKDDLNWLKGIGCYVWDTPQILHA |
| | | | KKSYDLQSQIKYTAAGKENFQNFGV |
| | | | VTDTPVYVTAVQSGINASDVKYKED |
| | | | YHKTKDKYTTVTETADSERVQNLKH |
| | | | LFSNNLYKEAWDRVKATSYIMPSDA |
| | | | VSLARAKELKHNASIVKYREEYDKF |
| | | | KALYTLPRSVEDDPNTARCLRVGKL |
| | | | NIDRLYKETYEKNKAKVHIIPDMVDI |
| | | | IAAKDAQKKISEIDYRTHLHDWICLP |
| | | | DLQINAHVRKVADQISDVVYKDDLN |
| | | | WLKGIGCFVWDTPEILHAKHAYDLR |
| | | | SDIKYKSDADKMKSKYTVVMDTPV |
| | | | YVQNILSGLNASEVIYRGDYLKKVR |
| | | | GKMIPTDKTVDLQRAHHANKIQSEN |
| | | | LYRWAGLKALPTGYSLPKDTPGFQH |
| | | | AKHVQHIGSDLKYKEAYEHMKAKG |
| | | | YTLGPNDVGFENVKKVNQVINERLY |
| | | | RATYHKNKDKIHTTPDTPEIRQVRAT |
| | | | QEAVSDLIYKSDFFKLQGHLISLPFTP |
| | | | QVLHCRYVGDITSDNKYKEDLKWL |
| | | | QGLGCFLYDTPDMVRARQLRKLWS |
| | | | NYVYTDSAKKMRDKYSVVLDTPGY |
| | | | RTVQELKTHLSDLVYRAAGKELKTK |
| | | | YTSVLNTPDFLRAKEGQRIQSQYLYV |
| | | | ALATKERPHHHAGNQTPAFTHARHV |
| | | | KDMVSETKYKIQYEKMKDKYTPVL |
| | | | DTPILIRAKRAYLNASDLRYKETFEN |
| | | | TKGKYHTVKDALDIVYHRKVTDDIS |
| | | | SVKYKENYMSQLGIWRSIPDRPEHFH |
| | | | HRAVTDAISDVKYKEDLSWLRGIGC |
| | | | YAWDTPDFALAEKNKVLYSGHKYK |
| | | | ETFEKTKSHFKYVADSPINRHFKHAT |
| | | | QLLDANSYKSLAKMLLKQGCNEILR |
| | | | PDILTALYNSYLWSQVEYKKDYEKK |
| | | | KDKYTTWDTPENIRTAKVNKQISDI |
| | | | IYKLEYNKAKPKGYTTIHDTPMLLH |
| | | | VRKVKDRISDLKYKELYERNKSHCN |
| | | | VVADSVHIKTPRHAYKLNSNLDYKK |
| | | | KYEAAKAHWHWIADRPDFVQAAKS |
| | | | SLQQSDYEYKLDREYLKGCKLSVTD |
| | | | DKNTVLALNNAILASDIKYKEKHNK |
| | | | ARGTCLVVPDTPQILLAKNVSSLVSE |
| | | | NKYKEHSKKQLPRGSYTTLPETRDT |
| | | | AHVKEVTKNVSDTNYKKKFVKEKG |
| | | | KSNYSIMLEPPEVKHAMEVAKKQST |
| | | | VEYKKDAKSKLHYTPIADRPDIKKA |
| | | | TQAAKLISDIEYKKRGEAGLGVTML |
| | | | GRPDIELAKEVSKLTSQVKYKENFSK |
| | | | EKGKKPKYDLKEAKIYKTMKDAHNI |
| | | | ASEVKYKADLKKLHKPVTDMSESLI |
| | | | MNHVLNTSQLASAVKYKEKYEKEK |
| | | | GKPMLDFETPTYLTAKESQLMQSEK |
| | | | EYRKDLEEGVKGKGLSVLEETPDML |
| | | | RAKNATQILNEKEYKKALELEIKGK |
| | | | GLSELALETPDFVRAKNATDIASQIK |
| | | | YKQLAEMEKANYTSVVDTPEIIHAQ |
| | | | QVKNLSSQKKYKEEAEKTMPYYVP |
| | | | VADTPEMQRVRENQKNFSTLQYQW |
| | | | DLQNSKGKVTVVQDTPEMLRVKEN |
| | | | QKNFSSIKYKESIGKGTPIPDLPEVKR |
| | | | VKETQKHISSVLYKEHLAKGTPTPM |
| | | | TPEMERAKRNQENISSVLYSDSFRKQ |
| | | | VQGKAAYVLDTPEMRRVRETQKHIS |
| | | | TVKYHEDFEKNKGTFTPVVTDPITER |
| | | | VKKNMHDFSDISYRGIQRRVVEMEQ |
| | | | KRVDQDQENLTGLRVWRTNPGSVF |
| | | | DYDPAEDNIQSRSLHMISVQAQRRSR |
| | | | EHSRSASALSISGGDEKSEPSEGVNQ |
| | | | HLSYYSSGGFFTTTATVGYKHAKTIE |
| | | | LPQQRSASVATQQTTVSSVPSHPSTA |
| | | | GKTYRAMYDYTAADADEVSFKDGD |
| | | | TIVNVQAIDEGWMYGTVQRTGKTG |
| | | | MLPANYVEAV |
| SEQ ID | XP_025008310.1 | Nebulin isoform | MEEEEYEEVVEYYIEETIVEEGEPYE |
| NO: 92 | (NCBI) | X17/Gallus | VVTEITDSTSTEFTGPTTITRTIEYEKT |
| | | gallus | SGEGAATPVRKKTIRTKMDTSKFLTP |
| | | | YLQHSNKMKDLFSENKYKEKFNKER |
| | | | GKPYASTIDTPEIRRIKKVQEQLSEVK |
| | | | YRMAGEAARTICHVDEKAWDIEHA |
| | | | KKVSQQVSKVLYKQNWEENKDKYL |
| | | | LPPDAPELVNAIKNTAMFSKKLYTED |
| | | | WEGDKTLFYPYNDSPELRRVAQAQK |
| | | | ALSDIVYKKGHDERKSKYTSLPDPPD |
| | | | VEQAKKVTRQLSDIIYHDDYKNKIK |
| | | | GKWSQTPCYDVVIAKMNAENLSMK |
| | | | KYQEDFENVKDQIYFMQTETPEYEA |
| | | | NKRVSDNVSKIKYRADYEKNKAIAD |
| | | | YNVLPATENPLLRQLKTAGDVLSDK |
| | | | LYKEAYERSKGTSMNYCETPKFQTD |
| | | | NALKNFSDVKYKDAYQKNILGHYL |
| | | | GSFEDPHQIHCMKVEAMKSDKNYK |
| | | | ADYEEEKTKCYFPQTITQEYEAIKKL |
| | | | EQCKDHTYKKHPDQIKFTPVTDSPV |
| | | | QKQAEINSKQLSDKLYRSSGEEVKH |
| | | | KYTLPPDVPQFIQARYNAANVSDAY |
| | | | YKQDYHDLIAKGNNVSLDAIPITRAK |
| | | | ASRNIASDYKYKEAYEKAKGQQVGF |
| | | | KSLQDDPKLVHYMHVAKIQSDREYK |
| | | | KDYEKSKTNYHTPPDTFSIQAAKKSQ |
| | | | DVASTAHYKNLIHHYTYLPDAMDVE |
| | | | LAKNMMQIQSDNVYKQDYNSWFKG |
| | | | IGWSPLGSLDVEKAKKAGDALNEKK |
| | | | YRQHPDTIKFTSVPDSMTMVLAQHN |
| | | | TKQLSDVAYKQEGEKVKHKYKLDP |
| | | | DVPQFIQARVNAFNLSDANYKADW |
| | | | KKTIAKGYDLKPDAIPIIAAKASRNIA |
| | | | SDYKYKESYEKDKGRQVGYRSLQD |
| | | | DPKLVHYMHVAKMQSDREYKKDYE |
| | | | VTKTKYHTPLDMFSVTAAKKAQEA |
| | | | VTNTGYKQLIHHYTLLPDSVNLELSR |
| | | | NMMQLQSDNMYKADFNNWLRGVG |
| | | | WLPIQSLEVEKAKKASEILSEKKYRQ |
| | | | HPDKLKYSIPLDAMEQVLAKQNAKT |
| | | | MNKRLYTDKWNKEKTSIHVMPDTP |
| | | | EILQSRVNQITMSNKLYKAGWEEDK |
| | | | KKGYDMRPDAIPIKAAKTSQDIASDY |
| | | | KYKLAHEKAKGKHIGFRSLEDDPKL |
| | | | VHFMQVAKMQSDREYKKDYEKAKT |
| | | | NFHTPVDMLSVVAAKKAQEVATNA |
| | | | NYKNLIHVYNVLPDAMSLELAKNM |
| | | | MQIQSNNQYRAEYDESMKGVGWMP |
| | | | LGSLEAEKNKKAMEILSEKKYRQHP |
| | | | DKLKYSVPVDSMNMALALHNAKIM |
| | | | DEHQYKQAWEEDKKKVHMTPDIPQ |
| | | | FALAKANAFNISDKMYRHSFEEARK |
| | | | KGYDLRSDAIPIKAAKASRDIASDYK |
| | | | YKLGYEQDKGKLVGFRSLQDDPKLV |
| | | | HYMQVAKMQSDREYKKAYETSKTH |
| | | | YQTPSDALSIMAAKEAQDRVTNANY |
| | | | KRLIHHYMLLPDAMSFELYRNMNQI |
| | | | QSNNEYKQDYNEWFKGIGWSPAGSL |
| | | | DVEKSKKATEIASDQKYRQHPSIFPF |
| | | | TKQIDAMDMVLAKHNADIMNKHAY |
| | | | TQAWEKDKTQVHVMPDTPDILQAK |
| | | | QNKANYSQKQYKLDWQEMIKKGYD |
| | | | LTPEAISVKAAKASRDIASDYKYKEG |
| | | | YRKQQGHHIGFRSLQDDPKMMWSM |
| | | | QVAKMQSEREYKKDFEKWKTKFNM |
| | | | PVDMLGFLLAKKCQELVSDIDYKHM |
| | | | LHRWTCLPDQNDVTQAKRVYELQS |
| | | | DNLYKSDLQWLRGIGWSPLGSLESE |
| | | | KNKKASEILSEKKYRQHPDTIKFTSIP |
| | | | DAMNIILAKSNAKNRSDILYREAWD |
| | | | KDKTQVHIMPDTPEILLAKSNLINTS |
| | | | DKHYKLGYEELRRKGYDLPPDAIPL |
| | | | KSAKASRDIASEYQYKTAYRKQLGH |
| | | | HVGARNIEDDPKMMWSMHVAKIQS |
| | | | DREYKKAFEKTKTHFSSPVDMLGIV |
| | | | LAKKCQELVSDVDYKHLLHRWTCL |
| | | | PDQNDVVQARKVYDLQSDNVYKSD |
| | | | LQWLRGIGWSPLGSLDEEKNKRASM |
| | | | ILSDKKYRQHPDTIKFTSLPDSMPMV |
| | | | LAKHNSEIMNHRSYIAAWEKDKTSI |
| | | | HIMPDTPGILLAQQNKVNYSEKMYR |
| | | | LAMEEDKKKGYDLRADAIPIKAAKA |
| | | | SRDIASDYKYKEGYRKQLGHHIGAR |
| | | | NIEDDPKMMWSMHVAKVQSDREYK |
| | | | KAFEKTKTHFSSPVDMLGIVLAKKC |
| | | | QELVSDVDYKHLLHRWTCLPDQND |
| | | | VVQARKVYDLQSDNVYKSDLQWLR |
| | | | GIGWSPLGSLDEEKNKRASMILSDKK |
| | | | YRQHPDTIKFTSLPDSMPMVLAKHN |
| | | | SEIMNHRSYIAAWEKDKTSIHIMPDT |
| | | | PGILLAQQNKVNYSEKMYRLAMEED |
| | | | KKKGYDLRADAIPIKAAKASRDIASD |
| | | | YKYKEGYRKQLGHHIGARNIEDDPK |
| | | | MMWSMHVAKVQSDREYKKAFEKT |
| | | | KTHFSSPVDMLGIVLAKKCQELVSD |
| | | | VDYKHLLHRWTCLPDQNDVVQARK |
| | | | VYDLQSDNVYKSDLQWLRGIGWSPL |
| | | | GSLDEEKNKRASMILSDKKYRQHPD |
| | | | TIKFTSLPDSMPMVLAKHNSEIMNHR |
| | | | SYIAAWEKDKTSIHIMPDTPGILLAQ |
| | | | QNKVNYSEKMYRLAMEEDKKKGY |
| | | | DLRADAIPIKAAKASRDIASDYKYKE |
| | | | GYRKQLGHHIGARNIEDDPKMMWS |
| | | | MHVAKVQSDREYKKAFEKTKTHFSS |
| | | | PVDMLGIVLAKKCQELVSDVDYKHL |
| | | | LHRWTCLPDQNDVVQARKVYDLQS |
| | | | DNVYKSDLQWLRGIGWSPLGSLDEE |
| | | | KNKRASMILSDKKYRQHPDTIKFTSL |
| | | | PDSMPMVLAKHNSEIMNHRSYIAAW |
| | | | EKDKTSIHIMPDTPGILLAQQNKVNY |
| | | | SEKMYRLAMEEDKKKGYDLRADAI |
| | | | PIKAAKASRDIASDYKYKEGYRKQL |
| | | | GHHIGARNIEDDPKMMWSMHVAKI |
| | | | QSDREYKKAFEKTKTHFSSPVDMLGI |
| | | | VLAKKCQELVSDVDYRHYLHQWIC |
| | | | LPDQNDVIHARKAYDLQSDNFYKSD |
| | | | LEWMRGIGWVPIGSLDVEKAKRAG |
| | | | QILSDKVYRQPPDTIKFTSVTDSLEM |
| | | | TLAKHNAETMNKRLYTEAWNKDKT |
| | | | TIHVMPDTPEILLAKQNQAHYSQKM |
| | | | YKLALEESKKKGHDLRFDAIPIQAAK |
| | | | ASREIASDYKYKEGYRKQLGHHIGA |
| | | | RNIEDDPKMMWSMHVAKIQSDREY |
| | | | KKAFEKTKTHFSSPVDMLGIVLAKK |
| | | | CQELVSDVDYRHYLHQWICLPDQND |
| | | | VIHARKAYDLQSDAVYKSDLEWLK |
| | | | GIGWVPIGSLDVEKAKKAGEILSDRK |
| | | | YRQPADQIKFTSVTDSLAMMLAKHN |
| | | | AEIMNKRLYTEAWDADKTSIHVMPD |
| | | | TPTILLAKANAANVSHKHYVQAWE |
| | | | DAKKKGYDMRADAIPIRSAKASRDI |
| | | | ASDYKYKEAHEKQKGHYIGCRTAKE |
| | | | DPKLSWAARAMLLQNDRIYRKAYN |
| | | | DSKAHIHMPVDAMSLQAAKECQTL |
| | | | VSDVDYRHYLHQWTCLPDQNDVMH |
| | | | ARKAYDLQSDNVYKSDLEWLRGIG |
| | | | WLTEGSVDVIKAKKAQEILSDRLYR |
| | | | TQPDKMKFTSITDTPDVVQAKINAM |
| | | | QLSNHLYREVWDKDKTQISIPSDTPE |
| | | | LLQSKLNALNISNKHYQKAWDEAK |
| | | | AKNYDLRADAIPIKHAKASRDIASEY |
| | | | KYKEAHEKQKGHYIGCRTAKEDPKL |
| | | | SWAARAMLLQNDRIYRKAYNDSKA |
| | | | HIHMPVDAMSLQAAKECQTLVSDV |
| | | | DYRHYLHQWTCLPDQNDVMHARK |
| | | | AYDLQSDNVYKSDLEWLRGIGWLTE |
| | | | GSVDVVKAKKAQEILSDRLYRTQPD |
| | | | KMKFTSVTDAPDVVQAKINAMQLS |
| | | | NRLYREAWDKDKTQISIPSDTPEMLQ |
| | | | SKVNALNISNKHYQKAWDEAKAKN |
| | | | YDLRADAIPIKHAKASRDIASEYKYK |
| | | | EAHEKQKGHYIGCRTAKEDPKLSWA |
| | | | ARAMLLQNDRIYRKAYNDSKAHIH |
| | | | MPVDAMSLQAAKECQTLVSDVDYR |
| | | | HYLHQWTCLPDHNDVVHARKAYDL |
| | | | QSDAVYKSDLEWLRGIGWLPNDSPG |
| | | | VQRVKHAQDLLSDKVYRTPIDSVKY |
| | | | TSVVDSPDILLAKMNAEQLSIPKYKE |
| | | | AWEKDKTMIHIMPDTPEITLARSNAH |
| | | | NYSQKLYKEAWDEVKMSYDLRADA |
| | | | IPIKAAKASREIASDYKYKLDHEKQK |
| | | | GHYVGVPNAKADTKIRFALGIGKVQ |
| | | | SELEYKKHFAKWKTQCHLPVDMLSI |
| | | | QSAKHGQSLVSDVDYRHYLHQWICL |
| | | | PDQNDVIHARKAYDLQSDAVYKSDL |
| | | | EWLRGIGWLPNDSLGINHVKHAGDL |
| | | | LNERKYRTKAETLHFTPVADRVDYV |
| | | | TAKKSGEILSDIKYHKDWNEVKSNY |
| | | | TLTDTPQLDMAREAARILNQSLYKES |
| | | | WEKEKATGYLLPPDTVQIRHAKHSN |
| | | | DVQSELKYKADYVKQRGHYVGVAS |
| | | | MRDDPKLVWFEHAGEIQNDRLYKS |
| | | | NYHKTKSKIHIPADIMSVVAAKECQ |
| | | | ALVSDVDYRHYLHQWTCHPDQNDC |
| | | | IQARKAYDLQSDNIYKSDLEWLRGC |
| | | | GWIPLGSVEHKKVKHAQELINKRAY |
| | | | TKDAIENFSKYTSVVDTPDIVLAKINS |
| | | | VNQSDLKYKETFNLEKGQYIGSDDT |
| | | | PELNHARDMSLLYSDKLYKRDWEV |
| | | | CKPIGYTLDAKYIPLVGAKHANYVN |
| | | | SELKYKEIYEKLKGHYLAGKDIGDFP |
| | | | SVVHSLAFQKIRSALAYRKNYEDTK |
| | | | TRVHIPSDMMNHVLAKKCQYILSDL |
| | | | EYRTYLHHWNCSPEEHDVIQARRAQ |
| | | | EILSDVVYKDDLNWLKGIGCYVWDT |
| | | | PQILHAKKSYDLQSQIKYTAAGKENF |
| | | | QNFGVVTDTPVYVTAVQSGINASDV |
| | | | KYKEDYHKTKDKYTTVTETADSERV |
| | | | QNLKHLFSNNLYKEAWDRVKATSYI |
| | | | MPSDAVSLARAKELKHNASIVKYRE |
| | | | EYDKFKALYTLPRSVEDDPNTARCL |
| | | | RVGKLNIDRLYKETYEKNKAKVHIIP |
| | | | DMVDIIAAKDAQKKISEIDYRTHLHD |
| | | | WICLPDLQINAHVRKVADQISDVVY |
| | | | KDDLNWLKGIGCFVWDTPEILHAKH |
| | | | AYDLRSDIKYKSDADKMKSKYTVV |
| | | | MDTPVYVQNILSGLNASEVIYRGDY |
| | | | LKKVRGKMIPTDKTVDLQRAHHAN |
| | | | KIQSENLYRWAGLKALPTGYSLPKD |
| | | | TPGFQHAKHVQHIGSDLKYKEAYEH |
| | | | MKAKGYTLGPNDVGFENVKKVNQV |
| | | | INERLYRATYHKNKDKIHTTPDTPEI |
| | | | RQVRATQEAVSDLIYKSDFFKLQGH |
| | | | LISLPFTPQVLHCRYVGDITSDNKYK |
| | | | EDLKWLQGLGCFLYDTPDMVRARQ |
| | | | LRKLWSNYVYTDSAKKMRDKYSVV |
| | | | LDTPGYRTVQELKTHLSDLVYRAAG |
| | | | KELKTKYTSVLNTPDFLRAKEGQRIQ |
| | | | SQYLYVALATKERPHHHAGNQTPAF |
| | | | THARHVKDMVSETKYKIQYEKMKD |
| | | | KYTPVLDTPILIRAKRAYLNASDLRY |
| | | | KETFENTKGKYHTVKDALDIVYHRK |
| | | | VTDDISSVKYKENYMSQLGIWRSIPD |
| | | | RPEHFHHRAVTDAISDVKYKEDLSW |
| | | | LRGIGCYAWDTPDFALAEKNKVLYS |
| | | | GHKYKETFEKTKSHFKYVADSPINR |
| | | | HFKHATQLLDANSYKSLAKMLLKQ |
| | | | GCNEILRPDILTALYNSYLWSQVEYK |
| | | | KDYEKKKDKYTTVVDTPENIRTAKV |
| | | | NKQISDIIYKLEYNKAKPKGYTTIHD |
| | | | TPMLLHVRKVKDRISDLKYKELYER |
| | | | NKSHCNVVADSVHIKTPRHAYKLNS |
| | | | NLDYKKKYEAAKAHWHWIADRPDF |
| | | | VQAAKSSLQQSDYEYKLDREYLKGC |
| | | | KLSVTDDKNTVLALNNAILASDIKY |
| | | | KEKHNKARGTCLVVPDTPQILLAKN |
| | | | VSSLVSENKYKEHSKKQLPRGSYTTL |
| | | | PETRDTAHVKEVTKNVSDTNYKKKF |
| | | | VKEKGKSNYSIMLEPPEVKHAMEVA |
| | | | KKQSTVEYKKDAKSKLHYTPIADRP |
| | | | DIKKATQAAKLISDIEYKKRGEAGLG |
| | | | VTMLGRPDIELAKEVSKLTSQAEYL |
| | | | KRHMRGHGAASYDTPWMRTFKKA |
| | | | NMLSSHVKYKENFSKEKGKKPKYDL |
| | | | KEAKIYKTMKDAHNIASEVKYKADL |
| | | | KKLHKPVTDMSESLIMNHVLNTSQL |
| | | | ASAYQYKKQYEKSKGHYHMVPDNL |
| | | | EQVHLREASELQSHVKYKEKYEKEK |
| | | | GKPMLDFETPTYLTAKESQLMQSEK |
| | | | EYKRDFEESIKGRNLTGLEITPSLLHV |
| | | | KYATKIASEKEYRKDLEEGVKGKGL |
| | | | SVLEETPDMLRAKNATQILNEKEYK |
| | | | KALELEIKGKGLSELALETPDFVRAK |
| | | | NATDIASQIKYKQLAEMEKANYTSV |
| | | | VDTPEIIHAQQVKNLSSQKKYKEEAE |
| | | | KTMPYYVPVADTPEMQRVRENQKN |
| | | | FSTLQYQWDLQNSKGKVTVVQDTPE |
| | | | MLRVKENQKNFSSILYKEDIGTGTTI |
| | | | EKTPEMQRVKRTQDAISSIKYKESIG |
| | | | KGTPIPDLPEVKRVKETQKHISSVLY |
| | | | KEDLGTGIPTPVTPEIERVKRNQEHIS |
| | | | SVLYKEELGTGTPIPVTPEVERVKRN |
| | | | QEHISSVLYKESVGTGTPTPITPEMER |
| | | | VKRNQEICSSVLYKENIGKATPTPVT |
| | | | PEMERVKRNQEIISSVLYKENVGKVT |
| | | | PTPITPEMERVKRNQEIISSVLYKESL |
| | | | GRATPTPITPEMERVKRNQEQISSVV |
| | | | YKEGLGKATPTPVTPEMERVRRNQE |
| | | | QISSVLYKEHLAKGTPTPMTPEMERA |
| | | | KRNQENISSVLYSDSFRKQVQGKAA |
| | | | YVLDTPEMRRVRETQKHISTVKYHE |
| | | | DFEKNKGTFTPVVTDPITERVKKNM |
| | | | HDFSDISYRGIQRRVVEMEQKRVDQ |
| | | | DQENLTGLRVWRTNPGSVFDYDPAE |
| | | | DNIQSRSLHMISVQAQRRSREHSRSA |
| | | | SALSISGGDEKSEPSEGVNQHLSYYS |
| | | | SGGFFTTTATVGYKHAKTIELPQQRS |
| | | | ASVATQQTTVSSVPSHPSTAGKTYR |
| | | | AMYDYTAADADEVSFKDGDTIVNV |
| | | | QAIDEGWMYGTVQRTGKTGMLPAN |
| | | | YVEAV |
| SEQ ID | F1MQI3 | Nebulin/Bos | MADDEEYEEVVEYYTEETVYEEVPG |
| NO: 93 | (UniProtKB) | taurus | ETRTRFYETTTTRTSDYEQSETSRPA |
| | | | LAQPVPEKPVERKRVIRKKVDPSKF |
| | | | MTPYIAHSQKMQNLFSTNKYKENYE |
| | | | KAKGKPYAITTDTPELRRIKKVQDQL |
| | | | SEVKYRVDGDVAKTICHVDEKAKDI |
| | | | EHAKKVSQQVSKVLYKQNWEDTKD |
| | | | KYLLPPDAPELVQAIKNTAMFSKKL |
| | | | YTEDWEADKTMFYPYNDSPELRRV |
| | | | AQAQKALSDIAYKKGLAEQQTQFTS |
| | | | LPDPPDIEFAKKVTNQVSKQKYKED |
| | | | YENKVKGKWSETPCFEIATARMNSN |
| | | | NISTKKYQEDFEHMKDQIYFMQTET |
| | | | PEYKVNKQAGVAASKVKYKQDYEK |
| | | | NKGKADYNVLPASENPLLRQLKAAG |
| | | | DALSDKLYKENYEKTKAKSINYCET |
| | | | PKFKLDTVLHNFSSDTKYKDSYLKDI |
| | | | LGHYVGSYEDPYHTHCMRVSAQNS |
| | | | DKNYKAEYEEDRGKGFFPQTITQEY |
| | | | EAIKKLDQCKDHTYKVHPDKTKFTQ |
| | | | VTDSPVLMQAQVNSKQLSDLNYKA |
| | | | KHENEKFKCHIPPDTPAFIQHKLNAY |
| | | | NLSDNIYKHDWEKTKAKKFDIKVDA |
| | | | IPLLAAKANTKIASDVMYKKDYEKS |
| | | | KGKMIGALSINDDPKMLHSLKTAKN |
| | | | QSDRLYKENYEKTKAKSMNYCETPK |
| | | | YQLDTLLKNFSEAKYKDSYVQNVLG |
| | | | HYIGSFEDPYQVHCLKISAQNSDKNY |
| | | | KAEYEEDKGKCYFPQTITQEYEAIKK |
| | | | LDQCKDHTYKVHPDKTKFTAVTDTP |
| | | | VLLQAQLNTKQLSDLNYKAKHEGE |
| | | | KFKCHIPADAPQFIQHRVNAYNLSDN |
| | | | IYKHDWEKTKAKKFDIKVDAIPLLA |
| | | | AKANTKIASDVMYKKDYEKSKGKM |
| | | | IGALSINDDPKMLHSLKTAKNQSDHE |
| | | | YRKDYEKSKTIYTAPLDMLPLTHAK |
| | | | KSQAIASDVDYKHLLHNYSYPPDSV |
| | | | NVDLAKKAYALQSDVEYKADYNSW |
| | | | MKGCGWMPFGSLEMEKAKRASDIL |
| | | | NEKKYRQHPDTLKFTSIEDAPIIVQSK |
| | | | INQAQRSDVAYKAKGEEVIHKYSLP |
| | | | ADLPQFIQAKVNAYNISENLYKADL |
| | | | KDLSKKGYDLRIDAIPIKAAKAARQA |
| | | | ASDVQYKKDYEKAKGKMVGFQSLQ |
| | | | DDPKLVHYMNVAKIQSDREYKKAY |
| | | | EKTKTRHNTPHDMVNIVAAKKAQD |
| | | | VASNVNYKHSLHHYTYLPDAMDLE |
| | | | LSKNMMHIQSDNVYKEDYNNWMK |
| | | | GIGWIPIGSLEVEKVKKAGDALNEKK |
| | | | YRQHPDTLKFTSIVDSPVMVQAKQN |
| | | | TQQVSDILYKAKGEDVKHKYTMSPD |
| | | | LPQFLQAKCNAYNLSDVCYKRDWH |
| | | | DLIAKGTNVLGDAIPITAAKASRNIAS |
| | | | DYKYKEAYEKAKGKQVGFRSLQDD |
| | | | PKLVHYMNVAKLQSDREYKKNYEN |
| | | | TKTSYHTPGDMVSITAAKMAQDVAT |
| | | | NVNYKQPIHHYTYLPDALSLEHIRNV |
| | | | NQIQSDNVYKDEYNHFFKGMGWIPI |
| | | | GSLEVEKVKKAGDALNEKKYRQHP |
| | | | DTIKFTSVPDSMGMVLAQHNTKQLS |
| | | | DLNYKVEGEKVKHKYTMDPDVPQFI |
| | | | QAKVNAYNMSDSHYKADWKKTLA |
| | | | KGYDLRPDAIPIVAAKSSRNIASDFK |
| | | | YKEAYEKTKGKQIGFRSLQDDPKLV |
| | | | HFMNVAKMQSDREYKKGYEASKTK |
| | | | YHTPLDMLSVTAAKKSQEVATNTNY |
| | | | KQPFHHYTLLPDALNVEHSRNAMQI |
| | | | QSDNLYKSDFTNWMKGIGWLPLESL |
| | | | EVEKAKKAGEILSEKKYRQHPEKLK |
| | | | FTYAMDTMEQALNKSNKLIMDKRL |
| | | | YTEKWNKDKTTIHVMPDTPDILLSR |
| | | | VNQITMSDKLYKAGWEEEKKKGYD |
| | | | LRPDAISIKAARASRDIASDYKYKQA |
| | | | YEQAKGKQIGFRSLEDDPKLVHFMQ |
| | | | VAKMQSDREYKKAYEKSKTSFQTPV |
| | | | DMLSVVAAKKSQEVATNANYRNVI |
| | | | HTYNMLPDAMSLELAKNMMQIQSD |
| | | | NQYKADYADFMKGIGWLPLGSLEA |
| | | | EKNKKAMEIISEKKYRQHPDTLKYST |
| | | | LMDSMNMVLAKNNAKIMNEHLYK |
| | | | QAWEADKTKVHIMPDIPQIILAKANA |
| | | | INISDKLYKLSLEEAKKKGYDLRTDA |
| | | | IPIKAAKASRDIASDYKYKHSYEKER |
| | | | GKMVGFRSLEDDPKLVHSMQVAKM |
| | | | QSDREYKKNYEKTKTSYHTPADMLS |
| | | | VTAAKDAQANITNTNYKHLIHKYILL |
| | | | PDAMNIQLSKNMNRIQSDNEYKQDY |
| | | | NEWYKGLGWSPAGSLEVEKAKKAT |
| | | | EYASDQKYRQHPSNFQFTKLNDSMD |
| | | | MVLAKQNAHTMNKYLYTVDWNKD |
| | | | KTKIHVMPDTPDILQAKQNQTMYSQ |
| | | | VKYKMGWPSGLERVVNLPSVLIKIF |
| | | | KSKQNKDLIWRFKYKQGYRKQIGHH |
| | | | IGFRSLQDDPKLVLSMNVAKMQSER |
| | | | EYKKDFEKWKTKFSSPVDMLGVVL |
| | | | AKKCQALVSDVDYKNYLHGWTCLP |
| | | | DQNDVIHAKKAYDLQSENLYKSDLE |
| | | | WLKGIGWSPLGSLEAEKNKRASEIIS |
| | | | EKKYRQPPDRNKFTSIPDAMDIVLAK |
| | | | TNAKNRSDILYREAWDKDKTQIHIM |
| | | | PDTPDIILAKANLINTSDKFYRMGYE |
| | | | ELRKKGYDLPVDAIPIKAAKASREIA |
| | | | SEYKYKEGFRMQLGHHIGARNIKDD |
| | | | PKMMWSMHVAKIQSDREYKKDFEK |
| | | | WKTKFSSPVDMLGVVLAKKCQTLV |
| | | | SDIDYKNYLHQWTCLPDQSDVIHAR |
| | | | RAYDLQSDNLYKSDLQWLRGIGWLP |
| | | | SGSLEDEKNKRASQILSDHVYRQHP |
| | | | DQFKFSSLMDSIPMVLAKNNAITMN |
| | | | HRLYTEAWDKDKTTVHIMPDTPEVL |
| | | | LAKQNQINYSEKLYKLGLDEAKRKG |
| | | | YDMRIDAIPIRAAKASRDIASEFKYK |
| | | | EGYRRQLGHHIGARAIHDDPKMMW |
| | | | SMHVAKIQSDREYKKDFEKWKTKFS |
| | | | SPVDMLGVVLAKKCQTLVSDIDYKN |
| | | | YLHQWTCLPDQSDVIHARQAYDLQS |
| | | | DNVYKSDLQWLRGIGWVPIGSLDVE |
| | | | KSKRASEILSDKLYRQPPDKFKFTSV |
| | | | TDSLEQVLAKNNAINMNKRLYTEA |
| | | | WDKDKTQVHIMPDTPEITLARTNKV |
| | | | NYSENLYKLAHEEAKKKGYDLRSD |
| | | | AIPIIAAGLEKDHISDYKYKDGYRKQ |
| | | | LGHHIGARDIKDDPKMMWSMHVAK |
| | | | IQSDREYKKDFEKWKTKFSSPVDML |
| | | | GVVLAKKCQTLVSDIDYKNYLHQW |
| | | | TCLPDQSDVIHARQAYDLQSDNVYK |
| | | | SDLQWLRGIGWVPIGSVDVVKCKRA |
| | | | AEILSDNLYRQPPDTLKFTSVPDSLE |
| | | | QVLAKNNAINMNKRLYTEAWDKDK |
| | | | TQIHIMPDTPEIMLARQNKLNYSETL |
| | | | YKLANEEAKKKGYDLRSDAIPIVAA |
| | | | KASRDIISDYKYKDGYRKQLGHHIG |
| | | | ARDIKDDPKMMWSMHVAKIQSDRE |
| | | | YKKDFEKWKTKFSSPVDMLGVVLA |
| | | | KKCQTLVSDIDYKNYLHQWTCLPDQ |
| | | | SDVIHARRAYDLQSDNIYKSDLQWL |
| | | | RGIGWVPIGSVDVVKCKRAAEILSDN |
| | | | LYRQRPDTLKFTSVPDSLEQVLAKN |
| | | | NAINMNKRLYTEAWDKDKTQIHMM |
| | | | PDTPEITLARQNKINYSENLYRQAME |
| | | | EAKKEGYDLRSDAIPIVAAKASREIA |
| | | | SDYKYKEAYRKQLGHHIGARAIHDD |
| | | | PKMMWSVHVAKMQSDREYKKDFE |
| | | | KYKTRFSSPVDMLGIVLAKKCQTLV |
| | | | SDVDYKHPLHEWTCLPDQNDIIHAR |
| | | | KAYDLQSDNLYKSDLEWLKGIGWV |
| | | | PIGSVEVLKAKRAGEILSDNIYRQRP |
| | | | DTLKFTSVTDSPEQVLAKNNAINMN |
| | | | KRLYTEAWDNDKKTIHVMPDTPEIM |
| | | | LAKLNRINYSDKLYKLALEESRKEG |
| | | | YDLRLDAIPIQAAKASREIASDYKYK |
| | | | EGYRKQLGHHIGARNIKDDPKMMW |
| | | | SIHAGKLQSDLEYKKDFEKWKTKFS |
| | | | SPVDMMGLVQAKKCQILVSDIDYKN |
| | | | LLHEWTCLPDQNDIIQARKAYDLQS |
| | | | DAIYKADLEWLRGIGWVPIDSVGVE |
| | | | HAKRAGEILSERKYRQPAVQLKFTSI |
| | | | TDTPEIVLAKNNALNVSKHLYTEAW |
| | | | DADKTSIHVMPDTPEILLAKSNSANIS |
| | | | HKLYTKGWDESKMKDYDLRADAISI |
| | | | KSAKASRDIASDYKYKEAYEKHKGH |
| | | | HIGARSVEDDPRIMCAMNAGRIQSER |
| | | | EYKKEFQKWKTKFSSPVDMLGILLA |
| | | | KKCQTLVSDIDYRNYLHHWTCLPDQ |
| | | | NDIIQARKAYDLQSDAIYKADLEWL |
| | | | RGIGWMPEGSPEVLRVKNAQHIFRD |
| | | | SVYRTPVVKLKYTSIVDTPEVVLAKS |
| | | | NAENISIPKYREVWDKDKTSIHIMPD |
| | | | TPEINLARTNALNVSNKLYREGWDE |
| | | | MKMSCDVRLDAIPIQAAKASREIASD |
| | | | YKYKLDHEKQKGHYVGTRTARDDN |
| | | | KIRWALIAGKIQNEREYRLHWAKWK |
| | | | SKFQSPPDMLSIEHSKNSQTLVSDIDY |
| | | | RHYLHQWTCMPDQNDVIQARKAYD |
| | | | LQSDAIYKADLEWLRGIGWMPADSV |
| | | | SVNHAKHMADIFNEKKYRTKIETLSF |
| | | | TPVDDRVDYVTAKHSGEILNDIKYR |
| | | | KDWNDTKSKYTLTETPQLHTAQEAA |
| | | | RILDQYLYKESWEKQKATGYILPPD |
| | | | AVPFVHAHHSGDVQSELKYKAEHV |
| | | | KQKGHYVGVPTMRDDPKLVWFEHA |
| | | | GQIQNDRLYKENYHKTKAKIHIPPD |
| | | | MVSVLAAKEGQALASDIDYRNYLH |
| | | | QWICHPDQNDVIQARKAYDLQSDNI |
| | | | YKADLEWLRGIGWIPLDSVDHVRVT |
| | | | RNQEMMNQIKYKKDALANYPNFTS |
| | | | VVDPPEIVLAKINSVNQSDVKYKETF |
| | | | NKLIKGKYIFSPDTPYITHSKDMEKL |
| | | | YSTILYKRAWDGTKAYGYTLDERYI |
| | | | PIVGAKHADFVNSELKYKETYEKLK |
| | | | GHYLAGKEISEFPNVVHCLDFQKMR |
| | | | SLLNYRRHYEDTKANVHIPQDMMN |
| | | | HVLAKRCQYILSDLEYRHYFHQWTS |
| | | | LPEEPNVVRVRHAQEILSDNVYKDD |
| | | | LNWLKGIGCYVWDTPQILHAKKSYD |
| | | | LQSQILYTAAGKENLKNYNLVTDTP |
| | | | LYVTALQSGINASEVKYKENYHQTK |
| | | | DKYTTVLETVDYDRIKNLKDLFSSNL |
| | | | YKEAWDKVKATSYILPPNTVSLTHA |
| | | | KNQKYMASHIKYREEYEKFKALYTL |
| | | | PRSVEDDPNTARCLRVGKFNIDRLYR |
| | | | SVYEKNKMKIHIVPDMVEMVTAKDS |
| | | | QKKVSEIDYRLHLHEWICHPDLQVN |
| | | | SHVRKVTDQISDIVYKDDLTWLKGI |
| | | | GCYVWDTPEILHAKHAYDLRNDIKY |
| | | | KAHVLKTRNNYKLVTDTPVYVQAV |
| | | | KSGKQLSDAVYHYDYVHSIRGRVAP |
| | | | TTKTVDLDRALHAYKLQSENLYRKA |
| | | | GLHALPTGYRLPVDTPHFKHTKDTR |
| | | | YMSSYFKYKEVYEHMKAYGYTLGP |
| | | | NDVPFVNVRRVNNITSERLYRQLYH |
| | | | KLKDKIHTTPDTPEIRQVKKTQEAVS |
| | | | ELIYKSDFFKMQGHMISLPYTPQVLH |
| | | | CRYVGDITSDIKYKEDLQVLRGMGC |
| | | | FLYDTPDMVRSRHLRKLWSHYLYTD |
| | | | KARKMRDKYKVVLDTPEYRKVQEL |
| | | | KTHLSELVYRAAGRKQKSIFTSVPDT |
| | | | PDLTRAKRGQKLQSQYLYVELATKE |
| | | | RPHHHAGNQTTALKHARHVKDMVS |
| | | | ENKYKIQYEKMKDKYTPVPDTPILIR |
| | | | AKRAYWNASDLRYKETFQKTKGKY |
| | | | HTVKDALDIVYHRTVTDHISKIKYKE |
| | | | NYMSQLGIWRSIPDRPEHFHHRAVT |
| | | | DAVSDVKYKQDLTWLKGIGCYAYD |
| | | | TPDFTLAEKNKTLYSKYKYKEVFER |
| | | | TKSNFKYVADCPINRHFKFATQLMN |
| | | | EKKYRADYEQRKDKYHLVVDEPRH |
| | | | LLAKIAGDQISQIKYRKNYEETKDKF |
| | | | TSIVDTPEHLRTTKVNKQISDILYKLE |
| | | | YNKAKPRGYTTIHDTPMLLHVRKVK |
| | | | DEVSDLKYKEVYQRTKSNCTIEPDA |
| | | | VHIKAAKDAYKVNTNLDYKKKYEA |
| | | | TKAYWKWTPDRPDFIQAAKSTLQQS |
| | | | DFEYKLDREYLKGCKLSVTDDKDM |
| | | | VLALKNSIIESDLKYKEKHVKERGSC |
| | | | HAVPDTPQILLAKTVSSLVSENKYKS |
| | | | YVKKHLAQGSYTTLPETRDTIHVKE |
| | | | VTKNVSDTNYKKKFVKEKGKSNYSI |
| | | | MLEPPDVKHAMDVAKKQSNVAYKK |
| | | | DAKENLHYTTVADRPDIKKATQAAK |
| | | | QASEVEYRAKHRKEGSHGLSMLGRP |
| | | | DIEMAKKAAKLSSQVQYRENFNKEK |
| | | | GKTPKYNPKDSQLYKVMKDANTLA |
| | | | SEVKYKADLKKLHKPVTDMKESLIM |
| | | | NHVLNTSHLASSYQYKKNYEKSKGH |
| | | | YHTIPDNLEQLHLKEATELQSIVKYK |
| | | | EKYEKERGKPMLDFETPTYITAKESQ |
| | | | QMQSGKEYRKDYEESIKGRNLTGLE |
| | | | VTPALLHVKYATKIASEKEYRKDLE |
| | | | ESIRGKGLSEMEDTPDMLRAKNATQI |
| | | | LNEKEYKRDLELEVKGRGLNAMAN |
| | | | ETPDFLRARNATDIASQIKYKQSAEM |
| | | | EKANFTSVVDTPEIIHAQQVKNLSSQ |
| | | | KKYKEDAEKCMSYYETVLDTPEMQ |
| | | | RVRENQKNFSLLQYQYDLKNSKGKI |
| | | | TVVQDTPEILRVKENQKNFSSVLYKE |
| | | | DVSPGTAIGKTPEMMRVKQTQDHIS |
| | | | SVKYKEVIGQGTPIPDLPEVKRVKQT |
| | | | QKHISSVMYKENLGTGIPTPVTPEIER |
| | | | VKRNQENFSSVLYKENLGKGTPTPIT |
| | | | PEMERVKRNQENFSSILYKENLSKGT |
| | | | PLPVTPEMERVKRNQENFSSVLYKE |
| | | | NVGKGTPTPVTPEMQRVKRNQENIS |
| | | | SVLYKENLGKATPTPFTPEMERVKR |
| | | | NQENFSSVLYKENMRKATPTPVTPE |
| | | | MERAKRNQENISSVLYSDSFRKQIQG |
| | | | KAAYVLDTPEMRRVRETQRHISTVK |
| | | | YHEDFEKHKGCFTPVVTDPITERVKK |
| | | | NTQDFSDICYRGIQRKVVEMEQKRN |
| | | | DQDQETITGLRVWRTNPGSVFDYDP |
| | | | AEDNIQSRSLHMINAQAQRRSREQSR |
| | | | SASGLSISGGEEKSEHSEAAHLSTYS |
| | | | DGGVFFSAASTGYKHARTTELPQQR |
| | | | SSSVATQQTTVSSIPSHPSTAGKIFRA |
| | | | MYDYMAADADEVSFKDGDAIVNVQ |
| | | | AIDEGWMYGTVQRTGRTGM |
| | | | LPANYVEAI |
| SEQ ID | F1MPU4 | Myotilin/Bos | MFNYERPKHFIQSQNPCGSRLQPPGP |
| NO: 94 | (UniProtKB) | taurus | EISSYSSQTKQSSITIQPRQCTEQRYS |
| | | | ASSTVSSHITMSSSAFPASPQQLAGSN |
| | | | PAQRVTATYNQSPASFLSSILPSQPDY |
| | | | SSSKNPSTVDSNYQQPSVGQPINVKS |
| | | | SQNANAKLTPKTPDHEIQGSKEALIQ |
| | | | DLERKLKCKDSLLHNGNQRLTYEEK |
| | | | MARRLLGPQNAAAVFQGQNDSEAQ |
| | | | DSAQQHNIEHARLQVPTSQVRSRSSS |
| | | | RGDVNDQDAIQEKFYPPRFIQVPEN |
| | | | MSIEEGRFCRMDFKVSGLPAPDVSW |
| | | | YLNGRPVQSDDFHKMIVSEKGFHSLI |
| | | | FEVVRASDAGAYACVAKNRAGEAT |
| | | | FTVQLDVLAKEHRRAPMFIYKPQSK |
| | | | KVFEGESVKLECQISAVPPPKLFWKR |
| | | | NNEMVQFNTDRISLYHDNSGRVTLLI |
| | | | KDVNKKDAGWYTVSAVNEAGVTTC |
| | | | NTRLDVTARPNQTLPAPKQLRVRPTF |
| | | | SKYLALNGKGLNVKQAFNPEGEFQR |
| | | | LAAQSGLYESEEL |
| SEQ ID | A0A1D5NT92 | Myotilin | MSVRNLPSVSSMCQPTMFNYERPKH |
| NO: 95 | (UniProtKB) | OS = Gallus | FIQSKNVCQGQQQPPGSSTSTESSRQI |
| | | gallus | KQSSILIQPRNPSGQKFSSSSSLSSSITL |
| | | | SSPSCSAPKESTYPVTPASAQSPASSS |
| | | | SGQRLISMPNQTPAAFLCSVLPSQPD |
| | | | YNSQTPPMEPHYSKPMYKKQASINS |
| | | | MQKTSDQEIRGTKEALIQDLEKKLRC |
| | | | KDNLLQNGNQRLTYEERMARRLLGP |
| | | | ENAASVLEAQSEDMQNTQNAENVR |
| | | | LQVPTTHVRSRPSSRGDERGHDSIQE |
| | | | KFFQPRFTQVPEDVIIEEGRFCRLDFK |
| | | | VSGLPTPDVMWYQNGRMVHQDQFH |
| | | | KMIVSEKGFHSFIFEAVKSSDAGTYE |
| | | | CVAVNRAGESSFAVKVEVIAKEHHT |
| | | | PPTFIFKPQSKKVFEGDTARLECQISA |
| | | | IPTPRIYWKRNNEMVQYNTDRISLLH |
| | | | DNTGRICLLIHNANKKDAGWYTVSA |
| | | | VNGAGVATCHARLEVATHTNKPVP |
| | | | APKQLRVRPTFSKYLALNGRGLDVK |
| | | | QAFSPEGEFQRLAEQSGLYESDEL |
| SEQ ID | F1RH92 | Myotilin/Sus | MFNYERPKHFIQSQNPCGSRLQPPGP |
| NO: 96 | (UniProtKB) | scrofa | ETSSYSSQTKQSSIIIQPRQCTEQRFSA |
| | | | SSTVSSHITMSSSAFPASPQQPAASNP |
| | | | GQRVTATYNQSPASFLSSILPSQPDYS |
| | | | SSKIPSTMDSNYQQPSVGQPVNAKPS |
| | | | QSLNAKPIPRTPDHEIQGSKEALIQDL |
| | | | ERKLKCKDSLLHNGNQRLTYEEKM |
| | | | ARRLLGPQNAAAVFQAQNDSAAQD |
| | | | SPQQHNSEHARLQVPTSQVRSRSSSR |
| | | | GDVNDQDAIQEKFYPPRFIQVPENMS |
| | | | VEEGRFCRMDFKVSGLPAPDVSWYL |
| | | | NGRPVQSDDLHKMIVSEKGFHSLIFE |
| | | | VVRASDAGAYACVAKNRAGEATFT |
| | | | VQLDVLAKEHRRAPMFIYKPQSKKV |
| | | | FEGDSVKLECQISAIPPPKLFWKRNN |
| | | | EMVQFNTDRISLYHDNSGRVTLLIKD |
| | | | VNKKDAGWYTVSAVNEAGVITCNT |
| | | | RLDVTARPNQTLPAPKQLRVRPTFSK |
| | | | YLALNGRGLDVKQAFNPEGEFQRLA |
| | | | AQSGLYESEEL |
| SEQ ID | XP_013837221.1 | Twinfilin-2/Sus | MAHQTGIHATEELKEFFAKARAGSV |
| NO: 97 | (NCBI) | scrofa | RLIKVVIEDEQLVLGASRELMGCWD |
| | | | QDYDRAVLPLLDAQQPCYLLYRLDT |
| | | | QNAQGFEWLFLAWSPDNSPVRLKM |
| | | | LYAATRATVKKEFGGGHIKDELFGT |
| | | | VKDDLSFAGYQKHLSSCAAPAPLTS |
| | | | AERELQQIRINEVKTEISVESKHQTLQ |
| | | | GLAFPLQPQAQRALQQLRQKMINYI |
| | | | QLKLDLERETIELVHTEPTDVAQLPS |
| | | | RVPRDAARYHFFLYKHTHEGDPLES |
| | | | VVFIYSMPGYKCSIKERMLYSSCKSR |
| | | | LLDSVEQDFQLEIAKKIEIGDGAELT |
| | | | AEFLYDEVHPKQHAFKQAFAKPKGP |
| | | | GGKRGHKRLIRGPGENGDDS |
| SEQ ID | XP_024844129.1 | Dystrophin | MLWWEEVEDCYEREDVQKKTFTK |
| NO: 98 | (NCBI) | isoform X4/Bos | WINAQFSKFGKQHIENLFSDLQDGRR |
| | | taurus | LLDLLEGLTGQKLPKEKGSTRVHAL |
| | | | NNVNKALQVLQKNNVDLVNIGSSDI |
| | | | VDGNHKLTLGLIWNIILHWQVKNVM |
| | | | KNIMAGLQQTNSEKILLSWVRQSTR |
| | | | NYPQVNVINFTTSWSDGLALNALIHS |
| | | | HRPDLFDWNSVVRQQSATQRLEHAF |
| | | | NIAKYQLGIEKLLDPEDVATTYPDKK |
| | | | SILMYVTSLFQVLPQQVSLEAIQEVE |
| | | | MLPRPSKVTREEHFQLHHQMHYSQQ |
| | | | ITVSLAQGYERTPSSPQPRFKSYAYT |
| | | | QAAYVSTSDPTRSPFPSQRLEAPEDK |
| | | | SFGSPLMETEVNLDSYQTALEEVLS |
| | | | WLLSAEDTLQAQGEISNDVEEVKEQ |
| | | | FHTHEGYMMDLTSHQGRVGNVLQL |
| | | | GSQLIGSGKLSEDEETEVQEQMNLLN |
| | | | SRWECLRVASMEKQSNLHKVLMDL |
| | | | QNQQLKELNAWLTKTEERTRKMEK |
| | | | EPLGPDLEDLKRQIQQHKVLQEDLE |
| | | | QEQVRVNSLTHMVVVVDESSGDHA |
| | | | TAALEEQLKVLGDRWANICRWTED |
| | | | RWVLLQDVLLKWQRFTEEQCLFST |
| | | | WLSDKEDALNKIPTSGFKDQSEMLSS |
| | | | LQKLAVLKTDLEKKKQTMDKLCSL |
| | | | NHDLLSTLKNTLVAQKMEAWLDNF |
| | | | AQRWDNLVQKLEKSSTQISQAVTTT |
| | | | QPSLTQTTVMETVTMVTTREQILVK |
| | | | HAQEELPPPPPQKKRQIIVDSEIKKRL |
| | | | DVDITELHSWITRSEAVLQSPEFAVY |
| | | | RKEGNFSDLKEKVNAIEREKAEKFR |
| | | | KLQDASRSAQALVEQMVNEGVNAD |
| | | | SIKQASEQLNSRWIEFCQLLSERLNW |
| | | | LEYQNNIITFYNQLQQLEQMTTTAEN |
| | | | WLKTQPTTPSEPTAVKSQLKNCKDE |
| | | | VNRLSALQPQIERLKIQSIALKEKGQ |
| | | | GPMFLDADFVAFTNHFNQVFADMQ |
| | | | AREKELQTILDTLPTVRYQETMSTIL |
| | | | TWIQQSETKLSIPQVTVTEYEIMEQR |
| | | | LEELQALQSSLQEHQNDLNYLSTTV |
| | | | KEMSRKAPSHISQRYQSEFENIEGRW |
| | | | KKLSAQLNERCQKLEEQMAKLRKL |
| | | | QNHIKTLKKWMAEVDVFLKDEWPA |
| | | | LGDSEILKKQLKQCRLLVSDIQTIQPS |
| | | | LNSVNEGGQKIKTEAEPEFASRLETE |
| | | | LRELNTQWDYICRQVYARKEALKG |
| | | | GLDKTVSLQKDLSEMHEWMTQAEE |
| | | | EYLERDFEYKTPDELQTAVEEMKRA |
| | | | KEEAQQKEAKVQLLTESVNSVIAQA |
| | | | PPAAQEALRKELDTLTTNYQWLCTR |
| | | | LNGKCKTLEEVWACWHELLSYLEK |
| | | | ANKWLNEVELKLKTTENIPGGAEEIS |
| | | | EVLSSLENLMQHSEDNPNQIRILAQT |
| | | | LTDGGVMDELINEELETFNSRWREL |
| | | | HEEAVRRQKLLEQSIQSAQEIEKSLQ |
| | | | LIQESLSSIDKQLAAYIADKVDAAQM |
| | | | PQEAQKIQSDLTSHEISLEEMKKHYQ |
| | | | GKETAQRVLSQIEVAQKKMQDVSM |
| | | | KFRLFQKPANFEQRLQESKMILDEVK |
| | | | MHLPALETKSVEQEVVQSQLNHCVN |
| | | | LYKSLSEVKSEVEMVIKTGRQIVQKK |
| | | | QTENPKELDERVTALKLHYNELGAK |
| | | | VTERKQQLEKCLKLSRKMRKEMNA |
| | | | LTEWLAATDLELTKRSAVEGMPSNL |
| | | | DSEVAWGKATQKETEKQKVHLKSIT |
| | | | ELGEALKTVLGKKETLVEDKLSLLN |
| | | | SNWIAVTSRAEEWLNLLLEYQKHME |
| | | | TFDQNVDHITKWIIQADALLDESEKK |
| | | | KPQQKEDMLKRLKAELNDIRPKVDS |
| | | | TRDQAANLMANRGDHCRKVIEPKIS |
| | | | ELNHRFAAISHRIKTGKASIPLKELEQ |
| | | | FNSDIQKLLEPLEAEIQQGVNLKEED |
| | | | FNKDMSEDNEGTVKELLQRGDNLQ |
| | | | QRITDERKREEIKIKQQLLQTKHNAL |
| | | | KDLRSQRRKKALEISHQWYQYKRQ |
| | | | ADDLLKCLDDIEKKLASLPEPRDERK |
| | | | IKEIDRELQKKKEELNAVHRQAEGLS |
| | | | EDGAAMAVESTQIQLSKRWREIESKF |
| | | | AQFRRLNFAQIHTVHEESVMVMTED |
| | | | MPLEISYVPSTYLTEITHVLQALSEVE |
| | | | QLLNAPDLCAKDFQDLFKQEESLKNI |
| | | | KDNLQQISGRIDVIHNKKAAALQSTT |
| | | | PPEKARLQEALSRLDFQWERVNKMY |
| | | | KDRQGQFDRSVEKWRRFHYDMKIF |
| | | | NQWLTEAEHFLKKTQIPENWEHAKY |
| | | | KWYLKELQDGIGQRQTVVRVLNAT |
| | | | GEEIIQQSSKIDASILQEKLGSLNLRW |
| | | | QEVCKQLAERKKRLEEQKNILSEFQR |
| | | | DLNEFVLWLEEAGNISSIPLEPGNEQ |
| | | | QLKEKLEEVKLLAEELPLRQGILKQL |
| | | | NETGGTVLVSAPISPEEQDKLENKLK |
| | | | QTNLQWIKVSRSLPEKQGEIEAHIKD |
| | | | LGQLEEQLNHLLLWLSPIRSQLEIYN |
| | | | QPNQTGPFDIKETEVAVQAKQPDVE |
| | | | GILSKGQNLYKEKPATEPVKRKLEDL |
| | | | SSEWKAVTHLLQELRAKWPGPVPGL |
| | | | TATGAPPSQTVALVTQPVVAKETAV |
| | | | SKLEMPSSLLLEVPALADFNRAWTE |
| | | | LTDWLSLLDRVLKAQRVMVGDLEDI |
| | | | NEMIIKQKATLQDLEQRRPQLEELIT |
| | | | AAQNLKNKTSNQEARTIITDRIERIQS |
| | | | QWDEVQEHLQNRRQQLNEMLKDST |
| | | | QWLEAKEEAEQVVGQARAKLETWK |
| | | | EGPYTMDAIQRKITETKQLAKDLRQ |
| | | | WQINVDVANDLALKLLRDYSADDT |
| | | | RKVHMITENINASWASIHKRVSERET |
| | | | ALEETHRLLQQFPLDLEKFLAWLTE |
| | | | AETTANVLQDATHKERLLEDSKGVR |
| | | | ELMKQWQDLQGEIEAHTDIYHNLDE |
| | | | NGQKILRSLEGSDDAVLLQRRLDNM |
| | | | NFKWSELRKKSLNIRSHLEASSDQW |
| | | | KRLHLSLQELLVWLQLKDDELSRQA |
| | | | PIGGDFPAVQKQNDIHRAFKRELKTK |
| | | | EPVIMSTLETVRIFLTEQPLEGLEKLY |
| | | | QEPRELPPEEKAQNVTRLLRKQAEE |
| | | | VNTEWEKLNLHSADWQRKIDEALER |
| | | | LQELQEATDELDLKLRQAEVIKGSW |
| | | | QPVGDLLIDSLQDHLEKVKALRGEIA |
| | | | PLKENVSHVSDLARQLTTLGIQLSPY |
| | | | NLSTLEDLNTRWKLLQVAVEDRIRQ |
| | | | LHEAHRDFGPASQHFLSTSVQGPWE |
| | | | RAISPNKVPYYINHETQTTCWDHPK |
| | | | MTELYQSLADLNNVRFSAYRTAMK |
| | | | LRRLQKALCLDLLSLSAACDALDQH |
| | | | NLKQNDQPMDILQIINCLTTIYDRLE |
| | | | QEHNNLVNVPLCVDMCLNWLLNVY |
| | | | DTGRTGRIRVLSFKTGIISLCKAHLED |
| | | | KYRYLFKQVASSTGFCDQRRLGLLL |
| | | | HDSIQIPRQLGEVASFGGSNIEPSVRS |
| | | | CFQFANNKPEIEAALFLDWMRLEPQ |
| | | | SMVWLPVLHRVAAAETAKHQAKCN |
| | | | ICKECPIIGFRYRSLKHFNYDICQSCFF |
| | | | SGRVAKGHKMHYPMVEYCTPTTSG |
| | | | EDVRDFAKVLKNKFRTKRYFAKHPR |
| | | | MGYLPVQTVLEGDNMETPVTLINFW |
| | | | PVDSAPASSPQLSHDDTHSRIEHYAS |
| | | | RLAEMENSSGSYLNDSISPNESIDDE |
| | | | HLLIQHYCQSLNQDSPLSQPRSPAQIL |
| | | | ISLESEERGELERILADLEEENRNLQA |
| | | | EYDRLKEQHEHKGLSPLPSPPEMMP |
| | | | TSPQSPRDAELIAEAKLLRQHKGRLE |
| | | | ARMQILEDHNKQLESQLHRLRQLLE |
| | | | QPQAEAKVNGTTVSSPSTSLQRSDSS |
| | | | QPMLLRVVGSQTSESMGEEDLLSPP |
| | | | QDSSTGLEEVMEQLNNSFPSSRGRNT |
| | | | PGKPMREDTM |
| SEQ ID | Q05JF3 | Calsequestrin/ | MSAADRMGARAVPGLRLALLLLMV |
| NO: 99 | (UniProtKB) | Bos taurus | LGTPKSGVQGEEGLDFPEYDGVDRV |
| | | | VNVNAKNYKNVFKKYEVLALLYHE |
| | | | PPEDDKASQRQFEMDELILELAAQVL |
| | | | EDKGVGFGMVDSEKDAAVAKKLGL |
| | | | TEEDSVYVFKGDEVIEYDGEFSADTL |
| | | | VEFLLDVLEDPVELIEGERELQAFENI |
| | | | EDDNKLIGYFKNKDSEHYKAYEDAA |
| | | | EEFHPYIPFFATFDSKVAKKLTLKLN |
| | | | EIDFYEAFMEEPVTIPDKPNSEEEIVS |
| | | | FVEAHKRSTLRKLKPESMYETWEDD |
| | | | LDGIHIVAFAEETDPDGYEFLETLKA |
| | | | VAQDNTDNPDLSIIWIDPDDFPLLVP |
| | | | YWEKTFNIDLSAPQIGVVNVTDADS |
| | | | VWMEMDDEEDLPSAEELEDWLEDV |
| | | | LEGEINTEDDDEEDD |

Cardiac muscle tissue

| | | | |
|---|---|---|---|
| SEQ ID | P68034 | Actin, alpha | MCDDEETTALVCDNGSGLVKAGFA |
| NO: 100 | (UniProtKB) | cardiac muscle | GDDAPRAVFPSIVGRPRHQGVMVG |
| | | 1/Gallus gallus | MGQKDSYVGDEAQSKRGILTLKYPI |
| | | | EHGIITNWDDMEKIWHHTFYNELRV |
| | | | APEEHPTLLTEAPLNPKANREKMTQI |
| | | | MFETFNVPAMYVAIQAVLSLYASGR |
| | | | TTGIVLDSGDGVTHNVPIYEGYALPH |
| | | | AIMRLDLAGRDLTDYLMKILTERGY |
| | | | SFVTTAEREIVRDIKEKLCYVALDFE |
| | | | NEMATAASSSSEEKSYELPDGQVITI |
| | | | GNERFRCPETLFQPSFIGMESAGIHET |
| | | | TYNSIMKCDIDIRKDLYANNVLSGGT |
| | | | TMYPGIADRMQKEITALAPSTMKIKII |
| | | | APPERKYSVWIGGSILASLSTFQQMW |
| | | | ISKQEYDEAGPSIVHRKCF |
| SEQ ID | XP_004938861.1 | Gamma- | MVREQYTTTTPGTSLERPKNEYVYKI |
| NO: 101 | (NCBI) | sarcoglycan | GIYGWRKRCLYLFVLLLLIILVVNFS |
| | | isoform | LTIWILKVMWFSPTGMGHLRVTKEG |
| | | X2/Gallus gallus | LRLEGESEFLFPLYAKEIHSRVDSSLL |
| | | | LQSTHNVTVNARNSNGEVTGRLNVG |
| | | | PKMVEFHGQQFQINSKDGKPLFTVD |
| | | | ENEVVIGTDKLRVTGPEGALFEHSVE |
| | | | TPLVKAEAFKQLRLESPTRSLSMDAP |
| | | | RGIN |
| | | | IKAQAGNIEALSQMDIKLHSSDGVLL |
| | | | LDAETVRLPKLPEGTRGGPGVSQGL |
| | | | YEICVCPDGKLYLSVAGLGSTCQEYS |
| | | | RVCQ |
| SEQ ID | NP_990097.1 | Myosin heavy | MMDMTEFGEAAPFLRKSEKELMML |
| NO: 102 | (NCBI) | chain, cardiac | QTVAFDGKKKCWVPDDKKAYVEAE |
| | | muscle | ITESSGGKVTVETTDGRTMTIKEDDV |
| | | isoform/Gallus | QSMNPPKFDMIEDMAMLTHLNEASV |
| | | gallus | LYNLRKRYSNWMIYTYSGLFCVTIN |
| | | | PYKWLPVYKSEVVAAYKGKRRSEA |
| | | | PPHIFSIADNAYHDMLRNRENQSMLI |
| | | | TGESGAGKTVNTKRVIQYFATVAAL |
| | | | GEPGKKSQPATKTGGTLEDQIIQANP |
| | | | ALEAFGNAKTLRNDNSSRFGKFIRIH |
| | | | FGTTGKLSSADIEIYLLEKSRVIFQQP |
| | | | GERDYHIFYQILSGKKPELLDMLLVS |
| | | | TNPYDYHFCSQGVVTVDNLDDGEEL |
| | | | MATDQAMDILGFVPDEKYGAYKLT |
| | | | GAIMHFGNMKFKQRPREEQAEADGT |
| | | | ESADKAAYLMGINSSDLVKGLLHPR |
| | | | VKVGNEYVTKGQSVEQVLYAVGAL |
| | | | SKAVYDRMFKWLVVRINKTLDTKLP |
| | | | RQFFIGVLDIAGFEIFDFNSFEQLCINY |
| | | | TNEKLQQFFNHHMFVLEQEEYKKEG |
| | | | IEWVFIDFGMDLQACIDLIEKPLGILSI |
| | | | LEEECMFPKATDMTFKAKLYDNHLG |
| | | | KSPNLQKPRPDKKRKYEAHFELIHY |
| | | | AGSVPYNIIGWLEKNKDPLNETVVGI |
| | | | FQKSSNKLLASLFESYVGADSADQG |
| | | | GEKKRKKGASFQTVSSLHKENLNKL |
| | | | MTNLRSTAPHFVRCIIPNESKTPGEM |
| | | | DAFLVLHQLRCNGVLEGIRICRKGFP |
| | | | NRVLYADFKQRYRILNPGAIPEDKFV |
| | | | DSRKAAEKLLASLDIDHNQYRFGHT |
| | | | KVFFKAGLLGHLEEMRDERLAKILT |
| | | | MIQARARGRLMRIEFQKIVERRDALL |
| | | | VIQWNIRAFMAVKNWPWMKLFFKI |
| | | | KPLLKSAETEKEMANMKEEFLKLKE |
| | | | ALEKSEARRKELEEKQVSLVQEKND |
| | | | LLLQLQAEQDTLADAEERCDLLIKSK |
| | | | IQLEAKVKELTERVEDEEEMNSELTS |
| | | | KKRKLEDECSELKKDIDDLEITLAKV |
| | | | EKEKHATENKVKNLTEEMATLDENI |
| | | | SKLTKEKKSLQEAHQQVLDDLQAEE |
| | | | DKVNTLSKAKVKLEQQVDDLEGSLE |
| | | | QEKKVRMDLERAKRKLEGDLKLTQ |
| | | | ESVMDLENDKLQMEEKLKKKEFEM |
| | | | SQLNSKIEDEQAIVMQLQKKIKELQA |
| | | | RIEELEEELEAERAARAKVEKQRSDL |
| | | | ARELEVLSERLEEAGGATAAQLEMN |
| | | | KKREAEFLKLARDLEEATLHYEATA |
| | | | AALRKKHADSVAEMGEQLDNLQRV |
| | | | KQKLEKEKSELKMEVDDLTSNMEQT |
| | | | VKGKANAEKLCRTYEDHLNETKTKL |
| | | | DEMTRLMNDLTTQKTKLQSENGEFV |
| | | | RQLEEKESLISQLSRGKTSFTQQIEEL |
| | | | RRQLEEETKSKNALAHALQAARHDC |
| | | | DLLREQYEEEQEAKAELQRALSKGN |
| | | | AEVAQWRTKYETDAIQRTEELEDAK |
| | | | KKLAARLQEAEEAIEAANAKCSSLE |
| | | | KTKHRLQNELEDMMIDLEKANSAA |
| | | | ASLDKKQRGFDKIINDWKQKYEESQ |
| | | | AELEASQKEARSLSTELFKLKNAYEE |
| | | | TLDHLETLKRENKNLQEEISDLTNQI |
| | | | SEGNKNLHEIEKVKKQVEQEKSEVQ |
| | | | LALEEAEGALEHEESKTLRFQLELSQ |
| | | | LKADFERKLAEKDEEMENIRRNQQR |
| | | | TIDSLQSTLDSEARSRNEAIRLKKKM |
| | | | EGDLNEMEIQLSHANRHAAEATKSA |
| | | | RGLQTQIKELQVQLDDLGHLNEDLK |
| | | | EQLAVSDRRNNLLQSELDELRALLD |
| | | | QTERARKLAEHELLEATERVNLLHT |
| | | | QNTSLINQKKKLEGDISQMQNEVEES |
| | | | IQECRNAEEKAKKAITDAAMMAEEL |
| | | | KKEQDTSAHLERMKKNMEQTIKDL |
| | | | QKRLDEAEQIALKGGKKQIQKLESR |
| | | | VRELENELENELRRNSDAQKGARKF |
| | | | ERRIKEVTYQSEEDKKNLARMQDLI |
| | | | DKLQLKVKSYKHQAEEAEAQANLY |
| | | | LSKYRKQQHDLDDAEERAEIAESQV |
| | | | NKLRSKSRDIGMKKVHEEE |
| SEQ ID | Q90688 | Myosin-binding | MPEPAKKAVSAFTKKPKTTEVAAGS |
| NO: 103 | (UniProtKB) | protein C, | TAVFEAETEKTGIKVKWQRAGTEIT |
| | | cardiac- | DSEKYAIKAEGNKHSLTISNVGKDDE |
| | | type/Gallus | VTYAVIAGTSKVKFELKVKEPEKSEP |
| | | gallus | VAPAEASPAPAASELPAPPVESNQNP |
| | | | EVPPAETQPEEPVDPIGLFVTRPQDG |
| | | | EVTVGGNITFTAKVAGESLLKKPSVK |
| | | | WFKGKWMDLASKVGKHLQLHDNY |
| | | | DRNNKVYTFEMEIIEANMTFAGGYR |
| | | | CEVSTKDKFDSSNFNLIVNEAPVSGE |
| | | | MDIRAAFRRTSLAGGGRRMTSAFLS |
| | | | TEGLEESGELNFSALLKKRDSFLRTA |
| | | | NRGDGKSDSQPDVDVWEILRKAPPS |
| | | | EYEKIAFQYGITDLRGMLKRLKRIKK |
| | | | EEKKSTAFLKKLDPAYQVDKGQKIK |
| | | | LMVEVANPDADVKWLKNGQEIQVS |
| | | | GSKYIFEAIGNKRILTINHCSLADDAA |
| | | | YECVVAEEKSFTELFVKEPPILITHPL |
| | | | EDQMVMVGERVEFECEVSEEGATV |
| | | | KWEKDGVELTREETFKYRFKKDGK |
| | | | KQYLIINESTKEDSGHYTVKTNGGVS |
| | | | VAELIVQEKKLEVYQSIADLTVKAR |
| | | | DQAVFKCEVSDENVKGIWLKNGKE |
| | | | VVPDERIKISHIGRIHKLTIEDVTPGD |
| | | | EADYSFIPQGFAYNLSAKLQFLEVKI |
| | | | DFVPREEPPKIHLDCLGQSPDTIVVV |
| | | | AGNKLRLDVPISGDPTPTVIWQKVN |
| | | | KKGELVHQSNEDSLTPSENSSDLSTD |
| | | | SKLLFESEGRVRVEKHEDHCVFIIEG |
| | | | AEKEDEGVYRVIVKNPVGEDKADIT |
| | | | VKVIDVPDPPEAPKISNIGEDYCTVQ |
| | | | WQPPTYDGGQPVLGYILERKKKKSY |
| | | | RWMRLNFDLLKELTYEAKRMIEGV |
| | | | VYEMRIYAVNSIGMSRPSPASQPFMP |
| | | | IAPPSEPTHFTVEDVSDTTVALKWRP |
| | | | PERIGAGGLDGYIVEYCKDGSAEWT |
| | | | PALPGLTERTSALIKDLVTGDKLYFR |
| | | | VKAINLAGESGAAIIKEPVTVQEIMQ |
| | | | RPKICVPRHLRQTLVKKVGETINIMIP |
| | | | FQGKPRPKISWMKDGQTLDSKDVGI |
| | | | RNSSTDTILFIRKAELHHSGAYEVTL |
| | | | QIENMTDTVAITIQIIDKPGPPQNIKL |
| | | | ADVWGFNVALEWTPPQDDGNAQIL |
| | | | GYTVQKADKKTMEWYTVYDHYRR |
| | | | TNCVVSDLIMGNEYFFRVFSENLCGL |
| | | | SETAATTKNPAYIQKTGTTYKPPSYK |
| | | | EHDFSEPPKFTHPLVNRSVIAGYNTT |
| | | | LSCAVRGIPKPKIFWYKNKVDLSGD |
| | | | AKYRMFSKQGVLTLEIRKPTPLDGGF |
| | | | YTCKAVNERGEAEIECRLDVRVPQ |
| SEQ ID | F1NBZ9 | Myosin-binding | MPEPAKKAVSAFTKKPKTTEVAAGS |
| NO: 104 | (UniProtKB) | protein C, | TAVFEAETEKTGIKVKWQRAGTEIT |
| | | cardiac- | DSEKYAIKAEGNKHSLTISNVGKDDE |
| | | type/Gallus | VTYAVIAGTSKVKFELKVKEPEKSEP |
| | | gallus | VAPAEASAPAASELPAPPVESNQNPE |
| | | | VPPAETQPEEPVDPIGLFVTRPQDGE |
| | | | VTVGGNITFTAKVAGESLLKKPSVK |
| | | | WFKGKWMDLASKVGKHLQLHDNY |
| | | | DRNNKVYTFEMEIIEANMTFAGGYR |
| | | | CEVSTKDKFDSSNFNLIVNEAPVSGE |
| | | | MDIRAAFRRTSLAGGGRRMTSAFLS |
| | | | TEGLEESGELNFSALLKKSSSFLRTA |
| | | | NRGDGKSDSQPDVDVWEILRKAPPS |
| | | | EYEKIAFQYGITDLRGMLKRLKRIKK |
| | | | EEKKSTAFLKKLDPAYQVDKGQKIK |
| | | | LMVEVANPDADVKWLKNGQEIQVS |
| | | | GSRYIFEAIGNKRILTINHCSLADDAA |
| | | | YECVVAEEKSFTELFVKEPPILITHPL |
| | | | EDQMVMVGERVEFECEVSEEGATV |
| | | | KWEKDGVELTREETFKYRFKKDGK |
| | | | KQYLIINESTKEDSGHYTVKTNGGVS |
| | | | VAELIVQEKKLEVYQSIADLTVKAR |
| | | | DQAVFKCEVSDENVKGIWLKNGKE |
| | | | VVPDERIKISHIGRIHKLTIEDVTPGD |
| | | | EADYSFIPQGFAYNLSAKLQFLEVKI |
| | | | DFVPREEPPKIHLDCLGQSPDTIVVV |
| | | | AGNKLRLDVPISGDPTPTVIWQKVN |
| | | | KKGELVHQSNEDSLTPSENSSDLSTD |
| | | | SKLLFESEGRVRVEKHEDHCVFIIEG |
| | | | AEKEDEGVYRVIVKNPVGEDKADIT |
| | | | VKVIDVPDPPEAPKISNIGEDYCTVQ |
| | | | WQPPTYDGGQPVLGYILERKKKKSY |
| | | | RWMRLNFDLLKELTYEAKRMIEGV |
| | | | VYEMRIYAVNSIGMSRPSPASQPFMP |
| | | | IAPPSEPTHFTVEDVSDTTVALKWRP |
| | | | PERIGAGGLDGYIVEYCKDGSAEWT |
| | | | PALPGLTERTSALIKDLVTGDKLYFR |
| | | | VKAINLAGESGAAIIKEPVTVQEIMQ |
| | | | RPKIWLPRHLRQTLVKKVGETINIMI |
| | | | PFQGKPRPKISWMKDGQTLDSKDVG |
| | | | IRNSSTDTILFIRKAELHHSGAYEVTL |
| | | | QIENMTDTVAITIQIIDKPGPPQNIKL |
| | | | ADVWGFNVALEWTPPQDDGNAQIL |
| | | | GYTVQKADKKTMEWYTVYDHYRR |
| | | | TNCVVSDLIMGNEYFFRVFSENLCGL |
| | | | SETAATTKNPAYIQKTGTTYKPPSYK |
| | | | EHDFSEPPKFTHPLVNRSVIAGYNTT |
| | | | LSCAVRGIPKPKIFWYKNKVDLSGD |
| | | | AKYRMFSKQGVLTLEIRKPTPFDGGF |
| | | | YTCKAVNERGEAEIECRLDVRVPQ |
| SEQ ID | NP_998735.1 | Troponin 1, | MAEEEEPKPPPLRRKSSANYRGYAV |
| NO: 105 | (NCBI) | cardiac | EPHAKRQSKISASRKLQLKTLLLQRA |
| | | muscle/Gallus | KRELEREEQERAGEKQRHLGELCPPP |
| | | gallus | ELEGLGVAQLQELCRELHARIGRVD |
| | | | EERYDMGTRVSKNMAEMEELRRRV |
| | | | AGGRFVRPALRRVRLSADAMMAAL |
| | | | LGSKHRVGTDLRAGLRQVRKDDAE |
| | | | KESREVGDWRKNVDALSGMEGRKK |
| | | | KFEAPGGGQG |
| SEQ ID | A0A1D5PF28 | Troponin T, | MKQKHQEKGESKPKPKPFMPNLVPP |
| NO: 106 | (UniProtKB) | cardiac muscle | KIPDGERLDFDDIHRKRMEKDLNEL |
| | | isoforms/Gallus | QALIEAHFESRKKEEEELISLKDRIEQ |
| | | gallus | RRAERAEQQRIRSEREKERQARMAE |
| | | | ERARKEEEEARKKAEEEARKKKAFS |
| | | | NMLHFGGYMQKSEKKGGKKQTERE |
| | | | KKKKILSERRKPLNIDHLSEDKLRDK |
| | | | AKELWQTIRDLEAEKFDLQEKFKRQ |
| | | | KYEINVLRNRVSDHQKV |
| SEQ ID | F1RRT2 | Myosin light | MPPKKPEPKKEAAKAAPAPAPAPAP |
| NO: 107 | (UniProtKB) | chain 4/Sus | APAPPPEPAKEPTFDPKSIKIDFTADQI |
| | | scrofa | EEFKEAFSLFDRTPTGEMKITYGQCG |
| | | | DVLRALGQNPTNAEVLRVLGKPKPE |
| | | | EMNAKMLDFETFLPILQHISRNKEQG |
| | | | TYEDFVEGLRVFDKESNGTVMGAEL |
| | | | RHVLATLGEKMTEAEVEQLLAGQED |
| | | | ANGCINYEAFVKHIMSG |
| SEQ ID | Q910C5 | Atrial myosin | MEALLGAAAPFLRAPEGPRPTPAGD |
| NO: 108 | (UniProtKB) | heavy | TRGLCFVPHPQLEFIRARVTARAGNG |
| | | chain/Gallus | VTVTTEMGETLTVPEADVHPQNPPK |
| | | gallus | FDRIEDMAMLTFLHEPAVLYNLKER |
| | | | YASWMIYTYSGLFCVTVNPYKWLPV |
| | | | YNAEVVAAYRGKKRTEVPPHIFSISD |
| | | | NAYQNMLTDRENQSILITGESGAGK |
| | | | TVNTKRVIQYFASIAAIGHRKKEVAN |
| | | | SSKGTLEDQIIQANPALEAFGNAKTV |
| | | | RNDNSSRFGKFIRIHFGATGKLASADI |
| | | | ETYLLEKSRVIFQLKAERNYHIFYQIL |
| | | | SNKKPELLEMLLITNNPYDYSYVSQG |
| | | | EVTVASIDDSEELLATDSAFDVLGFT |
| | | | AEEKAGVYKLTGAIMHFGNMKFKQ |
| | | | KQREEQAEPDGTEDCDKSAYLMGL |
| | | | NSADLLKGLCHPRVKVGNEYVTKG |
| | | | QSVQQVYYSIGALAKAVYEKMFNW |
| | | | MVVRINNSLETKQPRQYFIGVLDIAG |
| | | | FEIFDFNSFEQLCINFTNEKLQQFFNH |
| | | | HMFVLEQEEYKKEGIEWEFIDFGMD |
| | | | LQACIDLIEKPMGIMSILEEECMFPKA |
| | | | SDMTFKAKLFDNHLGKSANFGKPRN |
| | | | VKGKSEAHFSLIHYAGTVDYNIIGWL |
| | | | EKNKDPLNETVVGLYQKSALKLLAS |
| | | | LFSNYAGADAGGDGGKGKGAKKKG |
| | | | SSFQTVSALHRENLNKLMANLKTTH |
| | | | PHFVRCLIPNERKEPGVMDNPLVMH |
| | | | QLRCNGVLEGIRICRKGFPNRILYGD |
| | | | FRQRYRIPNPTAIPEGQFIDSRKGAEK |
| | | | LLGSLDIDHNQYKFGHTKVFFKAGL |
| | | | LGLLEEMRDERLSLIITRIQAQARGQ |
| | | | LMRIEFKKILERRDALLVIQWNIRAF |
| | | | MGVKNWPWMKLYFKIKPLLKSAET |
| | | | EKEMQTMKEEFGHLKEALEKSAARR |
| | | | KELEEKMVSMLQEKNDLQLQVQAE |
| | | | QDNLADAEERCDQLIKNKIQLEAKV |
| | | | KEMTERLEEEEEMNAELAAKKRKLE |
| | | | DECSELKKDIDDLELSLAKVEKEKH |
| | | | ATENKVKNLTEEMAGLDENITKLTK |
| | | | EKKILQESHQQALDDLQAEEDKVNT |
| | | | LAKAKGKLEQQVDDLESSLEQEKKI |
| | | | RMDLERAKRKLEGDLKLAQESIMDL |
| | | | ENDKQQLEERLKKKDFELNTLNARI |
| | | | EDEQAISAQLQKKLKELQARIEELEE |
| | | | ELEAERTGRAKVEKLRSELLQELEET |
| | | | SERLEEAGGATSVQLELNKKREAEF |
| | | | QKLRRDLEEATLQHEATAATLRKKH |
| | | | ADSVAELSEQLDNLQRVKQKLEKEK |
| | | | SELKLELDDVNSNTEQLIKAKTNLEK |
| | | | MCRTTEDQMNEHRSKLEEAQRTVT |
| | | | DLSTQRAKLQTENSELSRQLEEKEAF |
| | | | INQLTRGKLTYTQQLEDLKRQLEEEA |
| | | | KAKNALAHALQSAQHDCDLLREQY |
| | | | EEEMEAKAELQRALSKANSEVAQW |
| | | | RTKYETDAIQRTEELEEAKKKLAQR |
| | | | LQEAEEAVEAVNAKCSSLEKTKHRL |
| | | | QNEIEDLMADVERSNAAAAALDKK |
| | | | QRNFDKILSEWKQKFEESQTELEASQ |
| | | | KEARSLSTELFKLKNAYEESLEHLET |
| | | | FKRENKNLQEEILDLTEQLGASQKSI |
| | | | HELEKVRKQLDAEKLELQAALEEAE |
| | | | ASLEHEEGKILRAQLEFNQVKADYE |
| | | | RKLAEKDEEIEQSKRNHLRVVDSLQ |
| | | | TSLDAETRSRNEALRLKKKMEGDLN |
| | | | EMEIQLSHANRTAAEAQKQVKALQG |
| | | | YLKDTQLQLDDVVRANEDLKENIAI |
| | | | VERRNNLLQSELEELRAMVEQSERA |
| | | | RKLAEQELIEASERVQLLHSQNTSLI |
| | | | NQKKKMEADISQLQTEVEEAIQECR |
| | | | NAEEKAKKAITDAAMMAEELKKEQ |
| | | | DTSAHLERMKKNMEQTVKDLQLRL |
| | | | DEAEQLALKGGKKQLQKLEVRVREL |
| | | | ENELEAEQKRNAESIKGLRKSERRVK |
| | | | ELSYQTEEDRKNMVRLQDLVDKLQL |
| | | | KVKAYKRQAEEAEEQANSNLAKFR |
| | | | KVQHELDEAEERADMAESQVNKLR |
| | | | ARSRDIGAKKGLNEE |
| SEQ ID | Q8UWA0 | Myosin heavy | MSMLDMSEFGEAAEYLRKSYTEQLK |
| NO: 109 | (UniProtKB) | chain/Gallus | LQTIPFDGKKRAWIPDEKEAYIEVEIK |
| | | gallus | ESTGGKVTVETKDKQTRVVKEDELQ |
| | | | AMNPPKFDMIEDMAMLTHLNEASV |
| | | | LYNLKRRYSHWMIYTYSGLFCVTIN |
| | | | PYKWLPVYTAPVVAAYKGKRRSEA |
| | | | PPHIYSIADNAYNDMLRNRENQSMLI |
| | | | TGESGAGKTVNTKRVIQYFAIVAAL |
| | | | GDTPGKKVAALATKTGGTLEDQIIEA |
| | | | NPAMEAFGNAKTIRNDNSSRFGKFIR |
| | | | IHFGPSGKLASADIDIYLLEKSRVIFQ |
| | | | QPKERSYHIYYQILSGKKPELQDMLL |
| | | | LSLNPYDYHFCSQGVTTVDNLDDGE |
| | | | ELMATDHAMDILGFSNDEKYGSYKI |
| | | | VGAIMHFGNMKFKQKQREEQAEAD |
| | | | GTESADKAAYLMGISSADLIKGLLHP |
| | | | RVKVGNEYVTKGQNVEQVVYAVGA |
| | | | LAKATYDRMFKWLVTRINKTLDTKL |
| | | | ARQFFIGVLDIAGFEIFDFNSFEQLCIN |
| | | | FTNEKLQQFFNHHMFVLEQEEYKKE |
| | | | GIEWVFIDFGLDLQACIDLIEKPLGIL |
| | | | SILEEECMFPKASDMSFKAKLYDNH |
| | | | LGKSPNFQKPRPDKKRKYEAHFELV |
| | | | HYAGVVPYNIIGWLDKNKDPLNETV |
| | | | VAVFQKSQNKLLASLYENYVGSSSE |
| | | | EPHKPGSKEKRKKAASFQTVSQLHK |
| | | | ENLNKLMTNLRSTQPHFVRCIIPNET |
| | | | KTPGAMDAFLVLHQLRCNGVLEGIR |
| | | | ICRKGFPNRILYADFKQRYRILNPAAI |
| | | | PDDKFVDSRKATEKLLSSLELDHSQY |
| | | | KFGHTKVFFKAGLLGMLEEMRDERL |
| | | | AKILTMLQARIRGHLMRIEYQKIISRR |
| | | | EALYTIQWNIRAFNAVKNWSWMKL |
| | | | FFKIKPLLKSAQTEKEMSTLKEEFQK |
| | | | LKEALEKSEAKRKELEEKQVSMIQE |
| | | | KNDLALQLQAEQDNLADAEERCDLL |
| | | | IKSKIQLEAKVKELTERVEDEEEMNA |
| | | | DLTAKKRKLEDECAELKKDIDDLEIT |
| | | | LAKVEKEKHATENKVKNLIEEMAAL |
| | | | DEIIAKLTKEKKALQEAHQQALDDL |
| | | | QAEEDKVNTLTKAKVKLEQQVDDL |
| | | | ESSLEQEKKIRMDLERAKRKLEGDL |
| | | | KLTQESVMDLENDKQQLEEKLKKK |
| | | | DFEMSQLNSRIEDQQVTEAQLQKKIK |
| | | | ELQARIEELEEELEAERAARAKVEKQ |
| | | | RAEVSRELEELSERLEEAGGATSAQL |
| | | | EMNKKREVEFLKLRRDLEEATLQHE |
| | | | STAAALRKKHADSVAELSEQIDNLQ |
| | | | RVKQKLEKEKSEMKMEVDDLSSNIE |
| | | | YLTKNKANAEKLCRTYEDQLSEAKS |
| | | | KVDELQRQLTDVSTQRGRLQTENGE |
| | | | LSRLLEEKESFINQLSRGKTSFTQTIE |
| | | | ELKRQLEEETKSKNALAHALQASRH |
| | | | DCDLLREQYEEEVEAKSELQRNLSK |
| | | | ANAEVAQWRTKYETDAIQRTEELEE |
| | | | AKKKLAIRLQEAEEAVEAAHAKCSS |
| | | | LEKTKHRLQTEIEDLSVDLERANSAC |
| | | | AALDKKQRNFDRILAEWKQKYEETQ |
| | | | AELEASQKESRSLSTELFKLKNAYEE |
| | | | SLDNLETLKRENKNLQEEIADLTDQI |
| | | | SMSGKTIHELEKLKKALENEKSDIQA |
| | | | ALEEAEGALEHEESKTLRIQLELNQI |
| | | | KADVDRKLAEKDEEFENLRRNHQR |
| | | | AMDSMQATLDAEARAKNEAVRLRK |
| | | | KMEGDLNEMEIQLSHANRQAAEFQK |
| | | | LGRQLQAQIKDLQIELDDTQRQNDD |
| | | | LKEQAAALERRNNLLLAEVEELRAA |
| | | | LEQAERSRKLAEQELLEATERVNLL |
| | | | HSQNTGLINQKKKLETDISQLSSEVE |
| | | | DAVQECRNAEEKAKKAITDAAMMA |
| | | | EELKKEQDTSAHLERMKKNMEQTIK |
| | | | DLQMRLDEAEQIALKGGKKQIQKLE |
| | | | ARVRELEGELDMEQKKMAEAQKGI |
| | | | RKYERRIKELSYQTEEDRKNLTRMQ |
| | | | DLIDKLQSKVKSYKRQFEEAEQQAN |
| | | | SNLVKYRKVQHELDDAEERADIAET |
| | | | QVNKLRARTKEVITFKHE |
| SEQ ID | P79293 | Myosin-7/Sus | MVDAEMAAFGEAAPYLRKSEKERL |
| NO: 110 | (UniProtKB) | scrofa | EAQTRPFDLKKDVYVPDDKEEFVKA |
| | | | KILSREGGKVTAETEHGKTVTVKED |
| | | | QVLQQNPPKFDKIEDMAMLTFLHEP |
| | | | AVLYNLKERYASWMIYTYSGLFCVT |
| | | | INPYKWLPVYNAEVVAAYRGKKRSE |
| | | | APPHIFSISDNAYQYMLTDRENQSILI |
| | | | TGESGAGKTVNTKRVIQYFAVIAAIG |
| | | | DRSKKEQTPGKGTLEDQIIQANPALE |
| | | | AFGNAKTVRNDNSSRFGKFIRIHFGA |
| | | | TGKLASADIETYLLEKSRVIFQLKAE |
| | | | RDYHIFYQILSNKKPELLDMLLITNN |
| | | | PYDYAFISQGETTVASIDDAEELMAT |
| | | | DNAFDVLGFTSEEKNSMYKLTGAIM |
| | | | HFGNMKFKLKQREEQAEPDGTEEAD |
| | | | KSAYLMGLNSADLLKGLCHPRVKV |
| | | | GNEYVTKGQNVQQVMYATGALAK |
| | | | AVYEKMFNWMVTRINTTLETKQPR |
| | | | QYFIGVLDIAGFEIFDFNSFEQLCINFT |
| | | | NEKLQQFFNHHMFVLEQEEYKKEGI |
| | | | EWEFIDFGMDLQACIDLIEKPMGIMS |
| | | | ILEEECMFPKATDMTFKAKLYDNHL |
| | | | GKSNNFQKPRNIKGRPEAHFALIHYA |
| | | | GTVDYNIIGWLQKNKDPLNETVVDL |
| | | | YKKSSLKLLSNLFANYAGADTPVEK |
| | | | GKGKAKKGSSFQTVSALHRENLNKL |
| | | | MTNLRSTHPHFVRCIIPNETKSPGVID |
| | | | NPLVMHQLRCNGVLEGIRICRKGFPN |
| | | | RILYGDFRQRYRILNPAAIPEGQFIDS |
| | | | RKGAEKLLGSLDIDHNQYKFGHTKV |
| | | | FFKAGLLGLLEEMRDERLSRIITRIQA |
| | | | QSRGVLSRMEFKKLLERRDSLLIIQW |
| | | | NIRAFMSVKNWPWMKLYFKIKPLLK |
| | | | SAETEKEMATMKEEFGRLKEALEKS |
| | | | EARRKELEEKMVSLLQEKNDLQLQV |
| | | | QAEQDNLADAEERCDQLIKNKIQLE |
| | | | AKVKEMTERLEDEEEMNAELTAKK |
| | | | RKLEDECSELKRDIDDLELTLAKVEK |
| | | | EKHATENKVKNLTEEMAGLDEIIAK |
| | | | LTKEKKALQEAHQQALDDLQAEED |
| | | | KVNTLTKAKVKLEQHVDDLEGSLEQ |
| | | | EKKVRMDLERAKRKLEGDLKLTQES |
| | | | IMDLENDKQQLDERLKKKDFELNAL |
| | | | NARIEDEQALGSQLQKKLKELQARIE |
| | | | ELEEELEAERTARAKVEKLRSDLSRE |
| | | | LEEISERLEEAGGATSVQIEMNKKRE |
| | | | AEFQKMRRDLEEATLQHEATAAALR |
| | | | KKHADSVAELGEQIDNLQRVKQKLE |
| | | | KEKSEFKLELDDVTSNMEQIIKAKAN |
| | | | LEKMCRTLEDQMNEHRSKAEETQRS |
| | | | VNDLTSQRAKLQTENGELSRQLDEK |
| | | | EALISQLTRGKLTYTQQLEDLKRQLE |
| | | | EEVKAKNALAHALQSARHDCDLLRE |
| | | | QYEEETEAKAELQRVLSKANSEVAQ |
| | | | WRTKYETDAIQRTEELEEAKKKLAQ |
| | | | RLQDAEEAVEAVNAKCSSLEKTKHR |
| | | | LQNEIEDLMVDVERSNAAAAALDKK |
| | | | QRNFDKILAEWKQKYEESQSELESSQ |
| | | | KEARSLSTELFKLKNAYEESLEHLET |
| | | | FKRENKNLQEEISDLTEQLGSSGKTI |
| | | | HELEKVRKQLEAEKLELQSALEEAE |
| | | | ASLEHEEGKILRAQLEFNQIKAEMER |
| | | | KLAEKDEEMEQAKRNHLRVVDSLQ |
| | | | TSLDAETRSRNEALRVKKKMEGDLN |
| | | | EMEIQLSHANRMAAEAQKQVKSLQS |
| | | | LLKDTQIQLDDAVRANDDLKENIAIV |
| | | | ERRNNLLQAELEELRAVVEQTERSR |
| | | | KLAEQELIETSERVQLLHSQNTSLINQ |
| | | | KKKMEADLSQLQTEVEEAVQECRN |
| | | | AEEKAKKAITDAAMMAEELKKEQD |
| | | | TSAHLERMKKNMEQTIKDLQHRLDE |
| | | | AEQIALKGGKKQLQKLEARVRELEN |
| | | | ELEAEQKRNAESVKGMRKSERRIKE |
| | | | LTYQTEEDRKNLLRLQDLVDKLQLK |
| | | | VKAYKRQAEEAEEQANTNLSKFRKV |
| | | | QHELDEAEERADIAESQVNKLRAKS |
| | | | RDIGTKGLNEE |
| SEQ ID | E1BJ10 | Myosin binding | MEAATAPEAALRPTLKVKEASPADA |
| NO: 111 | (UniProtKB) | protein H | DGPQASPRRGTGSPLSQLLPPIEEHPK |
| | | like/Bos taurus | IWLPRALRQTYIRKVGDTVNLLIPFQ |
| | | | GKPKPQAIWTRDGCALDTSRVSVRN |
| | | | GERDSILFIREAQRADSGRYQLSVQL |
| | | | GGLEATATIDILVIERPGPPQSIKLVD |
| | | | VWGSSATLEWTPPQDTGNAALLGYT |
| | | | VQKADTKSGLWFTVLERCRRASCTV |
| | | | PNLIVGNSYTFRVFAENQCGLSENAP |
| | | | ITADLAHIQKAATVYRTEGFAQRDFS |
| | | | EAPKFTQPLADCTTVIGYDTQLFCCV |
| | | | RASPKPKIIWLKNKMDIQGNPKYRA |
| | | | LTHLGICSLEIRKPGPFDGGIYTCKAV |
| | | | NPLGEASVDCRVDVKAPN |
| SEQ ID | P02540 | Desmin/Sus | MSQAYSSSQRVSSYRRTFGGAPSFPL |
| NO: 112 | (UniProtKB) | scrofa | GSPLSSPVFPRAGFGTKGSSSSVTSRV |
| | | | YQVSRTSGGAGGLGPLRASRLGATR |
| | | | VPSSSYGAGELLDFSLADAVNQEFLT |
| | | | TRTNEKVELQELNDRFANYIEKVRFL |
| | | | EQQNAALAAEVNRLKGREPTRVAEI |
| | | | YEEELRELRRQVEVLTNQRARVDVE |
| | | | RDNLLDDLQRLKAKLQEEIQLKEEA |
| | | | ENNLAAFRADVDAATLARIDLERRIE |
| | | | SLNEEIAFLKKVHEEEIRELQAQLQE |
| | | | QQVQVEMDMSKPDLTAALRDIRAQ |
| | | | YETIAAKNISEAEEWYKSKVSDLTQA |
| | | | ANKNNDALRQAKQEMMEYRHQIQS |
| | | | YTCEIDALKGTNDSLMRQMRELEDR |
| | | | FASEASGYQDNIARLEEEIRHLKDEM |
| | | | ARHLREYQDLLNVKMALDVEIATYR |
| | | | KLLEGEESRINLPIQTFSALNFRETSPE |
| | | | QRGSEVHTKKTVMIKTIETRDGEVVS |
| | | | EATQQQHEVL |
| SEQ ID | F1SFP9 | Leiomodin | MSEHSRNSDQEEPFDREIDEDEILAN |
| NO: 113 | (UniProtKB) | 3/Sus scrofa | LSPEELKELQSEMDVMAPDPRLPVG |
| | | | MIQKDQTDKPPTGNFDHKSLVDYM |
| | | | YWQKASRRMLEDERVPVTFVPSEEK |
| | | | PQEQRKEIDKGNKNMSQYLKEKLNN |
| | | | EIAAHKRESKSSDNEQETNDDDEDN |
| | | | EDDEDDEEDDAEDEEDEGDESDEKT |
| | | | KGEEEGEVKEPIRNGESNCQQVPNK |
| | | | AFEEQKDRPEAQEKFEKKISKLDPKK |
| | | | LALDTSFLKVSARPSGNQTDLDGSLR |
| | | | RVRQNDPDMKELNLNNIENIPKEML |
| | | | LDFVNAMKKNKHIKTFSLANVGADE |
| | | | SVAFALANMLRENRSITTLNIESNFIT |
| | | | GKGIVAIMRCLQFNETLTELRFHNQR |
| | | | HMLGHHAEMEISRLLKANTTLLKMG |
| | | | YHFELPGPRMVVTNLLTRNQDKQRQ |
| | | | KRQEEQKQQQLKEQKKLIAMLENGL |
| | | | GLPPGMWEMLGGPMPDSQMQEFLQ |
| | | | PPPPKSLHPQTAPFSRRNEVMAKPAQ |
| | | | PPKYRTDPDSFRVVKLKRIQRKSRMP |
| | | | EAREPPEKTNLKDVIKTLKPVPRNRP |
| | | | PPLVEITPRDQLLNDIRHSNIAYLKPV |
| | | | QLPKELA |
| SEQ ID | Q5KR49 | Tropomyosin | MDAIKKKMQMLKLDKENALDRAEQ |
| NO: 114 | (UniProtKB) | alpha-1 | AEADKKAAEDRSKQLEDELVSLQKK |
| | | chain/Bos taurus | LKATEDELDKYSEALKDAQEKLELA |
| | | | EKKATDAEADVASLNRRIQLVEEEL |
| | | | DRAQERLATALQKLEEAEKAADESE |
| | | | RGMKVIESRAQKDEEKMEIQEIQLKE |
| | | | AKHIAEDADRKYEEVARKLVIIESDL |
| | | | ERAEERAELSEGKCAELEEELKTVTN |
| | | | NLKSLEAQAEKYSQKEDKYEEEIKV |
| | | | LSDKLKEAETRAEFAERSVTKLEKSI |
| | | | DDLEDELYAQKLKYKAISEELDHAL |
| | | | NDMTSI |
| SEQ ID | F1NK75 | Tropomyosin | MEAIKKKMQMLKLDKENAIDRAEQ |
| NO: 115 | (UniProtKB) | 4/Gallus gallus | AETDKKAAEDKCKQVEDELVALQK |
| | | | KLKGTEDELDKYSEALKDAQEKLEQ |
| | | | AEKKATDAEGEVAALNRRIQLVEEE |
| | | | LDRAQERLATALQKLEEAEKAADES |
| | | | ERGMKVIENRAMKDEEKMEIQEMQ |
| | | | LKEAKHIAEEADRKYEEVARKLVILE |
| | | | GELERAEERAEVSEVKCSDLEEELKN |
| | | | VTNNLKSLEAQSEKYSEKEDKYEEEI |
| | | | KILSDKLKEAETRAEFAERTVAKLEK |
| | | | SIDDLEDELYAQKLKYKAISEELDHA |
| | | | LNDMTSL |
| SEQ ID | H9L074 | Tropomyosin/ | MEAIKKKMQMLKLDKENALDRAEQ |
| NO: 116 | (UniProtKB) | Gallus gallus | AEAEQKQAEERSKQLEDELAAMQK |
| | | | KLKGTEDELDKYSEALKDAQEKLEL |
| | | | AEKKAADAEAEVASLNRRIQLVEEE |
| | | | LDRAQERLATALQKLEEAEKAADES |
| | | | ERGMKVIENRALKDEEKMELQEIQL |
| | | | KEAKHIAEEADRKYEEVARKLVIIEG |
| | | | DLERTEERAELAESKCSELEEELKNV |
| | | | TNNLKSLEAQAEKYSQKEDKYEEEI |
| | | | KILTDKLKEAETRAEFAERSVAKLEK |
| | | | TIDDLEDELYAQKLKYKAISEELDHA |
| | | | LNDMTSMARRALQESSFGEMGHLP |
| | | | WFPEPQRVYPVSGRVDFFSGMGGLT |
| | | | YGLKRT |
| SEQ ID | E1BTG2 | Leiomodin- | MSTFGYRRELSKYEDIDEDELLASLT |
| NO: 117 | (UniProtKB) | 2/Gallus gallus | EEELKELERELEDIEPDRNLPVGQRQ |
| | | | KSLTEKTPTGTFSREALMAYWERET |
| | | | RKLLEKERLGACEKDSEQEEDNSEDI |
| | | | QEECFTESNSEVSEEAYTEEDDEEEE |
| | | | EEEEEEEDEDDSDDEDEEKQNSAASE |
| | | | RPVNCEDGRSSSHVRHKKCSNAKNS |
| | | | ENLFNGHDGKDTENLSFKSSAIHPCG |
| | | | NPTVIEDALEKVRSNDPETTEVNLNN |
| | | | IENITSQMLIQFSQALRDNTVVKSFSL |
| | | | ANTHADDNVAIAIAGMLKVNQHITS |
| | | | LNIESNFITGKGVLAIMRALQHNKVL |
| | | | TELRFHNQRHIMGSQVEMDIVKLLK |
| | | | ENTTLVKLGYHFDLAGPRMSMTSIL |
| | | | TRNMDKQRQKRMQEQRQQEYGCD |
| | | | GAINPKTKVLQKGTPRSSPYTSPKSSP |
| | | | WSSPKLPRKSAPAKSQPPAPAPPPPPP |
| | | | PPPPPPPPPPPVIPDKKAPTRNIAEVIK |
| | | | QQESSRKALQNGQKKKKGKKGKKH |
| | | | ENSILKEIKDSLKSVSDRKSEEGSRPS |
| | | | TRPSTPQRSLHDNLMEAIRASSIKQL |
| | | | RRVEVPEALR |
| SEQ ID | Q6QGC0 | PDZ and LIM | MPQNVILPGPAPWGFRLSGGIDFNQP |
| NO: 118 | (UniProtKB) | domain protein | LIITRITPGSKAAAANLCPGDVILAID |
| | | 3/Sus scrofa | GYGTESMTHADAQDRIKAAAHQLC |
| | | | LKIDRAETRLWSPQVTEDGKAHPFKI |
| | | | NLESEPQDVNYFEHKHNIRPKPFIIPG |
| | | | RSSGCSTPSGIDGGSGRSTPSSVSTLS |
| | | | TICPGDLKVAAKMAPNIPLEMELPGV |
| | | | KIVHAQFNTPMQLYSDDNIMETLQG |
| | | | QVSTALGETPSMSEPTTASVPPQSDV |
| | | | YRMLHDNRNEPTQPRQSGSFRVLQE |
| | | | LVNDGPDDRPAGTRSVRAPVTKIHG |
| | | | GAGGTQKMPLCDKCGSGIVGAVVK |
| | | | ARDKYRHPECFVCADCNLNLKQKG |
| | | | YFFVEGELYCETHARARMRPPEGYD |
| | | | TVTLYPKA |
| SEQ ID | Q3MHM1 | Calsequestrin/ | MTPSKSSKAPFTFLPSAFLGKKHFAQ |
| NO: 119 | (UniProtKB) | Bos taurus | MKRAHLFVVGVYLLSSCRAEEGLNF |
| | | | PTYDGKDRVVSLTEKNFKQVLKKYD |
| | | | LLCLYYHEPLSSDKVVQKQFQLKEIV |
| | | | LELVAQVLEHKDIGFVMVDAKKEA |
| | | | KLAKKLGFDEEGSLYILKGDRTIEFD |
| | | | GEFAADVLVEFLLDLIEDPVEIINSKL |
| | | | EVQAFERIEDHIKLIGFFKSEESEHYK |
| | | | AFEEAAEHFQPYIKFFATFDKGVAKK |
| | | | LSLKMNEVDFYEPFMDEPIAIPDKPY |
| | | | TEEELVEFVKEHQRPTLRRLRPEDMF |
| | | | ETWEDDLNGIHIVAFAERSDPDGYEF |
| | | | LEILKQVARDNTDNPDLSIVWIDPDD |
| | | | FPLLVAYWEKTFKIDLFKPQIGVVNV |
| | | | TDADSVWMDIPDDDDLPTAEELED |
| | | | WIEDVLSGKINTEDDDNDDEEDEDD |
| | | | DDDDDNSDEEDNDDSDDDDE |

Smooth muscle tissue

| | | | |
|---|---|---|---|
| SEQ ID | P08023 | Actin, aortic | MCEEEDSTALVCDNGSGLCKAGFAG |
| NO: 120 | (UniProtKB) | smooth | DDAPRAVFPSIVGRPRHQGVMVGM |
| | | muscle/Gallus | GQKDSYVGDEAQSKRGILTLKYPIEH |
| | | gallus | GIITNWDDMEKIWHHSFYNELRVAP |
| | | | EEHPTLLTEAPLNPKANREKMTQIMF |
| | | | ETFNVPAMYVAIQAVLSLYASGRTT |
| | | | GIVLDSGDGVTHNVPIYEGYALPHAI |
| | | | MRLDLAGRDLTDYLMKILSERGYSF |
| | | | VTTAEREIVRDIKEKLCYVALDFENE |
| | | | MATAASSSSLEKSYELPDGQVITIGN |
| | | | ERFRCPETLFQPSFIGMESAGIHETTY |
| | | | NSIMKCDIDIRKDLYANNVLSGGTT |
| | | | MYPGIADRMQKEITALAPSTMKIKII |
| | | | APPERKYSVWIGGSILASLSTFQQMW |
| | | | ISKQEYDEAGPSIVHRKCF |
| SEQ ID | P63270 | Actin, gamma- | MCEEETTALVCDNGSGLCKAGFAGD |
| NO: 121 | (UniProtKB) | enteric smooth | DAPRAVFPSIVGRPRHQGVMVGMG |
| | | muscle/Gallus | QKDSYVGDEAQSKRGILTLKYPIEHG |
| | | gallus | IITNWDDMEKIWHHSFYNELRVAPE |
| | | | EHPTLLTEAPLNPKANREKMTQIMFE |
| | | | TFNVPAMYVAIQAVLSLYASGRTTGI |
| | | | VLDSGDGVTHNVPIYEGYALPHAIM |
| | | | RLDLAGRDLTDYLMKILTERGYSFV |
| | | | TTAEREIVRDIKEKLCYVALDFENEM |
| | | | ATAASSSSLEKSYELPDGQVITIGNER |
| | | | FRCPETLFQPSFIGMESAGIHETTYNS |
| | | | IMKCDIDIRKDLYANNVLSGGTTMY |
| | | | PGIADRMQKEITALAPSTMKIKIIAPP |
| | | | ERKYSVWIGGSILASLSTFQQMWISK |
| | | | PEYDEAGPSIVHRKCF |
| SEQ ID | P24032 | Myosin | MSSKRAKTKTTKKRPQRATSNVFAM |
| NO: 122 | (UniProtKB) | regulatory light | FDQSQIQEFKEAFNMIDQNRDGFIDK |
| | | chain 2, smooth | EDLHDMLASLGKNPTDEYLDAMMN |
| | | muscle minor | EAPGPINFTMFLTMFGEKLNGTDPED |
| | | isoform/Gallus | VIRNAFACFDEEATGFIQEDYLRELL |
| | | gallus | TTMGDRFTDEEVDELYREAPIDKKG |
| | | | NFNYIEFTRILKHGAKDKDD |
| SEQ ID | P10587 | Myosin- | MSQKPLSDDEKFLFVDKNFVNNPLA |
| NO: 123 | (UniProtKB) | 11/Gallus gallus | QADWSAKKLVWVPSEKHGFEAASIK |
| | | | EEKGDEVTVELQENGKKVTLSKDDI |
| | | | QKMNPPKFSKVEDMAELTCLNEASV |
| | | | LHNLRERYFSGLIYTYSGLFCVVINP |
| | | | YKQLPIYSEKIIDMYKGKKRHEMPPH |
| | | | IYAIADTAYRSMLQDREDQSILCTGE |
| | | | SGAGKTENTKKVIQYLAWASSHKG |
| | | | KKDTSITQGPSFSYGELEKQLLQANPI |
| | | | LEAFGNAKTVKNDNSSRFGKFIRINF |
| | | | DVTGYIVGANIETYLLEKSRAIRQAK |
| | | | DERTFHIFYYLIAGASEQMRNDLLLE |
| | | | GFNNYTFLSNGHVPIPAQQDDEMFQ |
| | | | ETLEAMTIMGFTEEEQTSILRVVSSV |
| | | | LQLGNIVFKKERNTDQASMPDNTAA |
| | | | QKVCHLMGINVTDFTRSILTPRIKVG |
| | | | RDVVQKAQTKEQADFAIEALAKAKF |
| | | | ERLFRWILTRVNKALDKTKRQGASF |
| | | | LGILDIAGFEIFEINSFEQLCINYTNEK |
| | | | LQQLFNHTMFILEQEEYQREGIEWNF |
| | | | IDFGLDLQPCIELIERPTNPPGVLALL |
| | | | DEECWFPKATDTSFVEKLIQEQGNH |
| | | | AKFQKSKQLKDKTEFCILHYAGKVT |
| | | | YNASAWLTKNMDPLNDNVTSLLNQ |
| | | | SSDKFVADLWKDVDRIVGLDQMAK |
| | | | MTESSLPSASKTKKGMFRTVGQLYK |
| | | | EQLTKLMTTLRNTNPNFVRCIIPNHE |
| | | | KRAGKLDAHLVLEQLRCNGVLEGIR |
| | | | ICRQGFPNRIVFQEFRQRYEILAANAI |
| | | | PKGFMDGKQACILMIKALELDPNLY |
| | | | RIGQSKIFFRTGVLAHLEEERDLKITD |
| | | | VIIAFQAQCRGYLARKAFAKRQQQL |
| | | | TAMKVIQRNCAAYLKLRNWQWWR |
| | | | LFTKVKPLLQVTRQEEEMQAKDEEL |
| | | | QRTKERQQKAEAELKELEQKHTQLC |
| | | | EEKNLLQEKLQAETELYAEAEEMRV |
| | | | RLAAKKQELEEILHEMEARIEEEEER |
| | | | SQQLQAEKKKMQQQMLDLEEQLEE |
| | | | EEAARQKLQLEKVTADGKIKKMED |
| | | | DILIMEDQNNKLTKERKLLEERVSDL |
| | | | TTNLAEEEEKAKNLTKLKNKHESMI |
| | | | SELEVRLKKEEKSRQELEKIKRKLEG |
| | | | ESSDLHEQIAELQAQIAELKAQLAKK |
| | | | EEELQAALARLEDETSQKNNALKKI |
| | | | RELESHISDLQEDLESEKAARNKAEK |
| | | | QKRDLSEELEALKTELEDTLDTTATQ |
| | | | QELRAKREQEVTVLKRALEEETRTH |
| | | | EAQVQEMRQKHTQAVEELTEQLEQF |
| | | | KRAKANLDKTKQTLEKDNADLANEI |
| | | | RSLSQAKQDVEHKKKKLEVQLQDL |
| | | | QSKYSDGERVRTELNEKVHKLQIEV |
| | | | ENVTSLLNEAESKNIKLTKDVATLGS |
| | | | QLQDTQELLQEETRQKLNVTTKLRQ |
| | | | LEDDKNSLQEQLDEEVEAKQNLERH |
| | | | ISTLTIQLSDSKKKLQEFTATVETMEE |
| | | | GKKKLQREIESLTQQFEEKAASYDKL |
| | | | EKTKNRLQQELDDLVVDLDNQRQL |
| | | | VSNLEKKQKKFDQMLAEEKNISSKY |
| | | | ADERDRAEAEAREKETKALSLARAL |
| | | | EEALEAKEELERTNKMLKAEMEDLV |
| | | | SSKDDVGKNVHELEKSKRTLEQQVE |
| | | | EMKTQLEELEDELQAAEDAKLRLEV |
| | | | NMQAMKSQFERDLQARDEQNEEKR |
| | | | RQLLKQLHEHETELEDERKQRALAA |
| | | | AAKKKLEVDVKDLESQVDSANKAR |
| | | | EEAIKQLRKLQAQMKDYQRDLDDA |
| | | | RAAREEIFATARENEKKAKNLEAELI |
| | | | QLQEDLAAAERARKQADLEKEEMA |
| | | | EELASANSGRTSLQDEKRRLEARIAQ |
| | | | LEEELDEEHSNIETMSDRMRKAVQQ |
| | | | AEQLNNELATERATAQKNENARQQL |
| | | | ERQNKELRSKLQEMEGAVKSKFKST |
| | | | IAALEAKIASLEEQLEQEAREKQAAA |
| | | | KTLRQKDKKLKDALLQVEDERKQA |
| | | | EQYKDQAEKGNLRLKQLKRQLEEAE |
| | | | EESQRINANRRKLQRELDEATESNDA |
| | | | LGREVAALKSKLRRGNEPVSFAPPRR |
| | | | SGGRRVIENATDGGEEEIDGRDGDFN |
| | | | GKASE |
| SEQ ID | P02607 | Myosin light | MCDFSEEQTAEFKEAFQLFDRTGDG |
| NO: 124 | (UniProtKB) | polypeptide | KILYSQCGDVMRALGQNPTNAEVM |
| | | 6/Gallus gallus | KVLGNPKSDEMNLKTLKFEQFLPMM |
| | | | QTIAKNKDQGCFEDYVEGLRVFDKE |
| | | | GNGTVMGAEIRHVLVTLGEKMTEEE |
| | | | VEQLVAGHEDSNGCINYEELVRMVL |
| | | | SG |
| SEQ ID | P02542 | Desmin/Gallus | QSYSSSQRVSSYRRTFGGGTSPVFPR |
| NO: 125 | (UniProtKB) | gallus | ASFGSRGSGSSVTSRVYQVSRTSAVP |
| | | | TLSTFRTTRVTPLRTYGSAYQGAGEL |
| | | | LDFSLADAMNQEFLQTRTNEKVELQ |
| | | | ELNDRFANYIEKVRFLEQQNALMVA |
| | | | EVNRLRGKQPTRVAEMYEEELRELR |
| | | | RQVDALTGQRARVEVERDNLLDNL |
| | | | QKLKQKLQEEIQLKQEAENNLAAFR |
| | | | ADVDAATLARIDLERRIESLQEEIAFL |
| | | | KKVHEEEIRELQAQLQEQHIQVEMDI |
| | | | SKPDLTAALRDIRAQYESIAAKNIAE |
| | | | AEEWYKSKVSDLTQAANKNNDALR |
| | | | QAKQEMLEYRHQIQSYTCEIDALKG |
| | | | TNDSLMRQMREMEERFAGEAGGYQ |
| | | | DTIARLEEEIRHLKDEMARHLREYQD |
| | | | LLNVKMALDVEIATYRKLLEGEENRI |
| | | | SIPMHQTFASALNFRETSPDQRGSEV |
| | | | HTKKTVMIKTIETRDGEVVSEATQQ |
| | | | QHEVL |
| SEQ ID | P19352-2 | Isoform 2 of | MEAIKKKMQMLKLDKENAIDRAEQ |
| NO: 126 | (UniProtKB) | Tropomyosin | AEADKKQAEDRCKQLEEEQQGLQK |
| | | beta | KLKGTEDEVEKYSESVKEAQEKLEQ |
| | | chain/Gallus | AEKKATDAEAEVASLNRRIQLVEEE |
| | | gallus | LDRAQERLATALQKLEEAEKAADES |
| | | | ERGMKVIENRAMKDEEKMELQEMQ |
| | | | LKEAKHIAEEADRKYEEVARKLVVL |
| | | | EGELERSEERAEVAESRVRQLEEELR |
| | | | TMDQSLKSLIASEEEYSTKEDKYEEE |
| | | | IKLLGEKLKEAETRAEFAERSVAKLE |
| | | | KTIDDLEESLASAKEENVGIHQVLDQ |
| | | | TLLELNNL |
| SEQ ID | Q2QLE2 | Caveolin-2/Sus | MGLETEKADVQLFMDDDSYSRHSG |
| NO: 127 | (UniProtKB) | scrofa | VDYADPKKFVDPGTDRDPHRLNSNL |
| | | | KVGFEDVIAEPVSTHSFDKVWICSHA |
| | | | LFEISKYVIYKFLTVFLAIPLAFAAGIL |
| | | | FATLSCLHIWIIIPFVKTCLMVLPSVQ |
| | | | TIWKSVTDVVIAPLCTSAGRSFSSVSL |
| | | | QLSHD |
| SEQ ID | Q90623 | Protein | MKMADAKQKRNEQLKRWIGSETDL |
| NO: 128 | (UniProtKB) | phosphatase 1 | EPPVVKRKKTKVKFDDGAVFLAACS |
| | | regulatory | SGDTEEVLRLLERGADINYANVDGL |
| | | subunit | TALHQACIDDNVDMVKFLVENGANI |
| | | 12A/Gallus | NQPDNEGWIPLHAAASCGYLDIAEY |
| | | gallus | LISQGAHVGAVNSEGDTPLDIAEEEA |
| | | | MEELLQNEVNRQGVDIEAARKEEER |
| | | | IMLRDARQWLNSGHINDVRHAKSGG |
| | | | TALHVAAAKGYTEVLKLLIQARYDV |
| | | | NIKDYDGWTPLHAAAHWGKEEACR |
| | | | ILVENLCDMEAVNKVGQTAFDVADE |
| | | | DILGYLEELQKKQNLLHSEKREKKSP |
| | | | LIESTANLDNNQTQKTFKNKETLIME |
| | | | QEKNASSIESLEHEKADEEEEGKKDE |
| | | | SSCSSEEEEDDDSESEAETDKAKTLA |
| | | | NANTTSTQSASMTAPSVAGGQGTPT |
| | | | SPLKKFPTSTTKVSPKEEERKDESPAS |
| | | | WRLGLRKTGSYGALAEITASKEAQK |
| | | | EKDSAGVIRSASSPRLSSSLDNKEKE |
| | | | KDGKGTRLAYVAPTIPRRLASTSDID |
| | | | EKENRDSSASSIRSGSSYARRKWEED |
| | | | VKKNSLNEGPTSLNTSYQRSGSFGRR |
| | | | QDDLVSSNVPSTASTVTSSAGLQKTL |
| | | | PASANTTTKSTTGSTSAGVQSSTSNR |
| | | | LWAEDSTEKEKDSVPTAVTVPVAPS |
| | | | VVNAAATTTAMTTATSGTVSSTSEV |
| | | | RERRRSYLTPVRDEESESQRKARSRQ |
| | | | ARQSRRSTQGVTLTDLQEAEKTIGRS |
| | | | RSTRTREQENEEKEKEEKEKQDKEK |
| | | | QEEKKESETKDDDYRQRYSRTVEEP |
| | | | YHRYRPTSTSTSTSSTSSLSTSTSSLSS |
| | | | SSQLNRPNSLIGITSAYSRSGTKESER |
| | | | EGGKKEEEKEEDKSQPKSIRERRRPR |
| | | | EKRRSTGVSFWTQDSDENEQEHQSD |
| | | | SEEGTNKKETQSDSLSRYDTGSLSVS |
| | | | SGDRYDSAQGRSGSQSYLEDRKPYC |
| | | | SRLEKEDSTDFKKLYEQILAENEKLK |
| | | | AQLHDTNMELTDLKLQLEKTTQRQE |
| | | | RFADRSLLEMEKRVSGKSQYLLGGK |
| | | | KSSRKKDI |
| SEQ ID | P26932-2 | Isoform Beta of | MSNANFNRGPAYGLSAEVKNKLAQ |
| NO: 129 | (UniProtKB) | Calponin- | KYDPQTERQLRVWIEGATGRRIGDN |
| | | 1/Gallus gallus | FMDGLKDGVILCELINKLQPGSVQK |
| | | | VNDPVQNWHKLENIGNFLRAIKHYG |
| | | | VKPHDIFEANDLFENTNHTQVQSTLI |
| | | | ALASQAKTKGNNVGLGVKYAEKQQ |
| | | | RRFQPEKLREGRNIIGLQMGTNKFAS |
| | | | QQGMTAYGTRRHLYDPKLGTDQPL |
| | | | DQATISLQMGTNKGASQGMTVYGLP |
| | | | RQVYDPKYCDAPGLLGEDGLNHSFY |
| | | | NSQ |
| SEQ ID | Q7YRL2 | Calponin-1/Ovis | MSSAHFNRGPAYGLSAEVKNKLAQ |
| NO: 130 | (UniProtKB) | aries | KYDHQREQELREWIEGVTGRRIGNN |
| | | | FMDGLKDGIILCEFINKLQPGSVKKV |
| | | | NESTQNWHQLENIGNFIKAITKYGVK |
| | | | PHDIFEANDLFENTNHTQVQSTLLAL |
| | | | ASMAKTKGNKVNVGVKYAEKQERK |
| | | | FEPEKLREGRNIIGLQMGTNKFASQQ |
| | | | GMTAYGTRRHLYDPKLGTDQPLDQ |
| | | | ATISLQMGTNKGASQAGMTAPGTKR |
| | | | QIFEPGLGMEHCDTLNVSLQMGSNK |
| | | | GASQRGMTVYGLPRQVYDPKYCLTP |
| | | | EYPELGEPAHNHHPHNYYNSA |
| SEQ ID | A0A212D0J2 | Calponin/Cervus | MSSTQFNKGPSYGLSAEVKNRLQSK |
| NO: 131 | (UniProtKB) | elaphus | YDPQKEAELRSWIEGLTGLSIGPDFQ |
| | | hippelaphus | KGLKDGIILCTLMNKLQPGSIPKINRS |
| | | | MQNWHQLENLSNFIKAMAKTKGLQ |
| | | | SGVDIGLKYSEKQERNFDDATMKAG |
| | | | QCVIGLQVGMTAPGTRRHIYDTKLG |
| | | | TDKCDNSSMSLQMGYTQGANQSGQ |
| | | | VFGLGRQIYDPKYCPQGPVADGAPA |
| | | | AAGDGPGPGEPSECPPYYQEEAGY |
| SEQ ID | Q5ZKU6 | Calponin/Gallus | MSSSQFNKGPSYGLSAEVKNRLAQK |
| NO: 132 | (UniProtKB) | gallus | YDPQKEAELRTWIESVTGRQIGADFQ |
| | | | KGLKDGVILCELMNKLQPNSVRKIN |
| | | | RSALNWHQLENLSNFIKAMVSYGM |
| | | | NPVDLFEANDLFESGNLTQVQVSLL |
| | | | ALAGMAKTKGLQSGVDIGVKYSER |
| | | | QQRNFDEAKMKAGQCVIGLQMGTN |
| | | | KCASQSGMTAYGTRRHLYDPKNQIL |
| | | | PPMDHSTISLQMGTNKCASQVGMTA |
| | | | PGTRRHIYDAKMGLEKCDNSSMSLQ |
| | | | MGSNQGANQSGQVFGLGRQIYDPK |
| | | | YCPQGTPGDAANAAGEPGADPPGYH |
| | | | YYHQEESC |
| SEQ ID | E1BSX2 | Calponin/Gallus | MTHFNKGPSYGLSAEVKNKIALKYD |
| NO: 133 | (UniProtKB) | gallus | PQIEEDLRNWIEEVTGLSIGANFQLG |
| | | | LKDGIILCELINKLQPGSVKKINQSKL |
| | | | NWHQLENIGNFIKAIQVYGMKPHDIF |
| | | | EANDLFENGNMTQVQTTLVALAGL |
| | | | AKTKGFHTTIDIGVKYAEKQARSFD |
| | | | AGKLKAGQSVIGLQMGTNKCASQA |
| | | | GMTAYGTRRHLYDPKMQTDKPFDQ |
| | | | TTISLQMGTNKGASQAGMLAPGTRR |
| | | | DIYDQKHILQPVDNSTISLQMGTNKV |
| | | | ASQKGMSVYGLGRQVYDPKYCAAP |
| | | | TEPVIHNGSQGTGTNGSEISDSDYQA |
| | | | EYPDDYHGEYQDDYQRDYHGQYSD |
| | | | QGIDY |
| SEQ ID | P19966 | Transgelin/ | MANKGPAYGMSRDVQSKIEKKYDD |
| NO: 134 | (UniProtKB) | Gallus gallus | ELEDRLVEWIVAQCGSSVGRPDRGR |
| | | | LGFQVWLKNGIVLSQLVNSLYPDGS |
| | | | KPVKIPDSPPTMVFKQMEQIAQFLKA |
| | | | AEDYGVVKTDMFQTVDLFEAKDMA |
| | | | AVQRTLVALGSLAVTKNDGHYHGD |
| | | | PNWFMKKAQEHKREFSESQLKEGK |
| | | | NIIGLQMGTNKGASQAGMSYGRPRQ |
| | | | IIS |
| SEQ ID | A0A1L1RTM1 | Caldesmon/ | RLSYQRNDDDEEEAARERRRRARQE |
| NO: 135 | (UniProtKB) | Gallus gallus | RLRQKEEGDVSGEVTEKSEVNAQNS |
| | | | VAEEETKRSTDDEAALLERLARREE |
| | | | RRQKRLQEALERQKEFDPTITDGSLS |
| | | | VPSRREVNNVEENETTGKEEKVETR |
| | | | QGRCEIEETETVTKSYQRNNWRQDG |
| | | | EEEGKKEEKDSEEEKPKEVPTEENQV |
| | | | DVAVEKSTDKEEVVETKTLAVNAEN |
| | | | DTNAMLEGEQSITDAADKEKEEAEK |
| | | | EREKLEAEEKERLKAEEEKKAAEEK |
| | | | QKAEEEKKAAEERERAKAEEEKRAA |
| | | | EERERAKAEEERKAAEERERAKAEE |
| | | | ERKAAEERAKAEEERKAAEERAKAE |
| | | | EERKAAEERAKAEKERKAAEERERA |
| | | | KAEEEKRAAEEKARLEAEKLKEKKK |
| | | | MEEKKAQEEKAQANLLRKQEEDKE |
| | | | AKVEAKKESLPEKLQPTSKKDQVKD |
| | | | NKDKEKAPKEEMKSVWDRKRGVPE |
| | | | QKAQNGERELTTPKLKSTENAFGRS |
| | | | NLKGAANAEAGSEKLKEKQQEAAV |
| | | | ELDELKKRREERRKILEEEEQKKKQE |
| | | | EAERKIREEEEKKRMKEEIERRRAEA |
| | | | AEKRQKVPEDGVSEEKKPFKCFSPK |
| | | | GSSLKIEERAEFLNKSAQKSGMKPAH |
| | | | TTAVVSKIDSRLEQYTSAVVGNKAA |
| | | | KPAKPAASDLPVPAEGVRNIKSMWE |
| | | | KGNVFSSPGGTGTPNKETAGLKVGV |
| | | | SSRINEWLTKTPEGNKSPAPKPSDLR |
| | | | PGDVSGKRNLWEKQSVEKPAASSSK |
| | | | VTATGKKSETNGLRQFEKEP |
| SEQ ID | E1C2S1 | Talin-1/Gallus | MVALSLKISIGNVVKTMQFEPSTMV |
| NO: 136 | (UniProtKB) | gallus | YDACRMIRERVPEAQMGQPNDFGLF |
| | | | LSDEDPKKGIWLEAGKALDYYMLR |
| | | | NGDTMEYKKKQRPLKIRMLDGTVK |
| | | | TVMVDDSKTVTDMLMTICARIGITN |
| | | | YDEYSLVREIMEEKKEEVTGTLKKD |
| | | | KTLLRDEKKMEKLKQKLHTDDELN |
| | | | WLDHGRTLREQGIDDNETLLLRRKF |
| | | | FYSDQNVDSRDPVQLNLLYVQARDD |
| | | | ILNGSHPVSFDKACEFAGYQCQIQFG |
| | | | PHNEQKHKPGFLELKDFLPKEYIKQK |
| | | | GERKIFMAHKNCGNMSEIEAKVRYV |
| | | | KLARSLKTYGVSFFLVKEKMKGKNK |
| | | | LVPRLLGITKECVMRVDEKTKEVIQE |
| | | | WSLTNIKRWAASPKSFTLDFGDYQD |
| | | | GYYSVQTTEGEQIAQLIAGYIDIILKK |
| | | | KKSKDHFGLEGDEESTMLEDSVSPK |
| | | | KSTVLQQQFNRVGKAELGSVALPAI |
| | | | MRTGAGGPENFQVGTMPQAQMQIT |
| | | | SGQMHRGHMPPLTSAQQALTGTINS |
| | | | SMQAVNAAQATLDDFETLPPLGQDA |
| | | | ASKAWRKNKMDESKHEIHSQVDAIT |
| | | | AGTASVVNLTAGDPADTDYTAVGC |
| | | | AVTTISSNLTEMSKGVKLLAALMED |
| | | | EGGNGRQLLQAAKNLASAVSDLLKT |
| | | | AQPASAEPRQNLLQAAGLVGQTSGE |
| | | | LLQQIGESDTDPRFQDMLMQLAKAV |
| | | | ASAAAALVLKAKNVAQKTEDSALQ |
| | | | TQVIAAATQCALSTSQLVACTKVVA |
| | | | PTISSPVCQEQLIEAGKLVAKSAEGC |
| | | | VEASKAATNDDQLLKQVGVAATAV |
| | | | TQALNDLLQHIKQHATGGQPIGRYD |
| | | | QATDTILNVTENIFSSMGDAGEMVR |
| | | | QARILAQATSDLVNAIKADAEGETD |
| | | | LENSRKLLSAAKILADATAKMVEAA |
| | | | KGAAAHPDSEEQQQRLREAAEGLR |
| | | | MATNAAAQNAIKKKLVHKLEHAAK |
| | | | QAAASATQTIAAAQHAAASNKNPAA |
| | | | QQQLVQSCKVVADQIPMLVQGVRG |
| | | | SQSQPDSPSAQLALIAASQNFLQPGG |
| | | | KMVAAAKATVPTITDQASAMQLSQ |
| | | | CAKNLAAALAELRTAAQKAQEACG |
| | | | PLEIDSALGLVQSLERDLKEAKAAAR |
| | | | DGKLKPLPGETMEKCAQDLGNSTKA |
| | | | VTSAIAHLLGEVAQGNENYTGIAAR |
| | | | EVAQALRSLSQAARGVAANSSDPQV |
| | | | QNAMLECASDVMDKANNLIEEARK |
| | | | AVAKPGDPDSQQRLVQVAKAVSQA |
| | | | LNRCVNCLPGQRDVDAAIRMVGEAS |
| | | | KRLLSDSFPPSNKTFQEAQSQLNRAA |
| | | | AGLNQSANELVQASRGTPQDLAKSS |
| | | | GKFGQDFNEFLQAGVEMASLSPTKE |
| | | | DQAQVVSNLKSISMSSSKLLLAAKA |
| | | | LSADPTSPNLKSQLAAAARAVTDSIN |
| | | | QLITMCTQQAPGQKECDNALRELET |
| | | | VKELLENPTQTVNDMSYFSCLDSVM |
| | | | ENSKVLGESMAGISQNAKNSKLPEF |
| | | | GESISAASKALCGLTEAAAQAAYLV |
| | | | GVSDPNSQAGQQGLVDPTQFARANQ |
| | | | AIQMACQNLVDPACTQSQVLSAATI |
| | | | VAKHTSALCNTCRLASSRTANPVAK |
| | | | RQFVQSAKEVANSTANLVKTIKALD |
| | | | GAFNEENRERCRAATAPLIEAVDNLT |
| | | | AFASNPEFATVPAQISPEGRRAMEPI |
| | | | VTSAKTMLESSAGLIQTARSLAVNPK |
| | | | DPPQWSVLAGHSRTVSDSIKKLITNM |
| | | | RDKAPGQRECDEAIDVLNRCMREVD |
| | | | QASLAAISQQLAPREGISQEALHNQM |
| | | | ITAVQEINNLIEPVASAARAEASQLG |
| | | | HKVSQMAQYFEPLILAAIGAASKTPN |
| | | | HQQQMNLLDQTKTLAESALQMLYT |
| | | | AKEAGGNPKQAAHTQEALEEAVQM |
| | | | MKEAVEDLTTTLNEAASAAGVVGG |
| | | | MVDSITQAINQLDEGPMGEPEGTFV |
| | | | DYQTTMVKTAKAIAVTVQEMVTKS |
| | | | TTNPDELGILANQLTNDYGQLAQQA |
| | | | KPAALTAENEEIGSHIKRRVQELGHG |
| | | | CAALVTKAGALQCSPSDAYTKKELI |
| | | | ESARKVSEKVSHVLAALQAGNRGTQ |
| | | | ACITAASAVSGIIADLDTTIMFATAGT |
| | | | LNRENSETFADHREGILKTAKALVED |
| | | | TKVLVQNATASQEKLAQAAQSSVST |
| | | | ITRLAEVVKLGAASLGSEDPETQVVL |
| | | | INAVKDVAKALGDLIGATKAAAGKA |
| | | | GDDPAVYQLKNSAKVMVTNVTSLL |
| | | | KTVKAVEDEATKGTRALEATIEHIRQ |
| | | | ELAVFSSPVPPAQVSTPEDFIRMTKGI |
| | | | TMATAKAVAAGNSCRQEDVIATAN |
| | | | LSRRAIADMLRACKEAAYHPEVSAD |
| | | | VRQRALRFGKECADGYLELLEHVLV |
| | | | ILQKPTHELKQQLAGYSKRVASSVTE |
| | | | LIQAAEAMKGTEWVDPEDPTVIAEN |
| | | | ELLGAAAAIEAAAKKLEQLKPRAKP |
| | | | KQADESLDFEEQILEAAKSIAAATSA |
| | | | LVKAASAAQRELVAQGKVGVIPANA |
| | | | VDDGQWSQGLISAARMVAAATNNL |
| | | | CEAANAAVQGHASEEKLISSAKQVA |
| | | | ASTAQLLVACKVKADHDSEAMKRL |
| | | | QAAGNAVKRASDNLVKAAQKAAAF |
| | | | QDHDETVVVKEKMVGGIAQIIAAQE |
| | | | EMLRKERELEEARKKLAMIRQQQYK |
| | | | FLPTELRDEEQN |
| SEQ ID | E1BXG1 | Profilin/Gallus | MNCWNYYTDCILSDKYIDDVAIVGL |
| NO: 137 | (UniProtKB) | gallus | SDNKYVWAAKPGGLLSAVSPREVDL |
| | | | ITGQDRITFLTAGISIAGKKCIVIRDSL |
| | | | LVEGDNVMDIRSRGGDSRSICIGKTP |
| | | | KALIFLMGNRGVHGGVLNLKIHDMI |
| | | | AGMTL |
| SEQ ID | E1C9A2 | Profilin/Gallus | MIQLQALLKESLIRTKHVENAAAIGI |
| NO: 138 | (UniProtKB) | gallus | NEREVCASTSGFYVPPENAINLIYAF |
| | | | YKNLLQVRKEGLYFRQKHYECVRA |
| | | | DEHSIYLKNAEGGLIVVKTNALILIAT |
| | | | YRVGMYPSVCVEAVEKLGKTNTCT |
| | | | CMAACQRFIGWLCSCKELQKCV |
| SEQ ID | 093256 | Keratin, type 1 | MATYSFRQTTSSVAGGPCGRSLRLG |
| NO: 139 | (UniProtKB) | cytoskeletal | GGSFRAPSIHGGSGGRGVSVSSARFV |
| | | 19/Gallus gallus | SSGLGSGLGGGYGGAFSSSFSAGFGG |
| | | | GYGGGLGSGDGLLSGNEKTTMQNL |
| | | | NDRLASYLDKVRALEEANSDLETKI |
| | | | REWYLKQGPGPARDYSPYYKAIEDL |
| | | | RDQILAATIDNSKVVLQIDNARLAAD |
| | | | DFKTKFETEQALRMSVEADINGLRR |
| | | | VLDELTLARTDLELQIENLKEELAYL |
| | | | KKNHEEEMSALGGQVASQVSVEVD |
| | | | SAPGIDLSKILADMRDQYEHMAEKN |
| | | | RKDAEAWFHSKTEELNRELAVNTEQ |
| | | | LQSSKSEVTDLRRTLQGLEIELQSQLS |
| | | | MKGALESTLADTEGRYGAQLAQIQD |
| | | | MIGSIEAQLAELRADMERQNSEYKM |
| | | | LMDIKTRLEQEIATYRQLLEGQESQL |
| | | | FGSLSGSPDKRDKPADGK |

Skeletal and cardiac muscle tissue

| | | | |
|---|---|---|---|
| SEQ ID | B5X7T1 | Troponin C, | MDDVYKAAVENLTEEQKNEFKAAF |
| NO: 140 | (UniProtKB) | slow skeletal | DIACQGAEDGCISTKELGKVMRMLG |
| | | and cardiac | QNPTPEELQEMIDEVDEDGSGTVDF |
| | | muscles/Salmo | DEFLVMMVRCMKEESKGKSEEELAE |
| | | salar | LFRMFDKNGDGYIDLEELKTMLEST |
| | | | GEAITEDDIEELMKDGDKNNDGKID |
| | | | YDEFLEFMKGVE |
| SEQ ID | P09860 | Troponin C, | MDDIYKAAVEQLTEEQKNEFKAAFD |
| NO: 141 | (UniProtKB) | slow skeletal | IFVLGAEDGCISTKELGKVMRMLGQ |
| | | and cardiac | NPTPEELQEMIDEVDEDGSGTVDFDE |
| | | muscles/Gallus | FLVMMVRCMKDDSKGKTEEELSDL |
| | | gallus | FRMFDKNADGYIDLEELKIMLQATG |
| | | | ETITEDDIEELMKDGDKNNDGRIDYD |
| | | | EFLEFMKGVE |
| SEQ ID | P63315 | Troponin C, | MDDIYKAAVEQLTEEQKNEFKAAFD |
| NO: 142 | (UniProtKB) | slow skeletal | IFVLGAEDGCISTKELGKVMRMLGQ |
| | | and cardiac | NPTPEELQEMIDEVDEDGSGTVDFDE |
| | | muscles/Bos | FLVMMVRCMKDDSKGKSEEELSDLF |
| | | taurus | RMFDKNADGYIDLEELKIMLQATGE |
| | | | TITEDDIEELMKDGDKNNDGRIDYDE |
| | | | FLEFMKGVE |
| SEQ ID | P14315-2 | Isoform 2 of F- | MSDQQLDCALDLMRRLPPQQIEKNL |
| NO: 143 | (UniProtKB) | actin-capping | SDLIDLVPSLCEDLLSSVDQPLKIARD |
| | | protein subunit | KVVGKDYLLCDYNRDGDSYRSPWS |
| | | beta isoforms 1 | NKYDPPLEDGAMPSARLRKLEVEAN |
| | | and 2/Gallus | NAFDQYRDLYFEGGVSSVYLWDLD |
| | | gallus | HGFAGVILIKKAGDGSKKIKGCWDSI |
| | | | HVVEVQEKSSGRTAHYKLTSTVML |
| | | | WLQTNKTGSGTMNLGGSLTRQMEK |
| | | | DETVSDSSPHIANIGRLVEDMENKIR |
| | | | STLNEIYFGKTKDIVNGLRSVQTFAD |
| | | | KSKQEALKNDLVEALKRKQQS |
| SEQ ID | E1BMP3 | Myosin light | MSGAPKESLGPQGLPGLGKACLTTM |
| NO: 144 | (UniProtKB) | chain kinase | DKKLNMLNEKVDKLLHFQEDVTEK |
| | | 3/Bos taurus | LQCVYRGMGHLEQGLHRLEASRGL |
| | | | GPAGADRSPPSDAQAGWPEVLELVR |
| | | | AGRQDAAQQGARLEALFRMVMAV |
| | | | DKAIALVGAVLQNSKVVDFIMQGSV |
| | | | PWRKGSLADNKEQVEEKEAKPKHTL |
| | | | STRGVQAELRGPWEESQKADLPEGT |
| | | | GSDLPTQTEAPPEQRDGISGPTQVRP |
| | | | EVEVQAPRASSKNPGIGLELSVVSER |
| | | | VSEAPTGQEAALSAGRGTSPSRPDPR |
| | | | PSVEGMRLTPAPPAQAKAAHGGGET |
| | | | PPRISIHVQETDTPGELLVTRGGSLRT |
| | | | SPPAETPAAVPPGEQDPPGPRCCPQA |
| | | | PGTESGKPILRGASVRKRSCDEGAKA |
| | | | KEKQGPGSELTMAPSRARRDKGADS |
| | | | GASGPQQDMNPGAGNPDPGKDCTA |
| | | | GGVGSAEAGSRTPPGAEASSLVLDD |
| | | | SPAPPAPFEHRVVSVRETSTSAGYTV |
| | | | CQHEVLGGGRFGQVHRCTEKATGLS |
| | | | LAAKIIKVKSAKDREDVKNEINIMNQ |
| | | | LSHVNLIQLYDAFESKNSFTLVMEYV |
| | | | DGGELFDRITEEKYHLTELDVVLFTK |
| | | | QICEGVHYLHQHYVLHLDLKPENILC |
| | | | VNQTGHQIKIIDFGLARRYKPREKLK |
| | | | VNFGTPEFLAPEVVNYEFVSFPTDM |
| | | | WSVGVITYMLLSGLSPFLGETDAET |
| | | | MNFIVNCNWDFDADTFEGLSEEAKD |
| | | | FVSRLLVKEKSCRMSATQCLKHEWL |
| | | | NNLPAKASKSKVHLKSQLLLQKYM |
| | | | AQRKWKKHFYVVTAANRLRKFPTC |
| | | | P |
| SEQ ID | B5X8Q3 | Troponin | MNDIYKAAVEQLTDEQKNEFKAAFD |
| NO: 145 | (UniProtKB) | C/Salmo salar | IFVQDAEDGCISTKELGKVMRMLGQ |
| | | | NPTPEELQEMIDEVDEDGSGTVDFDE |
| | | | FLVMMVRCMKDDSKGKTEEELADL |
| | | | FRMFDKNADGYIDLEELKVMLEATG |
| | | | EAITEDDIEELMKDGDKNNDGKIDY |
| | | | DEFLEFMKGVE |
| SEQ ID | A4GR69 | Telethonin/Sus | MATSELSCQVSEENCERREAFWAEW |
| NO: 146 | (UniProtKB) | scrofa | KDLTLSTRPEEGCSLHEEDAERRETY |
| | | | HQQGQCQALVQRSPWLVMRMGILG |
| | | | RGLQEYQLPYQRVLPLPIFTPAKVGA |
| | | | AKEEREETPIQLRELLALETALGGQC |
| | | | LDRQDVAEITKQLPPVVPVSKPGALR |
| | | | RSLSRSMSQEAQRG |
| SEQ ID | P04268 | Tropomyosin | MDAIKKKMQMLKLDKENALDRAEQ |
| NO: 147 | (UniProtKB) | alpha-1 | AEADKKAAEERSKQLEDELVALQKK |
| | | chain/Gallus | LKGTEDELDKYSESLKDAQEKLELA |
| | | gallus | DKKATDAESEVASLNRRIQLVEEELD |
| | | | RAQERLATALQKLEEAEKAADESER |
| | | | GMKVIENRAQKDEEKMEIQEIQLKE |
| | | | AKHIAEEADRKYEEVARKLVIIEGDL |
| | | | ERAEERAELSESKCAELEEELKTVTN |
| | | | NLKSLEAQAEKYSQKEDKYEEEIKV |
| | | | LTDKLKEAETRAEFAERSVTKLEKSI |
| | | | DDLEDELYAQKLKYKAISEELDHAL |
| | | | NDMTSI |
| SEQ ID | Q05706 | Beta- | MEAIKKKMQMLKLDKENAIDRAEQ |
| NO: 148 | (UniProtKB) | tropomyosin/ | AEADKKQAEDRCKQLEEEQQGLQK |
| | | Gallus gallus | KLKGTEDEVEKYSESVKEAQEKLEQ |
| | | | AEKKATDAEAEVASLNRRIQLVEEE |
| | | | LDRAQERLATALQKLEEAEKAADES |
| | | | ERGMKVIENRAMKDEEKMELQEMQ |
| | | | LKEAKHIAEEADRKYEEVARKLVVL |
| | | | EGELERSEERAEVAESKCGDLEEELK |
| | | | IVTNNLKSLEAQADKYSTKEDKYEE |
| | | | EIKLLGEKLKEAETRAEFAERSVAKL |
| | | | EKTIDDLEERSRQEAEKNRVLTNELR |
| | | | VILTELNN |
| SEQ ID | Q2KI43 | Caveolin-3/Bos | MMAEEHTDLEAQIVKDIHFKEIDLV |
| NO: 149 | (UniProtKB) | taurus | NRDPKNINEDIVKVDFEDVIAEPVGT |
| | | | YSFDGVWKVSYTTFTVSKYWCYRL |
| | | | LSTLLGVPLALLWGFLFACISFCHIW |
| | | | AVVPCIKSYLIEIQCISHIYSLCIRTFC |
| | | | NPLFAALGQVCSNIKVMLRKEV |
| SEQ ID | Q02173 | M-protein, | MSSVAVPFYQRRHKHFDQSYRNIQT |
| NO: 150 | (UniProtKB) | striated | RYVLEEYAARKAASRQAAHYESTGL |
| | | muscle/Gallus | GKTTCRLCARRARSLAHEAMQESRK |
| | | gallus | RTHEQKSHASDEKRIKFASELSSLER |
| | | | EIHMARHHAREQLDRLAIQRMVEEN |
| | | | MALERHVVEEKISRAPEILVRLRSHT |
| | | | VWEKMSVRLCFTVQGFPSPVVQWY |
| | | | KNEELITPASDPAKYSVENKYGVHV |
| | | | LHINRADFDDSATYSAVATNIHGQAS |
| | | | TNCAVVVRRFRESEEPHPAGIMPFHL |
| | | | PLSYDVCFTHFDVQFLEKFGVTFATE |
| | | | GETLTLKCSVLVTPELKRLRPRAEW |
| | | | YRDDVLIKDSKWTKLYFGEGQAALS |
| | | | FTHLNKDDEGLYTLRMVTKGGVNE |
| | | | CSAFLFVRDADALIAGAPGAPMDVK |
| | | | CHDANRDYVIVTWKPPNTTSQNPVI |
| | | | GYFVDKCEVGLENWVQCNDAPVKI |
| | | | CKYPVTGLYEGRSYIFRVRAVNSAGI |
| | | | SRPSRVSEPVAALDPVDLERTQTVHV |
| | | | DEGRKIVISKDDLEGDIQIPGPPTNVH |
| | | | ASEISKTYVVLSWDPPVPRGREPLTY |
| | | | FIEKSMVGSGSWQRVNAQVAVKSPR |
| | | | YAVFDLAEGKPYVFRVLSANKHGIS |
| | | | DPSEITEPIQPQDIVVVPSAPGRVVAT |
| | | | RNTKTSVVVQWDKPKHEENLYGYYI |
| | | | DYSVVGSNQWEPANHKPINYNRFVV |
| | | | HGLETGEQYIFRVKAVNAVGFSENS |
| | | | QESEAIKVQAALTCPSYPHGITLLNC |
| | | | DGHSMTLGWKAPKYSGGSPILGYYI |
| | | | DKREANHKNWHEVNSSVISRTIYTV |
| | | | EDLTEDAFYEFKIAAANVVGIGHPSD |
| | | | PSEHFKCKAWTMPEPGPAYDLTVCE |
| | | | VRNTSLVLLWKAPVYEGKSPITGYL |
| | | | VDYKEVDTEDWITANEKPTSHRYFK |
| | | | VTDLHQGHTYVFKVRAVNDAGVGK |
| | | | SSEISEPVFVEASPGTKEIFSGVDEEG |
| | | | NIYLGFECKEATDASHFLWGKSYEEI |
| | | | EDSDKFKIETKGDHSKLYFKHPDKSD |
| | | | LGTYCISVSDTDGVSSSFVLDEEELE |
| | | | RLMTLSNEIKNPTIPLKSELAYEVLD |
| | | | KGEVRFWIQAESLSPNSTYRFVINDK |
| | | | EVENGDRHKISCDHSNGIIEMVMDK |
| | | | FTIDNEGTYTVQIQDGKAKNQSSLVL |
| | | | IGDAFKAILAESELQRKEFLRKQGPH |
| | | | FSEFLYWEVTEECEVLLACKIANTKK |
| | | | ETVFKWYRNGSGIDVDEAPDLQKGE |
| | | | CHLTVPKLSRKDEGVYKATLSDDRG |
| | | | HDVSTLELSGKVYNDIILALSRVSGK |
| | | | TASPLKILCTEEGIRLQCFLKYYNEE |
| | | | MKVTWSHRESKISSGEKMKIGGGED |
| | | | VAWLQITEPTEKDKGNYTFEIFSDKE |
| | | | SFKRTLDLSGQAFDDALTEFQRLKA |
| | | | AAFAEKNRGKVIGGLPDVVTIMDGK |
| | | | TLNLTCTVFGNPDPEVVWFKNDKAL |
| | | | ELNEHYLVSLEQGKYASLTIKGVTSE |
| | | | DSGKYSIYVKNKYGGETVDVTVSVY |
| | | | RHGEKIPEVNQGQLAKPRLIPPSSST |
| SEQ ID | E1BE25 | Filamin C/Bos | MMNNSGYSEAPGFGLGDEVDDMPS |
| NO: 151 | (UniProtKB) | taurus | TEKDLAEDAPWKKIQQNTFTRWCNE |
| | | | HLKCVGKRLTDLQRDLSDGLRLIAL |
| | | | LEVLSQKRMYRKFHPRPNFRQMKLE |
| | | | NVSVALEFLEREHIKLVSIDSKAIVDG |
| | | | NLKLILGLIWTLILHYSISMPMWEDE |
| | | | DDEDARKQTPKQRLLGWIQNKVPQL |
| | | | PITNFNRDWQDGKALGALVDNCAPG |
| | | | LCPDWEAWDPNQPVENAREAMQQA |
| | | | DDWLGVPQVIAPEEIVDPNVDEHSV |
| | | | MTYLSQFPKAKLKPGAPVRSKQLNP |
| | | | KKAIAYGPGIEPQGNTVLQPAHFTVQ |
| | | | TVDAGIGEVLVYIEDPEGHTEEAKVV |
| | | | PNNDKNRTYAVSYVPKVAGLHKVT |
| | | | VLFAGQNIERSPFEVNVGMALGDAN |
| | | | KVSARGPGLEPVGNVANKPTYFDIY |
| | | | TAGAGTGDVAVVIVDPQGRRDTVEV |
| | | | ALEDKGDSTFRCTYRPVMEGPHTVH |
| | | | VAFAGAPITRSPFPVHVAEACNPNAC |
| | | | RASGRGLQPKGVRVKEVADFKVFTK |
| | | | GAGSGELKVTVKGPRGTEEPVKVRE |
| | | | AGDGVFECEYYPVVPGKYVVTITWG |
| | | | GYAIPRSPFEVQVSPEAGIQKVRAWG |
| | | | PGLETGQVGKSADFVVEAIGTEVGT |
| | | | LGFSIEGPSQAKIECDDKGDGSCDVR |
| | | | YWPTEPGEYAVHVICDDEDIRDSPFI |
| | | | AHIQPAPPDCFPDKVKAFGPGLEPTG |
| | | | CIVDKPAEFTIDARAAGKGDLKLYA |
| | | | QDADGCPIDIKVIPNGDGTFRCSYVP |
| | | | TKPIKHTIIVSWGGVNVPKSPFRVNV |
| | | | GEGSHPERVKVYGPGVEKTGLKANE |
| | | | PTYFTVDCSEAGQGDVSIGIKCAPGV |
| | | | VGPAEADIDFDIIKNDNDTFTVKYTP |
| | | | PGAGRYTIMVLFANQEIPASPFHIKV |
| | | | DPSHDASKVKAEGPGLNRTGVEVGK |
| | | | PTHFTVLTKGAGKAKLDVHFAGAA |
| | | | KGEAVRDFEIIDNHDYSYTVKYTAV |
| | | | QQGNMAVTVTYGGDPVPKSPFVVN |
| | | | VAPPLDLSKVKVQGLNSKVAVGQEQ |
| | | | AFSVNTRGAGGQGQLDVRMTSPSRR |
| | | | PIPCKLEPGGGAETQAVRYMPPEEGP |
| | | | YKVDITYDGHPVPGSPFAVEGVLPPD |
| | | | PSKVCAYGPGLKGGLVGTPAPFSIDT |
| | | | KGAGTGGLGLTVEGPCEAKIECQDN |
| | | | GDGSCAVSYLPTEPGEYTINILFAEA |
| | | | HIPGSPFKATIRPVFDPSKVRASGPGL |
| | | | ERGKAGEAATFTVDCSEAGEAELTIE |
| | | | ILSDAGVKAEVLIHNNADGTYHITYS |
| | | | PAFPGTYTITIKYGGHPVPKFPTRVH |
| | | | VQPAVDTSGVKVSGPGVEPHGVLRE |
| | | | VTTEFTVDARSLTATGGNHVTARVL |
| | | | NPSGAKTDTYVTDNGDGTYRVQYT |
| | | | AYEEGAHLVEVLYDDVAVPKSPFRV |
| | | | GVTEGCDPTRVRAFGPGLEGGLVNK |
| | | | ANRFTVETRGAGTGGLGLAIEGPSEA |
| | | | KMSCKDNKDGSCTVEYIPFTPGDYD |
| | | | VNITFGGRPIPGSPFRVPVKDVVDPG |
| | | | KVKCSGPGLGAGVRARVPQTFTVDC |
| | | | SQAGRAPLQVAVLGPTGVAEPVEVR |
| | | | DNGDGTHTVHYTPATDGPYTVAVK |
| | | | YADQEVPRSLSSPFKIKVLPAHDASK |
| | | | VRASGPGLNASGIPASLPVEFTIDAR |
| | | | DAGEGLLTVQILDPEGKPKKANIRDN |
| | | | GDGTYTVSYLPDMSGRYTITIKYGG |
| | | | DEIPYSPFRIHALPTGDASKCLVTVSI |
| | | | GGHGLGACLGPRIQIGEETVITVDAK |
| | | | AAGKGKVTCTVSTPDGAELDVDVV |
| | | | ENHDGTFDIYYTAPEPGKYVITIRFG |
| | | | GEHIPNSPFHVLACEAMPRVEEPPDV |
| | | | PQLHRPSAYPTHWATEEPVVPAEPM |
| | | | ESMLRPFNLVIPFTVQKGELTGEVRM |
| | | | PSGKTARPNITDNKDGTITVRYAPTE |
| | | | KGLHQMGIKYDGNHIPGSPLQFYVD |
| | | | AINSRHVSAYGPGLSHGMVNKPATF |
| | | | TIVTKDAGEGGLSLAVEGPSKAEITC |
| | | | KDNKDGTCTVSYLPTAPGDYSIIVRF |
| | | | DDKHIPGSPFTAKITGDDSMRTSQLN |
| | | | VGTSTDVSLKITESDLSQLTASIRAPS |
| | | | GNEEPCLLKRLPNRHIGISFTPKEVGE |
| | | | HVVSVRKSGKHVTNSPFKILVGPSEI |
| | | | GDASKVRVWGKGLSEGHTFQVAEFI |
| | | | VDTRNAGYGGLGLSIEGPSKVDINCE |
| | | | DMEDGTCKVTYCPTEPGTYIINIKFA |
| | | | DKHVPGSPFTVKVTGEGRMKESITR |
| | | | RRQAPSIATIGSTCDLNLKIPGNWFQ |
| | | | MVSAQERLTRTFTRSSHTYTRTERTE |
| | | | ISKTRGGETKREVRVEESTQVGGDPF |
| | | | PAVFGDFLGRERLGSFGSITRQQEGE |
| | | | ASSQDMTAQVTSPSGKTEAAEIVEGE |
| | | | DSAYSVRFVPQEMGPHTVTVKYRGQ |
| | | | HVPGSPFQFTVGPLGEGGAHKVRAG |
| | | | GTGLERGVAGVPAEFSIWTREAGAG |
| | | | GLSIAVEGPSKAEIAFEDRKDGSCGV |
| | | | SYVVQEPGDYEVSIKFNDEHIPDSPF |
| | | | VVPVASLSDDARRLTVTSLQETGLK |
| | | | VNQPASFAVQLNGARGVIDARVHTP |
| | | | SGAVEECYVSELDSDKHTIRFIPHEN |
| | | | GVHSIDVKFNGAHIPGSPFKIRVGEQ |
| | | | SQAGDPGLVSAYGPGLEGGTTGVSS |
| | | | EFIVNTLNAGSGALSVTIDGPSKVQL |
| | | | DCRECPEGHVVTYTPMAPGNYLIAIK |
| | | | YGGPQHIVGSPFKAKVTGPRLSGGHS |
| | | | LHETSTVLVETVTKSSSSRGSSYSSIP |
| | | | KFSSDASKVVTRGPGLSQAFVGQKN |
| | | | SFTVDCSKAGTNMMMVGVHGPKTP |
| | | | CEEVYVKHMGNRVYNVTYTVKEKG |
| | | | DYILIVKWGDESVPGSPFKVNVP |
| SEQ ID | Q5ZLY3 | Tropomodulin | MTLPFRKDLDKYKDLDEDDILGKLS |
| NO: 152 | (UniProtKB) | 3/Gallus gallus | EEELKQLETVLDDLDPENALLPAGFR |
| | | | QKDQTAKKASGPFDRERLLAYLEKQ |
| | | | ALEHKDREDYVPFTKEKKGKVFIPK |
| | | | QKPAQSYAEEKIALDPELEEALTSAT |
| | | | DTELCDLAAILGMSNLITNNQFCDIV |
| | | | GSSNGVGKDSFSNIVKGEKMLPVFD |
| | | | EPPNPTNVEETLQRIKDNDSRLVEVN |
| | | | LNNIKNIPIPTLKEFAKALETNTHVKN |
| | | | FSLAATRSNDPVAVALADMLRVNTK |
| | | | LKSLNIESNFITGVGILALVDALKDNE |
| | | | TLTEIKIDNQRQQLGTLAEVEIAKML |
| | | | EENTKILKFGYHFTQQGPRARAAAAI |
| | | | TKNNDLVRKRRVEGDGQ |
| SEQ ID | AAC14459.1 | Tropomodulin/ | MSYRKELEKYRDLDEDKILGALTEE |
| NO: 153 | (GenBank) | Gallus gallus | ELRKLENELEELDPDNALLPAGLRQR |
| | | | DQTQKPPTGPFKREELMAHLEQQAK |
| | | | DIKDREDLVPFTGEKRGKAWIPKQK |
| | | | PMDPVLESVTLEPELEEALANASDAE |
| | | | LCDIAAILGMHTLMSNQQYYEALGS |
| | | | STIVNKEGLNSVIKPTKYKPVPDEEP |
| | | | NSTDVEETLKRIQNNDPDLEEVNLN |
| | | | NIMNIPVPTLKALAEALKTNTYVKKF |
| | | | SIVGTRSNDPVAFALAEMLKVNNTL |
| | | | KSLNVESNFISGSGILALVEALQSNTS |
| | | | LIELRIDNQSQPLGNNVEMEIANMLE |
| | | | KNTTLLKFGYHFTQQGPRLRASNAM |
| | | | MNNNDLVRKRRLAELNGPIFPKCRT |
| | | | GV |
| SEQ ID | A0A287BJ41 | Tropomodulin | MSYRRELEKYRDLDEDEILGALTEEE |
| NO: 154 | (UniProtKB) | 1/Sus scrofa | LRTLENELDELDPDNALLPAGLRQK |
| | | | DQTTKAPTGPFKREELLDHLEKQAK |
| | | | EFKDREDLVPYTGEKRGKVWVPKQ |
| | | | KPMDPVLETVTLEPELEEALANASD |
| | | | AELCDIAAILGMHTLMSNQQYYQAL |
| | | | GSSSIVNKEGLNSVIKPTQYKPVPDE |
| | | | EPNATDVEETLERIKNNDPKLEEVNL |
| | | | NNIRNIPIPTLKAYAEALKENSYVKK |
| | | | FSIVGTRSNDPVAFALAEMLKVNKV |
| | | | LKTLNVESNFISGAGILRLVEALPYN |
| | | | TSLVELKIDNQSQPLGNKVEMEIVSM |
| | | | LEKNATLLKFGYHFTQQGPRLRASN |
| | | | AMMNNNDLVRKRRLADLTGPIIPKC |
| | | | RSGI |
| SEQ ID | F1NXA5 | Coronin/Gallus | MSRRVVRQSKFRHVFGQPVKADQM |
| NO: 155 | (UniProtKB) | gallus | YEDIRVSKVTWDSSFCAVNPKFVAII |
| | | | VEAGGGGAFMVLPLAKTGRVDKNH |
| | | | PLVTGHTAPVLDIDWCPHNDNVIAS |
| | | | ASEDTTVMVWQIPDYVPVRSITEPVV |
| | | | TLEGHSKRVGIICWHPTARNVLLSAG |
| | | | CDNLVILWNVGTGEMLLALEDMHT |
| | | | DLIYNVGWNRNGSLLVTTCKDKKV |
| | | | RVIDPRKQTVVAEITKPHDGARPIRAI |
| | | | FMADGKIFTTGFSKMSERQLGLWDL |
| | | | KNFEEPIALQEMDTSNGVLLPFYDPD |
| | | | TNIVYLCGKGDSSIRYFEITDEAPYV |
| | | | HYLNTYSSKEPQRGMGFMPKRGLD |
| | | | VSKCEIARFFKLHERKCEPIVMTVPR |
| | | | KSDLFQDDLYPDTPGPEPALEADEW |
| | | | LSGKDAEPILISLRDGYVPVKNRELK |
| | | | VVKKNILDSKPPPGPRRSHSTSNTDIS |
| | | | TPALDEVLEEIRVLKETVQAQEKRIS |
| | | | ALEHKLCQFTNGTD |
| SEQ ID | XP_015136760.1 | Nebulette | MYFSFMLAGLHRRKEFSLLFPPSIKM |
| NO: 156 | (NCBI) | isoform | RVSVTQEFTEDENENGEEERVFLKPV |
| | | X1/Gallus gallus | IEDRNMELARKCSEIISDVHYKEEFE |
| | | | KSKGKCIFVPDTPQLKHVKSVGAFIS |
| | | | EVKYKGAAKKDLSNSLYQQMPATID |
| | | | SAFAKELTQLQSKVLYKQKHDAEKG |
| | | | TSDYAHMKEPPDIKHAMEVNKYQS |
| | | | DVSYKRDVQDTHRYTEVLNRPDIKM |
| | | | ATEITKIISDAEYKKGRGEMNKEPAV |
| | | | LGRPDFEHAKGVSKLLSQVKYKEQF |
| | | | KKEMKSHQYNPLDSASFKQAQIAST |
| | | | LASNVNYKKDYKESLHDPASDLPNL |
| | | | LYLNHALNISKMHSDVKYRENYEKS |
| | | | KGKSMLEFVDTPLYQVSKDVQKMQ |
| | | | SEKIYRKDFEESLKGRPSLDLDKTPEF |
| | | | LHIKQVTNLLKEKEYRKDLEEWMK |
| | | | GKGMTVFEDTPDLIRVKNAAQILNE |
| | | | KQYKKDLETEIKGKGMQVGPDTPEI |
| | | | RRAKKASEIASTKEYKKDLENEIKGK |
| | | | GMEVGMDTPDIQRAKKASEIVSQKE |
| | | | YRKDLETEIIGKGMQVGPFTPEIQRV |
| | | | KRASEIASQKMYKDEAEKMLCNYSA |
| | | | VPDTPEMERIKSTQKNISSVFYKKVV |
| | | | GAGTAVKETPEIERVKKNQQNISSIK |
| | | | YKEETQHATPISDPPELRRIKENQKNI |
| | | | SNVHYKEQLCRATPVSVTPEIERVKR |
| | | | NQENVSMVHYREQPGKATAVSITPEI |
| | | | ERVKKNQDNISSVKYSSDQRQMKGR |
| | | | RSVILDTPELRHVKETQNNISMVKYH |
| | | | EDFEKTKGRGFTPVVDDPITERVRKN |
| | | | TQVVSDAAYKGVHPHIVEMDRRPGII |
| | | | VDLKVWRTDPGSIFDIDPLEDNIQSR |
| | | | SLHMLSERASRYSKQYLHSTSLGDY |
| | | | KSDGSDTNPTFSYCSEITRPSDEGAPV |
| | | | LPGAYQQSQSQGYGYMHQTSMSSM |
| | | | RSVHSQPHPAGLRTYRAMYDYSAQ |
| | | | DEDEVSFRDGDYIINVQPIDDGWMY |
| | | | GTVQRTGKTGMLPANYIEFVN |
| SEQ ID | F1MMX2 | Nebulin-related- | MNVQACSRCGYGVYPAEKINCLDQI |
| NO: 157 | (UniProtKB) | anchoring | WHKACFHCEVCKMMLSVNNFVSYQ |
| | | protein/Bos | KKPYCHAHNPKNNTFTSVYYTPLNL |
| | | taurus | NVRKPPEAICGIGGQEDGERFKSVFH |
| | | | WDMKSKDEAAAPNRQPQVDERAY |
| | | | WSGYREGDAWCPGALPDPEIVRMV |
| | | | EARKSLGEEYPEDYEQQRGKGSFPA |
| | | | MITPAYQRAKIANQLASQVEYKRGH |
| | | | DERISRFSTVADTPELLRAKAGGQLQ |
| | | | SDVRYTEAYEQQRGKGSFPAMITPA |
| | | | YQIAKRANELASDVRYHQQYQREM |
| | | | KGMAGPAAGAEGPLPKEYMDQYGQ |
| | | | GYSEEYGEHRGKGSFPAMITPAYQN |
| | | | AKKANELASDIKYRQDFNKMKGAA |
| | | | HYHSLPAQDNLVLKRAQSVNKLVSE |
| | | | VEYKKDLESSKGHSINYCETPQFRNV |
| | | | CKISKFTSDNKYKENYQNRMRGRYE |
| | | | GVGMDKRMLHALKVGSLASNIAYK |
| | | | ADYKHDVVDYNYPATLTPSYQTTV |
| | | | KLAPLKDVNYRQSIDKLKYSSVTNTP |
| | | | QIVQAKINAQQLSHVNYRADYEKNK |
| | | | LNYTLPQDVPQLVKARTNAELFSEV |
| | | | KYREGWEKTKGKGFEMKLDAMSLL |
| | | | AAKASGELASNIKYKEEYEKAKGKV |
| | | | LGTTDSRLLHSLQVAKMSSEVEYKK |
| | | | GFEKSKTHFHLPMDMVNIRHAKKAQ |
| | | | ALASDLDYRKRLHEYTVLPEDMKTL |
| | | | WAKKAYGLQSELQYKADLAWMKG |
| | | | VRWLTEGSLNLEQAKKAGQLVSEK |
| | | | NYRQRVDELKFTSVADSSQMEHAK |
| | | | KSQELQSGVAYKAEHEQSVHQYSIS |
| | | | KDEPLFLQARANAANLSEKLYKSSW |
| | | | ENQKAKAFDLRLDSLAFLAAKAKRD |
| | | | LASEVKYKEDYEKSRGKLIGAQGAQ |
| | | | GDSQMSHSLQMSKLQSELEYKKGFE |
| | | | DTKSQCHVPLDMIHLVHARKAQHLA |
| | | | TDIGYKTASHHFTALPTDMKVEWAK |
| | | | KAYGLQSDNQYRADVKWMKGTGW |
| | | | VATGSLNVEQAKKAGELISEKKYRQ |
| | | | HPDALKFTSIKDTPEMVQARISYTQA |
| | | | VDRLYREQGENLKHHYTQTTDLPEV |
| | | | LLAKLNAMNISETRYKESWSKLRDG |
| | | | GYKLRLDAIPFQAAKASGEIISDYKY |
| | | | KEAFEKMKGQMLGSRSLEDDISLAH |
| | | | SVYASSLQSQVNYKKDFEHSKAQFH |
| | | | LPLDMVTLVHAKKAQTLASDQDYR |
| | | | HPLPQYTSLAEDLRLSCAKRAHKLQ |
| | | | SENLYRSDLNFMRGVACVIPGTLEIE |
| | | | GRKRASELISESKYRQHPQSLKYTAV |
| | | | TDTPSLTHAKLSNQITNERLYKAAGE |
| | | | DARHQYTMTLGLPEFVRAKTNAAN |
| | | | LSDAKYKESWRNLHAQGYKLTIDAL |
| | | | PFQAARVSGDIASDFLYRHDFVKER |
| | | | GRLIGAQSVSDDPRLQHCQRVGQLQ |
| | | | SELQYRRQAAGSRAQCHLPMDMVP |
| | | | LVHARKAQALASDLDYRTQCHAFT |
| | | | ALPEDLRMAWAKKAHALQSELRYK |
| | | | SDLMGMKGTGWLALSSPQIESAKKA |
| | | | GELISETKYREKPDTIKFTTVVDSPDL |
| | | | VHAKNSYMHCNERLYRSGDAESRH |
| | | | RYTLVPDHPDFTRARLNALNLSDKV |
| | | | YRHSWEQTRAGGYDFRLDAIPFQTA |
| | | | RASREIASDFRYKEAFLRDRGLQIGY |
| | | | RSINDDPRTKHFLSVGRLQSDNEYKK |
| | | | DFAKSRSQFHSRPDQPGFLQAKRSQ |
| | | | QLASDVHYRQPLPQPTCDPEQLGLK |
| | | | HAQKAHRLQSDVKYKSDLNLTRGIG |
| | | | WTPPGSYKVEMARRAAELANARGL |
| | | | GLQGAYGGPEAVEPRDDQSGFVNPD |
| | | | ATEILHVKRRKAPLF |
| SEQ ID | XP_003641574.1 | nebulin-related- | MNVQPCARCGYGVYPAEKINCIDQT |
| NO: 158 | (NCBI) | anchoring | WHKACFHCEVCKMMLTVNNFVSHE |
| | | protein isoform | KKPYCQVHNPKNNAFTSIFETPINLN |
| | | X1/Gallus gallus | AKKLSEVVSEVKYREESEQFKSAFQ |
| | | | WDVRSRDIEAAYKAQHLSSQNAYY |
| | | | AAYEGGTSWYSGNMPDPQMVTVTQ |
| | | | AQKNLNDLQYTEEYELRQGKGSFPA |
| | | | MITPGYQVAKRATQLSSNVEYRRGH |
| | | | EERVSKFTSVVDTPDILHAKAGGQL |
| | | | ASDLKYTEDFEEQRGKGSFPAMITPA |
| | | | YQIAKRANELASDVKYHQTYEKEIK |
| | | | GKASHTAGTDVTFTRENVDQYGQD |
| | | | YMNEYEERRGKGSFPAMITPAYQNA |
| | | | KKANELASDIKYKKDLSKMKGAAH |
| | | | FHSLTAEDNLVLKQAQSANKLVSEV |
| | | | EYKKDLGNNRGYSVNYCDTPQFKN |
| | | | VSKISKYTSDIKYKETYQNQMKGHY |
| | | | MGIGMDKRMLHAMKVGNLASNIAY |
| | | | KSDYKHDGVDYNYPATLTPSYQTTR |
| | | | KLVPLKDVNYRQSIDKMKYSSVAST |
| | | | PEIAQAKINAQQLSDLNYRAQYEKT |
| | | | KTNYTLPQDIPQLVKAKANAELYSE |
| | | | VKYKEGWEKSKGQGFEMKLDSLPL |
| | | | LAAKASRDLASDIKYKEEYEKTKGK |
| | | | AIETKDSRLLHSLQVAKMSSEIAYKK |
| | | | DFEESKTHFHLPMDMVNLRHAKKA |
| | | | QALASDLDYRKRLHEYTVVPEDLKT |
| | | | KWAKKAYGLQSDLQYREDLMWMK |
| | | | GVGCITEGSLNIQQAKKAGDLVSEK |
| | | | KYRQKVDALKFTSVADSSHIKHAKK |
| | | | SQELQSDVAYRSGKEQFLHQYTITK |
| | | | DDPVFLLAKANAANISEKLYRSSWE |
| | | | KQKEKGFVLRLDALSFLTAKAKRDL |
| | | | ASDIKYKEGYEKMKGKLIGVKAVEE |
| | | | DSKMAHSLQMSKLQSDLEYKKAFE |
| | | | DTKNQFQVSMDMANLVHAKKAQN |
| | | | LASDKGYRTALHHYTVLPTDRKVA |
| | | | WAKWAYGLQSDNRYRADLNWMKG |
| | | | AGWIATGSLNVEQAKKAGELISEKK |
| | | | YRQHPYALKFTSIKDTPEMIQARISY |
| | | | NQAVDRLYKEHGESIKHQYTLTADL |
| | | | PEILRAKLNTMNISEIRYKESWQKMK |
| | | | DGGYQLRLDAIPFQAAKASGEIISDH |
| | | | KYKEAFEKMKGQMIGSCGLDGDIRI |
| | | | AHSVHASSLQSDVKYKQGFEDTKTH |
| | | | FHLPLDMINLVHARKAQSLVSEQEY |
| | | | RQLLHQYTSLTDDLRLQCAKNAYKL |
| | | | QSENLYRSDMNFMRGVGCITPGALEI |
| | | | EGKKKASELISESKYRQQPHSFKYTS |
| | | | VTDSPGLLHAKFSNMIANERLYKAA |
| | | | GEDIQHHYTPTLGLPEFTQARINAAN |
| | | | LSDVKYRESWHNLCAQGYKLTMEA |
| | | | LPFQTARASREIASDYQYKHNFVME |
| | | | RGKHIGARSVLDDTRLLHCLHAAKL |
| | | | QSEQEYKKGSQGVWSQYYLPMDMV |
| | | | NLVHARKAQALASDQEYRKRLHEFT |
| | | | ALPEDLKMKWAKRAHMLQSEHRYK |
| | | | SDLNFMKGVGWMALRSPQIESVKK |
| | | | AGELISETKYRQKPESLKFTAVVDSP |
| | | | DLIHAKNSYLQCNDRLYKAGDSEAR |
| | | | HRYTLPPDHPDFIRARQNALNISDKV |
| | | | YKTSWEQTRASGYDFRIDAIPFQTAK |
| | | | ASREIASDYKYREAFLRDRGQRIGFF |
| | | | SANDDAHTRHVLRVGKLQSDNLYRS |
| | | | GYAQNRGHFQSHLNQPGFLHAKRSQ |
| | | | QLASNVNYKQPLHQYTCDPEQLNVK |
| | | | HAKQAYKLQSDVKYKSDLNWLRGI |
| | | | GWTPPGSYKVERARRAAELAYLRE |
| | | | MGLQAASAQYGPEADQVGPEGSQQ |
| | | | VTVNPDASEILQVKRRKMQLYK |
| SEQ ID | P08728 | Keratin, type 1 | MTSYSYRQSSSTSSFGGMGGGSMRF |
| NO: 159 | (UniProtKB) | cytoskeletal | GAGGAFRAPSIHGGSGGRGVSVSSA |
| | | 19/Bos taurus | RFVSSSSGGYGGGYGGALATSDGLL |
| | | | AGNEKLTMQNLNDRLASYLEKVRA |
| | | | LEEANGDLEVKIRDWYQKQGPGPAR |
| | | | DYSHYFKTIEDLRDQILGATIENSKIV |
| | | | LQIDNARLAADDFRTKFETEQALRM |
| | | | SVEADINGLRRVLDELTLARTDLEM |
| | | | QIEGLKEELAYLKKNHEEEMSVLKG |
| | | | QVGGQVSVEVDSAPGIDLAKILSDM |
| | | | RSQYEVIAEKNRKDAEAWFISQTEEL |
| | | | NREVAGHTEQLQISKTEVTDLRRTLQ |
| | | | GLEIELQSQLSMKAALEGTLAETEAR |
| | | | FGAQLAQIQALISGIEAQLSDVRADT |
| | | | ERQNQEYQHLMDIKTRLEQEIATYR |
| | | | NLLEGQDAYFNDLSLAKAL |
| SEQ ID | E1BF23 | Myomesin | MSLVAVPFYQKRHKHFDQSYRNIQT |
| NO: 160 | (UniProtKB) | 2/Bos taurus | RYLLDEYSAKKKRASAQSSLQRSVT |
| | | | RKSSSQRESSRAALGRTTCRLCAKR |
| | | | MSSALEEEAQERKRRYQSMVAAYG |
| | | | EAKRERYLSELAQLEEDVHVARTHA |
| | | | RDQLDRLALQHAVDDRLAWERHSF |
| | | | EERISRAPEILVRLRSHTVWERMSVK |
| | | | LCFTVQGFPTPVVQWYKDGSLICQA |
| | | | GEPGKYRIDSKYGVHTLEINRADFDD |
| | | | TATYSAVATNVHGQVSTNAAVVVR |
| | | | RFRGDEEPFHSVGLPIGLPLSSVIPYT |
| | | | HFDVQFIEKFGVTFRREGETLTLKCT |
| | | | LLVTPDLKRVQPRAEWYRDDVLLKE |
| | | | SKWTKMFFGEGQASLSFSHLNKDDE |
| | | | GLYTLRIVSRGGITDHSAFLFVRDAD |
| | | | PLVTGAPGAPMDVHCHDVNRDYVI |
| | | | VTWKPPNATKESPVIGYFIDRCEVGT |
| | | | DNWIQCNDAPVKICKYPVTGLFEGR |
| | | | SYIFRVRAVNSAGISRPSRVSEAVAA |
| | | | LDPVDLRRLQAVHLEGEKDIVVYQE |
| | | | ELEGEVQIPGPPTNVHASETSRTYVV |
| | | | LSWDPPEPRGKEPLMYFIEKSMVGS |
| | | | GSWQRVNAQTAVRSPRYAVFDLAE |
| | | | GKPYVFRVLSANKHGLSDPSEITAPI |
| | | | QAQDTIVVPSAPGRVLASRNTRTSVV |
| | | | VQWDRPKHEEDLLGYYVDCSVAGS |
| | | | NVWEPCNHKPIGYNRFVVHGLTTGE |
| | | | QYIFRVKAVNAVGTSENSQESDVIKV |
| | | | QAALTVPSHPYGITLLNCDGHSMILG |
| | | | WKVPKFSGGSPILGYYVDKREAHHK |
| | | | NWHEVNSSPLKERILTVEGLTEGSLY |
| | | | EFKIAAANMAGIGQPSDPSELFKCEA |
| | | | WTMPEPGPAYDLTFCEVRDTSLVLL |
| | | | WKPPVYSGSSPVSGYFVDYKEEDSG |
| | | | EWLTVHETATPHRYLKVCDLHQGK |
| | | | TYVFRVRAVNASGVGRPSDTSEPVL |
| | | | VEARPGTKEVSAGVDEEGNVYLSFD |
| | | | CPEVTDASHFTWCKSYEDIADDDRF |
| | | | KVETAGDHSKLYFKNLDKEDLGTYS |
| | | | VSVSDTDGVSSSFVLDEEELERLKAL |
| | | | SNEIKNPTIPLKSELAYEIFDKGQVRF |
| | | | WLQAEHLSPDSSYRFIINDREVADSE |
| | | | THRIKCDKSTGIIEMVMDRFTIDNEG |
| | | | TYTVQIQDGKAKSQSSLVLIGDAFKA |
| | | | VLKEAEFQRKEFLRKQGPHFAEYLH |
| | | | WDVTEECEVRLVCKVANTKKETVF |
| | | | KWLKDDVLYETEKMPDLEKGVCEL |
| | | | LIPKLSKKDHGEYKATLKDDRGQDV |
| | | | STLEIAGKVYEDMILAMSRVCGASA |
| | | | SPLKILCTPEGIRLQCFMKYFTEEMK |
| | | | VNWYHKDAKITSSEHMRIGGNEQM |
| | | | AWLQICEPTEKDKGKYTFEILDGKK |
| | | | NHQRSLDLSGQAFDEAFAEFQQLKA |
| | | | AAFAEKNRGKVIGGLPDVVTIMEGK |
| | | | TLNLTCTVFGNPDPEVVWFKNDKDI |
| | | | ELSDHFSVKVEQGKYVSMTIKGVTS |
| | | | EDSGKYSIHVKNKYGGEKIDVTVSV |
| | | | YKHGEEIPDVVLPQQAKPKLLPAAA |
| | | | PSPAH |
| SEQ ID | F1N0L9 | Myopalladin/ | MQDDSLEASTSISQLLRESYLAETRH |
| NO: 161 | (UniProtKB) | Bos taurus | RGNNERSRAEPSSNPFHFGGSSGAAE |
| | | | GGGGQDDLPDLSAFLSQEELDESVN |
| | | | LARLAINYDPLEKADEAQARKRFSS |
| | | | DQTKHSSISSFDPNFCQDNSQSPTNS |
| | | | KESLQETKRPQYSSEAQSKKVFLNK |
| | | | AADFIEELSSLFKAHSSKRIRPRACKN |
| | | | HKSKLESQNQVMQENSSSFPDLSERR |
| | | | ERSSVPIPIPADTRDNEVNHALEQQE |
| | | | AKRREAEQAAGEAASGDTTPGSSPSS |
| | | | LYYEEPLGQPPRFTQKLRSREVPEGT |
| | | | RVQLDCIVVGIPPPQVRWYCEGKELE |
| | | | NSPDIHIIQAGNLHSLTIAEAFEEDTG |
| | | | RYSCFASNIYGTDSTSAEIYIEGVSSS |
| | | | DSEGDPNKDEMNRIQKPNEVSSPPTT |
| | | | SAGIPSAVPQTQHVVVQPRVSTIQQC |
| | | | QSPTNYLQGLDGKPIIAAPVFTKMLQ |
| | | | NLSASEGQLVVFECRVKGAPSPKVE |
| | | | WYREGTLIEDSPDFRILQKKPRSMAE |
| | | | PEEICTLVIAEVFAEDSGCFTCTASNK |
| | | | YGTVSSIAQLDVRGNEDLRNNGSLH |
| | | | SANSTTNLALTEQQPSPPNPEPPVEQP |
| | | | PKPKLEGVLVNHNEPRSSSRIGLRVH |
| | | | FNLPEDDKGSEESSEGGVVTTHQTRP |
| | | | DSFQERFNGQSAKISEPSSPVKEPPPV |
| | | | LAKPKLDSSQLQQLHNQVLLEQHHL |
| | | | QNPSPSSPKEFPFNMSVLNSTTVPAV |
| | | | TVSSKHAKAPPSQTFSLARPKHFFPS |
| | | | TSTTVASVSPSSSPVFTLSSTPQAMQR |
| | | | TMSKESLLVTHPSAQTKSLGGASIQN |
| | | | EPLPTPAPTEPAQLPLTFSISSGNQFQP |
| | | | RCVSPAPVSPTGRIQNPVAFLSSVLPS |
| | | | LPAIPPTNAMGLPKSAPSMPSQGLMK |
| | | | KNTKSSHPVSDDYIRETKNAVILDLG |
| | | | KKMNFSDVRSNQQEYKISSFEQRLM |
| | | | NEIEFRLERTPVDESDDEIQHDEIPTG |
| | | | KCIAPIFDKRLKHFRVTEGSPVTFTCK |
| | | | IVGIPVPKVYWFKDGKQISKRNEHFK |
| | | | MKREGDGTCSLHINSTSSDDDGNYTI |
| | | | MAANPQGRISCSGHLMVQGLPIRSRL |
| | | | TAAGQSHRGRSRVQERDKEPLQERF |
| | | | FRPHFLQAPGDMVAHEGRLCRLDCK |
| | | | VSGLPPPELTWLLNGQPVLPDTSHK |
| | | | MLVRETGVHSLLIDPLTQRDAGTYT |
| | | | CIATNKTGQNSFSLELTVVAKEVKK |
| | | | APVILEKLQNSGVPEGHPVRLECRVI |
| | | | GMPPPVFYWKKDNETIPFTRERISMH |
| | | | QDTTGYVCLLIQPAKKSDAGWYTLS |
| | | | AKNEAGIVSCTARLDIYAQWHHQIPP |
| | | | PMTVRPSGSRYGSLTSKGLDIFSAFSS |
| | | | MESTMVYSCSSRSVVESDEL |
| | Q5E9V3 | Myozenin-2/Bos | MLSHNTMVKQRKQQASAIMKEIHG |
| | (UniProtKB) | taurus | NDVDVMHLGKKVSIPRDIMLEELSH |
| | | | LSNRGARLFKMRQRRSDKYTFENFQ |
| | | | YETKAQINHNIAMQNEKLDGINLESG |
| | | | SQQAPFTPPNTPDPRSPPNPENIAPGY |
| | | | SGPLKEIPPERFNTTAVPKYYQSPWE |
| | | | QAISNDPELLEALYPKFFKPEGKAEL |
| | | | PDYRSFNRVATPFGGFEKASKMVKF |
| | | | KVPDFDLLLLTDPRFMAFANPLSGRR |
| | | | SFNRTPKGWISENIPIVITTEPTEDNTI |
| | | | PESEDL |
| SEQ ID | NP_001264756.1 | Myozenin 2/ | MLSHSAMVKERKQQASAIMDEIQRN |
| NO: 162 | (NCBI) | Gallus gallus | VSPSLNLGKKVSTPRDIMVEELSTLS |
| | | | NRGARLFKRRQRRSDKYTYENYHY |
| | | | VANKPRNRGEPIHSVKMDGLSVEGIP |
| | | | QHAPMTPPNTPDPRSPPHPDNIAPGY |
| | | | SGPLKEIPPEKFNTTSVPKYYQSPWIE |
| | | | AIRDDPELLEALYPKLFKPEAKPELP |
| | | | DYRSFNRVATPFGGFERASKLVKFK |
| | | | VPDYNLLMLNDPRFMILANPLATRR |
| | | | SFNRTPKGWTSENIPVVFIQPSDSNN |
| | | | VPETEDL |
| SEQ ID | Q1AG08 | Calsarcin 1/Sus | MLSHNTMVKQRKQQASAIMKEIHG |
| NO: 163 | (UniProtKB) | scrofa | NDVDGMDLGKKVSIPRDIMLEELSH |
| | | | LSNRGARLFKMRQRRSDKYTFENFQ |
| | | | YESKAQINHNIAMQNGKLDGNNLES |
| | | | GSQQAPFTPPNTPDPRSPPNPENIAPG |
| | | | YSGPLKEIPPERFNTTAVPKYYQSPW |
| | | | EQAISNDPELLEALYPKLFKPEGKAE |
| | | | LPDYRSFNRVATPFGGFEKASKIVKF |
| | | | KVPDFELLLLTDPRFMAFANPLSGRR |
| | | | SFNRTPKGWISENIPIVITTEPTEDTTI |
| | | | PESDDL |
| SEQ ID | A8E4L9 | SYNC | MASPEPRRGGNGSAQAARATRPEVI |
| NO: 164 | (UniProtKB) | protein/Bos | SPLQEENSASLYELGTWNPEVTLSLE |
| | | taurus | GTLNLEDILYLGDTGDLDEALYVEET |
| | | | EPPEETLHIEETRMPDEALYLEEPVRP |
| | | | EAALYVEEPVKLEGVLYVEEPVKTA |
| | | | SPEQIVHGGDRVLSEAKSKPKESLQA |
| | | | GPSPSTEGSLSIEDLELLEGRFQQCIE |
| | | | AVAQLEEERDQLIHELVLLREPALQE |
| | | | VQQVHRDILAAYKQHAQAELERDG |
| | | | LREEIRLVKQKLFKVTKECVAYQYQ |
| | | | LECRRQDVAQFADFREALTTRAAQL |
| | | | SEELTQLREAYQKQKEQLRQQLEAP |
| | | | QSQRDGHFLQESRRLSAQFESLMAES |
| | | | RQGLEEEYEPQLLRLLERKEAAAKA |
| | | | LQKTQAEIQEMKEALRPLQAEAQKL |
| | | | HLQNRNLEDQITLVRQKRDEEVQQY |
| | | | REQLEEMEERQRQLRSGVQLQQQKN |
| | | | KEMEQLRVSLAEELATYKAMLPKSL |
| | | | EQANAPTSEAGGIETPSQGAV |
| SEQ ID | F1NM11 | Syncoilin, | MDAEGAGTPQAPEGTERPCLSLEEL |
| NO: 165 | (UniProtKB) | intermediate | GEYFQECIEAVEQLEKERDALIEELT |
| | | filament | QLREPALQDIRHAHQEIQAACRLLAK |
| | | protein/Gallus | VELERDNLRDEIRQIKQKLFKVTKEC |
| | | gallus | VACQYQLESRRHDLSQHAAYRDELE |
| | | | SQAGRLTGELSRLRESCEKEKEALRQ |
| | | | RLEAPPCRQDPQYLQESRRLSAEFES |
| | | | LVTRSRRGLEEHYEPQLLRLLERREA |
| | | | GTRALQELQGEVQGMKEALRPLQGE |
| | | | VSRLRLQNRSLEEQIVLVKQKRDEEV |
| | | | GQYREQVEELEDRLKELKNSVQLQQ |
| | | | RKNQELEELRSSLHHELSIYKGCLEIY |
| | | | GHLCKSEEKPDQEC |
| SEQ ID | A0A287BKP7 | Syncoilin, | MASPEPRRGGDGAAQAARETRAEAT |
| NO: 166 | (UniProtKB) | intermediate | SPLQEGNSESLYQLGTWNPEVTLSLE |
| | | filament | GTLNLEDILYLGDPVDLDEALYVEET |
| | | protein/Sus | EKPEETLHIEETRLPDEALYVEEPVKP |
| | | scrofa | EEMLYVEETVKPEEIVCVEQTMKPG |
| | | | ETTSPEPITYGGETVPSEEKPNPEESL |
| | | | RAKPNPSTDGSLSLEDLELLEGRFQQ |
| | | | CVQAVAQLEEERDQLIHELVLLREPA |
| | | | LQEVQQVHQDILAAYKLHAQAELER |
| | | | DGLREEIRLVKQKLFKVTKECVAYQ |
| | | | YQLECRRQDVAQFADFREVLTARAA |
| | | | QLSEELAQLRDAYQKQKEQLRQQLE |
| | | | APPSQRDGHFLQESRRLSARFENLM |
| | | | AESRQGLEEEYEPQLLRLLERKEAAA |
| | | | KALQKTQAEIQEMKEALRPLQAEAR |
| | | | QLHLQNRNLEDQITLVRQKRDEEVQ |
| | | | QYREQLEEMEERQRQLKSGVQLQQ |
| | | | QKNKEMEQLRISLAEELSTYKAMLP |
| | | | KSLERASAPTSEAGGIETQSQGAV |
| SEQ ID | A4FV66 | Sarcoglycan | MAAALIWISLLVGLLEGLGGTEAQQ |
| NO: 167 | (UniProtKB) | alpha/Bos taurus | TTLHPLVGRVFVHTLDHESFLQRPEH |
| | | | VFSVSAPIPITYHAHLQGHPDLPRWL |
| | | | RYTQRSPYQPGFLYGTATPEDRGHQI |
| | | | IEVTAYNRDSFNTTQQMLVLLIGDPE |
| | | | GPLLPYQAEFLVRSHDVEEVLPSIPAS |
| | | | RFLTALGGLWEPAELQLVNITSALDR |
| | | | GGRVPLPIEGRKEGVYIKVGSASPFS |
| | | | TCLKMVVSPDSHARCAQGQPPLLSC |
| | | | YDTLAPHFRVDWCNVSLVDKSVPEP |
| | | | ADEAPAPGDGILEHDPFFCPPTEATN |
| | | | RDFLTDALVTLLVPLLVALLLTLLLA |
| | | | YVMCCRREGRLKRDLATSDIQMVH |
| | | | HRTIRGNTEELRQMASSREVPRPLST |
| | | | LPMFNVHTGERLPPQVDSAQVPLILD |
| | | | QH |
| SEQ ID | Q0VCU7 | Gamma- | MVREQYTTITEGTHIERPENQAVYKI |
| NO: 168 | (UniProtKB) | sarcoglycan/Bos | GIYGWRKRCLYLFVLLLLIVLLVNFA |
| | | taurus | LTIWILRVMWFSPVGMGHLHVTADG |
| | | | LHLEGESEFLFPLYVKEIRSRVDSSLL |
| | | | LQSTQNVTMNARNTEGEVTGRLKV |
| | | | GPQMVEVQSQQFQIHSKDGKPLFTV |
| | | | DEEKVMVGTDKLRVTGPEGALFEHS |
| | | | VETPLVRPDPPQDLRLESPTRSLSMD |
| | | | APKGIHIQAPAGKIEALTQMDIVLQS |
| | | | SDGTVVLDAETVCLPELALGSQGPA |
| | | | GSSQGLYEVCVCPDGKLYLSVAGM |
| | | | GTTCHEHSHLCL |

Skeletal and smooth muscle tissue

| | | | |
|---|---|---|---|
| SEQ ID | P12003 | Metavinculin/ | MPVFHTRTIESILEPVAQQISHLVIMH |
| NO: 169 | (UniProtKB) | Gallus gallus | EEGEVDGKAIPDLTAPVSAVQAAVS |
| | | | NLVRVGKETVQTTEDQILKRDMPPA |
| | | | FIKVENACTKLVRAAQMLQADPYSV |
| | | | PARDYLIDGSRGILSGTSDLLLTFDEA |
| | | | EVRKIIRVCKGILEYLTVAEVVETME |
| | | | DLVTYTKNLGPGMTKMAKMIDERQ |
| | | | QELTHQEHRVMLVNSMNTVKELLP |
| | | | VLISAMKIFVTTKNTKSQGIEEALKN |
| | | | RNFTVEKMSAEINEIIRVLQLTSWDE |
| | | | DAWASKDTEAMKRALALIDSKMNQ |
| | | | AKGWLRDPNAPPGDAGEQAIRQILD |
| | | | EAGKAGELCAGKERREILGTCKTLG |
| | | | QMTDQLADLRARGQGATPMAMQK |
| | | | AQQVSQGLDLLTAKVENAARKLEA |
| | | | MTNSKQAIAKKIDAAQNWLADPNG |
| | | | GSEGEEHIRGIMSEARKVAELCEEPK |
| | | | ERDDILRSLGEISALTAKLSDLRRHG |
| | | | KGDSPEARALAKQIATSLQNLQSKT |
| | | | NRAVANTRPVKAAVHLEGKIEQAQR |
| | | | WIDNPTVDDRGVGQAAIRGLVAEGR |
| | | | RLANVMMGPYRQDLLAKCDRVDQL |
| | | | AAQLADLAARGEGESPQARAIAAQL |
| | | | QDSLKDLKARMQEAMTQEVSDVFS |
| | | | DTTTPIKLLAVAATAPSDTPNREEVF |
| | | | EERAANFENHAARLGATAEKAAAV |
| | | | GTANKTTVEGIQATVKSARELTPQV |
| | | | VSAARILLRNPGNQAAYEHFETMKN |
| | | | QWIDNVEKMTGLVDEAIDTKSLLDA |
| | | | SEEAIKKDLDKCKVAMANMQPQML |
| | | | VAGATSIARRANRILLVAKREVENSE |
| | | | DPKFREAVKAASDELSKTISPMVMD |
| | | | AKAVAGNISDPGLQKSFLDSGYRILG |
| | | | AVAKVREAFQPQEPDFPPPPPDLEHL |
| | | | HLTDELAPPKPPLPEGEVPPPRPPPPE |
| | | | EKDEEFPEQKAGEAINQPMMMAAR |
| | | | QLHDEARKWSSKPVTVINEAAEAGV |
| | | | DIDEEDDADVEFSLPSDIEDDYEPELL |
| | | | LMPTNQPVNQPILAAAQSLHREATK |
| | | | WSSKGNDIIAAAKRMALLMAEMSRL |
| | | | VRGGSGNKRALIQCAKDIAKASDEV |
| | | | TRLAKEVAKQCTDKRIRTNLLQVCE |
| | | | RIPTISTQLKILSTVKATMLGRTNISD |
| | | | EESEQATEMLVHNAQNLMQSVKET |
| | | | VREAEAASIKIRTDAGFTLRWVRKTP |
| | | | WYQ |
| SEQ ID | E1BPV6 | Smoothelin like | MEQKAGKSSEDGATVPPAAEAPEPA |
| NO: 170 | (UniProtKB) | 1/Bos taurus | GSGGSAEEETAGPAESAARAGPATA |
| | | | GQQERAPAEDAVSAECQADGLGEV |
| | | | KAESQGEVELQQEDPGQGETAAAHG |
| | | | KTDRKDQTDSEPEGAEGDRETASAS |
| | | | EEQSADEKEARLGSRETVDASREVQ |
| | | | AEPKQASGAQEAEAGGGEAEGPQEE |
| | | | GGKTEEPQAEAREEAGAPAAQPDSE |
| | | | PGSPDEEQDQGAGAEAEDGAGGPPS |
| | | | SPEGWPESPTEEGGSASPEGLSPDTA |
| | | | ASEELGPSASDSSPSDVPQSPTEPPPS |
| | | | EEKKKEKAPERRVSAPTRPRGPRAQ |
| | | | NRKAIVDKFGGAAAGPTALFRNTKA |
| | | | AGAAVGGVRNMLLEWCRAMTRSYE |
| | | | HVDIQNFSSSWGSGMAFCALIHKFFP |
| | | | DAFDYAALDPAQRRHNFTLAFSTAE |
| | | | KLADCAQLLEVDDMVRLAVPDSKC |
| | | | VYTYIQELYRSLVQKGLVKTKKK |

Cardiac and smooth muscle tissue

| | | | |
|---|---|---|---|
| SEQ ID | F1NHA9 | PDZ and LIM | MPQNVILPGPAPWGFRLSGGIDFNQP |
| NO: 171 | (UniProtKB) | domain protein | LIITRITPGSKASTANLCPGDIIVAING |
| | | 3/Gallus gallus | LSTENMTHNDAQERIKAAAHQLSLRI |
| | | | ERAETKLWSPQVSEDGKANPYKINL |
| | | | EAEPQEFKPIGTAHNRRAQPFVAAA |
| | | | NIDDKRQVVSSSYNSPIGLYSSGNIQ |
| | | | DALHGQLRSLIPNASQNDPAPTAVPQ |
| | | | SDVYRMLHSNQEEPSQPRQSGSFKV |
| | | | LQNLVSEEDGRPVGTRSVKAPVTKIP |
| | | | TGLPGAQKVPQCDKCGSGILGTVVK |
| | | | ARDKYRHPECFVCSDCNLNLKQKGY |
| | | | FFVEGQLYCETHARARMRPPEGYEA |
| | | | VTVYPKC |

Skeletal, cardiac, and smooth muscle tissue

| | | | |
|---|---|---|---|
| SEQ ID | Q04205 | Tensin/Gallus | MDFGSVMNQAATPCSPAVNYELPSP |
| NO: 172 | (UniProtKB) | gallus | GQSITKQVDTPDATRSPRGGQAHCK |
| | | | ASRSMSVTAAMESSCELDLVYITERII |
| | | | AVSYPSTAEEQSFRSNLREVAHMLK |
| | | | SKHGDNYVLFNLSERRHDISKLHPK |
| | | | VLDFGWPDLHTPALEKICSICKAMDT |
| | | | WLNAAAHNVVVLHNKGNRGRLGV |
| | | | VVAAYMHYSNISASADQALDRFAM |
| | | | KRFYEDKVVPVGQPSQKRYIHYFSG |
| | | | LLSGSIKMNNKPLFLHHVIMHGIPNF |
| | | | ESKGGCRPFLKIYQAMQPVYTSGIYN |
| | | | VQGDSQTGICITIEPGLLLKGDILLKC |
| | | | YHKKFRSPTRDVIFRVQFHTCAVHD |
| | | | LDIVFGKEDLDEAFRDERFPEYGKVE |
| | | | FVFSYGPEKIQGMEHLENGPSVSVDY |
| | | | NTSDPLIRWDSYENFNIQREDSAEGT |
| | | | WAEPALPGKHLEKEVGHTQGPLDGS |
| | | | LYAKVKKKDSLHGSIGAVNTARLPL |
| | | | SAAPNHVEHTLSVSSDSGNSTASTKT |
| | | | DRTDEPGAPGAPTGHAVLSPEEKRD |
| | | | VDRLLVGFGLESAAPMHNHAPGPAP |
| | | | ARLPAGPGRHVVPAQVHVNGAGTPL |
| | | | LAERETDILDDELPNQDGHSVGSLGT |
| | | | LSSLDGTTTASEAGFHEAPRVGSLSS |
| | | | LPNGPASYNGAEKMLKEGLYEAEPL |
| | | | SNGAYPYSNQNTLMGHHLRDPLAHL |
| | | | RPSRSAQEHLAGYPQRQPASASPAW |
| | | | LQPPVPQPYLYGYDLPSAHRSQSFPA |
| | | | VGTAKYEANLALPQAPARSTSSREA |
| | | | VQRGLNSWQQQGGSRPPSQLHDGG |
| | | | LESHSPSLSSCSPQPSPLQPMPPHSHS |
| | | | MPEFPRAPSRREIEQSIEALDVLMLD |
| | | | LAPSVHKSQSVPSAATRQDKPAAML |
| | | | SSLSAQRLSGHYAQPTPQVVQPRSFG |
| | | | TSVGTDPLAKPYSPGPLVPAARSTAE |
| | | | PDYTVHEYRETYTPYSYQPVPEPRSY |
| | | | GSAPASILPLSASYSPAGSQQLLVSSP |
| | | | PSPTAPAQSQLPHKGLESYEDLSRSG |
| | | | EEPLNLEGLVAHRVAGVQSREKSPE |
| | | | ESTVPARRRTPSDSHYEKSSPEPGSPR |
| | | | SPTVLSPEVVSTIAANPGGRPKEPHL |
| | | | HSYKEAFEEMESASPSSLTSGGVRSP |
| | | | PGLAKTPLSALGLKPHNPADILLHPV |
| | | | GELEGEAGADSEEEPRSYVESVART |
| | | | ATTGRAGNLPAAQPVGLEVPARNGA |
| | | | FGNSFTVPSPVSTSSPIHSVDGASLRS |
| | | | YPSEGSPHGTVTPPHAVAETAYRSP |
| | | | MVSQTPSAHSSYQTSSPSSFQAGTLG |
| | | | SPYASPDYPDGRGGFQPDPQARQQP |
| | | | QVSVVGVHALPGSPRTLHRTVATNT |
| | | | PPSPGFGRRAANPAVASVPGSPGLGR |
| | | | HTVSPHAPPGSPSLARHQMAAVPPG |
| | | | SPMYGYSSPEERRPTLSRQSSASGYQ |
| | | | PPSTPSFPVSPAYYPGTSTPHSSSPDS |
| | | | AAYRQGSPTPQPALPEKRRMSAGER |
| | | | SNSLPNYATVNGKASSPLSSGMSSPS |
| | | | SGSAVAFSHTLPDFSKFSMPDISPETR |
| | | | ANVKFVQDTSKYWYKPDISREQAIA |
| | | | LLKDREPGAFIIRDSHSFRGAYGLAM |
| | | | KVASPPPTVMQQNKKGDITNELVRH |
| | | | FLIETSPRGVKLKGCPNEPNFGCLSA |
| | | | LVYQHSIMPLALPCKLVIPDRDPMEE |
| | | | KKDAASTTNSATDLLKQGAACNVLF |
| | | | INSVEMESLTGPQAISKAVAETLVAD |
| | | | PTPTATIVHFKVSAQGITLTDNQRKL |
| | | | FFRRHYPLNTVTFCDLDPQERKWTK |
| | | | TDGSGPAKLFGFVARKQGSTTDNVC |
| | | | HLFAELDPDQPAAAIVNFVSRVMLG |
| | | | SGQKR |
| SEQ ID | A0A1D5PNS4 | Gelsolin/Gallus | MSEVGEEQNGGRGAGLGGITAALGL |
| NO: 173 | (UniProtKB) | gallus | VLIPIPIPILVPVPIPIPIPIPPPQGRKGQ |
| | | | SRHRALAGAGPGWGGVRAVTAPGA |
| | | | ARCARPRAPQLRPARPERQQQPVSM |
| | | | VEHAEFSKAGKEPGLQIWRIEKFDLV |
| | | | PVPKNLYGDFFTGDSYLVLNTIRQRS |
| | | | GNLQYDLHFWLGDESSQDERGAAAI |
| | | | FTVQMDDYLQGKAVQHREVQGHES |
| | | | STFLGYFKSGIKYKAGGVASGFRHV |
| | | | VPNEVTVQRLLQVKGRRTVRATEVP |
| | | | VSWESFNTGDCFILDLGSNIYQWCGS |
| | | | NSNRQERLKATVLAKGIRDNERNGR |
| | | | AKVFVSEEGAEREEMLQVLGPKPSL |
| | | | PQGASDDTKTDTANRKLAKLYKVSN |
| | | | GAGNMAVSLVADENPFSQAALNTE |
| | | | DCFILDHGTDGKIFVWKGRSANSDE |
| | | | RKAALKTATDFIEKMGYPKHTQVQV |
| | | | LPESGETPLFKQFFKNWRDKDQTEG |
| | | | LGEAYISGHVAKIEKVPFDAATLHTS |
| | | | RAMAAQHGMEDDGSGKKQIWRIEG |
| | | | SEKVPVDPATYGQFYGGDSYIILYDY |
| | | | RHAGKQGQIIYTWQGAHSTQDEIAT |
| | | | SAFLTVQLDEELGGSPVQKRVVQGK |
| | | | EPPHLMSMFGGKPLIVYKGGTSREG |
| | | | GQTTPAQTRLFQVRSSTSGATRAVEL |
| | | | DPVASQLNSNDAFVLKTPSAAYLWV |
| | | | GRGSNSAELSGAQELLKVLGARPVQ |
| | | | VSEGREPDNFWVALGGKAPYRTSPR |
| | | | LKDKKMDAHPPRLFACSNKSGRFTIE |
| | | | EVPGDLTQDDLATDDVMILDTWDQ |
| | | | VFVWIGKDAQEEEKTEALKSAKRYI |
| | | | ETDPASRDKRTPVTLVKQGLEPPTFS |
| | | | GWFLGWDDDYWSVDPLQRAMADV |
| | | | DV |
| SEQ ID | XP_025010159.1 | Dystroglycan | MTVGCVPQPPFLGRTLLPVLLLAASA |
| NO: 174 | (NCBI) | isoform | RCHWPSEPAEVVRDWENQLEASMH |
| | | X1/Gallus gallus | SVLSDLRETVPAVVGIPDSSAVVGRF |
| | | | FRVSIPTDLIASNGEAVQVSEAGKES |
| | | | LPSWLHWNAESSSLEGLPLDTDKGV |
| | | | HYISVTTLQPFPNGSYVPQAANVFSV |
| | | | EVHQEDHSEPQSVRAAAQEAGDAAP |
| | | | FVCGAEEPVTILTVILDADLTKMTPK |
| | | | QRIELLNRMRSFSEVELHNMKLVPV |
| | | | VNNRLFDMSAFMAGPGNAKKVVEN |
| | | | GALLSWKLGCSLSQNSVPNISKVEAP |
| | | | AKEGTMSARLGYPVVGWHIANKKP |
| | | | HLPKRMRRQINATPTPLTAIGPPTTA |
| | | | AQEPPTRIVPTPTSPAIAPPTETTAPPV |
| | | | REPIPLPRKPTVTIRTRGPIVQTPTLGP |
| | | | IQPTRLVEGTGTVSVPIRPTVPGYVEP |
| | | | TAVITPPTTTTKKPRVSTLKPATPSTT |
| | | | DSSTATTRRPTRRPKTPRPTKPPSTTR |
| | | | STISKLTTASPPTRVRTTASGVPRPWE |
| | | | PNEPPKLTNHIDRVDAWEGTYFEVKI |
| | | | PSDTFYDKEDTTTDKLQLTLKLKEQ |
| | | | QMIEENSWVQFNSTSQLMYGMPDRS |
| | | | HVGKHEYFMYATDKGGLFAVDAFEI |
| | | | HVHKRPHGDKSPVKFKARLEGDHSA |
| | | | VANDIHKKIMLVKKLALAFGDRNSS |
| | | | TITVQDIAKGSIVVEWTNNTLPLEPCP |
| | | | REQIRTLSKKIADDSGGPSPAFSNILQ |
| | | | PEFKPLNVSVVGSGSCRHIQFVPVTK |
| | | | DGRVISEATPTLAAGKDPEKSSEDDV |
| | | | YLHTVIPAVVVAAILLVAGIIAMICY |
| | | | RKKRKGKLTIEDQATFIKKGVPIIFAD |
| | | | ELDDSKPPPSSSMPLILQEEKAPLPPP |
| | | | EYPNQSMPETTPLNQDTIGEYTPLRD |
| | | | EDPNAPPYQPPPPFTAPMEGKGSRPK |
| | | | NMTPYRSPPPYVPP |
| SEQ ID | P12003-1 | Vinculin | MPVFHTRTIESILEPVAQQISHLVIMH |
| NO: 175 | (UniProtKB) | isoform 1/Gallus | EEGEVDGKAIPDLTAPVSAVQAAVS |
| | | gallus | NLVRVGKETVQTTEDQILKRDMPPA |
| | | | FIKVENACTKLVRAAQMLQADPYSV |
| | | | PARDYLIDGSRGILSGTSDLLLTFDEA |
| | | | EVRKIIRVCKGILEYLTVAEVVETME |
| | | | DLVTYTKNLGPGMTKMAKMIDERQ |
| | | | QELTHQEHRVMLVNSMNTVKELLP |
| | | | VLISAMKIFVTTKNTKSQGIEEALKN |
| | | | RNFTVEKMSAEINEIIRVLQLTSWDE |
| | | | DAWASKDTEAMKRALALIDSKMNQ |
| | | | AKGWLRDPNAPPGDAGEQAIRQILD |
| | | | EAGKAGELCAGKERREILGTCKTLG |
| | | | QMTDQLADLRARGQGATPMAMQK |
| | | | AQQVSQGLDLLTAKVENAARKLEA |
| | | | MTNSKQAIAKKIDAAQNWLADPNG |
| | | | GSEGEEHIRGIMSEARKVAELCEEPK |
| | | | ERDDILRSLGEISALTAKLSDLRRHG |
| | | | KGDSPEARALAKQIATSLQNLQSKT |
| | | | NRAVANTRPVKAAVHLEGKIEQAQR |
| | | | WIDNPTVDDRGVGQAAIRGLVAEGR |
| | | | RLANVMMGPYRQDLLAKCDRVDQL |
| | | | AAQLADLAARGEGESPQARAIAAQL |
| | | | QDSLKDLKARMQEAMTQEVSDVFS |
| | | | DTTTPIKLLAVAATAPSDTPNREEVF |
| | | | EERAANFENHAARLGATAEKAAAV |
| | | | GTANKTTVEGIQATVKSARELTPQV |
| | | | VSAARILLRNPGNQAAYEHFETMKN |
| | | | QWIDNVEKMTGLVDEAIDTKSLLDA |
| | | | SEEAIKKDLDKCKVAMANMQPQML |
| | | | VAGATSIARRANRILLVAKREVENSE |
| | | | DPKFREAVKAASDELSKTISPMVMD |
| | | | AKAVAGNISDPGLQKSFLDSGYRILG |
| | | | AVAKVREAFQPQEPDFPPPPPDLEHL |
| | | | HLTDELAPPKPPLPEGEVPPPRPPPPE |
| | | | EKDEEFPEQKAGEAINQPMMMAAR |
| | | | QLHDEARKWSSKGNDIIAAAKRMAL |
| | | | LMAEMSRLVRGGSGNKRALIQCAK |
| | | | DIAKASDEVTRLAKEVAKQCTDKRI |
| | | | RTNLLQVCERIPTISTQLKILSTVKAT |
| | | | MLGRTNISDEESEQATEMLVHNAQN |
| | | | LMQSVKETVREAEAASIKIRTDAGFT |
| | | | LRWVRKTPWYQ |
| SEQ ID | E1C8N4 | Supervillin/ | MKRKERIARRLEGIENDTQPMLLQN |
| NO: 176 | (UniProtKB) | Gallus gallus | CPGSVTHRLLEEDTPRYMRATDPYS |
| | | | HHLGRSNEEEEASDSSVEKQPRSSRY |
| | | | RTETTGGNTESLYSTGNMDTHELES |
| | | | KAERIARYKAERRRQLAEKYGLSLD |
| | | | SDLDSDYSSRYSRARKDPDSVERKA |
| | | | LRSERHDDENKDSGSLYLSRTEVKES |
| | | | KSVLSESREYSSREKDGIPAKEELSNE |
| | | | KSDKRADDDSAIRQASDPSATLDSSV |
| | | | SLTLSGRESSSCNEVPISPKQSPRESLS |
| | | | SPKRAASPIHLQNDQPLHSNIRQSESR |
| | | | FEMSTSGSALSAGGDRERGPRRPRR |
| | | | YFTPGENRKTSERFKTQPITSAERKET |
| | | | DRSIMCAEIPTADDEEKLDDRAKLSV |
| | | | AAKRLLFREMEKSFEMKNIPKPPTRS |
| | | | SAVERRMRRLQDRSHTQPITSEEVVI |
| | | | AATLQASAHQKILAKEEIRAAKEAM |
| | | | GQDQSDEPDSSTLSLAEKMALFNKL |
| | | | SQPVSRAISTRSRGDIRHRRMNTRYQ |
| | | | TQPVTLGEVEQVQNESGKLTPLSSTV |
| | | | STSVSAMASALSALFAGDMHAKSRS |
| | | | DGSVSAATKDELRFHASAESSDSSGK |
| | | | RTQKSEQWQPSMEVLESKRASKKHE |
| | | | EEGKRFLAHEANEIRKYSSFEDETTH |
| | | | PVLEKTGDYHKEAAYSFLRKGSMEL |
| | | | FSSQPLFQPLERKEIDTIMQDHVEPTS |
| | | | ELTSTPATRTLSQTTAAASCRLQELS |
| | | | EQLEGKFYKNSCEMFSAGENKIQTTE |
| | | | DATDSSSKTMSIKERLALLKKSGEED |
| | | | WKSRLSKKQEYAKVSATDRSAQMQ |
| | | | EVEQLLKKRIADNQESQMTIEERKHL |
| | | | ITAREEAWKSRGKGAANDSTQFTVA |
| | | | GRMVKKGLASPSALTPVASPYGNRQ |
| | | | KSTTPVTKPLEEIEARPDMQLESDMK |
| | | | LDKLESFLGRLNNKVAGMQETVLTV |
| | | | TGKSVKEVMKLDDDETFSKFYRRVD |
| | | | FPSSSVPLDLDEDFDAIFDPYAPKLIS |
| | | | SVAEHKRAVRPMRRVQSSRNPLKML |
| | | | AAREDILHEYTEQRLNVAFMESKRM |
| | | | KVEKMSANSNFSEVALAGLASKENF |
| | | | SNVSLRSVNLTEQNSNNSAVPYKKL |
| | | | MLLQIKGRRHVQTRLVEPRASSLNS |
| | | | GDCFLLLTPHLCFLWVGEFANVIEKA |
| | | | KASELATLIQTKRELGCRASYIQTIEE |
| | | | GINTHTHAAKDFWKLLGGQANYQS |
| | | | AGRPEEDEMYEAAIIETNCIYRLVED |
| | | | KLIPEDDYWGKMPKCTLLQPKEVLV |
| | | | FDFGSEVYVWHGKEVTLAQRKVAF |
| | | | QLAKHLWNGTFDYSNCDINPLDPGE |
| | | | CNPLIPRKGQGRPDWAVFGRLTEHN |
| | | | ETILFKEKFLDWTELKKLNEKNSSES |
| | | | LHQKEESKSDSKPYDVMLMVPVPQT |
| | | | AVGTVLDGMNIGRGYGLVEGEDGR |
| | | | QFEIITASVDVWHILEFDYSRLPKQSI |
| | | | GQFHEGDTYVVKWKYMVSTTVGSR |
| | | | QKGEQQVRAVGKEKCVYFFWQGRH |
| | | | STVSEKGTSALMTVELDEERGAQVQ |
| | | | VLQGKELPCFLQCFQGGMIVHAGRR |
| | | | EEEEENAQSDWRLYCVRGEVPNEGN |
| | | | LLEVACHCSSLRSRTSMIVLNINKALI |
| | | | YLWHGCKAQSHTKDVGRTAANKIK |
| | | | EQCPLEAGLHSSSKVTIHECDEGSEP |
| | | | LGFWDALGRRDRKAYDCMLQDPGK |
| | | | FNFTPRLFSLSSSSGEFSATEYVYPSR |
| | | | DPTVINSMPFLQEDLYTAPQPALFLV |
| | | | DNHHEVYLWQGWWPVENKITGSAR |
| | | | IRWATDRKCAMETVLQYCKGKNVK |
| | | | KPPKSYLIHAGLEPLTFTNMFPSWEH |
| | | | REDIAEITEMDADVSNQIILVEDVLA |
| | | | KLCKTVYPLADLLARPLPEGVDPLK |
| | | | LEIYLSDEDFEVALEMTREEYNALPS |
| | | | WKQVNLKKAKGLF |
| SEQ ID | Q5ZL50 | Profilin/Gallus | MAGWQSYVDNLMCDGCCQEAAIVG |
| NO: 177 | (UniProtKB) | gallus | YCDAKYVWAATAGGIFQSITPVEID |
| | | | MIVGKDREGFFTNGLTLGAKKCSVIR |
| | | | DSLYVDGDCTMDIRTKSQGGEPTYN |
| | | | VAVGRAGRVLVFVMGKEGVHGGGL |
| | | | NKKAYSMAKYLRDSGF |
| SEQ ID | A5PJI6 | Caveolae- | MEHNGSASNADKIHQNRLSNVTEDE |
| NO: 178 | (UniProtKB) | associated | DQDAALTIVTVLDKVAAIVDSVQAS |
| | | protein 4/Bos | QKRIEERHRVMENAIKSVQIDLLKFS |
| | | taurus | QSHSNTGYVINKLFEKTRKVSAHIKD |
| | | | VKARVEKQQTHVKKVEAKQEEIMK |
| | | | KNKFRVVIFQEEVQCPTSLSVVKDRS |
| | | | LTESPEEVDEIFDTPVDLSSDEEYFVE |
| | | | ESRSARLKKSGKERIDNIKKAFSKEN |
| | | | MQKTRQNFDKKVNRIRTRIVTPERRE |
| | | | RLRQSGERLRQSGERLKQSGERFKKS |
| | | | ISNAAPSREAFKMRSLRKTKDRAVA |
| | | | EGPEEVREMGVDIIARGEALGPISEL |
| | | | YPEALSETDPEEASATHPPQEGGEVS |
| | | | TPEPLKVTFKPQVKVEDDESLLLDLK |
| | | | Q |
| SEQ ID | Q62234 | Myomesin- | MSLPFYQRSHQHYDLSYRNKDLRTT |
| NO: 179 | (UniProtKB) | 1/Mus musculus | MSHYQQEKKRSAVYTHGSTAYSSRS |
| | | | LAARRQESEAFSQASATSYQQQASQ |
| | | | TYSLGASSSSRHSQGSEVSRKTASAY |
| | | | DYGYSHGLTDSSLLLEDYSSKLSPQT |
| | | | KRAKRSLLSGEETGSLPGNYLVPIYS |
| | | | GRQVHISGIRDSEEERIKEAAAYIAQ |
| | | | KTLLASEEAIAASKQSTASKQSATSK |
| | | | RTTSTLQREETFEKKSRNIAIREKAEE |
| | | | LSLKKTLEETQTYHGKLNEDHLLHA |
| | | | PEFIIKPRSHTVWEKENVKLHCSVAG |
| | | | WPEPRLTWYKNQVPINVHANPGKYI |
| | | | IESRYGMHTLEISKCDFEDTAQYRAS |
| | | | AMNVQGELSAYASVVVKRYKGELD |
| | | | ESLLRGGVSMPLSFAVTPYGYASKFE |
| | | | IHFDDKFDVSFGREGETMSLGCRVVI |
| | | | TPEIKHFQPEVQWYRNGAPVSPSKW |
| | | | VQPHWSGDRATLTFSHLNKEDEGLY |
| | | | TIRVRMGEYYEQYSAYVFVRDADAE |
| | | | IEGAPAAPLDVVSLDANKDYIIISWK |
| | | | QPAVDGGSPILGYFIDKCEVGTDTWS |
| | | | QCNDTPVKFARFPVTGLIEGRSYIFR |
| | | | VRAVNKTGIGLPSRVSEPVAALDPAE |
| | | | KARLKSHPSAPWTGQIIVTEEEPTEG |
| | | | VIPGPPTDLSVTEATRSYVVLSWKPP |
| | | | GQRGHEGIMYFVEKCDVGAENWQR |
| | | | VNTELPVKSPRFALFDLVEGKSYRFR |
| | | | VRCSNSAGVGEPSETTEVTVVGDKL |
| | | | DIPKAPGKIIPSRNTDTSVVVSWEESR |
| | | | DAKELVGYYIEASVVGSGKWEPCNN |
| | | | NPVKGSRFTCHGLTTAQSYIFRVRAV |
| | | | NAAGLSEYSQDSEAIEVKAAIGGGVS |
| | | | PDVWPQLSDTPGGLTDSRGGMNGAS |
| | | | PPTSQKDALLGSNPNKPSPPSSPSSRG |
| | | | QKEVSTVSESVQEPLSSPPQEAAPEE |
| | | | EQSQSEPPKKKKDPVAVPSAPYDITC |
| | | | LESFRDSMVLGWKQPDTTGGAEITG |
| | | | YYVNYREVVGEVPGKWREANIKAV |
| | | | SDAAYKISNLKENTLYQFQVSAMNI |
| | | | AGLGAPSTVSECFKCEEWTIAVPGPP |
| | | | HSVKLSEVRKNSLVLQWKPPVYSGR |
| | | | TPVTGYFVDLKEASAKDDQWRGLN |
| | | | EAAIVNKYLRVQGLKEGTSYVFRVR |
| | | | AVNQAGVGKPSDLAGPVVAETRPGT |
| | | | KEVVVSVDDDGVISLNFECDQMTPK |
| | | | SEFVWSKDYVPTEDSPRLEVENKGD |
| | | | KTKMTFKDLGTDDLGTYSCDVTDTD |
| | | | GIASSYLIDEEEMKRLLALSQEHKFP |
| | | | TVPTKSELAVEILEKGQVRFWMQAE |
| | | | KLSSNAKVSYIFNEKEIFEGPKYKMH |
| | | | IDRNTGIIEMFMEKLQDEDEGTYTFQ |
| | | | IQDGKATGHSTLVLIGDVYKKLQKE |
| | | | AEFQRQEWIRKQGPHFAEYLSWEVT |
| | | | GECNVLLKCKVANIKKETHIVWYKD |
| | | | EREISVDEKHDFKDGICTLLITEFSKK |
| | | | DAGFYEVILKDDRGKDKSRLKLVDE |
| | | | AFQDLMTEVCKKIALSATDLKIQSTA |
| | | | EGIRLYSFVCYYLDDLKVNWSHNGT |
| | | | GIKYTDRVKSGVTGEQIWLQINEPTP |
| | | | NDKGKYVMELFDGKTGHQKTVDLS |
| | | | GQAFDEAFAEFQRLKQAAIAEKNRA |
| | | | RVLGGLPDVVTIQEGKALNLTCNVW |
| | | | GDPPPEVSWLKNEKPLTSDDHCSLKF |
| | | | EAGKTAFFTISGVSTADSGKYGLVV |
| | | | KNKYGSETSDFTVSVFIPEEELRKGA |
| | | | MEPPKGNQKSK |
| SEQ ID | F1MKE9 | Spectrin beta | QPADMTSATEFENVGNQPPYSRINA |
| NO: 180 | (UniProtKB) | chain/Bos taurus | RWDGPDDELDNDNSSARLFERSRIK |
| | | | ALADEREVVQKKTFTKWANSHLVH |
| | | | VSCRITDLYKDLRDGRMLIKLLEVLS |
| | | | GEMLPKPTKGKMRIHCLENVDKALQ |
| | | | FLREQRVHLENMGSHDIVDGNHRLV |
| | | | LGLIWTIILRFQIQDIVVQTQEGRETR |
| | | | SAKDALLLWCQMKTAGYPHVNVTN |
| | | | FTSSWKDGLAFNALIHKHRPDLIDFD |
| | | | KLKDSNARHNLEHAFEVAERELGIIP |
| | | | LLDPEDVFTENPDEKSIITYVVAFYH |
| | | | YFSKMKVLAVEGKRVGKVIDHAIET |
| | | | EKMIEKYSGLASDLLTWIEQTITILNS |
| | | | RKFANSLAGVQQQLQAFSTYRTVEK |
| | | | PPKFQEKGNLEVLLFTIQSRMRANNQ |
| | | | KVYTPHDGKLVSDINRAWESLEEAE |
| | | | YRRELALRDELIRQEKLEQLARRFDR |
| | | | KAAMRETWLNENQRLVAQDNFGYD |
| | | | LAAVEAAKKKHEAIETDTAAYEERV |
| | | | RALEDLAQELERENYHDQKRIMARK |
| | | | DNILRLWDYLQELLQARRQRLEKTL |
| | | | DLQKLFQDMLHSIDWMDGIKAHLLS |
| | | | AEFGKHLLEVEDLLQKHKLMEADIA |
| | | | IQGDKVKAITTATLQFTEGPGYQPCD |
| | | | PQVIQDRVSHLEQCFEELSNTAAGRK |
| | | | AQLEQSKQLCKFFWEMDEAESWIKE |
| | | | KEQIYSSLDYGKDLTSVLILQRKHKA |
| | | | FEDELRGLDAHLDQIFREAEGMVAR |
| | | | KQFGHERIEVRIKKVSDQWNELKDL |
| | | | AAFCKKNLQDTENFFQFQGDADDLK |
| | | | AWLQDAHRLLSGEDVGHDEGATRA |
| | | | LGKKHKDFLEELEESRGVMEHLEQQ |
| | | | AQAFPQGFQDSPDVTNRLQALRDLY |
| | | | QQVVAQADMRQQRLQDALDLYTVF |
| | | | GETDACELWMGEKGKWLAQMEIPD |
| | | | TLEDLEVVQHRFDILDQEMKTLMTQ |
| | | | IDGVNLAANSLVESNHPRSAEVKKY |
| | | | QDRLNTRWQDFQTMVSERREAVDS |
| | | | ALRVHNYCVDCEETGKWIADKTKV |
| | | | VESTKDLGRDLAGVIAIQRKLSGLER |
| | | | DVAAIQARLGTLERESQQLMASHPE |
| | | | LKEDIEQRQAYVEELWQGLMQALKS |
| | | | QEASLGEASQLQAFLQDLDAFQAWL |
| | | | STTQKDVASKDMPESLPEAEQLLQQ |
| | | | HAAIKDDIDRHQESYQKVRVSGEKV |
| | | | THGQTDPEYLLLGQRLDGLEKGWD |
| | | | ALSRMWESRSQALTQCLGFQEFQKD |
| | | | AKQAEAILSNQEYTLAHLEPPDSLEA |
| | | | AEAGIRKFEDFLLSMENNRDKVLSPV |
| | | | DSGNKLVAEGNLYSDKIKEKVQLIE |
| | | | DRHRKNNEKAQEASDLLKDNLELQ |
| | | | NFLQNCQELTLWINDKLLTSQDVSY |
| | | | DEARNLHNKWLKHQAFVAELASHQ |
| | | | GWLENIDAEGKQLMEEKPQFAVLVS |
| | | | QKLEALHQLWDELQANTQEMAQHL |
| | | | SAARSSDLRSQTQADLNKWIRAMED |
| | | | QLQSDDLGKDLTSVNRMLAKLKRV |
| | | | EDQVNVRKEELQELFAQMPSLGEEA |
| | | | GDEALSIEKRFLDLLEPLGRRKKQLE |
| | | | SSRAKLQISRDLEDETLWVEERLPLA |
| | | | QSTDYGTNLQTVQLFMKKNQTLQN |
| | | | EILGHTPRVEDVLHRGQQLVAAAEID |
| | | | CQDVEERLGKLQGSWDTLQEAAAS |
| | | | RLQHLREASEAQQYYLDAGEAEAWI |
| | | | GEQEFYVFSDENPQDEESAIVMLKR |
| | | | HLRQQRAVDEYGRNIKQLAGRAQG |
| | | | LLSAGHPEGEQIIRLQGQVDKQYAGL |
| | | | KDMAEERRRKLENMYHLFQLKREV |
| | | | DDLEQWITEKDTVASSSEMGQDFDH |
| | | | VTVLRDKFRDFARETGAIGQERVDN |
| | | | VNAIIERLIDAGHSEAATIAEWKDGL |
| | | | NEMWADLLELIDTRMQLLAASYDL |
| | | | QRYFYTSSEILGLIGEKHRELPEDVGL |
| | | | DASTAESFHRTHTAFERELHLLGEQV |
| | | | QQFQDVAARLQTAYAGEKADIIQNK |
| | | | EQEVSAAWQALLDACAGRRTQLVD |
| | | | TADKFRFFSMARDLLSWMESIVRQIE |
| | | | TQEKPRDVSSVELLMKYHQGIQAEIE |
| | | | TRSKNFSTCLELGESLLQRQHQASEE |
| | | | IREKREQVVSRREEMQEKWEARWE |
| | | | RLRMLLEVCQFFRDASVAEAWLIAQ |
| | | | EPYLASRDYGHTVDSVEKLIKRHEAF |
| | | | EKSAASWEGRFAALEKPTTLELKER |
| | | | QTPERTEEEPGLQEEEGETAGEAPRG |
| | | | PHQATTERTSPGEEERPWPQDLQPPP |
| | | | PPGPHEDGQEEKSSTDERPATEPLFK |
| | | | VLDTPLSEGDEPTTLPAQRDRGHSVQ |
| | | | MEGFLGRKHDLEGPNKKASNRSWN |
| | | | NLYCVLRNSELAFYKDAKNLALGVP |
| | | | YHGEEPLALRHAICEIAANYKKKKH |
| | | | VFKLRLSNGSEWLFHGKDEEEMLSW |
| | | | LQGVSTAINESQSIRVKAQSLPLPPIT |
| | | | GPEASLGKKDKEKRFSFFPKKK |
| SEQ ID | E1BIS6 | Synemin/Bos | MLSWRLHTGPEKAELQELNARLYD |
| NO: 181 | (UniProtKB) | taurus | YVCRVRELERENLLLEEELRSRRGQE |
| | | | GLWAEAEARCTEEARGLRRQLDELS |
| | | | WATALAQGERDALRRELRELQLLGE |
| | | | ETRAARGRLDAELDSQRRELQEELA |
| | | | ARAALEALLGRLQAERRGLDAARER |
| | | | DVRELRARAAGLTLRYRGRVAGPA |
| | | | APPLRLRELHEDCALLVTEAWRETV |
| | | | QRYEDEVRELEEALRRGRESRREAE |
| | | | EETRLCAQEAEALRREVLELEQLRAL |
| | | | LEEELLRVREASELQAEERQREIAYL |
| | | | EDEKAALTLAMADRLRDYQDLVQV |
| | | | KTGLSLEVATYRALLEGESNPEILIVE |
| | | | CIENMPQEFREKSYQYTTSVLQKENE |
| | | | RNLFPRQKAPPASFRQSLAPRSQTPV |
| | | | RSDARRDVLGSGYSSFTTAWQENAY |
| | | | QKTASGQTNFRTFSPTPGLLRNTEAQ |
| | | | LKTFPERPRTEGTKAPPASSAKESAA |
| | | | AAEPYQERRAEEAAAASEGTWSNER |
| | | | TVIWGKKTEAKATKEQERNRPGTIQ |
| | | | TKREEKMFDSKEKASEERNLRWEEL |
| | | | TKLDKEARKRESEQMREKAREKESL |
| | | | KEESVKGREIPIRLEASQDSTAKVAP |
| | | | QDLQTPLKEDAGDGTGRGVETREAG |
| | | | FTLGASDTTASLKGGSMTETIAENIV |
| | | | SSILKQFTHSPEAEASAESFPDTKVTY |
| | | | VDRKEVPGDRKAKTEIVVESKLTEEI |
| | | | DISDEAGLDYLLSKDAKEVELKGKS |
| | | | AERLIGDVIRLGLKGKEGRAKVVNV |
| | | | EIIEEPMSYVSSEKEDEFSTPFEVEEID |
| | | | DVSPSSRGLVEEREEAVYGESDVTCS |
| | | | GDAGQEARRPQESVTHVEEVTEAGD |
| | | | LEGEQSYFVSTPDEHPGAPEREEGSV |
| | | | YGQIHIEEESTIRYSWQDEIVPGSRRR |
| | | | VRRDDVPGEKVVKPVDVPEVPLEGD |
| | | | TASAPWKEQTRSGEFHAKPIVIEKEI |
| | | | KIPHEFHTSIKEGSKEPRHQLVEVIEQ |
| | | | LEENLPERMKEELSALTREGQGGPA |
| | | | GVSFDVKKVQSAGSGAVTLVAEVNL |
| | | | SQTVDADQLDLEELSKDEAGEIEKA |
| | | | VESVVRDSLARRRSPGPGSPEAEAGG |
| | | | GFRRWATQELYSSSAGEADAGLASP |
| | | | PGPVSATVEVTSPTGFVHAHVLEDVS |
| | | | QSGGRVQIASPGMWRTEQVSAEGRA |
| | | | AQAVEVSAEGQASWAEGSAGTSRSA |
| | | | RLITLGARQSPVSTEVIFPGPDAACPE |
| | | | AGGTEEPGPAELPTEPPRFGRHSPFGS |
| | | | QQFYAQREVIFQAPVSGVGKAGDSS |
| | | | QAEESAGTQTSVKHLQLGTREGLTE |
| | | | QTQLTAPLSGAVELGVREASVLMEA |
| | | | WSGDGTSIRHVTIGPQRHQATEPIVP |
| | | | PPLEFSDSESSTHREGSADETLATSSY |
| | | | TVGRNILVTEKSTFQRAVSESPQEAS |
| | | | AEDMSGNEVTSGVSRSFRHIQLGPAE |
| | | | AKTSEHIVFHGPISKTFALAGSVDSPE |
| | | | VSEVADSGRTLRHVALGPKETSFTFQ |
| | | | MDVSNVEATSRWTQEARVLFPAGTE |
| | | | AEAPSVSEPGAWRDASSRNDLAAGV |
| | | | SFQGSAGDRHQAPGERGKEQAEFDK |
| | | | TVQLQRMVDQRSVISDEKKVALLYL |
| | | | DSQEEENEGHWF |
| SEQ ID | E1BF59 | Plectin/Bos | MVAGMFMPLDQLRAIYEVLFREGV |
| NO: 182 | (UniProtKB) | taurus | MVAKKDRRPRSLHPHVPGVTNLQV |
| | | | MRAMASLRARGLVRETFAWRHFYW |
| | | | YLTNEGIAHLRQYLHLPPEIVPASLQ |
| | | | RVRRPVAMVMPARRTPHVQAVQGP |
| | | | LGCPPKRGPPPTEDPAREERCVYRRK |
| | | | EPEEGAPEPPVVPAATPGTLARQGLE |
| | | | PAPPTDERDRVQKKTFTKWVNKHLI |
| | | | KAQRHISDLYEDLRDGHNLISLLEVL |
| | | | SGDSLPREKGRMRFHKLQNVQIALD |
| | | | YLRHRQVKLVNIRNDDIADGNPKLT |
| | | | LGLIWTIILHFQISDIQVSGQSEDMTA |
| | | | KEKLLLWSQRMVEGYQGLRCDNFT |
| | | | SSWRDGRLFNAIIHRHKPMLIDMNK |
| | | | VYRQTNLENLDQAFSVAERDLGVTR |
| | | | LLDPEDVDVPQPDEKSIITYVSSLYD |
| | | | AMPRVPDVQDGVKANELQLRWQEY |
| | | | RELVLLLLQWIRAHTAAFEERRFPSS |
| | | | FEEIEILWCQFLKFKETELPAKEADK |
| | | | NRSKGIYQSLEGAVQAGQLKVPPGY |
| | | | HPLDVEKEWGKLHVAILEREKQLRS |
| | | | EFERLERLQRIVSKLQMEAGLCEEQL |
| | | | NQADALLQSQDVRLLAAGKAPQRA |
| | | | GEVERDLDKADGMIRLLFNDVQALK |
| | | | DGRHPQGEQMYRRVYRLHERLVAIR |
| | | | TEYNLRLRGTPRHPELEDSTLRYLQD |
| | | | LLAWVEENQRRLDGAEWGVDLPSV |
| | | | EAQLGSHRGLHQSVEEFRAKIERART |
| | | | DEGQLSPATRGAYRDCLGRLDLQYA |
| | | | KLLNSSKARLRSLESLHGFVAAATKE |
| | | | LMWLSEKEEEEVGFDWSERNSNMA |
| | | | AKKEAYSALMRELELKEKKIKEIQST |
| | | | GDRLLREDHPARPTVESFQAALQTQ |
| | | | WSWMLQLCCCIEAHLKENTAYFQFF |
| | | | SDVREAEEQLRKLQETLHRKYTCDR |
| | | | SITVTRLEDLLQDAQDEKDQLNEYR |
| | | | GHLSGLAKRAKAIVQLTPRNPTQPTR |
| | | | GRVPLLAVCDYKQVEATVHKGDEC |
| | | | QMLGPAQPFHWKVLSGSGSEAAVPS |
| | | | VCFLVPPPNQEALEAVARLEAQHQA |
| | | | LVTLWHQLHTDMKSLLAWQSLSRD |
| | | | VQLIRSWSLVTFRTLKPEEQRQALRS |
| | | | LELHYQAFLRDSQDAGGFGPEDRLQ |
| | | | AEREYGSCSRHYQQLLQSLEQGKCG |
| | | | QCGXLDKEPARECAQRIAEQQKAQA |
| | | | EVEGLGKGVARLSAEAEKVLALPEP |
| | | | SPAAPTLRSELELTLGKLEQVRSLSAI |
| | | | YLEKLKTISLVIRSTQGAEEALKAHE |
| | | | EQLKEAQAVPAALPELEATKAAMK |
| | | | KLRAQAEAQQPVFDALRDELRGAQE |
| | | | VGERLQQRHGERDVEVERWRERVT |
| | | | QLLERWQAVLAQTDVRQRELEQLG |
| | | | RQLRYYRESADPLGAWLQDARRRQ |
| | | | EQIQAVPLADSQAVREQLRQEKALL |
| | | | EEIERHAEKVEECQRFAKQYINAIKD |
| | | | YELQLVTYKAQLEPVASPAKKPKVQ |
| | | | SGSESVIQEYVDLRTRYSELSTLTSQ |
| | | | YIRFISETLRRMEEEERLAEQQRAEE |
| | | | RERLAEVEAALEKQRQLAEAHAQA |
| | | | KAQAEREAQELQRRMQEEVARREE |
| | | | VVVDAQQQKRSIQEELQQLRQSSEA |
| | | | EIQAKARQVEAAERSRLRIEEEIRVV |
| | | | RLQLETTERQRGGAEGELQALRARA |
| | | | EEAEAQKRQAQEEAERLRRQVQDET |
| | | | QRKRQAEAELGLRVKAEAEAAREK |
| | | | QRALQALEELRLQAEEAERRLRQAE |
| | | | AERARQVQVALETAQRSAQAELQSK |
| | | | HASFAEKTAQLERTLEEEHVTVVQL |
| | | | REEATRREQQQAEAERAREEAEREL |
| | | | ERWQLKANEALRLRLQAEEVAQQK |
| | | | SLAQAEAEKQKEAAEREARRRGKAE |
| | | | EQAVRQRELAEQELERQRQLAEGTA |
| | | | QQRLAAEQELIRLRAETEQGEQQRQ |
| | | | LLEEELARLQSEAAAATQKRQELEA |
| | | | ELAKVRAEMEVLLASKARAEEESRS |
| | | | SSEKSKQRLEAEAGRFRELAEEAARL |
| | | | RALAEEAKRQRQLAEEDAARQRAE |
| | | | AERVLSEKLAAISEATRLKTEAEIAL |
| | | | KEKEAENERLRRLAEDEAFQRRRLE |
| | | | EQAAQHKADIEERLAQLRKASESELE |
| | | | RQKGLVEDTLRQRRQVEEEILALKA |
| | | | SFEKAAAGKAELELELGRIRGNAEDT |
| | | | LRSKEQAEQEAARQRQLAAEEERRR |
| | | | REAEERVQKSLAAEEEAARQRKAAL |
| | | | EEVERLKAKVEEARRLRERAEQESA |
| | | | RQLQLAQEAAQKRLQAEEKAHAFA |
| | | | VQQKEQELQQTLQQEQSMLERLRGE |
| | | | AEAARRAAEEAEEARERAEGEAAQS |
| | | | RQRVEEAERLKQAAEEQAQAQAQA |
| | | | QAAAEKLRKEAEQEAARRAQAEQA |
| | | | ALRQKQAADAEMEKHKKFAEQTLR |
| | | | QKAQVEQELTALRLKLEETDHQKSIL |
| | | | DQELQRLKAEVTEAARQRSQVEEEL |
| | | | FSVRVQMEELGKLKARIEAENRALIL |
| | | | RDKDNTQRLLQEEAEKMKQVAEEA |
| | | | ARLSVAAQEAARLRQLAEEDLAQQR |
| | | | ALAEKMLKEKMQAVQEATRLKAEA |
| | | | ELLQQQKELAQEQARRLQEDKEQM |
| | | | AQQLAQETQGFQRTLETERQRQLEM |
| | | | SAEAERLRLRVAEMSRAQARAEEDA |
| | | | QRFRKQAEEISAKLHRTELATQEKVT |
| | | | LVQTLETQRQQSDRDADRLREAIAE |
| | | | LEREKDKLKKEAELLQLKSEEMQTV |
| | | | QQEQLLQETQALQQSFLSEKDSLLQR |
| | | | ERFIEEEKAKLERLFQDEVAKAQKLR |
| | | | EEQQRQQQQMQQEKQQLLASMEEA |
| | | | RRRQREAEEGVRRKQEELQLLEQQR |
| | | | QQQEQLLAEENRRLRERLEHLEEEH |
| | | | RAALAHSEEITAAQAAATRALPNGQ |
| | | | DATDGPAAEPEHAFEGLRQKVPAQQ |
| | | | LQEAGILSTEEVQRLVQGHTTVAELT |
| | | | QREDVRRYLQGHSSIAGLLLKPANE |
| | | | KLTIYAALRRQLLSPGTAVILLEAQA |
| | | | ASGFLLDPVRNRRLTVNEAVKEGVV |
| | | | GPELHHKLLSAERAVTGYKDPYTGE |
| | | | QISLFQAMKKDLIVREHGIRLLEAQI |
| | | | ATGGVIDPVHSHRVPVDVAYQRGYF |
| | | | DEEMNRVLQDPSDDTKGFFDPNTHE |
| | | | NLTYLQLLERCVEDPETGLRLLPLTD |
| | | | QAAKGGELVYTDSEARDVFEKATVS |
| | | | APFGKFQGKTVTIWELINSEYFTAEQ |
| | | | RRDLLRQFRTGKVTVEKIIKIVITVIE |
| | | | EHEQKGQLCFQGLRALVPAAELLES |
| | | | GVIDWDLFRQLQLGERSVQEVAEVE |
| | | | AVRRALRGSGVIAGVWLEEARQKLS |
| | | | IYEALKKELLQPEAAVALLEAQAGT |
| | | | GHVIDPATSARLTVDEAVRAGLVGP |
| | | | ELHEKLLSAEKAVTGYKDPYSGQSV |
| | | | SLFQALKKGLIPREQGLRLLDAQLST |
| | | | GGIVDPSKSHRLPLDVACARGYLDK |
| | | | ETSTALTAPRDDAKTYYNPRTWEPA |
| | | | TYSQLQQQCRPDPLTGLSLLPLSEEA |
| | | | ARARQQELYSEVQAREAFQKATVEV |
| | | | PVGSFQGRAVTIWELINSEYFTAEQR |
| | | | QELLRQFRTGKVTVEKIIKIIITIVEEV |
| | | | ETTRRERLSFSGLRAPVPASELLASGI |
| | | | LSSSQFEQLKDGKTSVKDLSELDSVR |
| | | | TLLQGSGCLAGIYLEESKEKVTIYEA |
| | | | MRRGLLRPSTAILLLEAQAATGFLVD |
| | | | PVRNQRLYVHEAVKAGVVGPELHE |
| | | | KLLSAEKAVTGYKDPYSGSTISLFQA |
| | | | MKKGLVVREHGIRLLEAQIATGGIID |
| | | | PVHSHRVPVDVAYQRGYFDKEMNR |
| | | | VLEDPSDDTKGFFDPNTRENLTYRQL |
| | | | LERCVEDPETGLRLLPLKGPEKAEVV |
| | | | ETTRVYTEEETRRAFEETQIDIPGGGS |
| | | | HGGSTMSLWEVMQSDLIPEEQRAQL |
| | | | MADFQAGRVTKERMIIIIIEIIEKTEIV |
| | | | RQQNLASYDYVRRRLTAEDLHEARV |
| | | | ISRESYSLLREGTRSLREVLEAESAW |
| | | | RYLYGTGCVAGVYLPGSRQTLTIYQ |
| | | | ALKKGLLSAEVARLLLEAQAATGFL |
| | | | LDPVKGDRLTVDEAVRKGLVGPELH |
| | | | DRLLSAERAVTGYRDPYTEQTISLFQ |
| | | | AMKKDLIPAEEALRLLDAQLATGGI |
| | | | VDPRLGFHLPLEVAYQRGYLNKDTH |
| | | | DQLSEPSEVRSYIDPSTDERLSYTQLL |
| | | | RRCRRDEASGLFLLPLSDARKLTFRG |
| | | | LRKQITVEELVRSHVMDEATAQRLQ |
| | | | EGLTSIEEVSKNLQKFLEGTSSIAGVL |
| | | | VDATKERLSVYQAMKKGIIRPGTAF |
| | | | ELLEAQAATGYVIDPIKGLKLTVEEA |
| | | | VRMGIVGPEFKDKLLSAERAVTGYK |
| | | | DPYSGKLISLFQAMKKGLILKDHGIR |
| | | | LLEAQIATGGIIDPEESHRLPVDVAY |
| | | | QRGLFDEEMNEILTDPSDDTKGFFDP |
| | | | NTEENLTYLQLMERCVTDPQTGLRL |
| | | | LPLKEKKRERKTSSKSSVRKRRVVIV |
| | | | DPETGKEMSVYEAYRKGLIDHQTYL |
| | | | ELSEQECEWEEITISSSDGVVKSMIID |
| | | | RRSGRQYDIDEAIAKSLIDRSALDQY |
| | | | RAGTLSITEFADMLSGNAGGFRSRSS |
| | | | SVGSSSSYPISPAVSRTQLASWSDPTE |
| | | | ETGPVAGILDTETLEKVSITEAMHRN |
| | | | LVDNITGQRLLEAQACTGGIIDPNTG |
| | | | ERFPVTEAVNKGLVDKIMVDRINLA |
| | | | QKAFCGFEDPRTKTKMSAAQALKK |
| | | | GWLYYEAGQRFLEVQYLTGGLIEPD |
| | | | TPGRVPLDEALQRGTVDARTAQKLR |
| | | | DVSAYSKYLTCPKTKLKISYKDALD |
| | | | RSMVEEGTGLRLLEAATQSSKGYYS |
| | | | PYSVSGSGSTTGSRSGSRTGSRAGSR |
| | | | RGSFDATGSGFSMTFSSSSYSSSGYG |
| | | | RRYASGPTSSLGGPESTAA |
| SEQ ID | Q63258-2 | Integrin alpha-7 | MARIPRCDFLGLPGICYLLSFLLAGLL |
| NO: 183 | (UniProtKB) | Isoform Alpha- | LPRASAFNLDVMGAIRKEGEPGSLFG |
| | | 7X1A/Rattus | FSVALHRQLQPRPQSWLLVGAPQAL |
| | | norvegicus | ALPGQQANRTGGLFACPLSLEETDC |
| | | | YRVDIDRGANVQKESKENQWLGVS |
| | | | VRSQGAGGKVVTCAHRYESRQRVD |
| | | | QVLETRDVIGRCFVLSQDLAIRDELD |
| | | | GGEWKFCEGRPQGHEQFGFCQQGT |
| | | | AATFSPDSHYLIFGAPGTYNWKGLLF |
| | | | VTNIDSSDPDQLVYKTLDPADRLTGP |
| | | | AGDLTLNSYLGFSIDSGKGLMRSEEL |
| | | | SFVAGAPRANHKGAVVILRKDSASR |
| | | | LIPEVVLSGERLTSGFGYSLAVTDLN |
| | | | SDGWADLIVGAPYFFERQEELGGAV |
| | | | YVYMNQGGHWADISPLRLCGSPDS |
| | | | MFGISLAVLGDLNQDGFPDIAVGAPF |
| | | | DGDGKVFIYHGSSLGVVTKPSQVLE |
| | | | GEAVGIKSFGYSLSGGLDVDGNHYP |
| | | | DLLVGSLADTAALFRARPVLHVSQEI |
| | | | FIDPRAIDLEQPNCADGRLVCVHVKV |
| | | | CFSYVAVPSSYSPIVVLDYVLDGDTD |
| | | | RRLRGQAPRVTFPGRGPDDLKHQSS |
| | | | GTVSLKHQHDRVCGDTVFQLQENV |
| | | | KDKLRAIVVTLSYGLQTPRLRRQAP |
| | | | DQGLPLVAPILNAHQPSTQRTEIHFL |
| | | | KQGCGDDKICQSNLQLAQAQFCSRIS |
| | | | DTEFQALPMDLDGTALFALSGQPFIG |
| | | | LELTVTNLPSDPARPQADGDDAHEA |
| | | | QLLATLPASLRYSGVRTLDSVEKPLC |
| | | | LSNENASHVECELGNPMKRGTQVTF |
| | | | YLILSTSGITIETTELKVELLLATISEQ |
| | | | DLHPVSVRAHVFIELPLSISGVATPQQ |
| | | | LFFSGKVKGESAMRSERDVGSKVKY |
| | | | EVTVSNQGQSLNTLGSAFLNIMWPH |
| | | | EIANGKWLLYPMRVELEGGQGPEKK |
| | | | GICSPRPNILHLDVDSRDRRRRELGQ |
| | | | PEPQEPPEKVEPSTSWWPVSSAEKRN |
| | | | VTLDCAQGTAKCVVFSCPLYSFDRA |
| | | | AVLHVWGRLWNSTFLEEYMSVKSL |
| | | | EVIVRANITVKSSIKNLLLRDASTVIP |
| | | | VMVYLDPVAVVAEGVPWWVILLAV |
| | | | LAGLLVLALLVLLLWKCGFFRRNSP |
| | | | SSSFPANYHRAHLAVQPSAMEAGGP |
| | | | GTVGWDSSSGRSTLRPLYPSTTQ |
| SEQ ID | A0A140T8D2 | Integrin | MNLQLIFWIGLISSVCCVFGQADENR |
| NO: 184 | (UniProtKB) | beta/Bos taurus | CLKANAKSCGECIQAGPNCGWCTNS |
| | | | TFLQEGMPTSARCDDLEALKKKGCH |
| | | | PNDIENPRGSKDIKKNKNVTNRSKGT |
| | | | AEKLQPEDITQIQPQQLVLQLRSGEP |
| | | | QTFTLKFKRAEDYPIDLYYLMDLSYS |
| | | | MKDDLENVKSLGTDLMNEMRRITSD |
| | | | FRIGFGSFVEKTVMPYISTTPAKLRNP |
| | | | CTNEQNCTSPFSYKNVLSLTDKGEVF |
| | | | NELVGKQRISGNLDSPEGGFDAIMQ |
| | | | VAVCGSLIGWRNVTRLLVFSTDAGF |
| | | | HFAGDGKLGGIVLPNDGQCHLENDV |
| | | | YTMSHYYDYPSIAHLVQKLSENNIQT |
| | | | IFAVTEEFQPVYKELKNLIPKSAVGT |
| | | | LSANSSNVIQLIIDAYNSLSSEVILENS |
| | | | KLPEGVTINYKSYCKNGVNGTGENG |
| | | | RKCSNISIGDEVQFEISITANKCPNKN |
| | | | SETIKIKPLGFTEEVEIILQFICECECQ |
| | | | GEGIPGSPKCHDGNGTFECGACRCN |
| | | | EGRVGRHCECSTDEVNSEDMDAYC |
| | | | RKENSSEICSNNGECVCGQCVCRKR |
| | | | DNTNEIYSGKFCECDNFNCDRSNGLI |
| | | | CGGNGVCKCRVCECNPNYTGSACD |
| | | | CSLDTTSCMAVNGQICNGRGVCECG |
| | | | ACKCTDPKFQGPTCEMCQTCLGVCA |
| | | | EHKECVQCRAFNKGEKKDTCAQECS |
| | | | HFNITKVENRDKLPQPGQVDPLSHC |
| | | | KEKDVDDCWFYFTYSVNGNNEATV |
| | | | HVVETPECPTGPDIIPIVAGVVAGIVL |
| | | | IGLALLLIWKLLMIIHDRREFAKFEKE |
| | | | KMNAKWDTQENPIYKSPINNFKNPN |
| | | | YGRKAGL |
| SEQ ID | P07228 | Integrin beta- | MAETNLTLLTWAGILCCLIWSGSAQ |
| NO: 185 | (UniProtKB) | 1/Gallus gallus | QGGSDCIKANAKSCGECIQAGPNCG |
| | | | WCKKTDFLQEGEPTSARCDDLAALK |
| | | | SKGCPEQDIENPRGSKRVLEDREVTN |
| | | | RKIGAAEKLKPEAITQIQPQKLVLQL |
| | | | RVGEPQTFSLKFKRAEDYPIDLYYLM |
| | | | DLSYSMKDDLENVKSLGTALMREM |
| | | | EKITSDFRIGFGSFVEKTVMPYISTTP |
| | | | AKLRNPCTGDQNCTSPFSYKNVLSLT |
| | | | SEGNKFNELVGKQHISGNLDSPEGGF |
| | | | DAIMQVAVCGDQIGWRNVTRLLVFS |
| | | | TDAGFHFAGDGKLGGIVLPNDGKCH |
| | | | LENNMYTMSHYYDYPSIAHLVQKLS |
| | | | ENNIQTIFAVTEEFQAVYKELKNLIPK |
| | | | SAVGTLSSNSSNVIQLIIDAYNSLSSE |
| | | | VILENSKLPKEVTISYKSYCKNGVND |
| | | | TQEDGRKCSNISIGDEVRFEINVTAN |
| | | | ECPKKGQNETIKIKPLGFTEEVEIHLQ |
| | | | FICDCLCQSEGEPNSPACHDGNGTFE |
| | | | CGACRCNEGRIGRLCECSTDEVNSED |
| | | | MDAYCRRENSTEICSNNGECICGQC |
| | | | VCKKRENTNEVYSGKYCECDNFNC |
| | | | DRSNGLICGGNGICKCRVCECFPNFT |
| | | | GSACDCSLDTTPCMAGNGQICNGRG |
| | | | TCECGTCNCTDPKFQGPTCEMCQTC |
| | | | LGVCAEHKDCVQCRAFEKGEKKETC |
| | | | SQECMHFNMTRVESRGKLPQPVHPD |
| | | | PLSHCKEKDVGDCWFYFTYSVNSNG |
| | | | EASVHVVETPECPSGPDIIPIVAGVVA |
| | | | GIVLIGLALLLIWKLLMIIHDRREFAK |
| | | | FEKEKMNAKWDTGENPIYKSAVTTV |
| | | | VNPKYEGK |
| SEQ ID | Q9GLP0 | Integrin beta- | MNLQLIFWIGLISSVCYVFGQADENR |
| NO: 186 | (UniProtKB) | 1/Sus scrofa | CLKANAKSCGECIQAGPNCGWCTNS |
| | | | TFLQEGMPTSARCDDLEALRKKGCH |
| | | | PDDIENPRGSKNIKKNKNVTNRSKGT |
| | | | AEKLQPEDITQIQPQQLVLQLRSGEP |
| | | | QTFTLKFKRAEDYPIDLYYLMDLSYS |
| | | | MKDDLENVKSLGTDLMNEMRRITSD |
| | | | FRIGFGSFVEKTVMPYISTTPAKLRNP |
| | | | CTSEQNCTSPFSYKNVLSLTDKGEVF |
| | | | NELVGKQRISGNLDSPEGGFDAIMQ |
| | | | VAVCGSLIGWRNVTRLLVFSTDAGF |
| | | | HFAGDGKLGGIVLPNDGHCHLENDV |
| | | | YTMSHYYDYPSIAHLVQKLSENNIQT |
| | | | IFAVTEEFQPVYKELKNLIPKSAVGT |
| | | | LSANSSNVIQLIIDAYNSLSSEVILENS |
| | | | KLPEGVTINYKSYCKNGVNGTGENG |
| | | | RKCSNISIGDEVQFEISITANKCPNKN |
| | | | SETIKIKPLGFTEEVEIILQFICECECQS |
| | | | EGIPSSPKCHDGNGTFECGACRCNEG |
| | | | RVGRHCECSTDEVNSEDMDAYCRK |
| | | | ENSSEICTNNGECVCGQCVCRKRDN |
| | | | TNEIYSGKFCECDNFNCDRSNGLICG |
| | | | GNGVCKCRVCECNPNYTGSACDCSL |
| | | | DTTSCMAVNGQICNGRGVCECGVC |
| | | | KCTDPKFQGPTCEMCQTCLGVCAEH |
| | | | KECVQCRAFNKGEKKDTCAQECSHF |
| | | | NITKVENRDKLPQPGQVDPLSHCKE |
| | | | KDVDDCWFYFTYSVNGNNEATVHV |
| | | | VETPECPTGPDIIPIVAGVVAGIVLIGL |
| | | | ALLLIWKLLMIIHDRREFAKFEKEKM |
| | | | NAKWDTGENPIYKSAVTTVVNPKYE |
| | | | GK |
| SEQ ID | F1MX12 | Ankyrin repeat | WQKHLAGRGWGPWHIKPPGPAEAG |
| NO: 187 | (UniProtKB) | domain 2/Bos | CDGTMADSEEVQRATALIEERLAQE |
| | | taurus | EENEKLRGTTHQKLPMEMLVLEDEK |
| | | | HHRPESPSLQKVKGQERVRKTSLDL |
| | | | RREIIDVGGIQNLIQLRKKRKQKKRE |
| | | | ALAASQEPPPEPEEITGPVDEETFLKA |
| | | | AVEGKMKVIEKFLADGGSPDTCDQF |
| | | | RRTALHRASLEGHMEILEKLLESGAT |
| | | | VDFQDRLDCTAMHWACRGGHLEVV |
| | | | RLLQSRGADTNVRDKLLSTPLHVAV |
| | | | RTGQVEIVEHFLSLGLDINAKDREGD |
| | | | SALHDAVRLNRYKIIKLLLLHGADM |
| | | | MSKNLAGKTPTDLVQLWQADTRHA |
| | | | LENPEPGSEQNGLEGSTESGRETPQP |
| | | | VAAE |
| SEQ ID | F1NG08 | Ankyrin | MAAPAPPAPGGGTSPPAGPPRLRQSD |
| NO: 188 | (UniProtKB) | 2/Gallus gallus | SNASFLRAARAGNLDKVVEYLKSGI |
| | | | DINTCNQNGLNALHLAAKEGHVGLV |
| | | | QELLERGSAVDSATKKGNTALHIASL |
| | | | AGQAEVVKVLVKEGANINAQSQNG |
| | | | FTPLYMAAQENHIEVVKYLLENGAN |
| | | | QSTATEDGFTPLAVALQQGHNQAVA |
| | | | ILLENDTKGKVRLPALHIAARKDDTK |
| | | | SAALLLQNDHNADVQSKMMVNRTT |
| | | | ESGFTPLHIAAHYGNVNVATLLLNR |
| | | | GAAVDFTARNGITPLHVASKRGNTN |
| | | | MVKLLLDRGGQIDAKTRDGLTPLHC |
| | | | AARSGHDQVVELLLERGAPLLARTK |
| | | | NGLSPLHMAAQGDHVECVKHLLQH |
| | | | KAPVDDVTLDYLTALHVAAHCGHY |
| | | | RVTKLLLDKRANPNARALNGFTPLHI |
| | | | ACKKNRIKVMELLVKYGASIQAITES |
| | | | GLTPIHVAAFMGHLNIVLLLLQNGAS |
| | | | PDVTNIRGETALHMAARAGQVEVVR |
| | | | CLLRNGALVDARAREEQTPLHIASRL |
| | | | GKTEIVQLLLQHMAHPDAATTNGYT |
| | | | PLHISAREGQVDVASVLLEAGASHS |
| | | | MSTKKGFTPLHVAAKYGSLEVAKLL |
| | | | LQRRASPDSAGKNGLTPLHVAAHYD |
| | | | NQKVALLLLEKGASPHATAKNGYTP |
| | | | LHIAAKKNQMQIATTLLNYGAETNIL |
| | | | TKQGVTPLHLASQGGHTDMVTLLLE |
| | | | KGSNIHVATKTGLTSLHLAAQEDKV |
| | | | NVAEILTKHGANQDAQTKLGYTPLI |
| | | | VACHYGNIKMVNFLLKQGANVNAK |
| | | | TKNGYTPLHQAAQQGHTHIINVLLQ |
| | | | HGAKPNAITTNGNTALAIARRLGYIS |
| | | | VVDTLKVVTEEITTTTTTVTEKHKLN |
| | | | VPETMTEVLDVSDEEAFKHSDDERF |
| | | | SDGEVYNGTGVISRNSWSDDTMTGD |
| | | | GGEYLRPEDLKELGDDSLPSSQFLDG |
| | | | MNYLRYSLEGGRSDSLRSFSSDRSHT |
| | | | LSHASYLRDSAMIDDTVVIPSQQVTT |
| | | | LAKEAERNSYRLSWGPENLDNVALS |
| | | | SSPIHSGCSSPCLDHDNSSFLVSFMVD |
| | | | ARGGAMRGCRHNGLRIIIPPRKCTAP |
| | | | TRVTCRLVKRHRLATMPPMVEGEGL |
| | | | ASRLIEVGPSGAQFLGPVIVEIPHFAA |
| | | | LRGKERELVILRSENGDSWKEHFCE |
| | | | YTEDELNEILNGMDEVLDTPEELEKK |
| | | | RICRIITRDFPQYFAVVSRIKQDSNLIG |
| | | | PEGGVLSSTVVPQVQAVFPEGALTK |
| | | | RIRVGLQAQPMHTELIKKILGNKATF |
| | | | SPIVTLEPRRRKFHKPITMTIPVPKAS |
| | | | SDGIMNGYGGDTPTLRLLCSITGGTT |
| | | | PAQWEDITGTTPLTFVNECVSFTTNV |
| | | | SARFWLIDCRQTQESVTFASQVYREII |
| | | | CVPYMAKFVVFAKSHDPIEARLRCF |
| | | | CMTDDKVDKTLEQQENFAEVARSR |
| | | | DVEVLEGKPIYVDCFGNLVPLTKSG |
| | | | QHHIFSFFAFKENRLPLFVKVRDTTQ |
| | | | EPCGRLSFMKEPKSTRGLVHQAICNL |
| | | | NITLPVYTKESESDQEQEEEVDMTSE |
| | | | KNQQDDRERTEERLAHIADHLGFSW |
| | | | TELARELDFTEEQIHQIRIENPNSLQD |
| | | | QSHALLKYWLERDGKHATDTSLTQC |
| | | | LTKINRMDIVHLMETSGIDSMQVHG |
| | | | TRTYTEIEQTIGLDHSEGFSVLQEELY |
| | | | SSRHKPDERHRISKDGDPTEHPPIVSE |
| | | | EDVSVSYSPFQDSTPRSEAELSMAEL |
| | | | LRQTHKEQVEAEFSGKPQDVIETTSS |
| | | | QHEYFVTTPGTEQRAASDTSARFSAT |
| | | | KEEREKTSPQSPSSAQRGGSPIIQEPE |
| | | | ELHLHQDDPSPRRTSLVIVESIDEQPE |
| | | | KLGSGYEEESLEKELAEELGELENSS |
| | | | DEDEMVTTRVVRRRVIIQADSMPEM |
| | | | PPETVTEEQYTDEHGHTVVKKVTRK |
| | | | IIRRYVSPDGTEKEDIIMQGTPQKPVT |
| | | | VEEGDGYSKVVKRVVLKSDSEQSEV |
| | | | TLSEPGVLPSASNFQSEPVEGRKVSK |
| | | | VIKTTVVQGERMEKHLGDASLATDL |
| | | | PSAKEDFEEALSYTGNQIKIQLPALV |
| | | | EKEIMKEDGSIIKRTTLSKASTQKRT |
| | | | VMKDRYGKHVHIEELDDTPEALPQD |
| | | | DLQHDLQQLLRHFCKEDWKQEAK |
| SEQ ID | A0A287AUI5 | Ankyrin 2/Sus | EGQSQDKGSKSGSSIQSLFFFSQSDSN |
| NO: 189 | (UniProtKB) | scrofa | ASFLRAARAGNLDKVVEYLKGGIDI |
| | | | NTCNQNGLNALHLAAKEGHVGLVQ |
| | | | ELLGRGSSVDSATKKGNTALHIASLA |
| | | | GQAEVVKVLVKEGANINAQSQNGFT |
| | | | PLYMAAQENHIDVVKYLLENGANQS |
| | | | TATEDGFTPLAVALQQGHNQAVAIL |
| | | | LENDTKGKVRLPALHIAARKDDTKS |
| | | | AALLLQNDHNADVQSKMMVNRTTE |
| | | | SGFTPLHIAAHYGNVNVATLLLNRG |
| | | | AAVDFTARNGITPLHVASKRGNTNM |
| | | | VKLLLDRGGQIDAKTRDGLTPLHCA |
| | | | ARSGHDQVVELLLERGAPLLARTKN |
| | | | GLSPLHMAAQGDHVECVKHLLQHK |
| | | | APVDDVTLDYLTALHVAAHCGHYR |
| | | | VTKLLLDKRANPNARALNGFTPLHI |
| | | | ACKKNRIKVMELLVKYGASIQAITES |
| | | | GLTPIHVAAFMGHLNIVLLLLQNGAS |
| | | | PDVTNIRGETALHMAARAGQVEVVR |
| | | | CLLRNGALVDARAREEQTPLHIASRL |
| | | | GKTEIVQLLLQHMAHPDAATTNGYT |
| | | | PLHISAREGQVDVASVLLEAGAAHS |
| | | | LATKKGFTPLHVAAKYGSLDVAKLL |
| | | | LQRRAAADSAGKNGLTPLHVAAHY |
| | | | DNQKVALLLLEKGASPHATAKNGYT |
| | | | PLHIAAKKNQMQIASTLLNYGAETNI |
| | | | VTKQGVTPLHLASQEGHTDMVTLLL |
| | | | DKGANIHMSTKSGLTSLHLAAQEDK |
| | | | VNVADILTKHGADQDAHTKLGYTPL |
| | | | IVACHYGNVKMVNFLLKQGANVNA |
| | | | KTKNGYTPLHQAAQQGHTHIINVLL |
| | | | QHGAKPNATTANGNTALAIAKRLGY |
| | | | ISVVDTLKVVTEEVTTTTTTITEKHK |
| | | | LNVPETMTEVLDVSDEEGDDTMTGD |
| | | | GGEYLRPEDLKELGDDSLPSSQFLDG |
| | | | MNYLRYSLEGGRSDSLRSFSSDRSHT |
| | | | LSHASYLRDSAMIDDTVVIPSHQVSA |
| | | | LAKEAERNSYRLSWGTENLDNVALS |
| | | | SSPIHSGFLVSFMVDARGGAMRGCR |
| | | | HNGLRIIIPPRKCTAPTRVTCRLVKRH |
| | | | RLATMPPMVEGEGLASRLIEVGPSG |
| | | | AQFLGKLHLPTAPPPLNEGESLVSRIL |
| | | | QLGPPGTKFLGPVIVEIPHFAALRGK |
| | | | ERELVVLRSENGDSWKEHYCEYTED |
| | | | ELNEILNGMDEVLDSPEDLEKKRICR |
| | | | IITRDFPQYFAVVSRIKQDSNLIGPEG |
| | | | GVQAVFPEGALTKRIRVGLQAQPMH |
| | | | SELVKKILGNKATFSPIVTLEPRRRKF |
| | | | HKPITMTIPVPKASSDVMLNGFGGD |
| | | | APTLRLLCSITGGTTPAQWEDITGTTP |
| | | | LTFVNECVSFTTNVSARFWLIDCRQI |
| | | | QESVTFASQVYREIICVPYMAKFVVF |
| | | | AKSHDPIEARLRCFCMTDDKVDKTL |
| | | | EQQENFAEVARSRDVEVLEGKPIYV |
| | | | DCFGNLVPLTKSGQHHIFSFFAFKEN |
| | | | RLPLFVKVRDSTQEPCGRLSFMKEPK |
| | | | STRGLVHQAICNLNITLPIYTKESESD |
| | | | QEQEEEVIVRHYDETESTETSVLKSH |
| | | | LVNEVPVLASPDLLSEVSEMKQDLIK |
| | | | MTAILTTDVSDRAGSLKVKELVKAA |
| | | | EEEPGEPFEIVERVKEDLEKVNEILRS |
| | | | GTCAGDEGSEPRSQPEREVVEEEWVI |
| | | | VSDEEIEEAKRKAPLEITEYPCVEVRL |
| | | | DKDTKVKVEKDSLGLVNYLTEDLNS |
| | | | YVPPHGEPLQMEREKQEALGPGRSS |
| | | | ESEGKDAPSEETQSTQKQPKPSLGIK |
| | | | KPVRRKLKDKQKQKEDSSQASADKS |
| | | | ELKKGSSEESLDEDTGLAPEPLPAVK |
| | | | ATSPLIEETPIGSIKDKVKALQKRVED |
| | | | EQKGRSKLPVRVKGKEDVPKKITHR |
| | | | TQLAASPSLKSERHALASKPERHSSL |
| | | | SSPAKTERHPPVSPSSKTEKHSPVSPS |
| | | | AKTERHSPVSSSSKTEKHSPVSPSTKP |
| | | | DRHSPVSSATKTERHPPVSPSGKTDK |
| | | | RPPVSPSGRTEKHPPVSPGRTEKRLP |
| | | | VSPAGRMERHSPMSTSGKTEKHLPV |
| | | | SPSGKTDKQPPVSPTSKTERIEETMSV |
| | | | RELMKAFQSGQDPSKHKTGLFEHKS |
| | | | AKQKQPQEKGKVRGEKEKGLTVTQ |
| | | | KETQKTETQTIKRSQRFLVTGPQNPE |
| | | | EQPVVKREEGAGERGKALNHKTPEP |
| | | | VQSAPEEESHKVESPKEKLADEQGD |
| | | | MDLQISPDRKTSTDFSDVIKQELEDN |
| | | | DKYQEFCHNQVLTSPFNTTFPLDYM |
| | | | KEEFLPALSLQSGALDGSSESLKHEG |
| | | | AAGSPCGSLMEGTPQISSEESYKHEG |
| | | | LAETPETSPESLSFSPKKTEEQTEETK |
| | | | ETTKVESPPEIHSEKEDPSTKDVTDSS |
| | | | AGQGAVVTGGTEPSAECLLKEATLE |
| | | | PSKDTCPQPEDEGLDSQGESLAKETP |
| | | | KGLTEGVPHGEDPLTHVSSAHEKQT |
| | | | DTEAQKSTASKPSDETAASPLPDAVV |
| | | | KTSPGTESKPQGVIRSPQGLELALPSR |
| | | | DSEVFSPGADESFAVSHKDSLEASPV |
| | | | LEDNSSHKTPDSLEPSPLKESPCRDSL |
| | | | ESSPVEPKMKAGILPSQFPLPSAVAK |
| | | | TELITEVASMRSRLLRDPDGSAEDDS |
| | | | LEQTSLMESSGKSPLSPDTPSSEEISY |
| | | | EVTPKTTDAGTPKPAVIPECAEEDDL |
| | | | ENGEKKRFTPEEEMFKMVTKIKISKQ |
| | | | KRDYKKEPKQEDSSSSSDADVECSA |
| | | | DLDEPKCMESVEDKSNAPVVVTAES |
| | | | RKASSSSESEPELTQLKKGADSGLLP |
| | | | EPVIRVQPPSPLPSSIDSNSSPEEVQFQ |
| | | | PIVSKQYTFKMNEDTQEDSGKSEEEE |
| | | | GCESHLPEDSHTVSTSGPEMSYDDV |
| | | | NRDADQPKICGSYGCEAESPSSSAIP |
| | | | VTLGLHSSTGEDVHEQLVIHKEPLAL |
| | | | QDTGEKDTEGEELDVSRVEAPQVGY |
| | | | PTESSSSSSSLPHCPASEGKELDEDVV |
| | | | STSSATKMEVTKADQAFESLPDDYS |
| | | | TQDLPNTTQTDSSSLDVPVSDPTETD |
| | | | DISDPQVTSPYENVPSQSFFSSEESKT |
| | | | QTDTRHTSFHSSEVCSVTTTSSVEEV |
| | | | VVTSSSGRTVSSQESNLEDQNLECKQ |
| | | | ESPLCEVQPDGAPSSLEPAAPTTSTV |
| | | | VGEQISKVIITKTDVDSDSWSEIREDD |
| | | | EAFEARVKEEEQKIFGLMVDRQSQG |
| | | | TTPDTTPARTPTEEGTPTSEQNPFLFQ |
| | | | EGKLFEMTRSGAIDMTKRSYGDESF |
| | | | HFFQIGQESREETLTEDMKEGGTGTE |
| | | | LPQLETSAESLAPLESKETVNDEADL |
| | | | VPDDLSEEVEEVPTSDGQLNSQMGIS |
| | | | ASTETSMKEAASAGAEDLPCIQMSD |
| | | | TPSLSLVKQAALQLDFSTVTRSVYSD |
| | | | RDDESPDSSPEEQKSVIEIPTAPMENV |
| | | | PSTESRSKIPVRTMPTSTPAFPSMECE |
| | | | SAPSEGFLPSLDEESQEDETKPKSKIP |
| | | | VKAPVPRVEQQLLHLDSSLQETVAP |
| | | | QGQDVTSRAPDSRSKSESDASPLDA |
| | | | KITCPEKTGSYTETDLESSEGTEELEL |
| | | | ESEDETTRPKIFASRLPVKSKSTTSSC |
| | | | RGATSPTKESKEHFFDLYRNSIEFFEE |
| | | | ISDEASKLVDRLTQSEREQELVSDDE |
| | | | SSSALEVSVIENLPPVETEQSIPEDIFD |
| | | | TRPIWDESIETLIERIPDENGHDHAED |
| | | | QQDEQERIEERLAYIADHLGFSWTEL |
| | | | ARELDFTEEQIHQIRIENPNSLQDQSH |
| | | | ALLKYWLERDGKHATDTNLIECLTK |
| | | | INRMDIVHLMETITEPLQERISHSYAE |
| | | | IEQTITLDHSEGFSVLQEELCTAQNK |
| | | | QKEEQAASKEGESYDHPPIVSEEDIS |
| | | | VGYSTFQDSIPKTEGDGSVTELLPKT |
| | | | HEEQVQQDFSGKMQDLPEESSLEQQ |
| | | | eyfvttpgteasetpkataapgspsk |
| | | | TPEEIITPPEEERPYLQTPTASEQGDSP |
| | | | IVQEPEEPPEHREASLPQKTSLVIVES |
| | | | ADDQPQTFERLDEDAAFQKGDDMP |
| | | | DIPPETVTEEEYVDEHGHTVVKKVT |
| | | | RKIIRRYVSSDGTEKEEMTMQGAPQ |
| | | | DPISIEEGDGYSKVIKRVVLKSDTERS |
| | | | EVRADFVERCK |
| SEQ ID | P49024 | Paxillin/Gallus | MDDLDALLADLESTTSHISKRPVFLT |
| NO: 190 | (UniProtKB) | gallus | EETPYSYPTGNHTYQEIAVPPPVPPPP |
| | | | SSEALNGTVIDPLDQWQPSVSRYGH |
| | | | QQPQSQSPIYSSSAKSSSASVPRDGLS |
| | | | SPSPRASEEEHVYSFPNKQKSAEPSPT |
| | | | MTSTSLGSNLSELDRLLLELNAVQH |
| | | | NPPSGFSADEVSRSPSLPNVTGPHYVI |
| | | | PESSSSAGGKAAPPTKEKPKRNGGR |
| | | | GIEDVRPSVESLLDELESSVPSPVPAI |
| | | | TVSQGEVSSPQRVNASQQQTRISASS |
| | | | ATRELDELMASLSDFKFMAQGKAG |
| | | | GSSSPPSTTPKPGSQLDTMLGSLQSD |
| | | | LNKLGVATVAKGVCGACKKPIAGQ |
| | | | VVTAMGKTWHPEHFVCTHCQEEIGS |
| | | | RNFFERDGQPYCEKDYHNLFSPRCY |
| | | | YCNGPILDKVVTALDRTWHPEHFFC |
| | | | AQCGVFFGPEGFHEKDGKAYCRKD |
| | | | YFDMFAPKCGGCARAILENYISALNT |
| | | | LWHPECFVCRECFTPFINGSFFEHDG |
| | | | QPYCEVHYHERRGSLCSGCQKPITGR |
| | | | CITAMGKKFHPEHFVCAFCLKQLNK |
| | | | GTFKEQNDKPYCQNCFLKLFC |
| SEQ ID | F1MFD1 | Paxillin OS = Bos | ALLADLESTTSHISKRPVFLSEETPYS |
| NO: 191 | (UniProtKB) | taurus | YPTGNHTYQEIAVPPVPPPPSSEALN |
| | | | GSVLDPLDPWPPSTSRFTHQQPQSSS |
| | | | PVYGSSAKTSSASNPQDGGGPPCPRA |
| | | | GEEDHVYSFPNKQKSAEPSPTVMSSS |
| | | | LGSNLSELDRLLLELNAVQHNPPGFP |
| | | | ADEANSSPPLPGPLSTHYGVPENNSL |
| | | | LGGKAGALTKEKPKRNGGRGLEDLR |
| | | | PSVENLLDELESSVPSPVPTITVNQGE |
| | | | MSSPQRVTSSQQQTRISASSATRELD |
| | | | ELMASLSDLSKIQDLEQRADGELCW |
| | | | AAGWPLNGRQSGPEGQDMGGFMAQ |
| | | | GKTGSSSPPGGPPKPGSQLDSMLGSL |
| | | | QSDLNKLGVATVAKGVCGACKKPIA |
| | | | GQVVTAMGKTWHPEHFVCTHCQEEI |
| | | | GSRNFFERDGQPYCEKDYHNLFSPR |
| | | | CYYCNGPILDKVVTALDRTWHPEHF |
| | | | FCAQCGAFFGPEGFHEKDGKAYCRK |
| | | | DYFDMFAPKCGGCARAILENYISAL |
| | | | NTLWHPECFVCRECFTPFVHGSFFEH |
| | | | EGQPYCEAHYHERRGSLCSGCQKPIT |
| | | | GRCITAMAKKFHPEHFVCAFCLKQL |
| | | | NKGTFKEQNDKPYCQNCFLKLFC |
| SEQ ID | A0A287BTJ4 | Paxillin/Sus | DALLADLESTTSHISKRPVFLSEETPY |
| NO: 192 | (UniProtKB) | scrofa | SYPTGNHTYQEIAVPPPVPPPPSSEAL |
| | | | NGSVLDPIDQWQPSTSRFIHQQPQAP |
| | | | SPVYGSSAKTSSSSNPQDGIGLPCPRA |
| | | | SEEEHVYSFPNKQKSAEPSPTVMSSS |
| | | | LGSNLSELDRLLLELNAVQHNAPGFP |
| | | | TDEANSSPPLPGALSPHYGVLEHNSS |
| | | | LGGKAGPVTKEKPKRNGGRGLEDV |
| | | | RPSVESLLDELESSVPSPVPAITLNQG |
| | | | EMNSPQRVTSSQQQTRISASSATREL |
| | | | DELMASLSDFKFMAQGKTGSSSPPG |
| | | | GPPKPGSQLDSMLGSLQSDLNKLGV |
| | | | ATVAKGVCGACKKPIAGQVVTAMG |
| | | | KTWHPEHFVCTHCQEEIGSRNFFERD |
| | | | GQPYCEKDYHNLFSPRCYYCNGPIL |
| | | | DKVVTALDRTWHPEHFFCAQCGAFF |
| | | | GPEGFHEKDGKAYCRKDYFDMFAP |
| | | | KCGGCARAILENYISALNTLWHPECF |
| | | | VCRECFTPFVNGSFFEHDGQPYCEVH |
| | | | YHERRGSLCSGCQKPITGRCITAMAK |
| | | | KFHPEHFVCAFCLKQLNKGTFKEQN |
| | | | DKPYCQNCFLKLFC |
| SEQ ID | L8IF19 | Telethonin/Bos | MATSELSCLVSEENCERREAFWAEW |
| NO: 193 | (UniProtKB) | mutus | KDLTLSTRPEEGCSLHEEDTQRHETY |
| | | | HRQGQCQAL |
| | | | VQRSPWLVMRMGILGRGLQEYQLP |
| | | | YQRVLPLPIFTPAKVGTKEEREETPIQ |
| | | | LQELLALET |
| | | | ALGGQCVDRQDVAEITKQLPPVVPV |
| | | | SKPGTLRRSLSRSMSQEAQRG |
| SEQ ID | P81947 | Tubulin alpha- | MRECISIHVGQAGVQIGNACWELYC |
| NO: 194 | (UniProtKB) | 1B chain/Bos | LEHGIQPDGQMPSDKTIGGGDDSFNT |
| | | taurus | FFSETGAGKHVPRAVFVDLEPTVIDE |
| | | | VRTGTYRQLFHPEQLITGKEDAANN |
| | | | YARGHYTIGKEIIDLVLDRIRKLADQ |
| | | | CTGLQGFLVFHSFGGGTGSGFTSLLM |
| | | | ERLSVDYGKKSKLEFSIYPAPQVSTA |
| | | | VVEPYNSILTTHTTLEHSDCAFMVDN |
| | | | EAIYDICRRNLDIERPTYTNLNRLISQI |
| | | | VSSITASLRFDGALNVDLTEFQTNLV |
| | | | PYPRIHFPLATYAPVISAEKAYHEQLS |
| | | | VAEITNACFEPANQMVKCDPRHGKY |
| | | | MACCLLYRGDVVPKDVNAAIATIKT |
| | | | KRSIQFVDWCPTGFKVGINYQPPTVV |
| | | | PGGDLAKVQRAVCMLSNTTAIAEA |
| | | | WARLDHKFDLMYAKRAFVHWYVG |
| | | | EGMEEGEFSE |
| | | | AREDMAALEKDYEEVGVDSVEGEG |
| | | | EEEGEEY |
| SEQ ID | O57613 | Paranemin/ | MLSMEGFVGARALGEESLQMWDLN |
| NO: 195 | (UniProtKB) | Gallus gallus | KRLEAYLARVKFLEEENEGLRAEIQS |
| | | | TKENPAGDPRRARYEEELRSLRDAL |
| | | | HRAFTEKCAAELARDNLYEEVQHVR |
| | | | SRCQKEQAAREEAKRQLSSSKKELE |
| | | | EERRAQIWLKERAVQLEKEVEALLE |
| | | | VHEEEKAGLDQELASFSQSLEGFRCA |
| | | | PVAFQPVEVEDYSKRLSEIWRGAVE |
| | | | TYKAEVSQLERALGQAKENLWQVA |
| | | | EDNQQSQLQLRHLEKELVGLKVRKE |
| | | | MLEESLGQQWQEQHGEAEKFQLAIE |
| | | | ALEQEKQSLQVQIAQVLEDRQQLMH |
| | | | LKMSLSLEVATYRTLLEAESTRLQM |
| | | | PPGEFKLANSLRDVKLEASSSKHRAS |
| | | | LAAFPRPEGVAQLCRTPGDALKVLT |
| | | | PKSKSSSALEFQKISSVLQAPRSWEP |
| | | | AAPSPTVPVVSPEPGSGGAESPVHEC |
| | | | GAGKESPMLSPLSPEQLVNHALQDA |
| | | | LKEMQDDAEAKEVPTLSATQSTRDG |
| | | | DLEATMEEEEAAGTQGVGAEGETVS |
| | | | PPGLCFCSNEPTLLSATQSDVESQEE |
| | | | MWEEERSKEEMLNPLSSMESQEPGG |
| | | | EPWGGVTRRSRLQVGKEDMEATSTE |
| | | | ALHISEKKEQREIWSPSREDEECEFPD |
| | | | EEREMQEEGSLQMEIEAACAVPVGS |
| | | | HPVLPTGIHLQEDFLEREQESEHQET |
| | | | SLGELGAAAGEEREQEVCQELKASSI |
| | | | EEAMPAAEGSSGSGEGTTGRESTGR |
| | | | ARDDEGEEEDKGREALGEDDLQAGE |
| | | | ALGAKELGKESMGLEEAEGMWEES |
| | | | VDLQEEHRDLQEGHGDLQVEHEDL |
| | | | WEEHGHIQEEHGDTQEEYGDTQEEH |
| | | | GDLQVEGGDLQEEHGDTQEEHGDL |
| | | | QEEHGDTQEEHGDLQVEHEDLQVEH |
| | | | GDLWEEHRDVQEEHGDTQEEYGDT |
| | | | QEEHGDLQVEGGDLQEEHGDTQEEH |
| | | | EDLQEEHGDTQEEHGDLQVEHGDLQ |
| | | | EEHRDLQEGHGDLKEEHGDLQVEHE |
| | | | DLQVEHGDLQEEHGDTQEEHGDLQE |
| | | | EHGDLQVEHEDLQVEHGDLQVEHG |
| | | | DLQEEHGDTQEEHRDLQEVHGDQQ |
| | | | EEHRDLQEGHGDLQEEHGDLQVEH |
| | | | GDLQEEYGDTQEEHRDLQEVHGDQ |
| | | | QEEHRNLQEGHGDLQEGHGDLQEE |
| | | | HGDLWKEHGVLKEEHGDLQEGHGD |
| | | | LQEESGDPQEEPGEPWVQHGEQGSA |
| | | | GDGLEQDMVLQPGEGAWGREDNDI |
| | | | SQKQQAQDWEGTAEDEEETGVNTIT |
| | | | SQEPTQVDDNPHAEAAENEERDVTS |
| | | | PTAMEETQEGEDEGDAGSEVQSQQQ |
| | | | PQDTPGQEAEPAPGQKEVRYGDTGE |
| | | | APGDPQEPAEALEVEDEELSSEPIELE |
| | | | RGSPDTAVMQQDLGNGAESDEPME |
| | | | EDFQSEDAQLEEPKACRMELEDTLL |
| | | | NSTPLCAYSGEMLESDPNPPSSGGDG |
| | | | EAAPEMAQEEEGDLRGSDEAAVHAE |
| | | | PESCEELSPAPECTEEEEGYFIVSAPS |
| | | | QEGSSMEEAENSEEFEEIKVEAAEDR |
| | | | KDELTAPGVASLVPEDEGHSEPFVGE |
| | | | AEDVKMPLGEFEMPKEEDEEDAGGF |
| | | | AAELEEGLTVPVAEGLSEGHTDKTT |
| | | | LGDEGLGEEDVQDGDNPPATETSDT |
| | | | DPSNTDPPPGTMLEHGAGMEAAEYL |
| | | | PDVPTQLPVDIMKDSDILEIVEQALEF |
| | | | NQELVLGAKLAKDGQEEDGDAQPP |
| | | | QEEEGGSSPTSSCDEQPTVQEAVAEP |
| | | | ERTKNGEQNGLHRQASLEDLAEFTE |
| | | | EGLNGITHPGEAPAAHTLPLPSKHSG |
| | | | AEPVPELSPLQTTSCARSRSPGPLGRS |
| | | | DAVGPHSPATIGRLP |
| SEQ ID | E7EYD0 | Myomesin 1a | MSESLKLQKEQEERDLETRYESSYH |
| NO: 196 | (UniProtKB) | (skelemin)/ | QSSLSSVSRQSYTAYVSSHGHKISGK |
| | | Danio rerio | KKEKKLTPLSKKEKRSYLAVDKEDE |
| | | | VIGYVVPVFRSSHLATQDLMETQEE |
| | | | VKEEGVGYVVMRNLFARESGFKMR |
| | | | TVKKTRETSMRESAERMALSQKLHE |
| | | | KEQFQKKMNPDSLTHPPEFIVKPRGQ |
| | | | TVWEGKSVTLHCTVAGWPKPRVAW |
| | | | YKNNVLIDAKARPEKYFTESQYNMH |
| | | | SLEIKNCTFSDTAEYRISALNVKGESS |
| | | | AFAPVIIKRFKEEEELVERPHGYSPEY |
| | | | GVTFNTTIIDKFDVSFGREGETMSLG |
| | | | CTVIVYPTVKRYQPEIVWYRNGVAL |
| | | | SSSKWVHMHWSGEKATLTLVHLNK |
| | | | EDEGLYTLRVNTKSGFDTHSAYVFV |
| | | | RDADVEEEGVPVAPLDVRCHDANK |
| | | | DYVVVTWKQPAVEGSSAITGYFIDR |
| | | | LEVGNHHWTQCNDTPVKYARFPVT |
| | | | GLIEGRSYMFRVRALNNAGVSRPSR |
| | | | VSDPVVAMDPSDRARLRAGPSAPWT |
| | | | GVIKFTEEDPTVGVIPGPPTDLAVTE |
| | | | ATKSYVVLSWKPPVQRGHEGVMYY |
| | | | VEKCLVGTDAWQRVNTGIPVKSPRF |
| | | | ALFDLAEDKSYTFRVRSCNSAGVGE |
| | | | PSEETGATTVGDRLDLPSAPGPVIPIR |
| | | | NTDSSVVVCWGASKEVKDLVGYYIE |
| | | | VTVDGSGVWAPCNNKPVKGTRFVC |
| | | | HGLNATDKCTFRVKAVNAAGYSGSS |
| | | | AESEACLVKASIAVPSPPTGVTTLER |
| | | | LRDYMVIGWQAPAKTGGADIRGYY |
| | | | LDYRTVKDGVTSKWHELNLKAVTSs |
| | | | PYKVTDLKENEFYQFQVRAFNQAGI |
| | | | SEASIPTAPLECKEWTVAVPGPPHGL |
| | | | RVQEVRKDSMVVLWEPPTFNGRSPV |
| | | | TGYYVDFKEENGRWRCVQERSTKH |
| | | | TYMKVSGLQEGISYRLRIHAKNLAG |
| | | | VGVPSKATDAILAETRPGTNEIVVDV |
| | | | DDNGVISLIFECSDLKEDSQFVWSKN |
| | | | YEAFTDSSRLTIQTTGGKSRAIFNDPS |
| | | | LDDLGIYSCVVTNTDGVSASYTLTEE |
| | | | GLKRLLDISHDHQFPIIPFKSEMAIEL |
| | | | QEKGRVRFWAEVGKFTSNLQVEYVF |
| | | | NDNVIHEGKKYTMNFNKSTGIIEMF |
| | | | MDLLEVTDEGTFTFNLVDGKATGRT |
| | | | SLVLIGEEFAELQKKSEFERAEWVRR |
| | | | QGPHFVEYLSFEVTPECDVHLKCKV |
| | | | GNIKPATEIAWFKDGIEIEEDDEDAK |
| | | | KIGKSDEVLTFDIGKLVIKSEKAERK |
| | | | KKPATEESPSKPKISKKDAGVYEVKL |
| | | | KDERGKDKTLLNLTDAGYQAVLNE |
| | | | VFRVIANSSTELKVMSTEHGIILYSFV |
| | | | VHYLEDLRVGWLHKESKISHSDRVQ |
| | | | CGVTGEQLWLKINEPTEKDKGKYAI |
| | | | DIFDGKGSVKRVLDLSGQVWEEAFE |
| | | | EFKRLKAAAIAERNRARVVGGLPDV |
| | | | VTIQEGKSLNLTGNVWGDPAPEVSW |
| | | | IKNEKPLVCDEHHTLKYEHSKFASITI |
| | | | AAVTTTDSGKYALLVKNKYGTEAA |
| | | | EFTVSVYIPEDEAEKKE |

### 7.1. Table 8

TABLE 8
Sequences of codon-optimized genes

| SEQ ID | Protein | Donor | | |
|---|---|---|---|---|
| NO | name | animal | Host | DNA sequence |
| SEQ ID | Cofilin 2 | Chicken | S. cerevisiae | ATGGCATCAGGCGTCACAGTGAACGATG |
| NO: 197 | | | | AAGTTATCAAGGTTTTCAACGATATGAA |
| | | | | AGTTCGTAAGTCTAGCACCCCAGAGGAA |
| | | | | ATCAAAAAGAGAAAAAAAGCTGTCTTGT |
| | | | | TTTGTTTATCCGATGACAAAAAGCAGAT |
| | | | | TATTGTAGAGGAAGCTACTAGAATCCTT |
| | | | | GTGGGTGATATAGGTGACACTGTAGAGG |
| | | | | ATCCTTACACTGCCTTCGTCAAGTTGTTA |
| | | | | CCATTAAATGATTGCAGATATGCTCTCTA |
| | | | | CGACGCTACATACGAAACCAAGGAATCT |
| | | | | AAAAAAGAGGACTTGGTTTTCATCTTTT |
| | | | | GGGCCCCTGAATCCGCGCCACTGAAGAG |
| | | | | TAAGATGATATACGCATCTTCAAAGGAT |
| | | | | GCAATTAAAAAAAAGTTCACAGGTATTA |
| | | | | AGCATGAATGGCAAGTTAACGGGCTTGA |
| | | | | TGATATTAAAGATAGATCTACATTGGGT |
| | | | | GAAAAGCTAGGCGGAAATGTTGTGGTTT |
| | | | | CATTGGAAGGAAAGCCACTATAA |
| SEQ ID | Profilin | Chicken | S. cerevisiae | ATGGCTGGCTGGCAATCTTATGTGGATA |
| NO: 198 | | | | ACTTAATGTGTGATGGATGTTGTCAAGA |
| | | | | GGCTGCAATCGTGGGTTACTGCGACGCA |
| | | | | AAATACGTTTGGGCAGCAACAGCTGGGG |
| | | | | GCATATTCCAATCAATTACACCAGTTGA |
| | | | | AATTGATATGATCGTTGGTAAAGATAGA |
| | | | | GAGGGATTTTTCACTAATGGTCTAACTTT |
| | | | | AGGTGCCAAAAAGTGCAGTGTTATCAGA |
| | | | | GACTCACTGTACGTAGACGGGGATTGCA |
| | | | | CCATGGATATTCGTACAAAGTCTCAGGG |
| | | | | TGGAGAACCTACATACAACGTCGCGGTC |
| | | | | GGCAGAGCCGGGAGAGTTTTGGTTTTCG |
| | | | | TAATGGGCAAGGAAGGTGTCCATGGTGG |
| | | | | TGGACTTAACAAAAAGGCCTACTCTATG |
| | | | | GCTAAGTACTTGAGAGATTCCGGTTTTTA |
| | | | | A |
| SEQ ID | Coronin | Chicken | S. cerevisiae | ATGTCTCGTAGAGTTGTTAGACAATCCA |
| NO: 199 | | | | AGTTCCGTCACGTGTTCGGCCAACCAGT |
| | | | | TAAGGCAGATCAGATGTACGAAGATATC |
| | | | | AGAGTTTCAAAGGTTACCTGGGACTCAT |
| | | | | CTTTTTGCGCTGTTAACCCAAAGTTCGTA |
| | | | | GCAATAATTGTGGAAGCTGGCGGTGGGG |
| | | | | GAGCATTTATGGTTTTACCACTAGCCAA |
| | | | | GACTGGTAGAGTCGACAAAAATCACCCT |
| | | | | TTAGTCACTGGACATACAGCACCTGTAT |
| | | | | TAGATATTGACTGGTGTCCACATAACGA |
| | | | | CAATGTTATTGCAAGTGCATCTGAGGAT |
| | | | | ACAACTGTCATGGTATGGCAAATCCCAG |
| | | | | ACTACGTTCCAGTAAGATCAATCACAGA |
| | | | | ACCAGTTGTCACGCTCGAGGGTCACTCT |
| | | | | AAGAGAGTTGGCATTATCTGTTGGCATC |
| | | | | CTACAGCCAGAAATGTGTTGTTGTCTGC |
| | | | | CGGTTGCGATAACTTGGTAATTCTTTGGA |
| | | | | ACGTCGGTACAGGCGAAATGTTGCTGGC |
| | | | | GCTTGAAGATATGCACACTGACCTCATT |
| | | | | TACAACGTCGGATGGAACAGAAACGGGT |
| | | | | CGTTATTAGTCACCACATGTAAAGATAA |
| | | | | AAAGGTAAGGGTTATCGACCCTAGAAAG |
| | | | | CAAACAGTTGTTGCGGAAATCACAAAGC |
| | | | | CACATGATGGTGCTAGACCAATTAGAGC |
| | | | | TATATTCATGGCCGATGGTAAGATTTTCA |
| | | | | CAACCGGATTCTCAAAAATGTCCGAGAG |
| | | | | ACAACTTGGGTTGTGGGATCTTAAAAAC |
| | | | | TTCGAGGAACCAATTGCTCTGCAGGAAA |
| | | | | TGGATACTAGTAATGGTGTTTTGTTACCA |
| | | | | TTTTACGACCCAGACACAAACATCGTTT |
| | | | | ACCTCTGCGGCAAGGGTGATAGTAGCAT |
| | | | | CAGATATTTTGAGATAACAGATGAAGCT |
| | | | | CCTTACGTCCATTACTTGAATACTTACTC |
| | | | | CTCAAAGGAACCACAGAGAGGTATGGG |
| | | | | ATTCATGCCAAAGCGAGGACTAGATGTT |
| | | | | TCTAAGTGTGAAATCGCTAGATTTTTCAA |
| | | | | GTTACATGAGAGAAAATGCGAACCTATT |
| | | | | GTGATGACAGTGCCTAGAAAATCTGATT |
| | | | | TGTTCCAAGATGATCTATATCCAGATACT |
| | | | | CCTGGCCCAGAACCAGCCCTTGAAGCTG |
| | | | | ATGAATGGTTATCTGGTAAAGATGCAGA |
| | | | | GCCAATACTAATTTCTCTTAGAGATGGG |
| | | | | TACGTCCCAGTGAAAAACAGAGAGTTGA |
| | | | | AAGTTGTTAAAAAAAATATTTTGGATAG |
| | | | | CAAGCCTCCTCCAGGTCCTCGTAGATCTC |
| | | | | ACTCCACATCAAACACCGATATATCAAC |
| | | | | ACCAGCTTTGGATGAAGTTTTAGAGGAA |
| | | | | ATCCGGGTGTTGAAGGAAACTGTACAAG |
| | | | | CACAAGAGAAGAGAATCTCAGCACTGG |
| | | | | AACATAAGCTATGTCAATTTACTAATGG |
| | | | | TACCGACTAA |
| SEQ SEQ | Myozenin | Turkey | S. cerevisiae | ATGCCTTTAGCCGGAACCCCAGCACCAT |
| ID NO: | 1 | | | TGAAGAGAAAAAAGCCAACAAAACTTA |
| 200 | | | | TTGGTAAGCTGACACACGAAGTTAT |
| | | | | GCCACAGGAAGTTACCAAGTTGAATCTA |
| | | | | GGTAAAAAGATTTCTATCCCTAGAGATG |
| | | | | TCATGTTGGAAGAGTTATCGTTAT |
| | | | | TGACGAACAAAGGTTCCAAAATGTTCAA |
| | | | | GTTGAGACAATTAAGAGTCGAGAAATTC |
| | | | | ATTTACGAAAACAATCCAGACGCA |
| | | | | TTCTCCGATAACAGTGTTGATCATTTTCA |
| | | | | ACGTTTTATCCCATCTGGTGGACATTATG |
| | | | | GTGAAGATGCCCATGGGTACGG |
| | | | | TCATGGTCGTATGGTTGGGGGCGTTACA |
| | | | | GCCGGGCAACATGGTTCATCAAAGCAAC |
| | | | | ATTACAGTACCGTGCCTCCTCGAC |
| | | | | CTGGTTCTAAGGGTGGTCCAGGTAACTC |
| | | | | TGAGGGTGAACATGCTGAAAAGTCAGCT |
| | | | | GGGTCTGCTGGAGAGGGCGGCCAC |
| | | | | GGTACAGAAAAGGATGGTAAGAGTGGT |
| | | | | GGCAAAAAGCCTCTACTTAAGACTTACA |
| | | | | TCAGCCCATGGGAGAGAGCGATGGG |
| | | | | AATCTCACCAGAGGATAAGAGCCAGTTA |
| | | | | ACTATTGATCTTCTATCATATTCACCAAA |
| | | | | GGCAGACTTCCCACACTACAAAT |
| | | | | CTTTTAACAGAACAGCAATGCCATACGG |
| | | | | CGGATACGAAAAAGCTGCTAAGAGAAT |
| | | | | GACATTTAAGGTACCTCAATTCGAT |
| | | | | ATCTGTCCACTGTTGCCAGAATCCATAGT |
| | | | | ACTCTACAACCAAAATTTCAGAAACAGA |
| | | | | CCATCATTCAATAGAACTCCTAT |
| | | | | ACCTTGGATGCCATCTGGCGAATCTTCC |
| | | | | GAATACCACACTGACATTAACGTGCCAA |
| | | | | GATCTGGAGAAACAGAGGAATTGT |
| | | | | AA |
| SEQ ID | Troponin | Pig | S. cerevisiae | ATGACTGATCAACAAGCTGAAGCAAGAT |
| NO: 201 | C, | | | CTTACCTTAGTGAAGAGATGATAGCAGA |
| | skeletal | | | GTTTAAGGCAGCGTTCGATATGTTCGAT |
| | muscle | | | GCCGACGGTGGTGGCGATATCTCTGTGA |
| | | | | AGGAACTCGGTACAGTTATGAGAATGCT |
| | | | | GGGGCAAACACCAACCAAGGAAGAGTT |
| | | | | GGATGCAATCATCGAAGAGGTCGACGAA |
| | | | | GATGGGTCAGGTACAATTGATTTTGAAG |
| | | | | AGTTTTTGGTTATGATGGTAAGACAGAT |
| | | | | GAAAGAGGATGCTAAGGGTAAGTCAGA |
| | | | | AGAGGAATTAGCTGAATGTTTTAGAATT |
| | | | | TTCGATAGAAATGCTGATGGATACATTG |
| | | | | ACGCTGAGGAACTAGCCGAAATTTTCCG |
| | | | | TGCCTCTGGAGAACATGTCACTGATGAG |
| | | | | GAATTGGAATCCTTAATGAAAGATGGCG |
| | | | | ACAAAAACAACGAGGGTAGAATCGACTT |
| | | | | CGACGAATTCCTTAAGATGATGGAAGGC |
| | | | | GTTCAATAA |
| SEQ ID | Cofilin 2 | Chicken | K. phaffii | ATGGCTTCTGGTGTGACTGTTAACGACG |
| NO: 202 | | | | AAGTCATCAAGGTATTCAATGATATGAA |
| | | | | AGTTAGAAAATCATCCACTCCAGAGGAA |
| | | | | ATCAAAAAGAGAAAAAAAGCCGTTCTAT |
| | | | | TTTGCCTGTCGGACGACAAAAAGCAGAT |
| | | | | CATCGTTGAGGAAGCCACACGTATTTTG |
| | | | | GTCGGTGACATTGGTGACACAGTCGAAG |
| | | | | ATCCTTATACTGCTTTTGTTAAGCTGTTG |
| | | | | CCCTTAAATGATTGTAGGTACGCTCTGTA |
| | | | | CGACGCAACTTACGAAACCAAAGAGTCC |
| | | | | AAAAAAGAGGATTTGGTGTTCATCTTCT |
| | | | | GGGCACCTGAAAGTGCTCCACTTAAGAG |
| | | | | CAAGATGATTTATGCATCCTCTAAAGAT |
| | | | | GCTATTAAAAAAAAGTTTACAGGTATAA |
| | | | | AGCATGAGTGGCAAGTGAACGGATTGGA |
| | | | | TGACATTAAAGATAGATCTACGTTGGGC |
| | | | | GAAAAGCTTGGTGGAAATGTTGTAGTGT |
| | | | | CATTAGAGGGAAAGCCACTCTAA |

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

### 9. REFERENCES CITED

[1] H. F. Gemede and N. Ratta, “Antinutritional factors in plant foods: Potential health benefits and adverse effects,” International Journal of Nutrition and Food Sciences, vol. 3, no. 4, pp. 284-289, 2014.
[2] G. Rimbach, J. Pallauf, J. Moehring, K. Kraemer and A. M. Minihane, “Effect of dietary phytate and microbial phyatse on mineral and traceelement bioavailability—a literature review,” Current Topics in Nutraceutical Research, vol. 6, no. 3, pp. 131-144, 2008.
[3] R. M. Yamka, U. Jamikorn, A. D. True and D. L. Harmon, “Evaluation of soyabean meal as a protein source in canine foods,” Animal Feed Science and Technology, vol. 109, pp. 121-132, 2003.
[4] K. E. Michel, “Unconventional Diets for Dogs and Cats,” Veterinary Clinics: Small Animal Practice, vol. 36, no. 6, p. 1269-1281, 2006.
[5] K. Kanakubo, A. J. Fascetti and J. A. Larsen, “Assessment of protein and amino acid concentrations and labeling adequacy of commercial vegetarian diets formulated for dogs and cats,” Journal of the American Veterinary Medical Association, vol. 247, no. 4, pp. 385-392, 2015.
[6] M. Friedman, “Nutritional Value of Proteins from Different Food Sources. A Review,” Journal of Agricultural and Food Chemistry, vol. 44, pp. 6-29, 1996.
[7] C. M. Gray, R. K. Sellon and L. M. Freeman, “Nutritional adequacy of two vegan diets for cats,” Timely Topics in Nutrition, vol. 225, no. 11, pp. 1670-1675, 2004.
[8] A. Knight and M. Leitsberger, “Vegetarian versus Meat-Based Diets for Companion Animals,” Animals, vol. 6, no. 57, pp. 1-20, 2016.
[9] J. L. Kaplan, J. A. Stern, A. J. Fascetti, J. A. Larsen, H. Skolnik, G. D. Peddle, R. D. Kienle, A. C. M. Waxman, C. T. Gunther-Harrington, T. Klose, K. LaFauci and B. Lefbom, “Taurine deficiency and dilated cardiomyopathy in golden retrievers fed commercial diets,” PLOS ONE, 13 Dec. 2018.
[10] M. M. Rojas-Downing, A. P. Nejadhashemi, T. Harrigan and S. A. Woznicki, “Climate change and livestock: Impacts, adaptation, and mitigation,” Climate Risk Management, vol. 16, pp. 145-163, 2017.
[11] Y. M. Bar-On, R. Phillips and R. Milo, “The biomass distribution on Earth,” PNAS, vol. 115, no. 25, pp. 6506-6511, 2018.
[12] “Living Planet Report 2016,” World Wide Fund For Nature, Gland, Switzerland, 2016.
[13] F. P. J. van Bree, G. C. A. M. Bokken, R. Mineur, F. Franssen, M. Opsteegh, J. W. B. van der Giessen, L. J. A. Lipman and P. A. M. Overgaauw, “Zoonotic bacteria and parasites found in raw meat-based diets for cats and dogs,” Veterinary Record, p. 10.1136/vr.104535, 2018.
[14] A. G. Mathew, R. Cissell and S. Liamthong, “Antibiotic Resistance in Bacteria Associated with Food Animals: A United States Perspective of Livestock Production,” FOODBORNE PATHOGENS AND DISEASE, vol. 4, no. 2, pp. 115-133, 2007.
[15] S. A. McEwen and P. J. Fedorka-Cray, “Antimicrobial Use and Resistance in Animals,” Clinical Infectious Diseases, vol. 34, no. Suppl 3, pp. S93-S106, 2002.
[16] W. Witte, “Selective pressure by antibiotic use in livestock,” International Journal of Antimicrobial Agents, vol. 16, pp. S19-S24, 2000.
[17] “Summary Report On Antimicrobials Sold or Distributed for Use in Food-Producing Animals,” Food and Drug Administration, 2014.
[18] J. E. Markovich, C. R. Heinze and L. M. Freeman, “Thiamine deficiency in dogs and cats,” Journal of the American Veterinary Medical Association, vol. 243, no. 5, pp. 649-656, 2013.
[19] M. Steer, “A COMPARISON OF LAND, WATER AND ENERGY USE BETWEEN CONVENTIONAL AND YEAST-DERIVED DAIRY PRODUCTS: AN INITIAL ANALYSIS,” University of the West of England, 2015.
[20] X. Zheng, K. Diraviyam and D. Sept, “Nucleotide Effects on the Structure and Dynamics of Actin,” Biophysical Journal, vol. 93, no. 4, p. 1277-1283, 2007.
[21] R. Dominguez, “A Common Binding Site for Actin-Binding Proteins on the Actin Surface,” in Actin-Monomer-Binding Proteins, New York, N.Y.: Springer, 2007.
[22] T. D. Pollard and G. G. Borisy, “Cellular Motility Driven by Assembly and Disassembly of Actin Filaments,” Cell, vol. 112, no. 4, pp. 453-465, 2003.

### 10. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

1.-176. (canceled)

177. A pet food or animal feed composition comprising a yeast cell or filamentous fungal cell that expresses at least one recombinant animal protein.

178. The composition of claim 177, wherein the composition is substantially free of antibiotics, animal growth hormones, and animal meat.

179. The composition of claim 177, wherein the recombinant animal protein has a maximum amino acid sequence identity of 70% to any protein naturally encoded in the genome of the yeast cell or filamentous fungal cell that expresses the recombinant animal protein.

180. The composition of claim 177, wherein the recombinant protein is profilin, cofilin, coronin, myozenin, troponin, radixin, myotubularin, gamma-sarcoglycan, alpha-actinin, caveolin, desmin, tropomyosin, titin, connectin, transgelin, smoothelin, actin or myosin.

181. The composition of claim 177, wherein the recombinant animal protein is a protein of a chicken, a pig, a turkey, a pheasant, a quail, a horse, cattle, a fish, a shrimp, a prawn, a crab, a sheep, a deer, a duck, a rabbit, an elk, a moose, a kangaroo, an alligator, a wild boar, a goat, a bison, a buffalo, a bear, or an elephant.

182. The composition of claim 177, wherein the recombinant animal protein is a poultry protein, even-toed ungulate protein, or fish protein.

183. The composition of claim 177, wherein the recombinant animal protein is a cytoskeletal protein.

184. The composition of claim 177, wherein the recombinant animal protein is a muscle protein.

185. The composition of claim 177, wherein the recombinant animal protein is a skeletal muscle protein.

186. The composition of claim 177, wherein the composition comprises a yeast cell or a filamentous fungal cell that expresses at least two different recombinant animal proteins.

187. The composition of claim 177, wherein the composition comprises at least two different yeast or filamentous fungal cells that each express at least one different recombinant animal protein.

188. The composition of claim 177, wherein the composition comprises about 1% to 30% of the recombinant protein based on the dry weight of the composition.

189. The composition of claim 177, wherein the composition comprises about 5% to 40% of the recombinant protein based on the dry weight of the composition.

190. The composition of claim 177, wherein the composition further comprises a fat, a carbohydrate, a non-recombinant protein, a fiber, a nutritional supplement, a palatability agent, or any combination thereof.

191. The composition of claim 177, wherein the composition comprises 5-50% protein, 0-50% fat, 0-75% carbohydrate, 0-40% dietary fiber, and 0-15% of other nutrients.

192. The composition of claim 177, wherein the composition is nutritionally balanced for a companion animal, optionally wherein the composition is a kibble.

193. A method for producing a pet food or animal feed composition according to claim 1, the method comprising culturing yeast or filamentous fungal cells that express an animal protein and processing the cells to provide the pet food or animal feed composition.

194. The method of claim 193, wherein processing the cells comprises combining the cells with a fat, a carbohydrate, a non recombinant protein, a fiber, a nutritional supplement, a palatability agent, or any combination thereof;
or dehydrating, baking, and/or extruding the yeast or filamentous fungal cells, optionally combined with a fat, a carbohydrate, a non recombinant protein, a fiber, a nutritional supplement, a palatability agent, or any combination thereof.

195. The method of claim 194, wherein the method comprises lysing the yeast or filamentous fungal cells.

196. The method of claim 193, wherein the method comprises isolating, purifying, and/or concentrating, the animal protein.
